(19) United States
(12) Patent Application Publication
(10) Pub. No.: US 2025/0002609 A1
(43) Pub. Date: Jan. 2, 2025

(54) ANTI-KLK5 ANTIBODIES AND METHODS OF USE

(71) Applicant: Genentech, Inc., South San Francisco, CA (US)

(72) Inventors: Cecilia P.C. CHIU, Redwood City, CA (US); Hilda Y. HERNANDEZ-BARRY, Martinez, CA (US); David B. IAEA, San Mateo, CA (US); Moulay Hicham ALAOUI-ISMAILI, San Mateo, CA (US); James T. KOERBER, San Mateo, CA (US); Wei Yu LIN, Millbrae, CA (US); Kelly LOYET, South San Francisco, CA (US); Yonglian SUN, Foster City, CA (US); Benjamin T. WALTERS, South San Francisco, CA (US); Jawahar SUDHAMSU, San Francisco, CA (US)

(73) Assignee: Genentech, Inc., South San Francisco, CA (US)

(21) Appl. No.: 18/740,409

(22) Filed: Jun. 11, 2024

Related U.S. Application Data
(63) Continuation of application No. 16/950,418, filed on Nov. 17, 2020, now Pat. No. 12,037,412.
Application No. 16/950,418 is a continuation of application No. 16/352,619, filed on Mar. 13, 2019, now abandoned.
(60) Provisional application No. 62/643,034, filed on Mar. 14, 2018.

(51) Int. Cl. C07K 16/40; A61K 47/68

(52) U.S. Cl. CPC C07K 16/40; A61K 47/6871; C07K 2317/24; C07K 2317/34; C07K 2317/55; C07K 2317/565; C07K 2317/76; C07K 2317/92

(57) ABSTRACT

The invention provides anti-KLK5 antibodies and methods of using the same.

### CROSS-REFERENCE TO RELATED APPLICATIONS

This application is a continuation of U.S. patent application Ser. No. 16/352,619, filed Mar. 13, 2019, now U.S. Pat. No. 12,037,412, which claims benefit under 35 U.S.C. § 119(e) to U.S. Provisional Application No. 62/643,034, filed Mar. 14, 2018, the entire contents of which are incorporated herein by reference.

### SEQUENCE LISTING

The instant application contains a Sequence Listing which has been submitted electronically in XML format and is hereby incorporated by reference in its entirety. Said XML copy, created on Aug. 30, 2024, is named P34707-US-3_Sequence_Listing.xml and is 524,800 bytes in size.

### FIELD OF THE INVENTION

The present invention relates to anti-KLK5 antibodies and methods of using the same.

### BACKGROUND

Human kallikrein-related peptidases (KLKs) are (chymo)-trypsin-like serine proteases that are expressed in a variety of tissues such as prostate, ovary, breast, testis, brain, and skin. KLKs belong to a subgroup of the chymotrypsin-like serine protease family S1A of clan PA(S). The 15 human KLK genes are located on chromosome 19q13.4 and constitute the largest contiguous serine protease cluster in the human genome. These genes, generally composed of five coding exons and in some cases one or two 5′ non-coding exons, encode the kallikrein-related peptidases KLK1 to KLK15. All KLK genes encode single-chain pre-pro-proteins containing a chymotrypsin- or trypsin-like catalytic domain of 224-237 residues with an amino acid sequence identity of approximately 40% among KLK4 to KLK15. KLK1 and its close homologs KLK2 and KLK3 form a clade of their own, KLK4, 5, and 7 belong to another subgroup, whereas KLK6 shares more similarity with KLK13 and KLK14. See Debela et al., Biol Chem 389, 623-632 (2008).

KLK5 appears to be most abundantly expressed in human skin, specifically in the upper spinous and granular layers of the skin, where keratinocytes undergo terminal differentiation and are transformed into flattened brick-like structures that form the stratum corneum, the outermost epidermal layer and the barrier to the outside environment. See Debela et al., J Mol Biol, 373, 1017-1031 (2007); and Tan et al., J Med Chem. 2015 Jan. 22; 58(2):598-612 (2014). KLK5 is described to play pathological roles in skin disorders such as Netherton Syndrome. See Furio et al., PLOS Genet 11(9), e1005389 (2015). Netherton Syndrome is caused by loss-of-function mutations in the SPINK5 gene, encoding the serine protease inhibitor Kazal-type 5 (SPINK5). See Descargues et al., Nat Genet. 2005 January; 37(1):56-65 (2004). SPINK5 has been shown to inhibit several members of the KLK serine protease family (e.g. KLK5 and KLK7). See Wang et al., Exp Dermatol. July; 23(7):524-6 (2014). The absence of SPINK5 in Netherton Syndrome results in unopposed KLKs activities. KLK5 hyperactivity is thought to be a key element in the pathophysiology of Netherton Syndrome as KLK5 is a regulator of proteolysis in the epidermis. Ablation of KLK5 and KLK7 rescues lethality of Netherton Syndrome-like phenotype. See Briot et al., J Exp Med. May 11; 206(5):1135-47 (2009); Furio et al., J Exp Med. March 10; 211(3):499-513 (2014); and Kasparek et al., PLoS Genet. 2017 Jan. 17; 13(1):e1006566 (2017). Netherton Syndrome is a complex systemic disease with multiple effects for which currently no satisfactory treatment is available.

Asthma is a clinically heterogeneous disorder associated with both genetic and environmental risk factors. Estimates of heritability from asthma twin studies vary from 35% to 80%, indicating an important role for genetic risk. See e.g., Ullemar et al., Allergy 71, 230-238 (2016). Several large scale GWAS have been performed for asthma and asthma related phenotypes, and many of the loci identified such as those near ORMDL3, IL13, ILIRL1 and TSLP genes have been confirmed in multiple study populations. See e.g., Bonnelykke et al., Nat Genet 46, 51-55 (2014). Recent studies identified a SNP at the KLK4/5 locus which is protective for the risk regarding periostin low, or type 2 low inflammation asthma. In the same study, KLK5 levels were found to be elevated in bronchoalveolar lavage of severe asthma patients supporting the hypothesis that KLK5 plays a role in bronchial obstruction and asthma pathogenesis.

Despite the advances in the field of diseases such as Netherton Syndrome and asthma, there remains a need to identify targets and develop means that can supplement or enhance the efficacy of existing therapies.

### SUMMARY

Provided herein are anti-KLK5 antibodies and methods of using the same.

Further provided herein is an isolated antibody that binds to KLK5, wherein the antibody comprises (a) HVR-H1 comprising the amino acid sequence of SEQ ID NO:28; (b) HVR-H2 comprising the amino acid sequence selected from the group consisting of SEQ ID NO:38, SEQ ID NO:45, and SEQ ID NO:54; (c) HVR-H3 comprising the amino acid sequence selected from the group consisting of SEQ ID NO:65, SEQ ID NO:69, and SEQ ID NO:72; (d) HVR-L1 comprising the amino acid sequence of SEQ ID NO:96; (e) HVR-L2 comprising the amino acid sequence of SEQ ID NO:109; and (f) HVR-L3 comprising the amino acid sequence of SEQ ID NO:127.

In some embodiments, the antibody comprises (a) HVR-H1 comprising the amino acid sequence selected from the group consisting of SEQ ID NO:17, SEQ ID NO:22, and SEQ ID NO:24; (b) HVR-H2 comprising the amino acid sequence selected from the group consisting of SEQ ID NO:35, SEQ ID NO:36, SEQ ID NO:37, SEQ ID NO:42, SEQ ID NO:43, SEQ ID NO:44, SEQ ID NO:48, SEQ ID NO:49, SEQ ID NO:50, SEQ ID NO:51, SEQ ID NO:52, and SEQ ID NO:53; (c) HVR-H3 comprising the amino acid sequence selected from the group consisting of SEQ ID NO:65, SEQ ID NO:69, and SEQ ID NO:72; (d) HVR-L1 comprising the amino acid sequence selected from the group consisting of SEQ ID NO:82, SEQ ID NO:87, and SEQ ID NO:91; (e) HVR-L2 comprising the amino acid sequence selected from the group consisting of SEQ ID NO:99, SEQ ID NO:101, and SEQ ID NO:105; and (f) HVR-L3 comprising the amino acid sequence selected from the group consisting of SEQ ID NO:115, SEQ ID NO:119, and SEQ ID NO:122.

In some embodiments of any of the antibodies, the antibody comprises (i) (a) HVR-H1 comprising the amino acid sequence of SEQ ID NO:17; (b) HVR-H2 comprising the amino acid sequence of SEQ ID NO:35; (c) HVR-H3 comprising the amino acid sequence of SEQ ID NO:65; (d) HVR-L1 comprising the amino acid sequence of SEQ ID NO:82; (e) HVR-L2 comprising the amino acid sequence of SEQ ID NO:101; and (f) HVR-L3 comprising the amino acid sequence of SEQ ID NO:115; (ii) (a) HVR-H1 comprising the amino acid sequence of SEQ ID NO:22; (b) HVR-H2 comprising the amino acid sequence of SEQ ID NO:42; (c) HVR-H3 comprising the amino acid sequence of SEQ ID NO:69; (d) HVR-L1 comprising the amino acid sequence of SEQ ID NO:87; (e) HVR-L2 comprising the amino acid sequence of SEQ ID NO:105; and (f) HVR-L3 comprising the amino acid sequence of SEQ ID NO:119; or (iii) (a) HVR-H1 comprising the amino acid sequence of SEQ ID NO:24; (b) HVR-H2 comprising the amino acid sequence of SEQ ID NO:52; (c) HVR-H3 comprising the amino acid sequence of SEQ ID NO:72; (d) HVR-L1 comprising the amino acid sequence of SEQ ID NO:91; (e) HVR-L2 comprising the amino acid sequence of SEQ ID NO:99; and (f) HVR-L3 comprising the amino acid sequence of SEQ ID NO:122.

In some embodiments of any of the antibodies, the antibody comprises a) a VH sequence of SEQ ID NO:202 and a VL sequence of SEQ ID NO:140; b) a VH sequence of SEQ ID NO:225 and a VL sequence of SEQ ID NO:151; or c) a VH sequence of SEQ ID NO:257 and a VL sequence of SEQ ID NO:162.

In some embodiments of any of the antibodies, the antibody comprises a) a VH sequence of SEQ ID NO:201 and a VL sequence of SEQ ID NO:139; b) a VH sequence of SEQ ID NO:221 and a VL sequence of SEQ ID NO:149; or c) a VH sequence of SEQ ID NO:248 or SEQ ID NO:254, and a VL sequence of SEQ ID NO:160.

Further provided herein is an isolated antibody that binds to KLK5, wherein the antibody comprises a) a VH sequence having at least 95% sequence identity to the amino acid sequence of SEQ ID NO:201 and a VL sequence having at least 95% sequence identity to the amino acid sequence of SEQ ID NO:139; b) a VH sequence having at least 95% sequence identity to the amino acid sequence of SEQ ID NO:221 and a VL sequence having at least 95% sequence identity to the amino acid sequence of SEQ ID NO:149; or c) a VH sequence having at least 95% sequence identity to the amino acid sequence of SEQ ID NO:248 or SEQ ID NO:254, and a VL sequence having at least 95% sequence identity to the amino acid sequence of SEQ ID NO:160.

Further provided herein is an isolated antibody that binds to KLK5, wherein the antibody comprises (i) (a) HVR-H1 comprising the amino acid sequence of SEQ ID NO:14; (b) HVR-H2 comprising the amino acid sequence of SEQ ID NO:32; (c) HVR-H3 comprising the amino acid sequence of SEQ ID NO:62; (d) HVR-L1 comprising the amino acid sequence of SEQ ID NO:79; (e) HVR-L2 comprising the amino acid sequence of SEQ ID NO:99; and (f) HVR-L3 comprising the amino acid sequence of SEQ ID NO:112; (ii) (a) HVR-H1 comprising the amino acid sequence of SEQ ID NO:15; (b) HVR-H2 comprising the amino acid sequence of SEQ ID NO:33; (c) HVR-H3 comprising the amino acid sequence of SEQ ID NO:63; (d) HVR-L1 comprising the amino acid sequence of SEQ ID NO:80; (e) HVR-L2 comprising the amino acid sequence of SEQ ID NO:100; and (f) HVR-L3 comprising the amino acid sequence of SEQ ID NO:113; (iii) (a) HVR-H1 comprising the amino acid sequence of SEQ ID NO:16; (b) HVR-H2 comprising the amino acid sequence of SEQ ID NO:34; (c) HVR-H3 comprising the amino acid sequence of SEQ ID NO:64; (d) HVR-L1 comprising the amino acid sequence of SEQ ID NO:81; (e) HVR-L2 comprising the amino acid sequence of SEQ ID NO:99; and (f) HVR-L3 comprising the amino acid sequence of SEQ ID NO:114; (iv) (a) HVR-H1 comprising the amino acid sequence of SEQ ID NO:18; (b) HVR-H2 comprising the amino acid sequence of SEQ ID NO:39; (c) HVR-H3 comprising the amino acid sequence of SEQ ID NO:66; (d) HVR-L1 comprising the amino acid sequence of SEQ ID NO:83; (e) HVR-L2 comprising the amino acid sequence of SEQ ID NO:102; and (f) HVR-L3 comprising the amino acid sequence of SEQ ID NO:116; (v) (a) HVR-H1 comprising the amino acid sequence of SEQ ID NO:19; (b) HVR-H2 comprising the amino acid sequence of SEQ ID NO:40; (c) HVR-H3 comprising the amino acid sequence of SEQ ID NO:67; (d) HVR-L1 comprising the amino acid sequence of SEQ ID NO:84; (e) HVR-L2 comprising the amino acid sequence of SEQ ID NO:103; and (f) HVR-L3 comprising the amino acid sequence of SEQ ID NO:117; (vi) (a) HVR-H1 comprising the amino acid sequence of SEQ ID NO:20; (b) HVR-H2 comprising the amino acid sequence of SEQ ID NO:33; (c) HVR-H3 comprising the amino acid sequence of SEQ ID NO:68; (d) HVR-L1 comprising the amino acid sequence of SEQ ID NO:85; (e) HVR-L2 comprising the amino acid sequence of SEQ ID NO:103; and (f) HVR-L3 comprising the amino acid sequence of SEQ ID NO:118; (vii) (a) HVR-H1 comprising the amino acid sequence of SEQ ID NO:21; (b) HVR-H2 comprising the amino acid sequence of SEQ ID NO:41; (c) HVR-H3 comprising the amino acid sequence of SEQ ID NO:69; (d) HVR-L1 comprising the amino acid sequence of SEQ ID NO:86; (e) HVR-L2 comprising the amino acid sequence of SEQ ID NO:104; and (f) HVR-L3 comprising the amino acid sequence of SEQ ID NO:118; (viii) (a) HVR-H1 comprising the amino acid sequence of SEQ ID NO:22; (b) HVR-H2 comprising the amino acid sequence of SEQ ID NO:46; (c) HVR-H3 comprising the amino acid sequence of SEQ ID NO:69; (d) HVR-L1 comprising the amino acid sequence of SEQ ID NO:88; (e) HVR-L2 comprising the amino acid sequence of SEQ ID NO:105; and (f) HVR-L3 comprising the amino acid sequence of SEQ ID NO:120; (ix) (a) HVR-H1 comprising the amino acid sequence of SEQ ID NO:22; (b) HVR-H2 comprising the amino acid sequence of SEQ ID NO:42; (c) HVR-H3 comprising the amino acid sequence of SEQ ID NO:70; (d) HVR-L1 comprising the amino acid sequence of SEQ ID NO:89; (e) HVR-L2 comprising the amino acid sequence of SEQ ID NO:105; and (f) HVR-L3 comprising the amino acid sequence of SEQ ID NO:118; or (x) (a) HVR-H1 comprising the amino acid sequence of SEQ ID NO:23; (b) HVR-H2 comprising the amino acid sequence of SEQ ID NO:47; (c) HVR-H3 comprising the amino acid sequence of SEQ ID NO:71; (d) HVR-L1 comprising the amino acid sequence of SEQ ID NO:90; (e) HVR-L2 comprising the amino acid sequence of SEQ ID NO:106; and (f) HVR-L3 comprising the amino acid sequence of SEQ ID NO:121.

In some embodiments, the antibody comprises a VH sequence selected from the group consisting of SEQ ID NO:170, SEQ ID NO:171, SEQ ID NO:172, SEQ ID NO:203, SEQ ID NO:204, SEQ ID NO:205, SEQ ID NO:206, SEQ ID NO:226, SEQ ID NO:227, and SEQ ID NO:228, and a VL sequence selected from the group consisting of SEQ ID NO:131, SEQ ID NO:132, SEQ ID NO:133, SEQ ID NO:141, SEQ ID NO:142, SEQ ID NO:143, SEQ ID NO:144, SEQ ID NO:152, SEQ ID NO:153, and SEQ ID NO:154.

In some embodiments, the antibody comprises (a) a VH sequence of SEQ ID NO:170 and a VL sequence of SEQ ID NO:131; (b) a VH sequence of SEQ ID NO:171 and a VL sequence of SEQ ID NO:132; (c) a VH sequence of SEQ ID NO:172 and a VL sequence of SEQ ID NO:133; (d) a VH sequence of SEQ ID NO:203 and a VL sequence of SEQ ID NO:141; (e) a VH sequence of SEQ ID NO:204 and a VL sequence of SEQ ID NO:142; (f) a VH sequence of SEQ ID NO:205 and a VL sequence of SEQ ID NO:143; (g) a VH sequence of SEQ ID NO:206 and a VL sequence of SEQ ID NO:144; (h) a VH sequence of SEQ ID NO:226 and a VL sequence of SEQ ID NO:152; (i) a VH sequence of SEQ ID NO:227 and a VL sequence of SEQ ID NO:153; or (j) a VH sequence of SEQ ID NO:228 and a VL sequence of SEQ ID NO:154.

Further provided herein is an isolated antibody that binds to KLK5, wherein the antibody comprises (a) a VH sequence having at least 95% sequence identity to the amino acid sequence of SEQ ID NO:170 and a VL sequence having at least 95% sequence identity to the amino acid sequence of SEQ ID NO:131; (b) a VH sequence having at least 95% sequence identity to the amino acid sequence of SEQ ID NO:171 and a VL sequence having at least 95% sequence identity to the amino acid sequence of SEQ ID NO:132; (c) a VH sequence having at least 95% sequence identity to the amino acid sequence of SEQ ID NO:172 and a VL sequence having at least 95% sequence identity to the amino acid sequence of SEQ ID NO:133; (d) a VH sequence having at least 95% sequence identity to the amino acid sequence of SEQ ID NO:203 and a VL sequence having at least 95% sequence identity to the amino acid sequence of SEQ ID NO:141; (e) a VH sequence having at least 95% sequence identity to the amino acid sequence of SEQ ID NO:204 and a VL sequence having at least 95% sequence identity to the amino acid sequence of SEQ ID NO:142; (f) a VH sequence having at least 95% sequence identity to the amino acid sequence of SEQ ID NO:205 and a VL sequence having at least 95% sequence identity to the amino acid sequence of SEQ ID NO:143; (g) a VH sequence having at least 95% sequence identity to the amino acid sequence of SEQ ID NO:206 and a VL sequence having at least 95% sequence identity to the amino acid sequence of SEQ ID NO:144; (h) a VH sequence having at least 95% sequence identity to the amino acid sequence of SEQ ID NO:226 and a VL sequence having at least 95% sequence identity to the amino acid sequence of SEQ ID NO:152; (i) a VH sequence having at least 95% sequence identity to the amino acid sequence of SEQ ID NO:227 and a VL sequence having at least 95% sequence identity to the amino acid sequence of SEQ ID NO:153; or (j) a VH sequence having at least 95% sequence identity to the amino acid sequence of SEQ ID NO:228 and a VL sequence having at least 95% sequence identity to the amino acid sequence of SEQ ID NO:154.

In some embodiments of any of the antibodies, the antibody is an IgG1 or IgG4.

In some embodiments of any of the antibodies, the antibody inhibits the biological activity of KLK5 by at least 50% as measured by one or more methods as described in the Examples herein below. In some embodiments, the one or more methods is selected from the group consisting of a recombinant KLK5 direct activity assay, coupled pro-KLK1 fluorescent peptide assay, a coupled pro-KLK7 fluorescent peptide assay, a pro-KLK1 LC/MS assay, a pro-KLK7 LC/MS assay, and a Kᵢ₍ₐₚₚ₎ assay. In some embodiments, the biological activity is the serine protease activity of KLK5.

In some embodiments of any of the antibodies, the antibody is a monoclonal antibody.

In some embodiments of any of the antibodies, the antibody is a human, humanized, or chimeric antibody.

In some embodiments of any of the antibodies, the antibody is an antibody fragment that binds KLK5.

Further provided herein is an antibody that forms a thermodynamic epitope when bound to KLK5 comprising one or more of the sequences selected from the group consisting of SEQ ID NO:316, SEQ ID NO:317, SEQ ID NO:318, and SEQ ID NO:319 as measured by hydrogen exchange mass spectrometry.

Further provided herein is an antibody that competes for binding with any of the antibodies described herein.

Further provided herein is an antibody that binds to the same epitope as any of the antibodies described herein.

Further provided herein is an isolated nucleic acid encoding any of the antibodies described herein.

Further provided herein is a host cell comprising the nucleic acid described herein.

Further provided herein is a method of producing an antibody comprising culturing the host cell described herein so that the antibody is produced.

Further provided herein is an immunoconjugate comprising the antibody described herein.

Further provided herein is a pharmaceutical formulation comprising the antibody described herein and a pharmaceutically acceptable carrier.

Further provided herein is the antibody as described herein for use as a medicament.

Further provided herein is the antibody as described herein for use in treating a disease selected from the group consisting of Netherton Syndrome, asthma, atopic dermatitis, psoriasis, and rosacea. In some embodiments, the disease is selected from the group consisting of Netherton Syndrome, asthma, atopic dermatitis, psoriasis, rosacea and eosinophilic esophagitis. In some embodiments, the asthma is selected from the group consisting of atopic asthma, allergic asthma, non-allergic asthma, exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, T helper lymphocyte type 2 (Th2) or type 2 (Th2) high, or Type 2 (T2)-driven asthma, eosinophilic asthma, periostin-high asthma, eosinophil-high asthma, Th2-low asthma or nonTh2-driven asthma, periostin-low asthma, and eosinophil-low asthma. In some embodiments, the asthma is TH2-low asthma.

Further provided is the antibody as described herein for use in inhibiting the biological activity of KLK5.

Further provided is the use of the antibody as described herein in the manufacture of a medicament. In some embodiments, the medicament is for treating a disease selected from the group consisting of Netherton Syndrome, asthma, atopic dermatitis, psoriasis, and rosacea. In some embodiments, the disease is selected from the group consisting of Netherton Syndrome, asthma, atopic dermatitis, psoriasis, rosacea and eosinophilic esophagitis. In some embodiments, the asthma is selected from the group consisting of atopic asthma, allergic asthma, non-allergic asthma, exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, T helper lymphocyte type 2 (Th2) or type 2 (Th2) high, or Type 2 (T2)-driven asthma, eosinophilic asthma, periostin-high asthma, eosinophil-high asthma, Th2-low asthma or nonTh2-driven asthma, periostin-low asthma, and eosinophil-low asthma. In some embodiments, the asthma is TH2-low asthma.

Further provided is the use of the antibody described herein in the manufacture of a medicament for inhibiting the biological activity of KLK5.

Further provided is a method of treating an individual having a disease, wherein the disease is selected from the group consisting of Netherton Syndrome, asthma, atopic dermatitis, psoriasis, and rosacea comprising administering to the individual an effective amount of the antibody described herein. In some embodiments, the disease is selected from the group consisting of Netherton Syndrome, asthma, atopic dermatitis, psoriasis, rosacea and eosinophilic esophagitis. In some embodiments, the asthma is selected from the group consisting of atopic asthma, allergic asthma, non-allergic asthma, exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, T helper lymphocyte type 2 (Th2) or type 2 (Th2) high, or Type 2 (T2)-driven asthma, eosinophilic asthma, periostin-high asthma, eosinophil-high asthma, Th2-low asthma or nonTh2-driven asthma, periostin-low asthma, and eosinophil-low asthma. In some embodiments, the asthma is Th2-low asthma.

Further provided is a method of inhibiting the biological activity of KLK5 in an individual comprising administering to the individual an effective amount of the antibody described herein to inhibit the biological activity of KLK5.

Further provided herein is an antibody that specifically binds to human KLK5, wherein the antibody binds to an epitope on human KLK5 comprising one or more amino acid residues selected from the group consisting of Pro130, Ser131, Ala132, Gly133, Val162, Leu163, Ser164, Gln165, Lys166, Arg167, Glu169, Asp170, Ala171, Tyr172, Pro173, Arg174, Gln174A, Ile176, Asp177, Asp178, Gly184, Asp185, Lys186, Ala186A, Arg188, Asn223, Arg224, Pro225, and Lys233 according to standard protease numbering. In some embodiments, the antibody binds to an epitope on human KLK5 comprising one or more amino acid residues selected from the group consisting of Pro130, Ser131, Ala132, Val162, Leu163, Ser164, Gln165, Lys166, Arg167, Glu169, Asp170, Ala171, Tyr172, Pro173, Arg174, Gln174A, Ile176, Asp177, Asp178, Arg224, and Lys233 according to standard protease numbering. In some embodiments, the antibody binds to an epitope on human KLK5 comprising one or more amino acid residues selected from the group consisting of Pro130, Ser131, Ala132, Gly133, Val162, Leu163, Ser164, Gln165, Lys166, Arg167, Glu169, Asp170, Ala171, Tyr172, Pro173, Arg174, Gln174A, Ile176, Asp177, and Lys233 according to standard protease numbering. In some embodiments, the antibody binds to an epitope on human KLK5 comprising one or more amino acid residues selected from the group consisting of Ser131, Ala132, Gly133, Leu163, Ser164, Gln165, Lys166, Arg167, Glu169, Asp170, Ala171, Pro173, Arg174, Gly184, Asp185, Lys186, Ala186A, Arg188, Asn223, Arg224, and Pro225 according to standard protease numbering.

Further provided herein is an antibody when bound to human KLK5 results in a conformational change of human KLK5, wherein the conformational change allosterically results in the disruption of the substrate binding site and/or the active site of human KLK5.

### BRIEF DESCRIPTION OF THE FIGURES

FIG. 1A-1N show the evaluation of each of the KLK5 inhibitors using fluorescent peptide substrate in the direct assay. 5 nM recombinant human KLK5 and 0.19-100 nM KLK5 inhibitors were pre-incubated for 30 minutes prior to addition of 50 μM Boc-VPR-AMC. Plates were examined every 102 s for 30-60 minute using a PHERAstar® Plus reader using a 340 nm excitation/460 nm emission module. The results for the KLK5 inhibitors are depicted as follows: FIG. 1A: Spink9.SRE.Fc (Spink9), FIG. 1B: mAb1108, FIG. 1C: 3-3F5, FIG. 1D: 10C8, FIG. 1E: 9B6, FIG. 1F: 9F2, FIG. 1G: 9H3, FIG. 1H: 9H5, FIG. 1I: 8B7, FIG. 1J: 2B11, FIG. 1K: 8F5, FIG. 1L: 10C5, FIG. 1M: 10H3, and FIG. 1N: 2-3F4.

FIG. 2A-2N show the evaluation of KLK5 inhibitors in pro-KLK7 coupled assay. 5 nM recombinant human KLK5 and 0.19-100 nM anti-KLK5 inhibitors were pre-incubated for 30 minutes prior to addition of 125 nM pro-KLK7 and 100 μM suc-LLVY-AMC. After 24 hours, fluorescent readings were made every 102 s for 30-60 min and the RFU endpoint value was calculated by averaging the last 5 readings. The results for the KLK5 inhibitors are depicted as follows: FIG. 2A: Spink9.SRE.Fc, FIG. 2B: mAb1108, FIG. 2C: 3-3F5, FIG. 2D: 10C8, FIG. 2E: 9B6, FIG. 2F: 9F2, FIG. 2G: 9H3, FIG. 2H: 9H5, FIG. 2I: 8B7, FIG. 2J: 2B11, FIG. 2K: 8F5, FIG. 2L: 10C5, FIG. 2M: 10H3, and FIG. 2N: 2-3F4.

FIG. 3A-3N show the evaluation of KLK5 inhibitors in pro-KLK1 coupled assay. 0.5 nM recombinant human KLK5 and 0.019-10 nM KLK5 inhibitors were pre-incubated for 30 minutes prior to addition of 31.25 nM pro-KLK1 and 50 μM PFR-AMC. Plates were examined every 102 s for 120 minute using a PHERAstar® Plus reader using a 340 nm excitation/460 nm emission module. The results for the KLK5 inhibitors are depicted as follows: FIG. 3A: Spink9.SRE.Fc, FIG. 3B: mAb1108, FIG. 3C: 3-3F5, FIG. 3D: 10C8, FIG. 3E: 9B6, FIG. 3F: 9F2, FIG. 3G: 9H3, FIG. 3H: 9H5, FIG. 3I: 8B7, FIG. 3J: 2B11, FIG. 3K: 8F5, FIG. 3L: 10C5, FIG. 3M: 10H3, and FIG. 3N: 2-3F4.

FIG. 4A-4N shows the results of an LC/MS assay measuring the inhibition of proteolysis of pro-KLK7 by recombinant KLK5 by monitoring the KLK5-derived cleavage product peptides. A pre-incubation of SPINK9.SRE.Fc, mAb1108 and the 12 selected antibodies and KLK5 preceded a two-hour incubation of 5 nM KLK5 with 15 nM pro-KLK7. The results for the KLK5 inhibitors are depicted as follows: FIG. 4A: SPINK9.SRE.Fc, FIG. 4B: mAb1108, FIG. 4C: 10C8, FIG. 4D: 9B6, FIG. 4E: 2B11, FIG. 4F: 9F2, FIG. 4G: 2-3F4, FIG. 4H: 10H3, FIG. 4I: 9H3, FIG. 4J: 8B7, FIG. 4K: 8F5, FIG. 4L: 3-3F5, FIG. 4M: 10C5, and FIG. 4N: 9H5.

FIG. 5A-5N shows the results of an LC/MS assay measuring the inhibition of proteolysis of pro-KLK1 by recombinant KLK5 by monitoring the KLK5-derived cleavage product peptides. A pre-incubation of SPINK9.SRE.Fc, mAb1108 and 12 selected antibodies and KLK5 preceded a 20 minute incubation of 0.5 nM KLK5 with 300 nM pro-KLK1 (FIG. 5A-N). The results for the KLK5 inhibitors are depicted as follows: FIG. 5A: SPINK9.SRE.Fc, FIG. 5B: mAb1108, FIG. 5C: 10C8, FIG. 5D: 9B6, FIG. 5E: 2B11, FIG. 5F: 9F2, FIG. 5G: 2-3F4, FIG. 5H: 10H3, FIG. 5I: 9H3, FIG. 5J: 8B7, FIG. 5K: 8F5, FIG. 5L: 3-3F5, FIG. 5M: 10C5, and FIG. 5N: 9H5.

FIG. 6A-6N shows the Specificity of KLK5 inhibitors on KLK7 activity. 5 nM recombinant human KLK7 and 0.19-100 nM anti-KLK5 inhibitors were pre-incubated for 30 minutes prior to addition of 100 μM suc-LLVY-AMC. Plates were examined every 102 s for 75 minutes using a PHERAstar® Plus reader using a 340 nm excitation/460 nm emission module. IC₅₀ values are summarized in Table 6. The results for the KLK5 inhibitors are depicted as follows: FIG. 6A: Spink9.SRE.Fc, FIG. 6B: mAb1108, FIG. 6C: 3-3F5, FIG. 6D: 10C8, FIG. 6E: 9B6, FIG. 6F: 9F2, FIG. 6G: 9H3, FIG. 6H: 9H5, FIG. 6I: 2B11, FIG. 6J: 8B7, FIG. 6K: 8F5, FIG. 6L: 10C5, FIG. 6M: 10H3 and FIG. 6N: 2-3F4.

FIG. 7A-7N shows the specificity of KLK5 inhibitors on KLK1 activity. 3 nM recombinant human KLK1 and 0.19-100 nM anti-KLK5 inhibitors were pre-incubated for 30 minutes prior to addition of 100 μM PFR-AMC. Plates were examined every 102 s for 60 minutes using a PHERAstar® Plus reader using a 340 nm excitation/460 nm emission module. IC₅₀ values are summarized in Table 7. The results for the KLK5 inhibitors are depicted as follows: FIG. 7A: Spink9.SRE.Fc, FIG. 7B: mAb1108, FIG. 7C: 3-3F5, FIG. 7D: 10C8, FIG. 7E: 9B6, FIG. 7F: 9F2, FIG. 7G: 9H3, FIG. 7H: 9H5, FIG. 7I: 8B7, FIG. 7J: 2B11, FIG. 7K: 8F5, FIG. 7L: 10C5, FIG. 7M: 10H3 and FIG. 7N: 2-3F4.

FIG. 8A-8N shows the specificity of KLK5 inhibitors on KLK4 activity. 2 nM recombinant human KLK4 and 0.19-100 nM anti-KLK5 inhibitors were pre-incubated for 30 minutes prior to addition of 50 μM Boc-VPR-AMC. Plates were examined every 102 s for 60 minutes using a PHERAstar® Plus reader using a 340 nm excitation/460 nm emission module. IC₅₀ values are summarized in Table 8. The results for the KLK5 inhibitors are depicted as follows: FIG. 8A: Spink9.SRE.Fc, FIG. 8B: mAb1108, FIG. 8C: 3-3F5, FIG. 8D: 10C8, FIG. 8E: 9B6, FIG. 8F: 9F2, FIG. 8G: 9H3, FIG. 8H: 9H5, FIG. 8I: 8B7, FIG. 8J: 2B11, FIG. 8K: 8F5, FIG. 8L: 10C5, FIG. 8M: 10H3 and FIG. 8N: 2-3F4.

FIG. 9A-9N shows the specificity of KLK5 inhibitors on Trypsin activity. 0.25 nM trypsin isolated from bovine pancreas and 0.19-100 nM anti-KLK5 inhibitors were pre-incubated for 30 minutes prior to addition of 50 μM Boc-VPR-AMC. Plates were examined every 102 s for 60 minutes using a PHERAstar® Plus reader using a 340 nm excitation/460 nm emission module. IC₅₀ values for selected antibodies (FIG. 9C-N) are summarized in Table 9. The results for the KLK5 inhibitors are depicted as follows: FIG. 9A: Spink9.SRE.Fc, FIG. 9B: mAb1108, FIG. 9C: 3-3F5, FIG. 9D: 10C8, FIG. 9E: 9B6, FIG. 9F: 9F2, FIG. 9G: 9H3, FIG. 9H: 9H5, FIG. 9I: 8B7, FIG. 9J: 2B11, FIG. 9K: 8F5, FIG. 9L: 10C5, FIG. 9M: 10H3 and FIG. 9N: 2-3F4.

FIG. 10A-10AB shows the analysis of KLK5 inhibitor potency by determination of Kiₐₚₚ. KLK5 at various concentrations (0.5, 0.25, 0.125, and 0.0625 nM) and 0.0019-10 nM anti-KLK5 inhibitors were pre-incubated for 30 minutes prior to addition of 300 μM Z-VPR-pNA. Plates were read in a Versamax tunable microplate reader with measurements at 405 nm taken every 102 seconds for 3 hours. The derived Kiₐₚₚ values are summarized in Table 10. The results for the KLK5 inhibitors are depicted as follows (respective left panels show IC₅₀ values of KLK5 inhibitors at various KLK5 concentrations (0.5, 0.25, 0.125, and 0.0625 nM); respective right panels show IC₅₀ values determined and plotted as a function of KLK5 concentration): FIG. 10A and FIG. 10B: Spink9.SRE.Fc, FIG. 10C and FIG. 10D: mAb1108, FIG. 10E and FIG. 10F: 3-3F5, FIG. 10G and FIG. 10H: 10C8, FIG. 10I and FIG. 10J: 9B6, FIG. 10K and FIG. 10L: 9F2, FIG. 10M and FIG. 10N: 9H3, FIG. 10O and FIG. 10P: 9H5, FIG. 10Q and FIG. 10R: 8B7, FIG. 10S and FIG. 10T: 2B11, FIG. 10U and FIG. 10V: 8F5, FIG. 10W and FIG. 10X: 10C5, FIG. 10Y and FIG. 10Z: 10H3, FIG. 10AA and FIG. 10AB: 2-3F4.

FIG. 11 shows a table summarizing the IC₅₀ values as evaluated and shown in FIGS. 1-10.

FIGS. 12A and 12B. FIG. 12A (depicting human KLK5 used in HDX experiments as SEQ ID NO: 353) shows sequence regions that are identified by hydrogen exchange measurements to be affected when in complex with each antibody, these sequence regions (underlined) are: region 1 (56-68, pHₑₓₚ 8.0), region 2 (107-124, pHₑₓₚ 6.0), region 3 (184-195, pHₑₓₚ 8.0), region 4 (232-246, pHₑₓₚ 6.0), effective time correction uses pH_{ref}-7.5. FIG. 12B shows the actual hydrogen exchange data for a representative peptide for each sequence region underlined in FIG. 12A.

FIGS. 13A-1 to 13A-3 and 13B-1 to 13B-4. FIG. 13A-1 to 13A-3 shows alignment of the light chain variable sequences of anti-KLK5 antibody clones 14C8 (SEQ ID NO: 128), 14E12 (SEQ ID NO: 129), 8E11 (SEQ ID NO: 130), 8G10 (SEQ ID NO: 131), 9B6 (SEQ ID NO: 132), 2-3F4 (SEQ ID NO: 133), 10C5 (SEQ ID NO: 134), 2B11 (SEQ ID NO: 141), 10H3 (SEQ ID NO: 142), 9H3 (SEQ ID NO: 143), 8B7 (SEQ ID NO: 144), 9H5 (SEQ ID NO: 145), 9F2 (SEQ ID NO: 152), 10C8 (SEQ ID NO: 153), 8F5 (SEQ ID NO: 154), 3-3F5 (SEQ ID NO: 155), 9E3 (SEQ ID NO: 163), 10D10 (SEQ ID NO: 164), 12B3 (SEQ ID NO: 165), 1D10 (SEQ ID NO: 166). FIG. 13B-1 to 13B-4 shows alignment of the heavy chain variable sequences of anti-KLK5 antibody clones 14C8 (SEQ ID NO: 167), 14E12 (SEQ ID NO: 168), 8E11 (SEQ ID NO: 169), 8G10 (SEQ ID NO: 170), 9B6 (SEQ ID NO: 171), 2-3F4 (SEQ ID NO: 172), 10C5 (SEQ ID NO: 173), 2B11 (SEQ ID NO: 203), 10H3 (SEQ ID NO: 204), 9H3 (SEQ ID NO: 205), 8B7 (SEQ ID NO: 206), 9H5 (SEQ ID NO: 207), 9F2 (SEQ ID NO: 226), 10C8 (SEQ ID NO: 227), 8F5 (SEQ ID NO: 228), 3-3F5 (SEQ ID NO: 229), 9E3 (SEQ ID NO: 258), 10D10 (SEQ ID NO: 259), 12B3 (SEQ ID NO: 260), 1D10 (SEQ ID NO: 261). Shown are also the CDR regions according to Kabat numbering.

FIGS. 14A and 14B-1 to 14B-3. FIG. 14A shows alignment of the light chain variable sequences of the anti-KLK5 antibody clone 10C5 (SEQ ID NO: 134) and six humanized 10C5 clones (hu10C5-L1 (SEQ ID NO: 135), hu10C5-L2 (SEQ ID NO: 136), hu10C5-L3 (SEQ ID NO: 137), hu10C5-L4 (SEQ ID NO: 138), hu10C5-L5 (SEQ ID NO: 139), and hu10C5-L6 (SEQ ID NO: 321)). FIG. 14B-1 to 14B-3 shows alignment of the heavy chain variable sequences of the anti-KLK5 antibody clone 10C5 (SEQ ID NO: 173) and 28 humanized 10C5 clones (hu10C5-H1 (SEQ ID NO: 174), hu10C5-H2 (SEQ ID NO: 175), hu10C5-H3 (SEQ ID NO: 176), hu10C5-H4 (SEQ ID NO: 177), hu10C5-H5 (SEQ ID NO: 178), hu10C5-H6 (SEQ ID NO: 179), hu10C5-H7 (SEQ ID NO: 180), hu10C5-H8 (SEQ ID NO: 181), hu10C5-H9 (SEQ ID NO: 182), hu10C5-H10 (SEQ ID NO: 183), hu10C5-H11 (SEQ ID NO: 184), hu10C5-H12 (SEQ ID NO: 185), hu10C5-H13 (SEQ ID NO: 186), hu10C5-H14 (SEQ ID NO: 187), hu10C5-H15 (SEQ ID NO: 188), hu10C5-H16 (SEQ ID NO: 189), hu10C5-H17 (SEQ ID NO: 190), hu10C5-H18 (SEQ ID NO: 191), hu10C5-H19 (SEQ ID NO: 192), hu10C5-H20 (SEQ ID NO: 193), hu10C5-H21 (SEQ ID NO: 194), hu10C5-H22 (SEQ ID NO: 194), hu10C5-H23 (SEQ ID NO: 196), hu10C5-H24 (SEQ ID NO: 197), hu10C5-H25 (SEQ ID NO: 198), hu10C5-H26 (SEQ ID NO: 199), hu10C5-H27 (SEQ ID NO: 200), and hu10C5-H28 (SEQ ID NO: 201). The amino acid residues highlighted in black are the residues which were changed. Shown are also the CDR regions according to Kabat numbering.

FIGS. 15A and 15B-1 to 15B-3. FIG. 15A shows alignment of the light chain variable sequences of the anti-KLK5 antibody clone 9H5 (SEQ ID NO: 145) and four humanized 9H5 clones (hu9H5-L1 (SEQ ID NO: 146), hu9H5-L2 (SEQ ID NO: 147), hu9H5-L3 (SEQ ID NO: 148), and hu9H5-L4 (SEQ ID NO: 149)). FIG. 15B-1 to 15B-3 shows alignment of the heavy chain variable sequences of the anti-KLK5 antibody clone 9H5 (SEQ ID NO: 207) and 17 humanized 9H5 clones (hu9H5-H1 (SEQ ID NO: 208), hu9H5-H2 (SEQ ID NO: 209), hu9H5-H3 (SEQ ID NO: 210), hu9H5-H4 (SEQ ID NO: 211), hu9H5-H5 (SEQ ID NO: 212), hu9H5-H6 (SEQ ID NO: 213), hu9H5-H7 (SEQ ID NO: 214), hu9H5-H8 (SEQ ID NO: 215), hu9H5-H9 (SEQ ID NO: 216), hu9H5-H10 (SEQ ID NO: 217), hu9H5-H11 (SEQ ID NO: 218), hu9H5-H12 (SEQ ID NO: 219), hu9H5-H13 (SEQ ID NO: 220), hu9H5-H14 (SEQ ID NO: 221), hu9H5-H15 (SEQ ID NO: 222), hu9H5-H16 (SEQ ID NO: 223), and hu9H5-H17 (SEQ ID NO: 224). The amino acid residues highlighted in black are the residues which were changed. Shown are also the CDR regions according to Kabat numbering.

FIGS. 16A and 16B-1 to 16B-3. FIG. 16A shows alignment of the light chain variable sequences of the anti-KLK5 antibody clone 3-3F5 (SEQ ID NO: 155) and five humanized 3-3F5 clones (hu3-3F5-L1 (SEQ ID NO: 156), hu3-3F5-L2 (SEQ ID NO: 157), hu3-3F5-L3 (SEQ ID NO: 158), hu3-3F5-L4 (SEQ ID NO: 159), and hu3-3F5-L5 (SEQ ID NO: 160)). FIG. 16B-1 to 16B-3 shows alignment of the heavy chain variable sequences of the anti-KLK5 antibody clone 3-3F5 (SEQ ID NO: 229) and 27 humanized 3-3F5 clones (hu3-3F5-H1 (SEQ ID NO: 230), hu3-3F5-H2 (SEQ ID NO: 231), hu3-3F5-H3 (SEQ ID NO: 232), hu3-3F5-H4 (SEQ ID NO: 233), hu3-3F5-H5 (SEQ ID NO: 234), hu3-3F5-H6 (SEQ ID NO: 235), hu3-3F5-H7 (SEQ ID NO: 236), hu3-3F5-H8 (SEQ ID NO: 237), hu3-3F5-H9 (SEQ ID NO: 238), hu3-3F5-H10 (SEQ ID NO: 239), hu3-3F5-H11 (SEQ ID NO: 240), hu3-3F5-H12 (SEQ ID NO: 241), hu3-3F5-H13 (SEQ ID NO: 242), hu3-3F5-H14 (SEQ ID NO: 243), hu3-3F5-H15 (SEQ ID NO: 244), hu3-3F5-H16 (SEQ ID NO: 245), hu3-3F5-H17 (SEQ ID NO: 246), hu3-3F5-H18 (SEQ ID NO: 247), hu3-3F5-H19 (SEQ ID NO: 248), hu3-3F5-H20 (SEQ ID NO: 249), hu3-3F5-H21 (SEQ ID NO: 250), hu3-3F5-H22 (SEQ ID NO: 251), hu3-3F5-H23 (SEQ ID NO: 252), hu3-3F5-H24 (SEQ ID NO: 253), hu3-3F5-H25 (SEQ ID NO: 254), hu3-3F5-H26 (SEQ ID NO: 255), and hu3-3F5-H27 (SEQ ID NO: 256)). The amino acid residues highlighted in black are the residues which were changed. Shown are also the CDR regions according to Kabat numbering.

FIG. 17 shows the evaluation of KLK5 inhibitors in the A549 cell-based IL-8 secretion assay. The KLK5-induced IL-8 secretion level (gray star) was set at 100% residual activity while the buffer (starvation media) alone (gray circle) was set at 0% residual activity. Dose response curves are shown for Spink5.Fc (black circle and dotted black line), humanized 3.3F5 (black diamond and solid black line) anti-KLK5 antibody, humanized 9H5 anti-KLK5 antibody (open gray square and solid gray line), and humanized 10C5 anti-KLK5 antibody (black open circle and dotted gray line). The data shown are the mean±standard deviation of at least three independent experiments.

FIGS. 18A and 18B. FIG. 18A shows an overlay of human KLK5 with KLK5-10C5 Fab complex. FIG. 18B shows the interface between human KLK5 and 10C5 Fab. The numbering of the amino acid residues of human KLK5 is based on the standard numbering for proteases. See Debela et al., J Mol Biol, 373, 1017-1031 (2007). The numbering of the amino acid residues of the Fab fragments is based on Kabat.

FIGS. 19A and 19B. FIG. 19A shows an overlay of human KLK5 with KLK5-9H5 Fab complex. FIG. 19B shows the interface between human KLK5 and 9H5 Fab. The numbering of the amino acid residues of human KLK5 is based on the standard numbering for proteases. The numbering of the amino acid residues of the Fab fragments is based on Kabat.

FIGS. 20A and 20B. FIG. 20A shows an overlay of human KLK5 with KLK5-3-3F5 Fab complex. FIG. 20B shows the interface between human KLK5 and 3-3F5 Fab. The numbering of the amino acid residues of human KLK5 is based on the standard numbering for proteases. The numbering of the amino acid residues of the Fab fragments is based on Kabat.

### DETAILED DESCRIPTION OF EMBODIMENTS OF THE INVENTION

### I. Definitions

The terms “anti-KLK5 antibody” and “an antibody that binds to KLK5” refer to an antibody that is capable of binding KLK5 with sufficient affinity such that the antibody is useful as a diagnostic and/or therapeutic agent in targeting KLK5. In some embodiments, the extent of binding of an anti-KLK5 antibody to an unrelated polypeptide (polypeptide other than KLK5) is less than about 10% of the binding of the antibody to KLK5 as measured, e.g., by a radioimmunoassay (RIA). In some embodiments, an antibody that binds to KLK5 has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In some embodiments, an antibody that binds to KLK5 has an IC₅₀ value (concentration of an inhibitor, e.g. an antibody or fragment thereof, required to reduce the rate of an enzymatic reaction by 50%) of ≤1 M, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In some embodiments, an anti-KLK5 antibody binds to a binding region (e.g. an epitope) of KLK5 that is conserved among different species of KLK polypeptides.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “isolated” as used in reference to antibody, binding polypeptide, polynucleotide or small molecule is one which has been separated from a component of its natural environment. In some embodiments, an antibody, binding polypeptide, polynucleotide or small molecule is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The term “monoclonal antibody” as used herein refers to an antibody obtained from a population of substantially homogeneous antibodies, i.e., the individual antibodies comprising the population are identical and/or bind the same binding region (e.g., epitope), except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies described herein may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies.

A “blocking antibody” or an “antagonist antibody” is one which inhibits or reduces biological activity of the antigen it binds. Preferred blocking antibodies or antagonist antibodies substantially or completely inhibit the biological activity of the antigen.

The term “chimeric” antibody refers to an antibody in which a portion of the heavy and/or light chain is derived from a particular source or species, while the remainder of the heavy and/or light chain is derived from a different source or species.

The terms “full length antibody,” “intact antibody,” and “whole antibody” are used herein interchangeably to refer to an antibody (e.g., an anti-KLK5 antibody) having a structure substantially similar to a native antibody structure or having heavy chains that contain an Fc region.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In some embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” or an “antibody that binds to the same binding region” as a reference antibody refers to an antibody that blocks binding of the reference antibody to its binding partner (e.g., an antigen) in a competition assay by 50% or more, and conversely, the reference antibody blocks binding of the antibody to its binding partner in a competition assay by 50% or more.

The term “thermodynamic epitope”, e.g. in the context of epitope mapping using hydrogen exchange mass spectrometry, refers to those portions of a protein whose backbone structural dynamics or local free energy of unfolding is altered in response to a specific binding event such as becoming bound by an antibody. The structural epitope may or may not be partially or entirely contained within the thermodynamic epitope.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

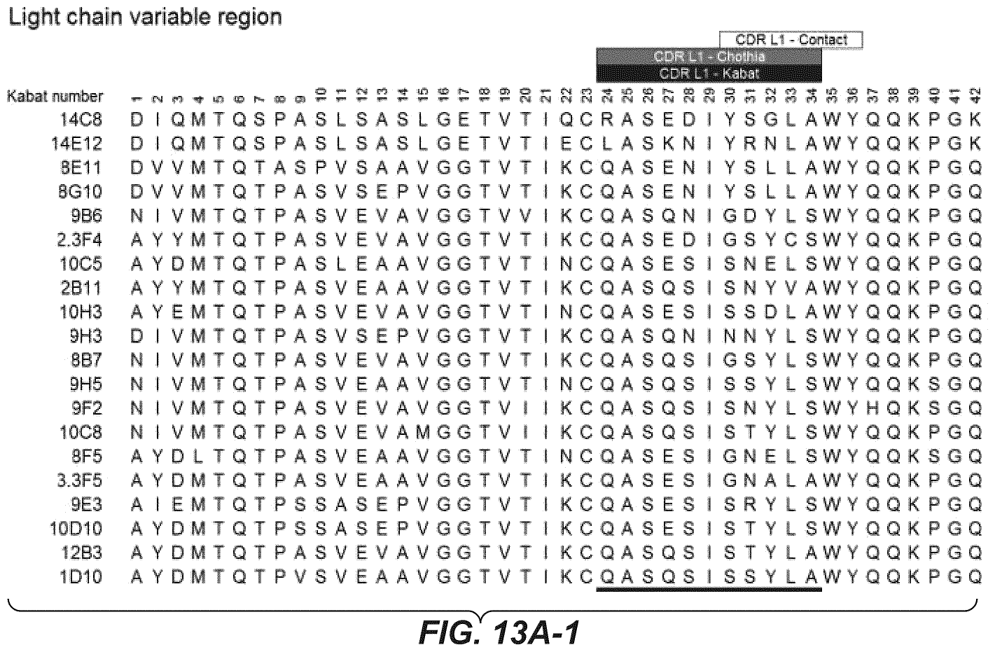

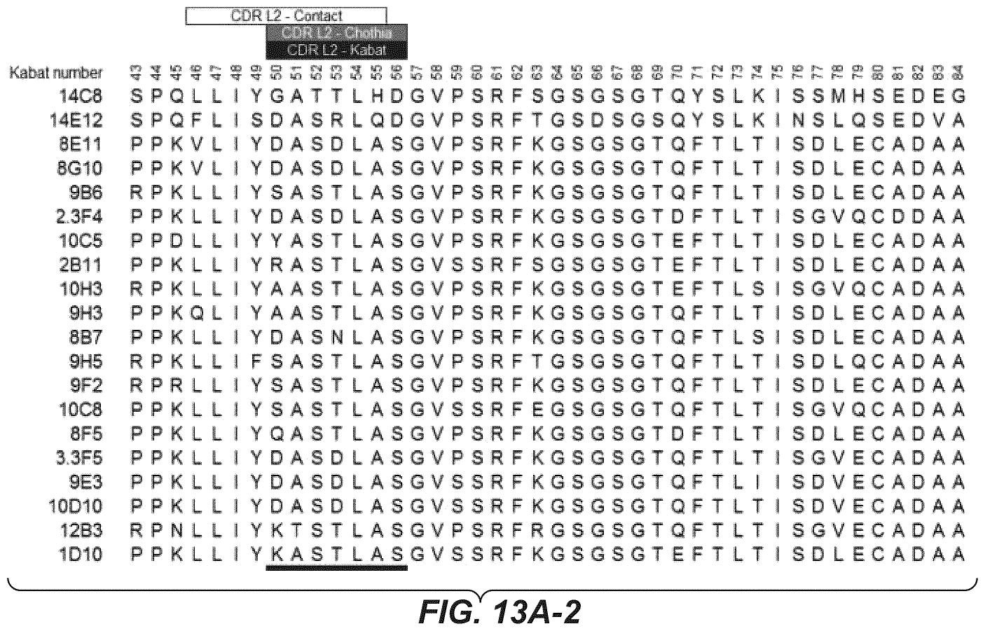

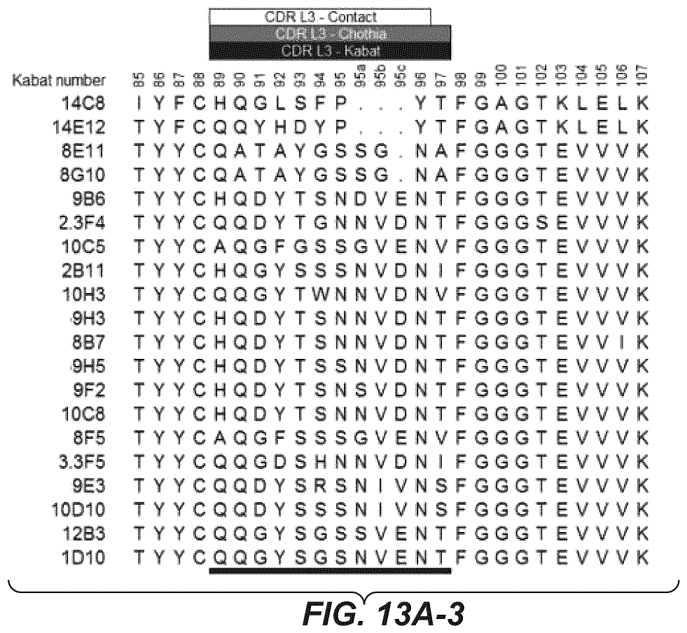

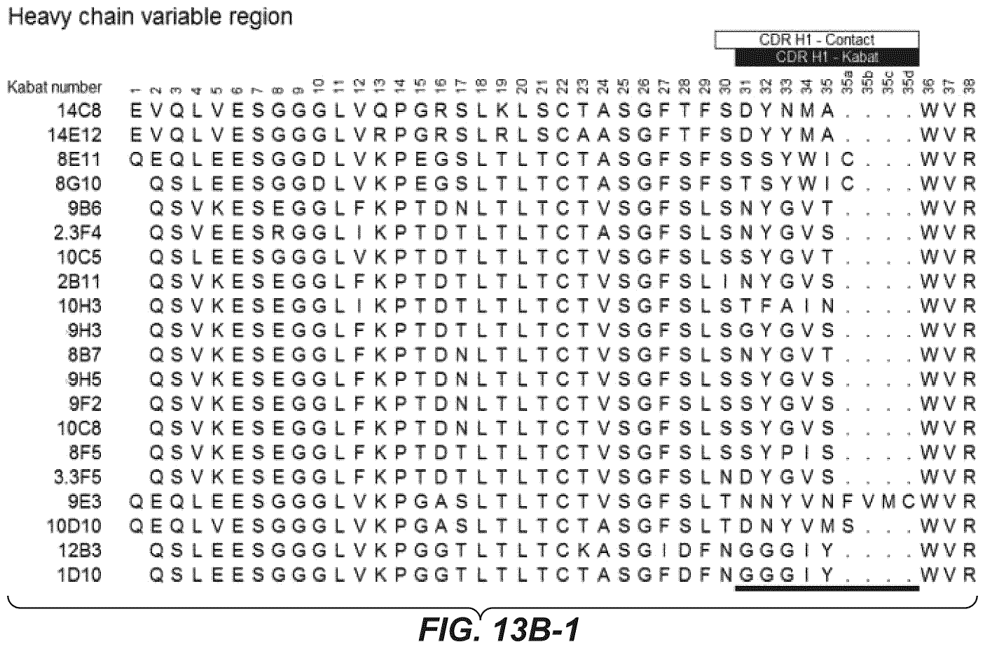

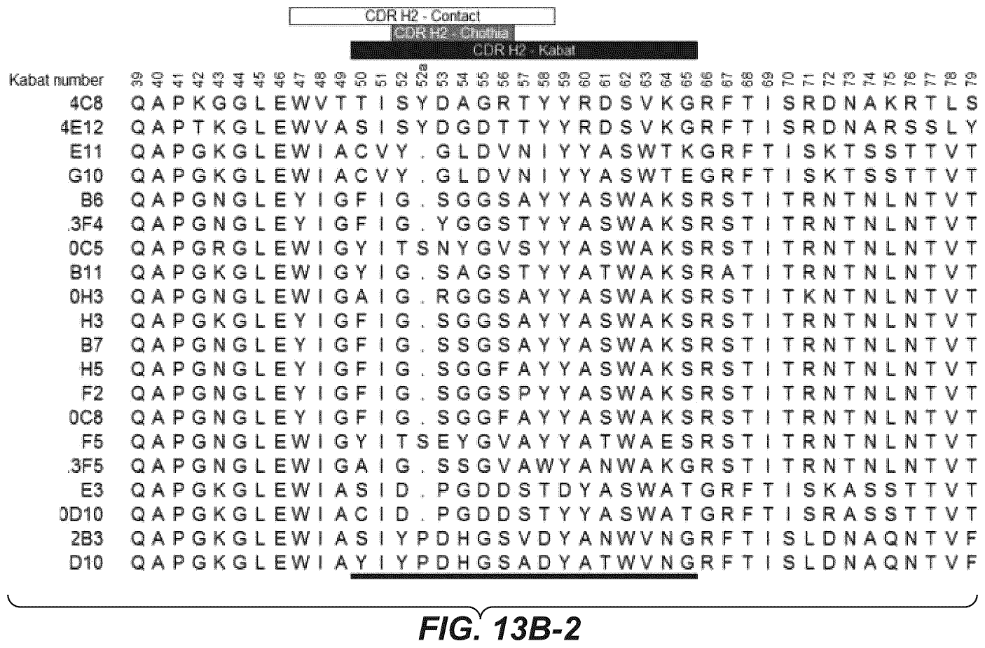

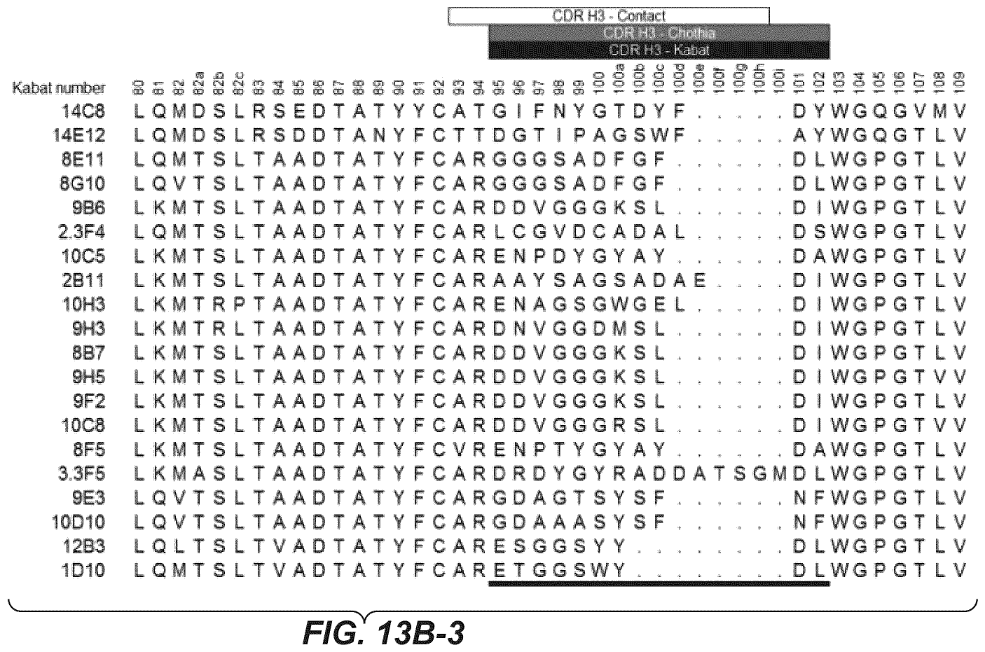

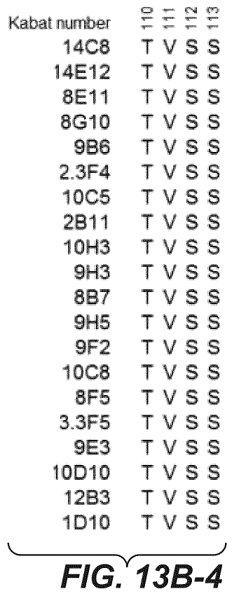

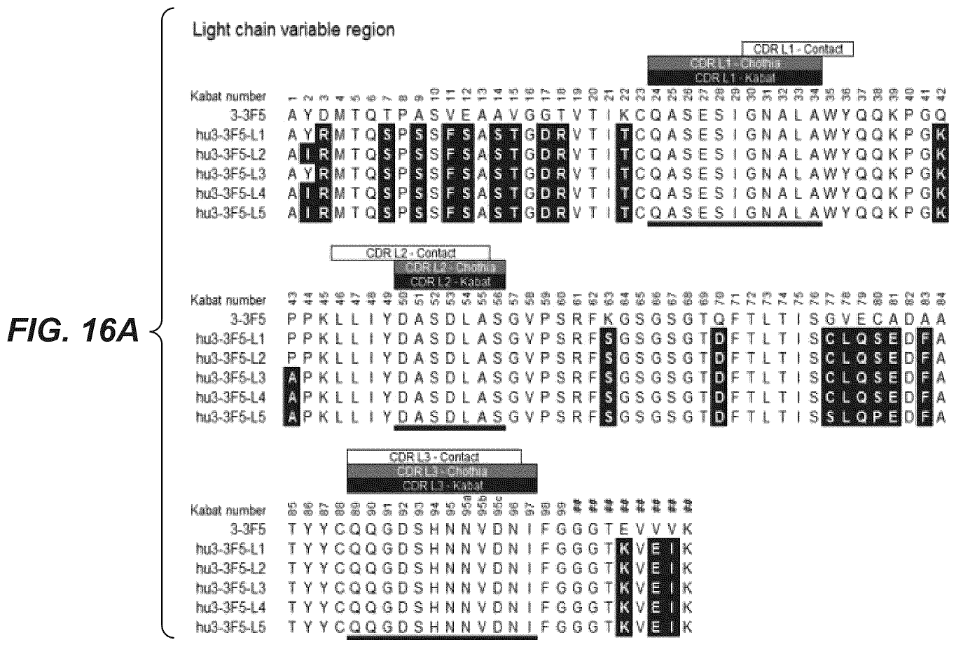

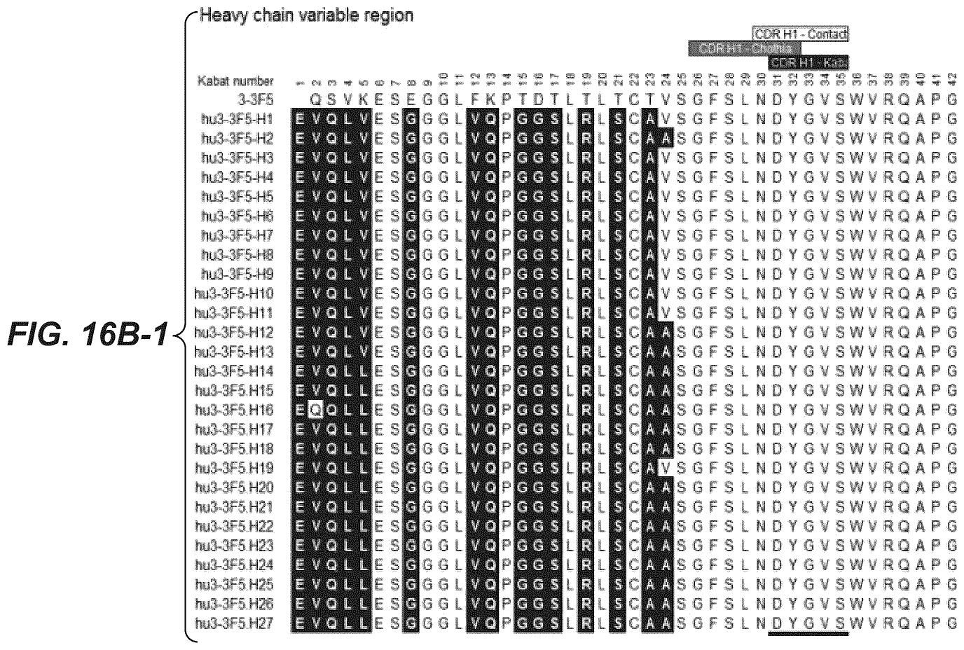

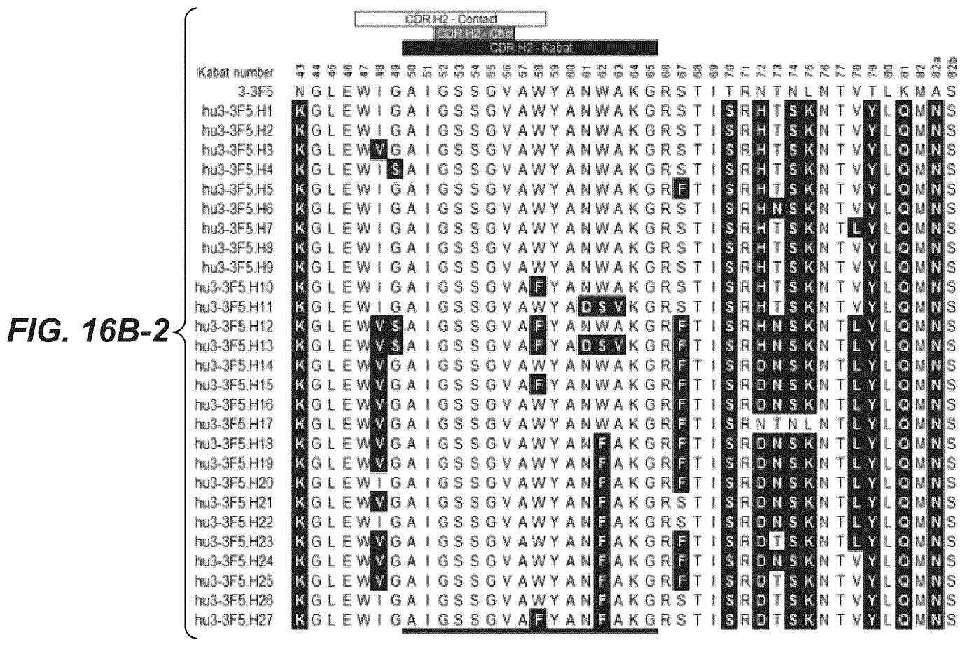

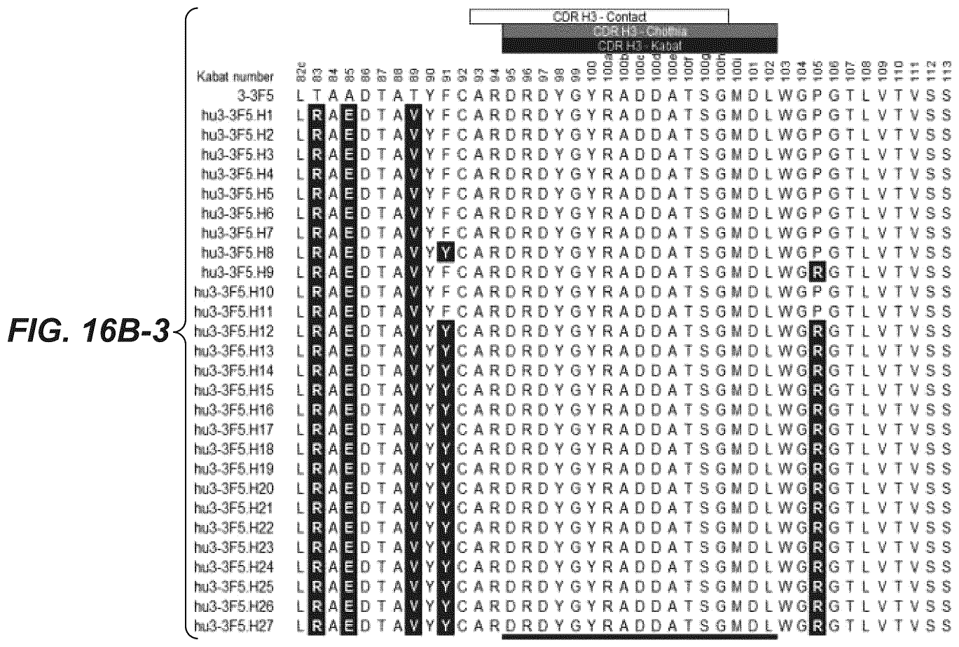

Unless otherwise indicated, HVR residues and other residues in the variable domain (e.g., FR residues) are numbered herein according to Kabat et al., supra, e.g. as set out in FIGS. 13-16 as well as in the Table of Sequences herein below. Unless otherwise indicated, the CDRs are determined according to Kabat et al., supra.

The term “variable region” or “variable domain” refers to the domain of an antibody heavy or light chain that is involved in binding the antibody to an antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

“Affinity” or “Binding Affinity” refers to the strength of the sum total of noncovalent interactions between a single binding site of a molecule (e.g., antibody, binding polypeptide, polynucleotide, small molecule) and its binding partner (e.g., an antigen). Unless indicated otherwise, as used herein, “binding affinity” refers to intrinsic binding affinity which reflects a 1:1 interaction between members of a binding pair (e.g., either of antibody, binding polypeptide, polynucleotide, small molecule and the antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

A “binding region” is the portion of the binding partner (e.g., an antigen) to which a KLK5 antibody selectively binds. For a binding polypeptide binding partner, a linear binding region can be a peptide portion of about 4-15 (e.g., 4, 5, 6, 7, 8, 9, 10, 11, 12, 13, 14 or 15) amino acid residues. A non-linear, conformational binding region may comprise residues of a polypeptide sequence brought to close vicinity in the three-dimensional (3D) structure of the binding polypeptide binding partner.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The terms “KLK5” and “Kallikrein-5,” as used herein, refers to any native KLK5 from any vertebrate source, including mammals such as primates (e.g., humans) and rodents (e.g., mice and rats), unless otherwise indicated. The term encompasses “full-length,” unprocessed KLK5 as well as any form of KLK5 that results from processing in the cell. The term also encompasses naturally occurring variants of KLK5, e.g., splice variants or allelic variants. In some embodiments, the amino acid sequence of an exemplary human KLK5 is UNIPROT Q9Y337. In some embodiments, the amino acid sequence of an exemplary human KLK5 is selected from the group consisting of SEQ ID NO:1, SEQ ID NO:2, SEQ ID NO:3, SEQ ID NO:4, SEQ ID NO:5, SEQ ID NO:6, SEQ ID NO:7, and SEQ ID NO:8. In some embodiments, the amino acid sequence of an exemplary human KLK5 is amino acid residues 23-293 (minus signal peptide) of UNIPROT Q9Y337 (G55, D153 variant) and is shown in SEQ ID NO:2. In some embodiments, the amino acid sequence of an exemplary human KLK5 is amino acid residues 23-293 (minus signal peptide) of the G55, N153 variant shown in SEQ ID NO:4. In some embodiments, the amino acid sequence of an exemplary human KLK5 is amino acid residues 23-293 (minus signal peptide) of the R55, N153 variant shown in SEQ ID NO:6. In some embodiments, the amino acid sequence of an exemplary human KLK5 is amino acid residues 23-293 (minus signal peptide) of the R55, D153 variant shown in SEQ ID NO:8.

The numbering in this paragraph below, relates to full-length unprocessed KLK5. In some embodiments, the amino acid sequence of the human KLK5 comprises the amino acid N at position 153. In some embodiments, the amino acid sequence of the human KLK5 comprises the amino acid D at position 153. In some embodiments, the amino acid sequence of the human KLK5 comprises the amino acid G at position 55. In some embodiments, the amino acid sequence of the human KLK5 comprises the amino acid R at position 55. In some embodiments, the amino acid sequence of the human KLK5 comprises the amino acid G at position 55 and the amino acid N at position 153. In some embodiments, the amino acid sequence of the human KLK5 comprises the amino acid G at position 55 and the amino acid D at position 153. In some embodiments, the amino acid sequence of the human KLK5 comprises the amino acid R at position 55 and the amino acid N at position 153. In some embodiments, the amino acid sequence of the human KLK5 comprises the amino acid R at position 55 and the amino acid D at position 153.

The numbering in this paragraph below, relates to full-length unprocessed KLK5. In some embodiments, the nucleic acid sequence of the human KLK5 comprises a sequence encoding an N at position 153. In some embodiments, the nucleic acid sequence of the human KLK5 comprises a sequence encoding a D at position 153. In some embodiments, the nucleic acid sequence of the human KLK5 comprises a sequence encoding a G at position 55. In some embodiments, the nucleic acid sequence of the human KLK5 comprises a sequence encoding an R at position 55. In some embodiments, the nucleic acid sequence of the human KLK5 comprises a sequence encoding a G at position 55 and an N at position 153. In some embodiments, the nucleic acid sequence of the human KLK5 comprises a sequence encoding G at position 55 and a D at position 153. In some embodiments, the nucleic acid sequence of the human KLK5 comprises a sequence encoding R at position 55 and an N at position 153. In some embodiments, the nucleic acid sequence of the human KLK5 comprises a sequence encoding an R at position 55 and a D at position 153.

The terms “SPINK5” and “Serine protease inhibitor Kazal-type 5,” as used herein, refers to any native SPINK5 from any vertebrate source, including mammals such as primates (e.g., humans) and rodents (e.g., mice and rats), unless otherwise indicated. The term encompasses “full-length,” unprocessed SPINK5 as well as any form of SPINK5 that result from processing in the cell. The term also encompasses naturally occurring variants of SPINK5, e.g., splice variants or allelic variants. In some embodiments, the amino acid sequence of an exemplary human SPINK5 is UNIPROT Q9NQ38 and is shown in SEQ ID NO:9. In some embodiments, the amino acid sequence of an exemplary human SPINK5 is amino acid residues 23-1064 (minus signal peptide) of UNIPROT Q9NQ38 and is shown in SEQ ID NO:10.

The term “SPINK fusion polypeptide” as used herein refers to a fusion polypeptide in which a SPINK polypeptide or a fragment thereof (e.g., certain domains of the SPINK polypeptide (e.g., SPINK5 and/or SPINK9) is linked, directly or indirectly, to another polypeptide (e.g., non-SPINK polypeptide).

The term “SPINK Fc fusion polypeptide” as used herein refers to a fusion polypeptide in which a SPINK polypeptide or a fragment thereof (e.g., certain domains of the SPINK polypeptide (e.g., SPINK5 and/or SPINK9) is linked, directly or indirectly, to an Fc region. In some embodiments, the Fc region is selected from the group consisting of an IgG1 Fc region, IgG2a Fc region and IgG4 Fc region. In some embodiments, the Fc region is an IgG2a Fc region. In some embodiments, the IgG2a Fc region is a mouse IgG2a Fc region. In some embodiments, the Fc region is an IgG1 Fc region. In some embodiments, the IgG1 Fc region is a human IgG1 Fc region. In some embodiments, the Fc region is an IgG4 Fc region. In some embodiments, the IgG4 Fc region is a human IgG4 Fc region. In some embodiments, the SPINK polypeptide or a fragment thereof is a human SPINK polypeptide or a fragment thereof. In some embodiments, the SPINK polypeptide or a fragment thereof is a mouse SPINK polypeptide or a fragment thereof. It is understood that minor sequence variations such as insertions, deletions, substitutions, especially conservative amino acid substitutions of the SPINK polypeptide, the SPINK domains or the Fe that do not affect the function and/or activity of the SPINK polypeptide, the SPINK domains or the SPINK Fc fusion polypeptide are provided herein. In some embodiments, the SPINK Fc fusion polypeptide provided herein can bind to KLK5, which can lead to inhibition of KLK5. In some embodiments, the SPINK polypeptide or a fragment thereof is SPINK 9. In some embodiments, the SPINK Fc fusion polypeptide is SPINK9.SRE.Fc (SEQ ID NO:320).

The term “polypeptide” as used herein, refers to any native polypeptide of interest (e.g., KLK5, or SPINK5) from any vertebrate source, including mammals such as primates (e.g., humans) and rodents (e.g., mice and rats), unless otherwise indicated. The term encompasses “full-length,” unprocessed polypeptide as well as any form of the polypeptide that results from processing in the cell. The term also encompasses naturally occurring variants of the polypeptide, e.g., splice variants or allelic variants.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

An “isolated” polynucleotide or nucleic acid refers to a molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-KLK5 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “KLK5 genomic sequence” as used herein, refers to either the cDNA and/or the genomic form of the KLK5 gene, which may include introns as well as upstream and downstream regulatory sequences.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject. In yet another embodiment, a reference sample, reference cell, reference tissue, control sample, control cell, or control tissue is obtained from a healthy and/or non-diseased part of the body (e.g., tissues or cells) of another subject. In even another embodiment, a reference sample, reference cell, reference tissue, control sample, control cell, or control tissue is obtained from an untreated tissue and/or cell of the body of another subject.

The term “sample,” as used herein, refers to a formulation that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebrospinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

An “effective amount” of an agent, e.g., a pharmaceutical formulation, refers to an amount effective, at dosages and for periods of time necessary, to achieve the desired therapeutic result.

A “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the subject is a human.

The term “patient” as used herein, refers to an animal, such as a mammal. In one embodiment, patient refers to a human.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “Th2-high asthma” as used herein, refers to asthma that exhibits high levels of one or more Th2 cell-related cytokines, for example, IL13, IL4, IL9, IL5, or that exhibits Th2 cytokine-associated inflammation. In some embodiments, the term Th2-high asthma may be used interchangeably with eosinophil-high asthma. In some embodiments, the Th2-high asthma is Th2 driven asthma. In some embodiments, the asthma patient has been determined to be Eosinophilic Inflammation Positive (EIP). See, e.g., International Patent Application Publication No. WO 2015/061441, which is incorporated by reference herein in its entirety. In some embodiments, the subject has been determined to have elevated levels of at least one of the eosinophilic signature genes as compared to a control or reference level. See WO2015/061441. In some embodiments, the Th2-high asthma is periostin-high asthma. In some embodiments, the subject has high serum periostin. In some embodiments, the subject is eighteen years or older. In some embodiments, the subject has been determined to have an elevated level of serum periostin as compared to a control or reference level. In some embodiments, the control or reference level is the median level of periostin in a population. In some embodiments, the subject has been determined to have 20 ng/ml or higher serum periostin. In some embodiments, the subject has been determined to have 25 ng/ml or higher serum periostin. In some embodiments, the subject has been determined to have 50 ng/ml or higher serum periostin. In some embodiments, the control or reference level of serum periostin is 20 ng/ml, 25 ng/ml, or 50 ng/ml. In some embodiments, the asthma is eosinophil-high asthma. In some embodiments, the subject has been determined to have an elevated eosinophil count as compared to a control or reference level. In some embodiments, the control or reference level is the median level of a population. In some embodiments, the subject has been determined to have 150 or higher eosinophil count/μl blood. In some embodiments, the subject has been determined to have 200 or higher eosinophil count/μl blood. In some embodiments, the subject has been determined to have 250 or higher eosinophil count/μl blood. In some embodiments, the subject has been determined to have 300 or higher eosinophil count/μl blood. In some embodiments, the subject has been determined to have 350 or higher eosinophil count/μl blood. In some embodiments, the subject has been determined to have 400 or higher eosinophil count/μl blood. In some embodiments, the subject has been determined to have 450 or higher eosinophil count/μl blood. In some embodiments, the subject has been determined to have 500 or higher eosinophil count/μl blood. In some preferred embodiments, the subject has been determined to have 300 or higher eosinophil count/μl blood. In some embodiments, the eosinophils are peripheral blood eosinophils. In some embodiments, the eosinophils are sputum eosinophils. In some embodiments, the subject exhibits elevated level of FeNO (fractional exhaled nitric acid) and/or elevated level of IgE. For example, in some instances, the subject exhibits a FeNO level above any of about 5 ppb (parts per billion), 10 ppb, 15 ppb, 20 ppb, 25 ppb, 30 ppb, 35 ppb, 40 ppb, 45 ppb, 50 ppb, 60 ppb, 70 ppb, 80 ppb, 90 ppb and 100 ppb. In some instances, the subject has an IgE level that is above 50 IU/ml.

The term “Th2-low asthma”, “non-Th2-high asthma”, “type 2-low asthma”, “T2-low asthma”, “non-eosinophilic asthma”, pauci-granulocytic asthma”, or “pauci-inflammatory asthma”, as used herein, refers to asthma that exhibits low levels of one or more Th2 cell-related cytokines, for example, IL13, IL4, IL9, IL5, or exhibits non-Th2 cytokine-associated inflammation. In some embodiments, the term Th2-low asthma may be used interchangeably with eosinophil-low asthma. In some embodiments, the asthma patient has been determined to be Eosinophilic Inflammation Negative (EIN). See, e.g., WO 2015/061441. In some embodiments, the Th2-low asthma is Th17-driven asthma. In some embodiments, the Th2-low asthma is periostin-low asthma. In some embodiments, the subject is eighteen years or older. In some embodiments, the subject has been determined to have a reduced level of serum periostin as compared to a control or reference level. In some embodiments, the control or reference level is the median level of periostin in a population. In some embodiments, the subject has been determined to have less than 20 ng/ml serum periostin. In some embodiments, the asthma is eosinophil-low asthma. In some embodiments, the subject has been determined to have a reduced eosinophil count as compared to a control or reference level. In some embodiments, the control or reference level is the medium level of a population. In some embodiments, the subject has been determined to have less than 150 eosinophil count/μl blood. In some embodiments, the subject has been determined to have less than 100 eosinophil count/μl blood. In certain preferred embodiments, the subject has been determined to have less than 300 eosinophil count/μl blood.

“Treatment” (and variations such as “treat” or “treating”) refers to clinical intervention in an attempt to alter the natural course of the subject or cell being treated. Desirable effects of treatment include one or more of preventing occurrence or recurrence of disease, alleviation of symptoms, diminishment of any direct or indirect pathological consequences of the disease, stabilized (i.e., not worsening) state of disease, decreasing the rate of disease progression, amelioration or palliation of the disease state, prolonging survival as compared to expected survival if not receiving treatment and improved prognosis.

The term “package insert” is used to refer to instructions customarily included in commercial packages of therapeutic products, that contain information about the indications, usage, dosage, administration, combination therapy, contraindications and/or warnings concerning the use of such therapeutic products.

The use of the terms “a” and “an” and “the” and similar terms in the context of describing embodiments herein are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. It is understood that aspects and embodiments provided herein include “consisting” and/or “consisting essentially of” aspects and embodiments.

As is understood by one skilled in the art, reference to “about” a value or parameter herein includes (and describes) embodiments that are directed to that value or parameter per se. For example, description referring to “about X” includes description of “X”.

The phrase “substantially different,” refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values may be, for example, greater than about 10%, greater than about 20%, greater than about 30%, greater than about 40%, and/or greater than about 50% as a function of the value for the reference/comparator molecule.

### II. Compositions and Methods

In one aspect, the invention is based, in part, on the discovery of KLK5 antibodies that inhibit the biological activity of KLK5. In some embodiments, antibodies that bind to KLK5 are provided. In some embodiments, an isolated antibody that binds to KLK5 is provided, i.e. an anti-KLK5 antibody. In some embodiments, the anti-KLK5 antibody inhibits the biological activity of KLK5. In some embodiments, the anti-KLK5 antibody substantially or completely inhibits the biological activity of KLK5. In some embodiments, the biological activity of KLK5 is serine protease activity. In some embodiments, the biological activity of KLK5 is tryptic-like serine protease activity. In some embodiments, the biological activity of KLK5 is KLK5 promoted human smooth muscle cell proliferation and contraction. In some embodiments, the biological activity of KLK5 is KLK5 induced epithelial expression of inflammatory cytokines, chemokines, and adhesion molecules. In some embodiments, the biological activity of KLK5 is KLK5 induced epithelium production of neutrophil chemotactic cytokines and neutrophil influx into the lung tissues. In some embodiments, the biological activity of KLK5 is inhibited by at least about any of 40%, 50%, 60%, 70%, 80%, 90% and/or more. In some embodiments, the biological activity of the KLK5 is inhibited by about any of 40%, 50%, 60%, 70%, 80%, 90% and/or more. In some embodiments, the biological activity of the KLK5 is inhibited by between any of 40-50%, 50-60%, 60-70%, 70-80%, 80-90%, and/or 90-100%.

In some embodiments of any of the anti-KLK5 antibodies, the anti-KLK5 antibody substantially or completely inhibits binding of SPINK5 to KLK5. In some embodiments, binding of SPINK5 to KLK5 is inhibited by at least about any of 40%, 50%, 60%, 70%, 80%, 90% and/or more. In some embodiments, binding of SPINK5 to KLK5 is inhibited by about any of 40%, 50%, 60%, 70%, 80%, 90% and/or more. In some embodiments, binding of SPINK5 to KLK5 is inhibited by between any of 40-50%, 50-60%, 60-70%, 70-80%, 80-90%, and/or 90-100%.

In some embodiments of any of the anti-KLK5 antibodies, the anti-KLK5 antibody has an IC₅₀ value of less than about any of 1000 nM, 500 nM, 100 nM, 50 nM, 10 nM, 5 nM, 1 nM, 500 pM, 100 pM, 50 pM, 10 pM, 5 pM, and/or 1 pM. In some embodiments, the anti-KLK5 antibody has an IC₅₀ value of less than any of 1000 nM, 500 nM, 100 nM, 50 nM, 10 nM, 5 nM, 1 nM, 500 pM, 100 pM, 50 pM, 10 pM, 5 pM, and/or 1 pM. In some embodiments, the anti-KLK5 antibody has an IC₅₀ value of between about any of 50 μM-1 μM, 1 μM-500 nM, 500 nM-100 nM, 100 nM-10 nM, 10 nM-1 nM, 1000 pM-500 pM, 500 pM-200 pM, 200 pM-150 pM, 150 pM-100 pM, 100 pM-10 pM, and/or 10 pM-1 pM.

Antibodies provided herein are useful, e.g., for the diagnosis or treatment of disease selected from the group consisting of Netherton Syndrome, asthma, atopic dermatitis, psoriasis, and rosacea. In some embodiments, the disease is selected from the group consisting of Netherton Syndrome, asthma, atopic dermatitis, psoriasis, rosacea and eosinophilic esophagitis. In some embodiments, the asthma is persistent chronic severe asthma with acute events of worsening symptoms (exacerbations or flares) that can be life threatening. In some embodiments, the asthma is atopic (also known as allergic) asthma, non-allergic asthma (e.g., often triggered by infection with a respiratory virus (e.g., influenza, parainfluenza, rhinovirus, human metapneumovirus, and respiratory syncytial virus) or inhaled irritant (air pollutants, smog, diesel particles, volatile chemicals and gases indoors or outdoors, or even by cold dry air). In some embodiments, the asthma is intermittent or exercise-induced, asthma due to acute or chronic primary or second-hand exposure to “smoke” (typically cigarettes, cigars, pipes), inhaling or “vaping” (tobacco, marijuana or other such substances), or asthma triggered by recent ingestion of aspirin or related NSAIDS. In some embodiments, the asthma is mild, or corticosteroid naïve asthma, newly diagnosed and untreated asthma, or not previously requiring chronic use of inhaled topical or systemic steroids to control the symptoms (cough, wheeze, shortness of breath/breathlessness, or chest pain). In some embodiments, the asthma is chronic, corticosteroid resistant asthma, corticosteroid refractory asthma, asthma uncontrolled on corticosteroids or other chronic asthma controller medications. In some embodiments, the asthma is moderate to severe asthma. In some embodiments, the asthma is Th2-high asthma. In some embodiments, the asthma is severe asthma. In some embodiments, the asthma is atopic asthma, allergic asthma, non-allergic asthma (e.g., due to infection and/or respiratory syncytial virus (RSV)), exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids. In some embodiments, the asthma is T helper lymphocyte type 2 (Th2) or type 2 (Th2) high, or Type 2 (T2)-driven asthma. In some embodiments, the asthma is eosinophilic asthma. In some embodiments, the asthma is allergic asthma. In some embodiments, the subject has been determined to be Eosinophilic Inflammation Positive (EIP). See WO2015/061441. In some embodiments, the asthma is periostin-high asthma (e.g., having periostin level at least about any of 20 ng/mL, 25 ng/mL, or 50 ng/mL serum). In some embodiments, the asthma is eosinophil-high asthma (e.g., at least about any of 150, 200, 250, 300, 350, 400 eosinophil counts/ml blood). In some embodiments, the asthma is Th2-low asthma or nonTh2-driven asthma. In some embodiments, the subject has been determined to be Eosinophilic Inflammation Negative (EIN). See WO2015/061441. In some embodiments, the asthma is periostin-low asthma (e.g., having periostin level less than about 20 ng/mL serum). In some embodiments, the asthma is eosinophil-low asthma (e.g., less than about 150 eosinophil counts/μl blood or less than about 100 eosinophil counts/μl blood).

### A. Exemplary Anti-KLK5 Antibodies

Provided herein are isolated antibodies that bind to KLK5. In one embodiment, the antibody inhibits the biological activity of KLK5 by at least 50%. KLK5 is a (chymo)-trypsin-like serine proteases that is expressed in human skin, specifically in the upper spinous and granular layers of the skin. KLK5 is known to play pathological roles in skin disorders such as Netherton Syndrome.

An exemplary naturally occurring human KLK5 precursor protein sequence, with signal peptide (amino acids 1-22) is provided in SEQ ID NO:1. The corresponding mature KLK5 protein sequence corresponding to amino acids 23-293 of SEQ ID NO:1 is provided in SEQ ID NO:2. Exemplary variants of human KLK5 precursor proteins with signal peptides containing either one or both of the amino acid exchanges R55 and N153 are provided in SEQ ID NO:3, SEQ ID NO:4, SEQ ID NO:5, SEQ ID NO:6, SEQ ID NO:7 and SEQ ID NO:8, respectively.

In certain embodiments, an anti-KLK5 antibody has one or more of the following characteristics, in any combination:
a) inhibits the biological activity of KLK5 by at least 50%, at least 60%, at least 70%, at least 80%, at least 90%, or 100%;
b) inhibits the serine protease activity of KLK5;
c) binds specifically to human KLK5;
d) has an IC₅₀ value of less than 10 nM, less than 5 nM, less than 3 nM, less than 2 nM, less than 1 nM, less than 0.5 nM, less than 0.1 nM; and/or
e) has an IC₅₀ value of less than 500 pM, less than 200 pM, less than 100 pM, less than 50 pM, less than 25 pM, less than 10 pM, less than 5 pM, less than 1 pM.

### Antibody 8G10, 9B6, 2-3F4, OCS, 2B11, 10H3, 9H3, 8B7, 9H5, 9F2, 10C8, 8F5, 3-3F5 and Other Embodiments

In one aspect, an anti-KLK5 antibody is provided comprising at least one, two, three, four, five, or six HVRs selected from (a) HVR-H1 comprising the amino acid sequence selected from any one of SEQ ID NOs:14-24; (b) HVR-H2 comprising the amino acid sequence selected from any one of SEQ ID NOs:32-53; (c) HVR-H3 comprising the amino acid sequence selected from any one of SEQ ID NOs:62-72; (d) HVR-L1 comprising the amino acid sequence selected from any one of SEQ ID NOs:79-91; (e) HVR-L2 comprising the amino acid sequence selected from any one of SEQ ID NOs:99-106; and (f) HVR-L3 comprising the amino acid sequence selected from any one of SEQ ID NOs:112-122.

In one aspect, an anti-KLK5 antibody is provided comprising at least one, at least two, or all three VH HVR sequences selected from (a) HVR-H1 comprising the amino acid sequence selected from any one of SEQ ID NOs:14-24; (b) HVR-H2 comprising the amino acid sequence selected from any one of SEQ ID NOs:32-53; (c) HVR-H3 comprising the amino acid sequence selected from any one of SEQ ID NOs:62-72. In one embodiment, the antibody comprises HVR-H3 comprising the amino acid sequence selected from any one of SEQ ID NOs:62-72. In another embodiment, the antibody comprises HVR-H3 comprising the amino acid sequence selected from any one of SEQ ID NOs:62-72 and HVR-L3 comprising the amino acid sequence selected from any one of SEQ ID NOs:112-122. In a further embodiment, the antibody comprises HVR-H3 comprising the amino acid sequence selected from any one of SEQ ID NOs:62-72, HVR-L3 comprising the amino acid sequence selected from any one of SEQ ID NOs:112-122, and HVR-H2 comprising the amino acid sequence selected from any one of SEQ ID NOs:32-53. In a further embodiment, the antibody comprises (a) HVR-H1 comprising the amino acid sequence selected from any one of SEQ ID NOs:14-24; (b) HVR-H2 comprising the amino acid sequence selected from any one of SEQ ID NOs:32-53; and (c) HVR-H3 comprising the amino acid sequence selected from any one of SEQ ID NOs:62-72.

In another aspect, an anti-KLK5 antibody is provided comprising at least one, at least two, or all three VL HVR sequences selected from (a) HVR-L1 comprising the amino acid sequence selected from any one of SEQ ID NOs:79-91; (b) HVR-L2 comprising the amino acid sequence selected from any one of SEQ ID NOs:99-106; and (c) HVR-L3 comprising the amino acid sequence selected from any one of SEQ ID NOs:112-122. In one embodiment, the antibody comprises (a) HVR-L1 comprising the amino acid sequence selected from any one of SEQ ID NOs:79-91; (b) HVR-L2 comprising the amino acid sequence selected from any one of SEQ ID NOs:99-106; and (c) HVR-L3 comprising the amino acid sequence selected from any one of SEQ ID NOs:112-122.

In another aspect, an anti-KLK5 antibody is provided comprises (a) a VH domain comprising at least one, at least two, or all three VH HVR sequences selected from (i) HVR-H1 comprising the amino acid sequence selected from any one of SEQ ID NOs:14-24, (ii) HVR-H2 comprising the amino acid sequence selected from any one of SEQ ID NOs:32-53, and (iii) HVR-H3 comprising the amino acid sequence selected from any one of SEQ ID NOs:62-72; and (b) a VL domain comprising at least one, at least two, or all three VL HVR sequences selected from (i) HVR-L1 comprising the amino acid sequence selected from any one of SEQ ID NOs:79-91, (ii) HVR-L2 comprising the amino acid sequence selected from any one of SEQ ID NOs:99-106, and (c) HVR-L3 comprising the amino acid sequence selected from any one of SEQ ID NOs:112-122.

In another aspect, an anti-KLK5 antibody is provided comprising (a) HVR-H1 comprising the amino acid sequence selected from any one of SEQ ID NOs:14-24; (b) HVR-H2 comprising the amino acid sequence selected from any one of SEQ ID NOs:32-53; (c) HVR-H3 comprising the amino acid sequence selected from any one of SEQ ID NOs:62-72; (d) HVR-L1 comprising the amino acid sequence selected from any one of SEQ ID NOs:79-91; (e) HVR-L2 comprising the amino acid sequence selected from any one of SEQ ID NOs:99-106; and (f) HVR-L3 comprising the amino acid sequence selected from any one of SEQ ID NOs:112-122.

In any of the above embodiments, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence selected from any one of SEQ ID NO:170-257. In certain embodiments, a VH sequence having at least any of 90%, 91%, 92%, 93%, 94%, 95%, 96%, 97%, 98%, or 99% identity to the amino acid sequence selected from any one of SEQ ID NO:170-257 contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in any one of SEQ ID NO:170-257. In certain embodiments, substitutions, insertions, or deletions occur in regions outside the HVRs (i.e., in the FRs). Optionally, the anti-KLK5 antibody comprises the VH sequence selected from any one of SEQ ID NO:170-257, including post-translational modifications of that sequence. In a particular embodiment, the VH comprises one, two or three HVRs selected from: (a) HVR-H1 comprising the amino acid sequence selected from any one of SEQ ID NOs:14-24; (b) HVR-H2 comprising the amino acid sequence selected from any one of SEQ ID NOs:32-53; (c) HVR-H3 comprising the amino acid sequence selected from any one of SEQ ID NOs:62-72.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence selected from any one of SEQ ID NO:131-161. In certain embodiments, a VL sequence having at least 90%, 91%, 92%, 93%, 94%, 95%, 96%, 97%, 98%, or 99% identity to the amino acid sequence selected from any one of SEQ ID NO:131-162 contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in the amino acid sequence selected from any one of SEQ ID NO:131-162. In certain embodiments, the substitutions, insertions, or deletions occur in regions outside the HVRs (i.e., in the FRs). Optionally, the anti-KLK5 antibody comprises the VL sequence of the amino acid sequence selected from any one of SEQ ID NO:131-162, including post-translational modifications of that sequence. In a particular embodiment, the VL comprises one, two or three HVRs selected from (a) HVR-L1 comprising the amino acid sequence selected from any one of SEQ ID NOs:79-91; (b) HVR-L2 comprising the amino acid sequence selected from any one of SEQ ID NOs:99-106; and (c) HVR-L3 comprising the amino acid sequence selected from any one of SEQ ID NOs:112-122.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:170 and SEQ ID NO:131, respectively, including post-translational modifications of those sequences. In one embodiment, the antibody comprises the VH and VL sequences in SEQ ID NO:171 and SEQ ID NO:132, respectively, including post-translational modifications of those sequences. In one embodiment, the antibody comprises the VH and VL sequences in SEQ ID NO:172 and SEQ ID NO:133, respectively, including post-translational modifications of those sequences. In one embodiment, the antibody comprises the VH and VL sequences in SEQ ID NO:201 and SEQ ID NO:139, respectively, including post-translational modifications of those sequences. In one embodiment, the antibody comprises the VH and VL sequences in SEQ ID NO:203 and SEQ ID NO:141, respectively, including post-translational modifications of those sequences. In one embodiment, the antibody comprises the VH and VL sequences in SEQ ID NO:204 and SEQ ID NO:142, respectively, including post-translational modifications of those sequences. In one embodiment, the antibody comprises the VH and VL sequences in SEQ ID NO:205 and SEQ ID NO:143, respectively, including post-translational modifications of those sequences. In one embodiment, the antibody comprises the VH and VL sequences in SEQ ID NO:206 and SEQ ID NO:144, respectively, including post-translational modifications of those sequences. In one embodiment, the antibody comprises the VH and VL sequences in SEQ ID NO:221 and SEQ ID NO:149, respectively, including post-translational modifications of those sequences. In one embodiment, the antibody comprises the VH and VL sequences in SEQ ID NO:226 and SEQ ID NO:152, respectively, including post-translational modifications of those sequences. In one embodiment, the antibody comprises the VH and VL sequences in SEQ ID NO:227 and SEQ ID NO:153, respectively, including post-translational modifications of those sequences. In one embodiment, the antibody comprises the VH and VL sequences in SEQ ID NO:228 and SEQ ID NO:154, respectively, including post-translational modifications of those sequences. In one embodiment, the antibody comprises the VH and VL sequences in SEQ ID NO:248 and SEQ ID NO:160, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of the amino acid sequence selected from any one of SEQ ID NO:170-257. In certain embodiments, an antibody is provided that, when bound to KLK5 results in a thermodynamic epitope comprising one or more of the sequences selected from the group consisting of SEQ ID NO:316, SEQ ID NO:317, SEQ ID NO:318, and SEQ ID NO:319 as measured by hydrogen exchange mass spectrometry.

In another aspect, an anti-KLK5 antibody is provided, wherein the antibody comprises a HC sequence of the amino acid sequence selected from any one of SEQ ID NO:288-306. In another aspect, an anti-KLK5 antibody is provided, wherein the antibody comprises a LC sequence of the amino acid sequence selected from any one of SEQ ID NO:265-280.

In one embodiment, the antibody comprises the HC and LC sequences in SEQ ID NO:288 and SEQ ID NO:265, respectively, including post-translational modifications of those sequences. In one embodiment, the antibody comprises the HC and LC sequences in SEQ ID NO:289 and SEQ ID NO:266, respectively, including post-translational modifications of those sequences. In one embodiment, the antibody comprises the HC and LC sequences in SEQ ID NO:290 and SEQ ID NO:267, respectively, including post-translational modifications of those sequences. In one embodiment, the antibody comprises the HC and LC sequences in SEQ ID NO:292 and SEQ ID NO:269, respectively, including post-translational modifications of those sequences. In one embodiment, the antibody comprises the HC and LC sequences in SEQ ID NO:293 and SEQ ID NO:269, respectively, including post-translational modifications of those sequences. In one embodiment, the antibody comprises the HC and LC sequences in SEQ ID NO:294 and SEQ ID NO:270, respectively, including post-translational modifications of those sequences. In one embodiment, the antibody comprises the HC and LC sequences in SEQ ID NO:295 and SEQ ID NO:271, respectively, including post-translational modifications of those sequences. In one embodiment, the antibody comprises the HC and LC sequences in SEQ ID NO:296 and SEQ ID NO:272, respectively, including post-translational modifications of those sequences. In one embodiment, the antibody comprises the HC and LC sequences in SEQ ID NO:297 and SEQ ID NO:273, respectively, including post-translational modifications of those sequences. In one embodiment, the antibody comprises the HC and LC sequences in SEQ ID NO:299 and SEQ ID NO:275, respectively, including post-translational modifications of those sequences. In one embodiment, the antibody comprises the HC and LC sequences in SEQ ID NO:300 and SEQ ID NO:275, respectively, including post-translational modifications of those sequences. In one embodiment, the antibody comprises the HC and LC sequences in SEQ ID NO:301 and SEQ ID NO:276, respectively, including post-translational modifications of those sequences. In one embodiment, the antibody comprises the HC and LC sequences in SEQ ID NO:302 and SEQ ID NO:277, respectively, including post-translational modifications of those sequences. In one embodiment, the antibody comprises the HC and LC sequences in SEQ ID NO:303 and SEQ ID NO:278, respectively, including post-translational modifications of those sequences. In one embodiment, the antibody comprises the HC and LC sequences in SEQ ID NO:305 and SEQ ID NO:280, respectively, including post-translational modifications of those sequences. In one embodiment, the antibody comprises the HC and LC sequences in SEQ ID NO:306 and SEQ ID NO:280, respectively, including post-translational modifications of those sequences.

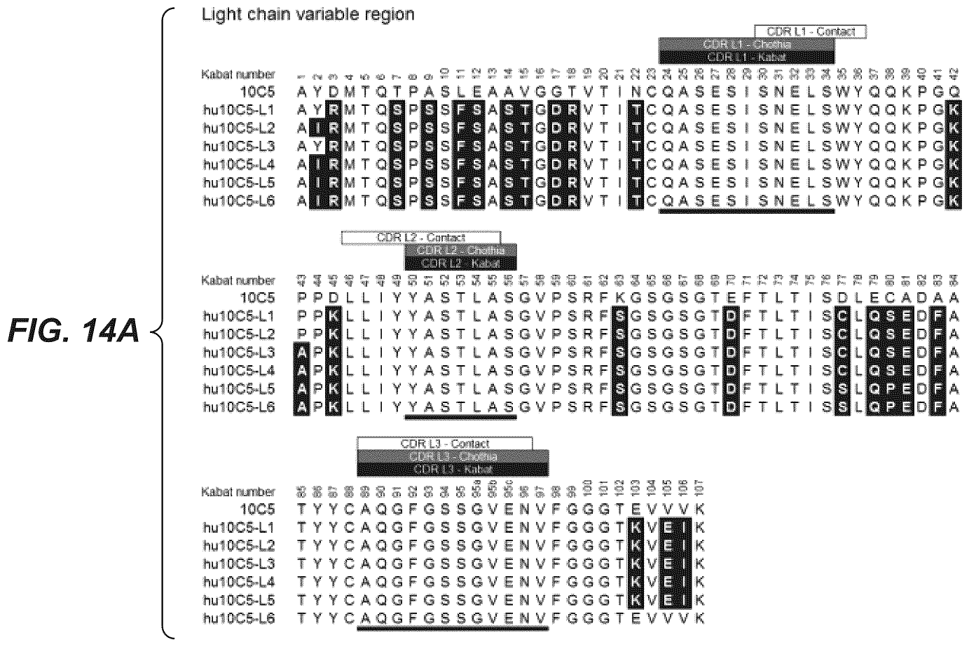

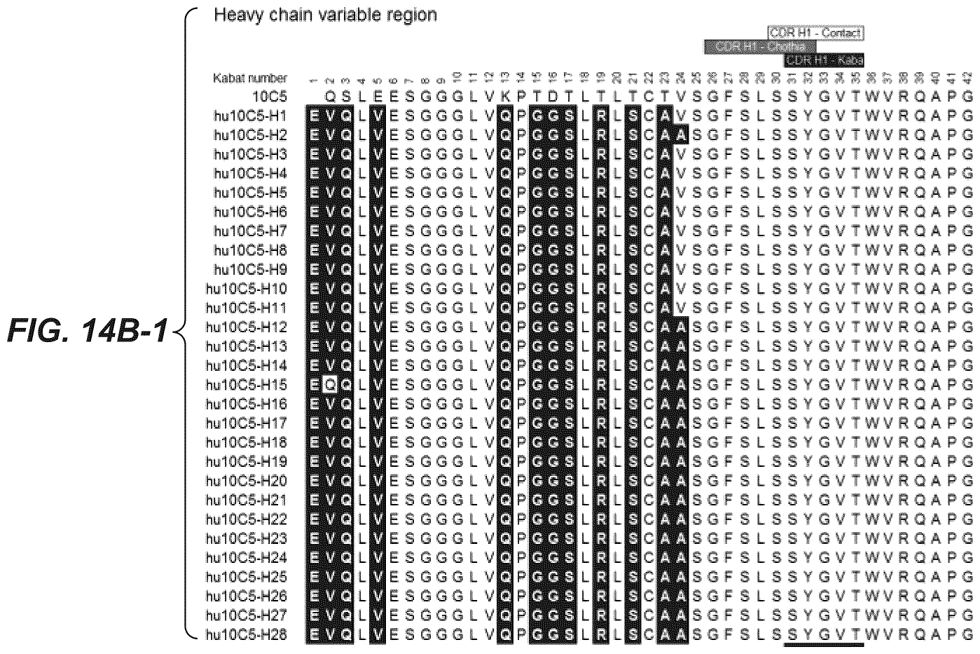

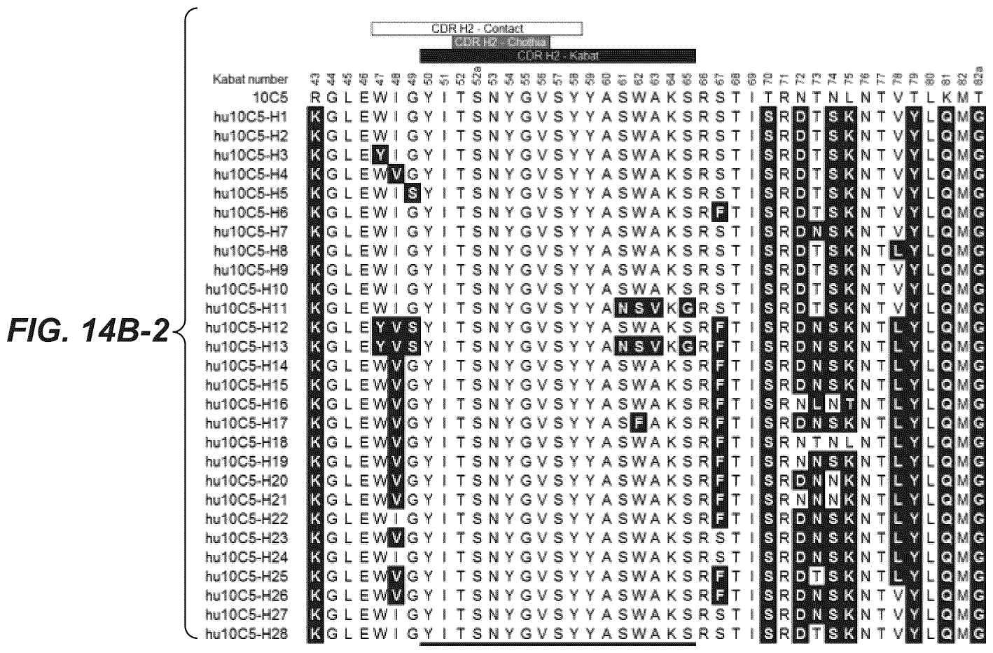

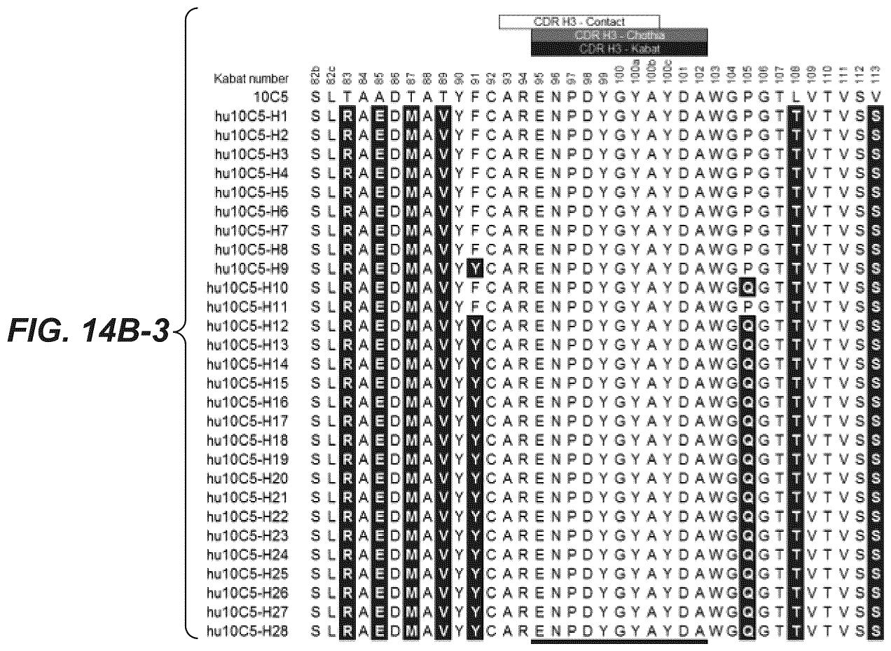

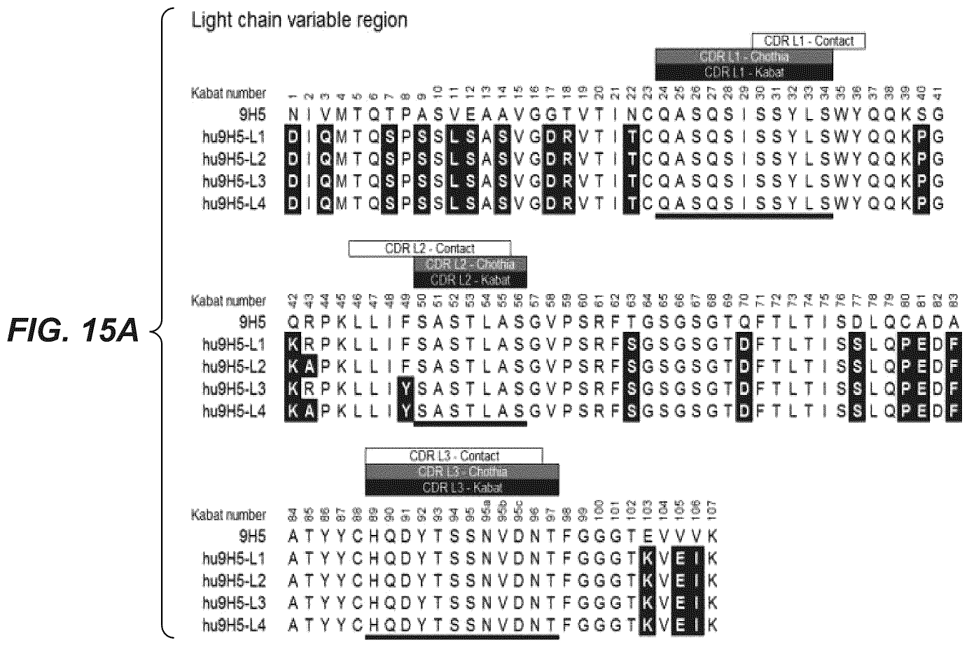

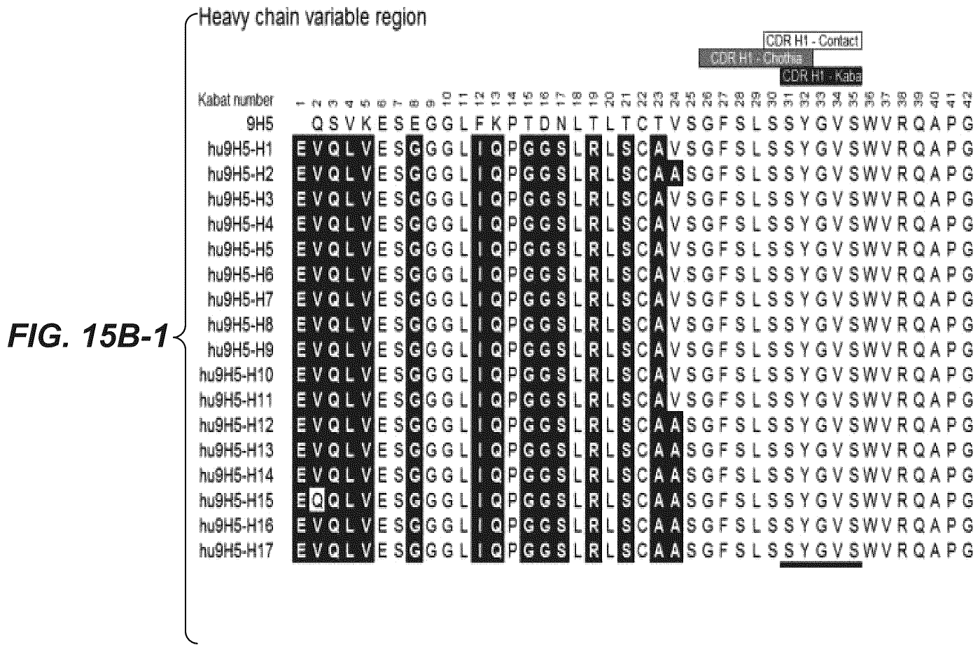

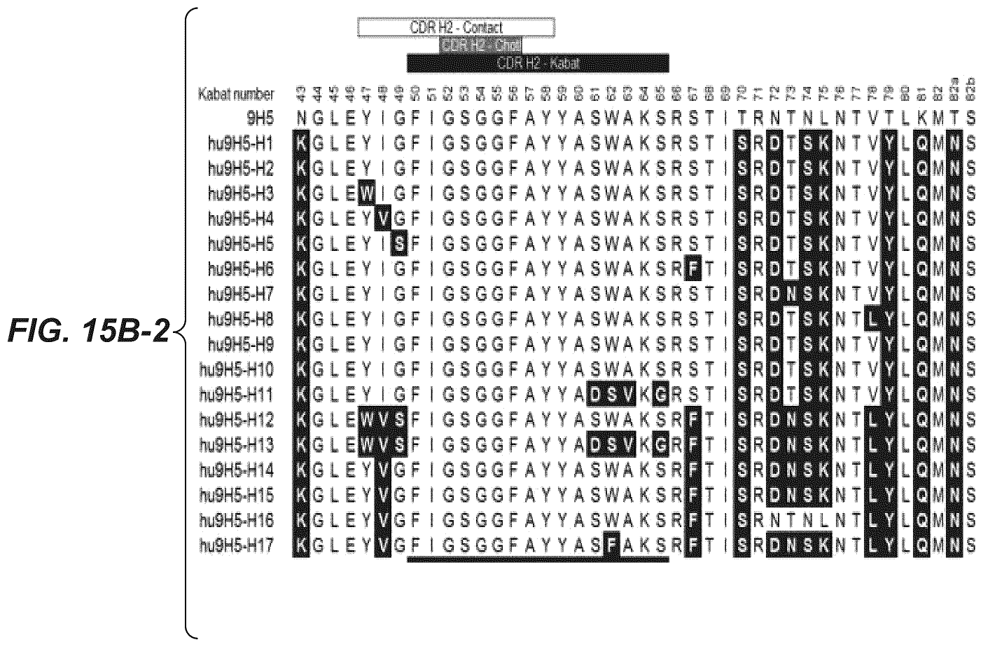

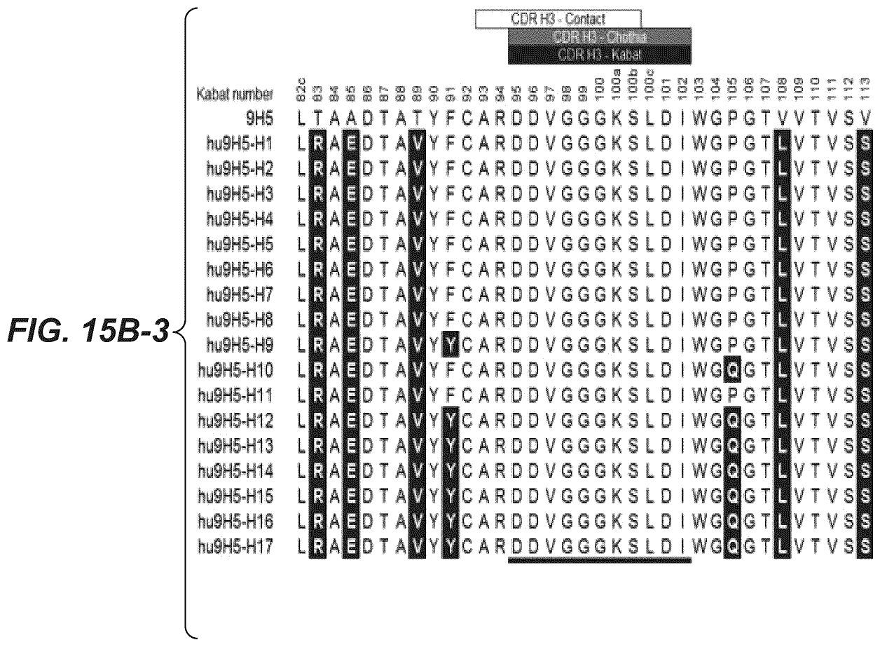

Provided herein are antibodies comprising a light chain variable domain comprising the HVR1-LC, HVR2-LC and HVR3-LC sequence according to Kabat numbering as depicted in FIGS. 14A, 15A and/or 16A and a heavy chain variable domain comprising the HVR1-HC, HVR2-HC and HVR3-HC sequence according to Kabat numbering as depicted in FIGS. 14B, 15B and/or 16B. In some embodiments, the antibody comprises a light chain variable domain comprising the HVR1-LC, HVR2-LC and/or HVR3-LC sequence, and the FR1-LC, FR2-LC, FR3-LC and/or FR4-LC sequence as depicted in FIGS. 14A, 15A and/or 16A. In some embodiments, the antibody comprises a heavy chain variable domain comprising the HVR1-HC, HVR2-HC and/or HVR3-HC sequence, and the FR1-HC, FR2-HC, FR3-HC and/or FR4-HC sequence as depicted in FIGS. 14B, 15B and/or 16B.

In a further aspect, an anti-KLK5 antibody is provided that binds to the same epitope as an anti-KLK5 antibody provided herein.

In a further aspect, an anti-KLK5 antibody is provided that, when bound to KLK5 results in a thermodynamic epitope comprising one or more of the sequences selected from the group consisting of SEQ ID NO:316, SEQ ID NO:317, SEQ ID NO:318, and SEQ ID NO:319 as measured by hydrogen exchange mass spectrometry.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework. In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

In a further aspect, an anti-KLK5 antibody according to any of the above embodiments may incorporate any of the features, singly or in combination, as described below.

### Antibody OCS, 9H5, 3-3F5 and Other Embodiments

In one aspect, an anti-KLK5 antibody is provided comprising at least one, two, three, four, five, or six HVRs selected from (a) HVR-H1 comprising the amino acid sequence of SEQ ID NO:28; (b) HVR-H2 comprising the amino acid sequence of SEQ ID NO:38, SEQ ID NO:45, or SEQ ID NO:54; (c) HVR-H3 comprising the amino acid sequence of SEQ ID NO:65, SEQ ID NO:69, or SEQ ID NO:72; (d) HVR-L1 comprising the amino acid sequence of SEQ ID NO:96; (e) HVR-L2 comprising the amino acid sequence of SEQ ID NO:109; and (f) HVR-L3 comprising the amino acid sequence of SEQ ID NO:127.

In one aspect, an anti-KLK5 antibody is provided comprising at least one, two, three, four, five, or six HVRs selected from (a) HVR-H1 comprising the amino acid sequence of SEQ ID NO:17, SEQ ID NO:22, or SEQ ID NO:24; (b) HVR-H2 comprising the amino acid sequence of SEQ ID NO:35, SEQ ID NO:36, SEQ ID NO:37, SEQ ID NO:42, SEQ ID NO:43, SEQ ID NO:44, SEQ ID NO:48, SEQ ID NO:49, SEQ ID NO:50, SEQ ID NO:51, SEQ ID NO:52, or SEQ ID NO:53; (c) HVR-H3 comprising the amino acid sequence of SEQ ID NO:65, SEQ ID NO:69, or SEQ ID NO:72; (d) HVR-L1 comprising the amino acid sequence of SEQ ID NO:82, SEQ ID NO:87, or SEQ ID NO:91; (e) HVR-L2 comprising the amino acid sequence of SEQ ID NO:99, SEQ ID NO:101, or SEQ ID NO:105; and (f) HVR-L3 comprising the amino acid sequence of SEQ ID NO:115, SEQ ID NO:119, or SEQ ID NO:122.

In one aspect, an anti-KLK5 antibody is provided comprising at least one, at least two, or all three VH HVR sequences selected from (a) HVR-H1 comprising the amino acid sequence of SEQ ID NO:17, SEQ ID NO:22, or SEQ ID NO:24; (b) HVR-H2 comprising the amino acid sequence of SEQ ID NO:35, SEQ ID NO:36, SEQ ID NO:37, SEQ ID NO:42, SEQ ID NO:43, SEQ ID NO:44, SEQ ID NO:48, SEQ ID NO:49, SEQ ID NO:50, SEQ ID NO:51, SEQ ID NO:52, or SEQ ID NO:53; and (c) HVR-H3 comprising the amino acid sequence of SEQ ID NO:65, SEQ ID NO:69, or SEQ ID NO:72. In one embodiment, the antibody comprises HVR-H3 comprising the amino acid sequence of SEQ ID NO:65, SEQ ID NO:69, or SEQ ID NO:72. In another embodiment, the antibody comprises HVR-H3 comprising the amino acid sequence of SEQ ID NO:65, SEQ ID NO:69, or SEQ ID NO:72 and HVR-L3 comprising the amino acid sequence of SEQ ID NO:115, SEQ ID NO:119, or SEQ ID NO:122. In a further embodiment, the antibody comprises HVR-H3 comprising the amino acid sequence of SEQ ID NO:65, SEQ ID NO:69, or SEQ ID NO:72, HVR-L3 comprising the amino acid sequence of SEQ ID NO:115, SEQ ID NO:119, or SEQ ID NO:122, and HVR-H2 comprising the amino acid sequence of SEQ ID NO:35, SEQ ID NO:36, SEQ ID NO:37, SEQ ID NO:42, SEQ ID NO:43, SEQ ID NO:44, SEQ ID NO:48, SEQ ID NO:49, SEQ ID NO:50, SEQ ID NO:51, SEQ ID NO:52, or SEQ ID NO:53. In a further embodiment, the antibody comprises (a) HVR-H1 comprising the amino acid sequence of SEQ ID NO:17, SEQ ID NO:22, or SEQ ID NO:24; (b) HVR-H2 comprising the amino acid sequence of SEQ ID NO:35, SEQ ID NO:36, SEQ ID NO:37, SEQ ID NO:42, SEQ ID NO:43, SEQ ID NO:44, SEQ ID NO:48, SEQ ID NO:49, SEQ ID NO:50, SEQ ID NO:51, SEQ ID NO:52, or SEQ ID NO:53; and (c) HVR-H3 comprising the amino acid sequence of SEQ ID NO:65, SEQ ID NO:69, or SEQ ID NO:72.

In another aspect, an anti-KLK5 antibody is provided comprising at least one, at least two, or all three VL HVR sequences selected from (a) HVR-L1 comprising the amino acid sequence of SEQ ID NO:82, SEQ ID NO:87, or SEQ ID NO:91; (b) HVR-L2 comprising the amino acid sequence of SEQ ID NO:99, SEQ ID NO:101, or SEQ ID NO:105; and (c) HVR-L3 comprising the amino acid sequence of SEQ ID NO:115, SEQ ID NO:119, or SEQ ID NO:122.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:17, (ii) HVR-H2 comprising the amino acid sequence of SEQ ID NO:35, and (iii) HVR-H3 comprising the amino acid sequence of SEQ ID NO:65; and (b) a VL domain comprising at least one, at least two, or all three VL HVR sequences selected from (i) HVR-L1 comprising the amino acid sequence of SEQ ID NO:82, (ii) HVR-L2 comprising the amino acid sequence of SEQ ID NO:101, and (c) HVR-L3 comprising the amino acid sequence of SEQ ID NO:115.

In another aspect, an anti-KLK5 antibody is provided comprising (a) HVR-H1 comprising the amino acid sequence of SEQ ID NO:17; (b) HVR-H2 comprising the amino acid sequence of SEQ ID NO:35; (c) HVR-H3 comprising the amino acid sequence of SEQ ID NO:65; (d) HVR-L1 comprising the amino acid sequence of SEQ ID NO:82; (e) HVR-L2 comprising the amino acid sequence of SEQ ID NO:101; and (f) HVR-L3 comprising the amino acid sequence of SEQ ID NO:115.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:22, (ii) HVR-H2 comprising the amino acid sequence of SEQ ID NO:42, and (iii) HVR-H3 comprising the amino acid sequence of SEQ ID NO:69; and (b) a VL domain comprising at least one, at least two, or all three VL HVR sequences selected from (i) HVR-L1 comprising the amino acid sequence of SEQ ID NO:87, (ii) HVR-L2 comprising the amino acid sequence of SEQ ID NO:105, and (c) HVR-L3 comprising the amino acid sequence of SEQ ID NO:119.

In another aspect, an anti-KLK5 antibody is provided comprising (a) HVR-H1 comprising the amino acid sequence of SEQ ID NO:22; (b) HVR-H2 comprising the amino acid sequence of SEQ ID NO:42; (c) HVR-H3 comprising the amino acid sequence of SEQ ID NO:69; (d) HVR-L1 comprising the amino acid sequence of SEQ ID NO:87; (e) HVR-L2 comprising the amino acid sequence of SEQ ID NO:105; and (f) HVR-L3 comprising the amino acid sequence of SEQ ID NO:119.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:24, (ii) HVR-H2 comprising the amino acid sequence of SEQ ID NO:52, and (iii) HVR-H3 comprising the amino acid sequence of SEQ ID NO:72; and (b) a VL domain comprising at least one, at least two, or all three VL HVR sequences selected from (i) HVR-L1 comprising the amino acid sequence of SEQ ID NO:91, (ii) HVR-L2 comprising the amino acid sequence of SEQ ID NO:99, and (c) HVR-L3 comprising the amino acid sequence of SEQ ID NO:122.

In another aspect, an anti-KLK5 antibody is provided comprising (a) HVR-H1 comprising the amino acid sequence of SEQ ID NO:24; (b) HVR-H2 comprising the amino acid sequence of SEQ ID NO:52; (c) HVR-H3 comprising the amino acid sequence of SEQ ID NO:72; (d) HVR-L1 comprising the amino acid sequence of SEQ ID NO:91; (e) HVR-L2 comprising the amino acid sequence of SEQ ID NO:99; and (f) HVR-L3 comprising the amino acid sequence of SEQ ID NO:122.

In another aspect, an anti-KLK5 antibody is provided comprising a heavy chain variable domain (VH) sequence having SEQ ID NO:202. In another aspect, an anti-KLK5 antibody is provided comprising a light chain variable domain (VL) sequence having SEQ ID NO:140.

In another aspect, an anti-KLK5 antibody is provided comprising a heavy chain variable domain (VH) sequence selected from any one of SEQ ID NOs:173-201. In another aspect, an anti-KLK5 antibody is provided comprising a light chain variable domain (VL) sequence selected from any one of SEQ ID NOs:134-139 and SEQ ID NO:321.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:201. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:201. In certain embodiments, substitutions, insertions, or deletions occur in regions outside the HVRs (i.e., in the FRs). Optionally, the anti-KLK5 antibody comprises the VH sequence in SEQ ID NO:201, including post-translational modifications of that sequence. In a particular embodiment, the VH comprises one, two or three HVRs selected from: (a) HVR-H1 comprising the amino acid sequence of SEQ ID NO:17, (b) HVR-H2 comprising the amino acid sequence of SEQ ID NO:35, and (c) HVR-H3 comprising the amino acid sequence of SEQ ID NO:65.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:139. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:139. In certain embodiments, the substitutions, insertions, or deletions occur in regions outside the HVRs (i.e., in the FRs). Optionally, the anti-KLK5 antibody comprises the VL sequence in SEQ ID NO:139, including post-translational modifications of that sequence. In a particular embodiment, the VL comprises one, two or three HVRs selected from (a) HVR-L1 comprising the amino acid sequence of SEQ ID NO:82; (b) HVR-L2 comprising the amino acid sequence of SEQ ID NO:101; and (c) HVR-L3 comprising the amino acid sequence of SEQ ID NO:115.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:201 and SEQ ID NO:139, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:201 and a VL sequence of SEQ ID NO:139. In certain embodiments, an antibody is provided that, when bound to KLK5 results in a thermodynamic epitope comprising one or more of the sequences selected from the group consisting of SEQ ID NO:316, SEQ ID NO:317, SEQ ID NO:318, and SEQ ID NO:319 as measured by hydrogen exchange mass spectrometry.

In another aspect, an anti-KLK5 antibody is provided comprising a heavy chain variable domain (VH) sequence having SEQ ID NO:225. In another aspect, an anti-KLK5 antibody is provided comprising a light chain variable domain (VL) sequence having SEQ ID NO:151.

In another aspect, an anti-KLK5 antibody is provided comprising a heavy chain variable domain (VH) sequence selected from any one of SEQ ID NOs:207-224. In another aspect, an anti-KLK5 antibody is provided comprising a light chain variable domain (VL) sequence selected from any one of SEQ ID NOs:145-150.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:221. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:221. In certain embodiments, substitutions, insertions, or deletions occur in regions outside the HVRs (i.e., in the FRs). Optionally, the anti-KLK5 antibody comprises the VH sequence in SEQ ID NO:221, including post-translational modifications of that sequence. In a particular embodiment, the VH comprises one, two or three HVRs selected from: (a) HVR-H1 comprising the amino acid sequence of SEQ ID NO:22, (b) HVR-H2 comprising the amino acid sequence of SEQ ID NO:42, and (c) HVR-H3 comprising the amino acid sequence of SEQ ID NO:69.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:149. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:149. In certain embodiments, the substitutions, insertions, or deletions occur in regions outside the HVRs (i.e., in the FRs). Optionally, the anti-KLK5 antibody comprises the VL sequence in SEQ ID NO:149, including post-translational modifications of that sequence. In a particular embodiment, the VL comprises one, two or three HVRs selected from (a) HVR-L1 comprising the amino acid sequence of SEQ ID NO:87; (b) HVR-L2 comprising the amino acid sequence of SEQ ID NO:105; and (c) HVR-L3 comprising the amino acid sequence of SEQ ID NO:119.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:221 and SEQ ID NO:149, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:221 and a VL sequence of SEQ ID NO:149. In certain embodiments, an antibody is provided that, when bound to KLK5 results in a thermodynamic epitope comprising one or more of the sequences selected from the group consisting of SEQ ID NO:316, SEQ ID NO:317, SEQ ID NO:318, and SEQ ID NO:319 as measured by hydrogen exchange mass spectrometry.

In another aspect, an anti-KLK5 antibody is provided comprising a heavy chain variable domain (VH) sequence having SEQ ID NO:257. In another aspect, an anti-KLK5 antibody is provided comprising a light chain variable domain (VL) sequence having SEQ ID NO:162.

In another aspect, an anti-KLK5 antibody is provided comprising a heavy chain variable domain (VH) sequence selected from any one of SEQ ID NOs:229-256. In another aspect, an anti-KLK5 antibody is provided comprising a light chain variable domain (VL) sequence selected from any one of SEQ ID NOs:155-161.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:248. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:248. In certain embodiments, substitutions, insertions, or deletions occur in regions outside the HVRs (i.e., in the FRs). Optionally, the anti-KLK5 antibody comprises the VH sequence in SEQ ID NO:248, including post-translational modifications of that sequence. In a particular embodiment, the VH comprises one, two or three HVRs selected from: (a) HVR-H1 comprising the amino acid sequence of SEQ ID NO:24, (b) HVR-H2 comprising the amino acid sequence of SEQ ID NO:52, and (c) HVR-H3 comprising the amino acid sequence of SEQ ID NO:72.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:160. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:160. In certain embodiments, the substitutions, insertions, or deletions occur in regions outside the HVRs (i.e., in the FRs). Optionally, the anti-KLK5 antibody comprises the VL sequence in SEQ ID NO:160, including post-translational modifications of that sequence. In a particular embodiment, the VL comprises one, two or three HVRs selected from (a) HVR-L1 comprising the amino acid sequence of SEQ ID NO:91; (b) HVR-L2 comprising the amino acid sequence of SEQ ID NO:99; and (c) HVR-L3 comprising the amino acid sequence of SEQ ID NO:122.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:248 and SEQ ID NO:160, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:248 and a VL sequence of SEQ ID NO:160.

Provided herein are antibodies comprising a light chain variable domain comprising the HVR1-LC, HVR2-LC and HVR3-LC sequence according to Kabat numbering as depicted in FIGS. 14A, 15A and/or 16A and a heavy chain variable domain comprising the HVR1-HC, HVR2-HC and HVR3-HC sequence according to Kabat numbering as depicted in FIGS. 14B, 15B and/or 16B. In some embodiments, the antibody comprises a light chain variable domain comprising the HVR1-LC, HVR2-LC and/or HVR3-LC sequence, and the FR1-LC, FR2-LC, FR3-LC and/or FR4-LC sequence as depicted in FIGS. 14A, 15A and/or 16A. In some embodiments, the antibody comprises a heavy chain variable domain comprising the HVR1-HC, HVR2-HC and/or HVR3-HC sequence, and the FR1-HC, FR2-HC, FR3-HC and/or FR4-HC sequence as depicted in FIGS. 14B, 15B and/or 16B.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

Further provided herein is an antibody that specifically binds to human KLK5, wherein the antibody binds to an epitope on human KLK5 comprising one or more amino acid residues selected from the group consisting of Pro130, Ser131, Ala132, Gly133, Val162, Leu163, Ser164, Gln165, Lys166, Arg167, Glu169, Asp170, Ala171, Tyr172, Pro173, Arg174, Gln174A, Ile176, Asp177, Asp178, Gly184, Asp185, Lys186, Ala186A, Arg188, Asn223, Arg224, Pro225, and Lys233 according to standard protease numbering. In some embodiments, the antibody binds to an epitope on human KLK5 comprising one or more amino acid residues selected from the group consisting of Pro130, Ser131, Ala132, Val162, Leu163, Ser164, Gln165, Lys166, Arg167, Glu169, Asp170, Ala171, Tyr172, Pro173, Arg174, Gln174A, Ile176, Asp177, Asp178, Arg224, and Lys233 according to standard protease numbering. In some embodiments, the antibody binds to an epitope on human KLK5 comprising one or more amino acid residues selected from the group consisting of Pro130, Ser131, Ala132, Gly133, Val162, Leu163, Ser164, Gln165, Lys166, Arg167, Glu169, Asp170, Ala171, Tyr172, Pro173, Arg174, Gln174A, Ile176, Asp177, and Lys233 according to standard protease numbering. In some embodiments, the antibody binds to an epitope on human KLK5 comprising one or more amino acid residues selected from the group consisting of Ser131, Ala132, Gly133, Leu163, Ser164, Gln165, Lys166, Arg167, Glu169, Asp170, Ala171, Pro173, Arg174, Gly184, Asp185, Lys186, Ala186A, Arg188, Asn223, Arg224, and Pro225 according to standard protease numbering.

Further provided herein is an antibody when bound to human KLK5 results in a conformational change of human KLK5, wherein the conformational change allosterically results in the disruption of the substrate binding site and/or the active site of human KLK5.

In a further aspect, an anti-KLK5 antibody according to any of the above embodiments may incorporate any of the features, singly or in combination, as described below.

### Antibody 8G10 and Other Embodiments

In one aspect, an anti-KLK5 antibody is provided comprising at least one, two, three, four, five, or six HVRs selected from (a) HVR-H1 comprising the amino acid sequence of SEQ ID NO:14; (b) HVR-H2 comprising the amino acid sequence of SEQ ID NO:32; (c) HVR-H3 comprising the amino acid sequence of SEQ ID NO:62; (d) HVR-L1 comprising the amino acid sequence of SEQ ID NO:79; (e) HVR-L2 comprising the amino acid sequence of SEQ ID NO:99; and (f) HVR-L3 comprising the amino acid sequence of SEQ ID NO:112.

In one aspect, an anti-KLK5 antibody is provided comprising at least one, at least two, or all three VH HVR sequences selected from (a) HVR-H1 comprising the amino acid sequence of SEQ ID NO:14; (b) HVR-H2 comprising the amino acid sequence of SEQ ID NO:32; and (c) HVR-H3 comprising the amino acid sequence of SEQ ID NO:62. In one embodiment, the antibody comprises HVR-H3 comprising the amino acid sequence of SEQ ID NO:62. In another embodiment, the antibody comprises HVR-H3 comprising the amino acid sequence of SEQ ID NO:62 and HVR-L3 comprising the amino acid sequence of SEQ ID NO:112. In a further embodiment, the antibody comprises HVR-H3 comprising the amino acid sequence of SEQ ID NO:62, HVR-L3 comprising the amino acid sequence of SEQ ID NO:112, and HVR-H2 comprising the amino acid sequence of SEQ ID NO:32. In a further embodiment, the antibody comprises (a) HVR-H1 comprising the amino acid sequence of SEQ ID NO:14; (b) HVR-H2 comprising the amino acid sequence of SEQ ID NO:32; and (c) HVR-H3 comprising the amino acid sequence of SEQ ID NO:62.

In another aspect, an anti-KLK5 antibody is provided comprising at least one, at least two, or all three VL HVR sequences selected from (a) HVR-L1 comprising the amino acid sequence of SEQ ID NO:79; (b) HVR-L2 comprising the amino acid sequence of SEQ ID NO:99; and (c) HVR-L3 comprising the amino acid sequence of SEQ ID NO:112. In one embodiment, the antibody comprises (a) HVR-L1 comprising the amino acid sequence of SEQ ID NO:79; (b) HVR-L2 comprising the amino acid sequence of SEQ ID NO:99; and (c) HVR-L3 comprising the amino acid sequence of SEQ ID NO:112.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:14, (ii) HVR-H2 comprising the amino acid sequence of SEQ ID NO:32, and (iii) HVR-H3 comprising the amino acid sequence of SEQ ID NO:62; and (b) a VL domain comprising at least one, at least two, or all three VL HVR sequences selected from (i) HVR-L1 comprising the amino acid sequence of SEQ ID NO:79, (ii) HVR-L2 comprising the amino acid sequence of SEQ ID NO:99, and (c) HVR-L3 comprising the amino acid sequence of SEQ ID NO:112.

In another aspect, an anti-KLK5 antibody is provided comprising (a) HVR-H1 comprising the amino acid sequence of SEQ ID NO:14; (b) HVR-H2 comprising the amino acid sequence of SEQ ID NO:32; (c) HVR-H3 comprising the amino acid sequence of SEQ ID NO:62; (d) HVR-L1 comprising the amino acid sequence of SEQ ID NO:79; (e) HVR-L2 comprising the amino acid sequence of SEQ ID NO:99; and (f) HVR-L3 comprising the amino acid sequence of SEQ ID NO:112.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:170. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:170. In certain embodiments, substitutions, insertions, or deletions occur in regions outside the HVRs (i.e., in the FRs). Optionally, the anti-KLK5 antibody comprises the VH sequence in SEQ ID NO:170, including post-translational modifications of that sequence. In a particular embodiment, the VH comprises one, two or three HVRs selected from: (a) HVR-H1 comprising the amino acid sequence of SEQ ID NO:14, (b) HVR-H2 comprising the amino acid sequence of SEQ ID NO:32, and (c) HVR-H3 comprising the amino acid sequence of SEQ ID NO:62.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:131. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:131. In certain embodiments, the substitutions, insertions, or deletions occur in regions outside the HVRs (i.e., in the FRs). Optionally, the anti-KLK5 antibody comprises the VL sequence in SEQ ID NO:131, including post-translational modifications of that sequence. In a particular embodiment, the VL comprises one, two or three HVRs selected from (a) HVR-L1 comprising the amino acid sequence of SEQ ID NO:79; (b) HVR-L2 comprising the amino acid sequence of SEQ ID NO:99; and (c) HVR-L3 comprising the amino acid sequence of SEQ ID NO:112.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:170 and SEQ ID NO:131, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:170 and a VL sequence of SEQ ID NO:131.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

In a further aspect, an anti-KLK5 antibody according to any of the above embodiments may incorporate any of the features, singly or in combination, as described below.

### Antibody 9B6 and Other Embodiments

In one aspect, an anti-KLK5 antibody is provided comprising at least one, two, three, four, five, or six HVRs selected from (a) HVR-H1 comprising the amino acid sequence of SEQ ID NO:15; (b) HVR-H2 comprising the amino acid sequence of SEQ ID NO:33; (c) HVR-H3 comprising the amino acid sequence of SEQ ID NO:63; (d) HVR-L1 comprising the amino acid sequence of SEQ ID NO:80; (e) HVR-L2 comprising the amino acid sequence of SEQ ID NO:100; and (f) HVR-L3 comprising the amino acid sequence of SEQ ID NO:113.

In one aspect, an anti-KLK5 antibody is provided comprising at least one, at least two, or all three VH HVR sequences selected from (a) HVR-H1 comprising the amino acid sequence of SEQ ID NO:15; (b) HVR-H2 comprising the amino acid sequence of SEQ ID NO:33; and (c) HVR-H3 comprising the amino acid sequence of SEQ ID NO:63. In one embodiment, the antibody comprises HVR-H3 comprising the amino acid sequence of SEQ ID NO:63. In another embodiment, the antibody comprises HVR-H3 comprising the amino acid sequence of SEQ ID NO:63 and HVR-L3 comprising the amino acid sequence of SEQ ID NO:113. In a further embodiment, the antibody comprises HVR-H3 comprising the amino acid sequence of SEQ ID NO:63, HVR-L3 comprising the amino acid sequence of SEQ ID NO:113, and HVR-H2 comprising the amino acid sequence of SEQ ID NO:33. In a further embodiment, the antibody comprises (a) HVR-H1 comprising the amino acid sequence of SEQ ID NO:15; (b) HVR-H2 comprising the amino acid sequence of SEQ ID NO:33; and (c) HVR-H3 comprising the amino acid sequence of SEQ ID NO:63.

In another aspect, an anti-KLK5 antibody is provided comprising at least one, at least two, or all three VL HVR sequences selected from (a) HVR-L1 comprising the amino acid sequence of SEQ ID NO:80; (b) HVR-L2 comprising the amino acid sequence of SEQ ID NO:100; and (c) HVR-L3 comprising the amino acid sequence of SEQ ID NO:113. In one embodiment, the antibody comprises (a) HVR-L1 comprising the amino acid sequence of SEQ ID NO:80; (b) HVR-L2 comprising the amino acid sequence of SEQ ID NO:100; and (c) HVR-L3 comprising the amino acid sequence of SEQ ID NO:113.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:15, (ii) HVR-H2 comprising the amino acid sequence of SEQ ID NO:33, and (iii) HVR-H3 comprising the amino acid sequence of SEQ ID NO:63; and (b) a VL domain comprising at least one, at least two, or all three VL HVR sequences selected from (i) HVR-L1 comprising the amino acid sequence of SEQ ID NO:80, (ii) HVR-L2 comprising the amino acid sequence of SEQ ID NO:100, and (c) HVR-L3 comprising the amino acid sequence of SEQ ID NO:113.

In another aspect, an anti-KLK5 antibody is provided comprising (a) HVR-H1 comprising the amino acid sequence of SEQ ID NO:15; (b) HVR-H2 comprising the amino acid sequence of SEQ ID NO:33; (c) HVR-H3 comprising the amino acid sequence of SEQ ID NO:63; (d) HVR-L1 comprising the amino acid sequence of SEQ ID NO:80; (e) HVR-L2 comprising the amino acid sequence of SEQ ID NO:100; and (f) HVR-L3 comprising the amino acid sequence of SEQ ID NO:113.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:171. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:171. In certain embodiments, substitutions, insertions, or deletions occur in regions outside the HVRs (i.e., in the FRs). Optionally, the anti-KLK5 antibody comprises the VH sequence in SEQ ID NO:171, including post-translational modifications of that sequence. In a particular embodiment, the VH comprises one, two or three HVRs selected from: (a) HVR-H1 comprising the amino acid sequence of SEQ ID NO:15, (b) HVR-H2 comprising the amino acid sequence of SEQ ID NO:33, and (c) HVR-H3 comprising the amino acid sequence of SEQ ID NO:63.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:132. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:132. In certain embodiments, the substitutions, insertions, or deletions occur in regions outside the HVRs (i.e., in the FRs). Optionally, the anti-KLK5 antibody comprises the VL sequence in SEQ ID NO:132, including post-translational modifications of that sequence. In a particular embodiment, the VL comprises one, two or three HVRs selected from (a) HVR-L1 comprising the amino acid sequence of SEQ ID NO:80; (b) HVR-L2 comprising the amino acid sequence of SEQ ID NO:100; and (c) HVR-L3 comprising the amino acid sequence of SEQ ID NO:113.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:171 and SEQ ID NO:132, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:171 and a VL sequence of SEQ ID NO:132.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

In a further aspect, an anti-KLK5 antibody according to any of the above embodiments may incorporate any of the features, singly or in combination, as described below.

### Antibody 2-3F4 and Other Embodiments

In one aspect, an anti-KLK5 antibody is provided comprising at least one, two, three, four, five, or six HVRs selected from (a) HVR-H1 comprising the amino acid sequence of SEQ ID NO:16; (b) HVR-H2 comprising the amino acid sequence of SEQ ID NO:34; (c) HVR-H3 comprising the amino acid sequence of SEQ ID NO:64; (d) HVR-L1 comprising the amino acid sequence of SEQ ID NO:81; (e) HVR-L2 comprising the amino acid sequence of SEQ ID NO:99; and (f) HVR-L3 comprising the amino acid sequence of SEQ ID NO:114.

In one aspect, an anti-KLK5 antibody is provided comprising at least one, at least two, or all three VH HVR sequences selected from (a) HVR-H1 comprising the amino acid sequence of SEQ ID NO:16; (b) HVR-H2 comprising the amino acid sequence of SEQ ID NO:34; and (c) HVR-H3 comprising the amino acid sequence of SEQ ID NO:64. In one embodiment, the antibody comprises HVR-H3 comprising the amino acid sequence of SEQ ID NO:64. In another embodiment, the antibody comprises HVR-H3 comprising the amino acid sequence of SEQ ID NO:64 and HVR-L3 comprising the amino acid sequence of SEQ ID NO:114. In a further embodiment, the antibody comprises HVR-H3 comprising the amino acid sequence of SEQ ID NO:64, HVR-L3 comprising the amino acid sequence of SEQ ID NO:114, and HVR-H2 comprising the amino acid sequence of SEQ ID NO:34. In a further embodiment, the antibody comprises (a) HVR-H1 comprising the amino acid sequence of SEQ ID NO:16; (b) HVR-H2 comprising the amino acid sequence of SEQ ID NO:34; and (c) HVR-H3 comprising the amino acid sequence of SEQ ID NO:64.

In another aspect, an anti-KLK5 antibody is provided comprising at least one, at least two, or all three VL HVR sequences selected from (a) HVR-L1 comprising the amino acid sequence of SEQ ID NO:81; (b) HVR-L2 comprising the amino acid sequence of SEQ ID NO:99; and (c) HVR-L3 comprising the amino acid sequence of SEQ ID NO:114. In one embodiment, the antibody comprises (a) HVR-L1 comprising the amino acid sequence of SEQ ID NO:81; (b) HVR-L2 comprising the amino acid sequence of SEQ ID NO:99; and (c) HVR-L3 comprising the amino acid sequence of SEQ ID NO:114.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:16, (ii) HVR-H2 comprising the amino acid sequence of SEQ ID NO:34, and (iii) HVR-H3 comprising the amino acid sequence of SEQ ID NO:64; and (b) a VL domain comprising at least one, at least two, or all three VL HVR sequences selected from (i) HVR-L1 comprising the amino acid sequence of SEQ ID NO:81, (ii) HVR-L2 comprising the amino acid sequence of SEQ ID NO:99, and (c) HVR-L3 comprising the amino acid sequence of SEQ ID NO:114.

In another aspect, an anti-KLK5 antibody is provided comprising (a) HVR-H1 comprising the amino acid sequence of SEQ ID NO:16; (b) HVR-H2 comprising the amino acid sequence of SEQ ID NO:34; (c) HVR-H3 comprising the amino acid sequence of SEQ ID NO:64; (d) HVR-L1 comprising the amino acid sequence of SEQ ID NO:81; (e) HVR-L2 comprising the amino acid sequence of SEQ ID NO:99; and (f) HVR-L3 comprising the amino acid sequence of SEQ ID NO:114.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:172. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:172. In certain embodiments, substitutions, insertions, or deletions occur in regions outside the HVRs (i.e., in the FRs). Optionally, the anti-KLK5 antibody comprises the VH sequence in SEQ ID NO:172, including post-translational modifications of that sequence. In a particular embodiment, the VH comprises one, two or three HVRs selected from: (a) HVR-H1 comprising the amino acid sequence of SEQ ID NO:16, (b) HVR-H2 comprising the amino acid sequence of SEQ ID NO:34, and (c) HVR-H3 comprising the amino acid sequence of SEQ ID NO:64.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:133. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:133. In certain embodiments, the substitutions, insertions, or deletions occur in regions outside the HVRs (i.e., in the FRs). Optionally, the anti-KLK5 antibody comprises the VL sequence in SEQ ID NO:133, including post-translational modifications of that sequence. In a particular embodiment, the VL comprises one, two or three HVRs selected from (a) HVR-L1 comprising the amino acid sequence of SEQ ID NO:81; (b) HVR-L2 comprising the amino acid sequence of SEQ ID NO:99; and (c) HVR-L3 comprising the amino acid sequence of SEQ ID NO:114.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:172 and SEQ ID NO:133, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:172 and a VL sequence of SEQ ID NO:133.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

In a further aspect, an anti-KLK5 antibody according to any of the above embodiments may incorporate any of the features, singly or in combination, as described below.

### Antibody OCS and Other Embodiments

In one aspect, an anti-KLK5 antibody is provided comprising at least one, two, three, four, five, or six HVRs selected from (a) HVR-H1 comprising the amino acid sequence of SEQ ID NO:17; (b) HVR-H2 comprising the amino acid sequence of SEQ ID NO:35; (c) HVR-H3 comprising the amino acid sequence of SEQ ID NO:65; (d) HVR-L1 comprising the amino acid sequence of SEQ ID NO:82; (e) HVR-L2 comprising the amino acid sequence of SEQ ID NO:101; and (f) HVR-L3 comprising the amino acid sequence of SEQ ID NO:115.

In one aspect, an anti-KLK5 antibody is provided comprising at least one, at least two, or all three VH HVR sequences selected from (a) HVR-H1 comprising the amino acid sequence of SEQ ID NO:17; (b) HVR-H2 comprising the amino acid sequence of SEQ ID NO:35; and (c) HVR-H3 comprising the amino acid sequence of SEQ ID NO:65. In one embodiment, the antibody comprises HVR-H3 comprising the amino acid sequence of SEQ ID NO:65. In another embodiment, the antibody comprises HVR-H3 comprising the amino acid sequence of SEQ ID NO:65 and HVR-L3 comprising the amino acid sequence of SEQ ID NO:115. In a further embodiment, the antibody comprises HVR-H3 comprising the amino acid sequence of SEQ ID NO:65, HVR-L3 comprising the amino acid sequence of SEQ ID NO:115, and HVR-H2 comprising the amino acid sequence of SEQ ID NO:35. In a further embodiment, the antibody comprises (a) HVR-H1 comprising the amino acid sequence of SEQ ID NO:17; (b) HVR-H2 comprising the amino acid sequence of SEQ ID NO:35; and (c) HVR-H3 comprising the amino acid sequence of SEQ ID NO:65.

In another aspect, an anti-KLK5 antibody is provided comprising at least one, at least two, or all three VL HVR sequences selected from (a) HVR-L1 comprising the amino acid sequence of SEQ ID NO:82; (b) HVR-L2 comprising the amino acid sequence of SEQ ID NO:101; and (c) HVR-L3 comprising the amino acid sequence of SEQ ID NO:115. In one embodiment, the antibody comprises (a) HVR-L1 comprising the amino acid sequence of SEQ ID NO:82; (b) HVR-L2 comprising the amino acid sequence of SEQ ID NO:101; and (c) HVR-L3 comprising the amino acid sequence of SEQ ID NO:115.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:17, (ii) HVR-H2 comprising the amino acid sequence of SEQ ID NO:35, and (iii) HVR-H3 comprising the amino acid sequence of SEQ ID NO:65; and (b) a VL domain comprising at least one, at least two, or all three VL HVR sequences selected from (i) HVR-L1 comprising the amino acid sequence of SEQ ID NO:82, (ii) HVR-L2 comprising the amino acid sequence of SEQ ID NO:101, and (c) HVR-L3 comprising the amino acid sequence of SEQ ID NO:115.

In another aspect, an anti-KLK5 antibody is provided comprising (a) HVR-H1 comprising the amino acid sequence of SEQ ID NO:17; (b) HVR-H2 comprising the amino acid sequence of SEQ ID NO:35; (c) HVR-H3 comprising the amino acid sequence of SEQ ID NO:65; (d) HVR-L1 comprising the amino acid sequence of SEQ ID NO:82; (e) HVR-L2 comprising the amino acid sequence of SEQ ID NO:101; and (f) HVR-L3 comprising the amino acid sequence of SEQ ID NO:115.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:201. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:201. In certain embodiments, substitutions, insertions, or deletions occur in regions outside the HVRs (i.e., in the FRs). Optionally, the anti-KLK5 antibody comprises the VH sequence in SEQ ID NO:201, including post-translational modifications of that sequence. In a particular embodiment, the VH comprises one, two or three HVRs selected from: (a) HVR-H1 comprising the amino acid sequence of SEQ ID NO:17, (b) HVR-H2 comprising the amino acid sequence of SEQ ID NO:35, and (c) HVR-H3 comprising the amino acid sequence of SEQ ID NO:65.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:139. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:139. In certain embodiments, the substitutions, insertions, or deletions occur in regions outside the HVRs (i.e., in the FRs). Optionally, the anti-KLK5 antibody comprises the VL sequence in SEQ ID NO:139, including post-translational modifications of that sequence. In a particular embodiment, the VL comprises one, two or three HVRs selected from (a) HVR-L1 comprising the amino acid sequence of SEQ ID NO:82; (b) HVR-L2 comprising the amino acid sequence of SEQ ID NO:101; and (c) HVR-L3 comprising the amino acid sequence of SEQ ID NO:115.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:201 and SEQ ID NO:139, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:201 and a VL sequence of SEQ ID NO:139. In certain embodiments, an antibody is provided that, when bound to KLK5 results in a thermodynamic epitope comprising one or more of the sequences selected from the group consisting of SEQ ID NO:316, SEQ ID NO:317, SEQ ID NO:318, and SEQ ID NO:319 as measured by hydrogen exchange mass spectrometry.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, the anti-KLK5 antibody comprises a VH domain comprising one or more heavy chain framework sequences selected from (a) a heavy chain frame work region 1 (HC-FR1) of SEQ ID NO:333, (b) a heavy chain frame work region 2 (HC-FR2) of SEQ ID NO:334, (c) a heavy chain frame work region 3 (HC-FR3) of SEQ ID NO:335, and (d) a heavy chain frame work region 4 (HC-FR4) of SEQ ID NO:336.

In one embodiment, the anti-KLK5 antibody comprises a VH domain comprising a HC-FR1 of SEQ ID NO:333. In one embodiment, the anti-KLK5 antibody comprises a VH domain comprising a HC-FR2 of SEQ ID NO:334. In one embodiment, the anti-KLK5 antibody comprises a VH domain comprising a HC-FR3 of SEQ ID NO:335. In one embodiment, the anti-KLK5 antibody comprises a VH domain comprising a HC-FR4 of SEQ ID NO:336.

In one embodiment, the anti-KLK5 antibody comprises a VL domain comprising one or more light chain framework sequences selected from (a) a light chain frame work region 1 (LC-FR1) of SEQ ID NO:329, (b) a light chain frame work region 2 (LC-FR2) of SEQ ID NO:330, (c) a light chain frame work region 3 (LC-FR3) of SEQ ID NO:331, and (d) a light chain frame work region 4 (LC-FR4) of SEQ ID NO:332.

In one embodiment, the anti-KLK5 antibody comprises a VL domain comprising a LC-FR1 of SEQ ID NO:329. In one embodiment, the anti-KLK5 antibody comprises a VL domain comprising a LC-FR2 of SEQ ID NO:330. In one embodiment, the anti-KLK5 antibody comprises a VL domain comprising a LC-FR3 of SEQ ID NO:331. In one embodiment, the anti-KLK5 antibody comprises a VL domain comprising a LC-FR4 of SEQ ID NO:332.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

Further provided herein is an antibody that specifically binds to human KLK5, wherein the antibody binds to an epitope on human KLK5 comprising one or more amino acid residues selected from the group consisting of Pro130, Ser131, Ala132, Val162, Leu163, Ser164, Gln165, Lys166, Arg167, Glu169, Asp170, Ala171, Tyr172, Pro173, Arg174, Gln174A, Ile176, Asp177, Asp178, Arg224, and Lys233 according to standard protease numbering. In one embodiment, the antibody comprises (a) HVR-H1 comprising the amino acid sequence of SEQ ID NO:17; (b) HVR-H2 comprising the amino acid sequence of SEQ ID NO:35; (c) HVR-H3 comprising the amino acid sequence of SEQ ID NO:65; (d) HVR-L1 comprising the amino acid sequence of SEQ ID NO:82; (e) HVR-L2 comprising the amino acid sequence of SEQ ID NO:101; and (f) HVR-L3 comprising the amino acid sequence of SEQ ID NO:115. In one embodiment, the antibody comprises the VH and VL sequences in SEQ ID NO:201 and SEQ ID NO:139

Further provided herein is an antibody when bound to human KLK5 results in a conformational change of human KLK5, wherein the conformational change allosterically results in the disruption of the substrate binding site and/or the active site of human KLK5. In one embodiment, the antibody comprises (a) HVR-H1 comprising the amino acid sequence of SEQ ID NO:17; (b) HVR-H2 comprising the amino acid sequence of SEQ ID NO:35; (c) HVR-H3 comprising the amino acid sequence of SEQ ID NO:65; (d) HVR-L1 comprising the amino acid sequence of SEQ ID NO:82; (e) HVR-L2 comprising the amino acid sequence of SEQ ID NO:101; and (f) HVR-L3 comprising the amino acid sequence of SEQ ID NO:115. In one embodiment, the antibody comprises the VH and VL sequences in SEQ ID NO:201 and SEQ ID NO:139.

In a further aspect, an anti-KLK5 antibody according to any of the above embodiments may incorporate any of the features, singly or in combination, as described below.

### Antibody 2B11 and Other Embodiments

In one aspect, an anti-KLK5 antibody is provided comprising at least one, two, three, four, five, or six HVRs selected from (a) HVR-H1 comprising the amino acid sequence of SEQ ID NO:18; (b) HVR-H2 comprising the amino acid sequence of SEQ ID NO:39; (c) HVR-H3 comprising the amino acid sequence of SEQ ID NO:66; (d) HVR-L1 comprising the amino acid sequence of SEQ ID NO:83; (e) HVR-L2 comprising the amino acid sequence of SEQ ID NO:102; and (f) HVR-L3 comprising the amino acid sequence of SEQ ID NO:116.

In one aspect, an anti-KLK5 antibody is provided comprising at least one, at least two, or all three VH HVR sequences selected from (a) HVR-H1 comprising the amino acid sequence of SEQ ID NO:18; (b) HVR-H2 comprising the amino acid sequence of SEQ ID NO:39; and (c) HVR-H3 comprising the amino acid sequence of SEQ ID NO:66. In one embodiment, the antibody comprises HVR-H3 comprising the amino acid sequence of SEQ ID NO:66. In another embodiment, the antibody comprises HVR-H3 comprising the amino acid sequence of SEQ ID NO:66 and HVR-L3 comprising the amino acid sequence of SEQ ID NO:116. In a further embodiment, the antibody comprises HVR-H3 comprising the amino acid sequence of SEQ ID NO:66, HVR-L3 comprising the amino acid sequence of SEQ ID NO:116, and HVR-H2 comprising the amino acid sequence of SEQ ID NO:39. In a further embodiment, the antibody comprises (a) HVR-H1 comprising the amino acid sequence of SEQ ID NO:18; (b) HVR-H2 comprising the amino acid sequence of SEQ ID NO:39; and (c) HVR-H3 comprising the amino acid sequence of SEQ ID NO:66.

In another aspect, an anti-KLK5 antibody is provided comprising at least one, at least two, or all three VL HVR sequences selected from (a) HVR-L1 comprising the amino acid sequence of SEQ ID NO:83; (b) HVR-L2 comprising the amino acid sequence of SEQ ID NO:102; and (c) HVR-L3 comprising the amino acid sequence of SEQ ID NO:116. In one embodiment, the antibody comprises (a) HVR-L1 comprising the amino acid sequence of SEQ ID NO:83; (b) HVR-L2 comprising the amino acid sequence of SEQ ID NO:102; and (c) HVR-L3 comprising the amino acid sequence of SEQ ID NO:116.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:18, (ii) HVR-H2 comprising the amino acid sequence of SEQ ID NO:39, and (iii) HVR-H3 comprising the amino acid sequence of SEQ ID NO:66; and (b) a VL domain comprising at least one, at least two, or all three VL HVR sequences selected from (i) HVR-L1 comprising the amino acid sequence of SEQ ID NO:83, (ii) HVR-L2 comprising the amino acid sequence of SEQ ID NO:102, and (c) HVR-L3 comprising the amino acid sequence of SEQ ID NO:116.

In another aspect, an anti-KLK5 antibody is provided comprising (a) HVR-H1 comprising the amino acid sequence of SEQ ID NO:18; (b) HVR-H2 comprising the amino acid sequence of SEQ ID NO:39; (c) HVR-H3 comprising the amino acid sequence of SEQ ID NO:66; (d) HVR-L1 comprising the amino acid sequence of SEQ ID NO:83; (e) HVR-L2 comprising the amino acid sequence of SEQ ID NO:102; and (f) HVR-L3 comprising the amino acid sequence of SEQ ID NO:116.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:203. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:203. In certain embodiments, substitutions, insertions, or deletions occur in regions outside the HVRs (i.e., in the FRs). Optionally, the anti-KLK5 antibody comprises the VH sequence in SEQ ID NO:203, including post-translational modifications of that sequence. In a particular embodiment, the VH comprises one, two or three HVRs selected from: (a) HVR-H1 comprising the amino acid sequence of SEQ ID NO:18, (b) HVR-H2 comprising the amino acid sequence of SEQ ID NO:39, and (c) HVR-H3 comprising the amino acid sequence of SEQ ID NO:66.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:141. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:141. In certain embodiments, the substitutions, insertions, or deletions occur in regions outside the HVRs (i.e., in the FRs). Optionally, the anti-KLK5 antibody comprises the VL sequence in SEQ ID NO:141, including post-translational modifications of that sequence. In a particular embodiment, the VL comprises one, two or three HVRs selected from (a) HVR-L1 comprising the amino acid sequence of SEQ ID NO:83; (b) HVR-L2 comprising the amino acid sequence of SEQ ID NO:102; and (c) HVR-L3 comprising the amino acid sequence of SEQ ID NO:116.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:203 and SEQ ID NO:141, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:203 and a VL sequence of SEQ ID NO:141.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

In a further aspect, an anti-KLK5 antibody according to any of the above embodiments may incorporate any of the features, singly or in combination, as described below.

### Antibody 10H3 and Other Embodiments

In one aspect, an anti-KLK5 antibody is provided comprising at least one, two, three, four, five, or six HVRs selected from (a) HVR-H1 comprising the amino acid sequence of SEQ ID NO:19; (b) HVR-H2 comprising the amino acid sequence of SEQ ID NO:40; (c) HVR-H3 comprising the amino acid sequence of SEQ ID NO:67; (d) HVR-L1 comprising the amino acid sequence of SEQ ID NO:84; (e) HVR-L2 comprising the amino acid sequence of SEQ ID NO:103; and (f) HVR-L3 comprising the amino acid sequence of SEQ ID NO:117.

In one aspect, an anti-KLK5 antibody is provided comprising at least one, at least two, or all three VH HVR sequences selected from (a) HVR-H1 comprising the amino acid sequence of SEQ ID NO:19; (b) HVR-H2 comprising the amino acid sequence of SEQ ID NO:40; and (c) HVR-H3 comprising the amino acid sequence of SEQ ID NO:67. In one embodiment, the antibody comprises HVR-H3 comprising the amino acid sequence of SEQ ID NO:67. In another embodiment, the antibody comprises HVR-H3 comprising the amino acid sequence of SEQ ID NO:67 and HVR-L3 comprising the amino acid sequence of SEQ ID NO:117. In a further embodiment, the antibody comprises HVR-H3 comprising the amino acid sequence of SEQ ID NO:67, HVR-L3 comprising the amino acid sequence of SEQ ID NO:117, and HVR-H2 comprising the amino acid sequence of SEQ ID NO:40. In a further embodiment, the antibody comprises (a) HVR-H1 comprising the amino acid sequence of SEQ ID NO:22; (b) HVR-H2 comprising the amino acid sequence of SEQ ID NO:40; and (c) HVR-H3 comprising the amino acid sequence of SEQ ID NO:67.

In another aspect, an anti-KLK5 antibody is provided comprising at least one, at least two, or all three VL HVR sequences selected from (a) HVR-L1 comprising the amino acid sequence of SEQ ID NO:84; (b) HVR-L2 comprising the amino acid sequence of SEQ ID NO:103; and (c) HVR-L3 comprising the amino acid sequence of SEQ ID NO:117. In one embodiment, the antibody comprises (a) HVR-L1 comprising the amino acid sequence of SEQ ID NO:84; (b) HVR-L2 comprising the amino acid sequence of SEQ ID NO:103; and (c) HVR-L3 comprising the amino acid sequence of SEQ ID NO:117.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:19, (ii) HVR-H2 comprising the amino acid sequence of SEQ ID NO:40, and (iii) HVR-H3 comprising the amino acid sequence of SEQ ID NO:67; and (b) a VL domain comprising at least one, at least two, or all three VL HVR sequences selected from (i) HVR-L1 comprising the amino acid sequence of SEQ ID NO:84, (ii) HVR-L2 comprising the amino acid sequence of SEQ ID NO:103, and (c) HVR-L3 comprising the amino acid sequence of SEQ ID NO:117.

In another aspect, an anti-KLK5 antibody is provided comprising (a) HVR-H1 comprising the amino acid sequence of SEQ ID NO:19; (b) HVR-H2 comprising the amino acid sequence of SEQ ID NO:40; (c) HVR-H3 comprising the amino acid sequence of SEQ ID NO:67; (d) HVR-L1 comprising the amino acid sequence of SEQ ID NO:84; (e) HVR-L2 comprising the amino acid sequence of SEQ ID NO:103; and (f) HVR-L3 comprising the amino acid sequence of SEQ ID NO:117.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:204. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:204. In certain embodiments, substitutions, insertions, or deletions occur in regions outside the HVRs (i.e., in the FRs). Optionally, the anti-KLK5 antibody comprises the VH sequence in SEQ ID NO:204, including post-translational modifications of that sequence. In a particular embodiment, the VH comprises one, two or three HVRs selected from: (a) HVR-H1 comprising the amino acid sequence of SEQ ID NO:19, (b) HVR-H2 comprising the amino acid sequence of SEQ ID NO:40, and (c) HVR-H3 comprising the amino acid sequence of SEQ ID NO:67.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:142. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:142. In certain embodiments, the substitutions, insertions, or deletions occur in regions outside the HVRs (i.e., in the FRs). Optionally, the anti-KLK5 antibody comprises the VL sequence in SEQ ID NO:142, including post-translational modifications of that sequence. In a particular embodiment, the VL comprises one, two or three HVRs selected from (a) HVR-L1 comprising the amino acid sequence of SEQ ID NO:84; (b) HVR-L2 comprising the amino acid sequence of SEQ ID NO:103; and (c) HVR-L3 comprising the amino acid sequence of SEQ ID NO:117.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:204 and SEQ ID NO:142, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:204 and a VL sequence of SEQ ID NO:142. In certain embodiments, an antibody is provided that, when bound to KLK5 results in a thermodynamic epitope comprising one or more of the sequences selected from the group consisting of SEQ ID NO:316, SEQ ID NO:317, SEQ ID NO:318, and SEQ ID NO:319 as measured by hydrogen exchange mass spectrometry.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

In a further aspect, an anti-KLK5 antibody according to any of the above embodiments may incorporate any of the features, singly or in combination, as described below.

### Antibody 9H3 and Other Embodiments

In one aspect, an anti-KLK5 antibody is provided comprising at least one, two, three, four, five, or six HVRs selected from (a) HVR-H1 comprising the amino acid sequence of SEQ ID NO:20; (b) HVR-H2 comprising the amino acid sequence of SEQ ID NO:33; (c) HVR-H3 comprising the amino acid sequence of SEQ ID NO:68; (d) HVR-L1 comprising the amino acid sequence of SEQ ID NO:85; (e) HVR-L2 comprising the amino acid sequence of SEQ ID NO:103; and (f) HVR-L3 comprising the amino acid sequence of SEQ ID NO:118.

In one aspect, an anti-KLK5 antibody is provided comprising at least one, at least two, or all three VH HVR sequences selected from (a) HVR-H1 comprising the amino acid sequence of SEQ ID NO:20; (b) HVR-H2 comprising the amino acid sequence of SEQ ID NO:33; and (c) HVR-H3 comprising the amino acid sequence of SEQ ID NO:68. In one embodiment, the antibody comprises HVR-H3 comprising the amino acid sequence of SEQ ID NO:68. In another embodiment, the antibody comprises HVR-H3 comprising the amino acid sequence of SEQ ID NO:68 and HVR-L3 comprising the amino acid sequence of SEQ ID NO:118. In a further embodiment, the antibody comprises HVR-H3 comprising the amino acid sequence of SEQ ID NO:68, HVR-L3 comprising the amino acid sequence of SEQ ID NO:118, and HVR-H2 comprising the amino acid sequence of SEQ ID NO:33. In a further embodiment, the antibody comprises (a) HVR-H1 comprising the amino acid sequence of SEQ ID NO:20; (b) HVR-H2 comprising the amino acid sequence of SEQ ID NO:33; and (c) HVR-H3 comprising the amino acid sequence of SEQ ID NO:68.

In another aspect, an anti-KLK5 antibody is provided comprising at least one, at least two, or all three VL HVR sequences selected from (a) HVR-L1 comprising the amino acid sequence of SEQ ID NO:85; (b) HVR-L2 comprising the amino acid sequence of SEQ ID NO:103; and (c) HVR-L3 comprising the amino acid sequence of SEQ ID NO:118. In one embodiment, the antibody comprises (a) HVR-L1 comprising the amino acid sequence of SEQ ID NO:85; (b) HVR-L2 comprising the amino acid sequence of SEQ ID NO:103; and (c) HVR-L3 comprising the amino acid sequence of SEQ ID NO:118.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:20, (ii) HVR-H2 comprising the amino acid sequence of SEQ ID NO:33, and (iii) HVR-H3 comprising the amino acid sequence of SEQ ID NO:68; and (b) a VL domain comprising at least one, at least two, or all three VL HVR sequences selected from (i) HVR-L1 comprising the amino acid sequence of SEQ ID NO:85, (ii) HVR-L2 comprising the amino acid sequence of SEQ ID NO:103, and (c) HVR-L3 comprising the amino acid sequence of SEQ ID NO:118.

In another aspect, an anti-KLK5 antibody is provided comprising (a) HVR-H1 comprising the amino acid sequence of SEQ ID NO:20; (b) HVR-H2 comprising the amino acid sequence of SEQ ID NO:33; (c) HVR-H3 comprising the amino acid sequence of SEQ ID NO:68; (d) HVR-L1 comprising the amino acid sequence of SEQ ID NO:85; (e) HVR-L2 comprising the amino acid sequence of SEQ ID NO:103; and (f) HVR-L3 comprising the amino acid sequence of SEQ ID NO:118.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:205. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:205. In certain embodiments, substitutions, insertions, or deletions occur in regions outside the HVRs (i.e., in the FRs). Optionally, the anti-KLK5 antibody comprises the VH sequence in SEQ ID NO:205, including post-translational modifications of that sequence. In a particular embodiment, the VH comprises one, two or three HVRs selected from: (a) HVR-H1 comprising the amino acid sequence of SEQ ID NO:20, (b) HVR-H2 comprising the amino acid sequence of SEQ ID NO:33, and (c) HVR-H3 comprising the amino acid sequence of SEQ ID NO:68.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:143. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:143. In certain embodiments, the substitutions, insertions, or deletions occur in regions outside the HVRs (i.e., in the FRs). Optionally, the anti-KLK5 antibody comprises the VL sequence in SEQ ID NO:143, including post-translational modifications of that sequence. In a particular embodiment, the VL comprises one, two or three HVRs selected from (a) HVR-L1 comprising the amino acid sequence of SEQ ID NO:85; (b) HVR-L2 comprising the amino acid sequence of SEQ ID NO:103; and (c) HVR-L3 comprising the amino acid sequence of SEQ ID NO:118.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:205 and SEQ ID NO:1143, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:205 and a VL sequence of SEQ ID NO:143.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

In a further aspect, an anti-KLK5 antibody according to any of the above embodiments may incorporate any of the features, singly or in combination, as described below.

### Antibody 8B7 and Other Embodiments

In one aspect, an anti-KLK5 antibody is provided comprising at least one, two, three, four, five, or six HVRs selected from (a) HVR-H1 comprising the amino acid sequence of SEQ ID NO:21; (b) HVR-H2 comprising the amino acid sequence of SEQ ID NO:41; (c) HVR-H3 comprising the amino acid sequence of SEQ ID NO:69; (d) HVR-L1 comprising the amino acid sequence of SEQ ID NO:86; (e) HVR-L2 comprising the amino acid sequence of SEQ ID NO:104; and (f) HVR-L3 comprising the amino acid sequence of SEQ ID NO:118.

In one aspect, an anti-KLK5 antibody is provided comprising at least one, at least two, or all three VH HVR sequences selected from (a) HVR-H1 comprising the amino acid sequence of SEQ ID NO:21; (b) HVR-H2 comprising the amino acid sequence of SEQ ID NO:41; and (c) HVR-H3 comprising the amino acid sequence of SEQ ID NO:69. In one embodiment, the antibody comprises HVR-H3 comprising the amino acid sequence of SEQ ID NO:69. In another embodiment, the antibody comprises HVR-H3 comprising the amino acid sequence of SEQ ID NO:69 and HVR-L3 comprising the amino acid sequence of SEQ ID NO:118. In a further embodiment, the antibody comprises HVR-H3 comprising the amino acid sequence of SEQ ID NO:68, HVR-L3 comprising the amino acid sequence of SEQ ID NO:118, and HVR-H2 comprising the amino acid sequence of SEQ ID NO:41. In a further embodiment, the antibody comprises (a) HVR-H1 comprising the amino acid sequence of SEQ ID NO:21; (b) HVR-H2 comprising the amino acid sequence of SEQ ID NO:41; and (c) HVR-H3 comprising the amino acid sequence of SEQ ID NO:69.

In another aspect, an anti-KLK5 antibody is provided comprising at least one, at least two, or all three VL HVR sequences selected from (a) HVR-L1 comprising the amino acid sequence of SEQ ID NO:86; (b) HVR-L2 comprising the amino acid sequence of SEQ ID NO:104; and (c) HVR-L3 comprising the amino acid sequence of SEQ ID NO:118. In one embodiment, the antibody comprises (a) HVR-L1 comprising the amino acid sequence of SEQ ID NO:86; (b) HVR-L2 comprising the amino acid sequence of SEQ ID NO:104; and (c) HVR-L3 comprising the amino acid sequence of SEQ ID NO:118.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:21, (ii) HVR-H2 comprising the amino acid sequence of SEQ ID NO:41, and (iii) HVR-H3 comprising the amino acid sequence of SEQ ID NO:69; and (b) a VL domain comprising at least one, at least two, or all three VL HVR sequences selected from (i) HVR-L1 comprising the amino acid sequence of SEQ ID NO:86, (ii) HVR-L2 comprising the amino acid sequence of SEQ ID NO:104, and (c) HVR-L3 comprising the amino acid sequence of SEQ ID NO:118.

In another aspect, an anti-KLK5 antibody is provided comprising (a) HVR-H1 comprising the amino acid sequence of SEQ ID NO:21; (b) HVR-H2 comprising the amino acid sequence of SEQ ID NO:41; (c) HVR-H3 comprising the amino acid sequence of SEQ ID NO:69; (d) HVR-L1 comprising the amino acid sequence of SEQ ID NO:86; (e) HVR-L2 comprising the amino acid sequence of SEQ ID NO:104; and (f) HVR-L3 comprising the amino acid sequence of SEQ ID NO:118.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:206. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:206. In certain embodiments, substitutions, insertions, or deletions occur in regions outside the HVRs (i.e., in the FRs). Optionally, the anti-KLK5 antibody comprises the VH sequence in SEQ ID NO:206, including post-translational modifications of that sequence. In a particular embodiment, the VH comprises one, two or three HVRs selected from: (a) HVR-H1 comprising the amino acid sequence of SEQ ID NO:21, (b) HVR-H2 comprising the amino acid sequence of SEQ ID NO:41, and (c) HVR-H3 comprising the amino acid sequence of SEQ ID NO:69.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:144. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:144. In certain embodiments, the substitutions, insertions, or deletions occur in regions outside the HVRs (i.e., in the FRs). Optionally, the anti-KLK5 antibody comprises the VL sequence in SEQ ID NO:144, including post-translational modifications of that sequence. In a particular embodiment, the VL comprises one, two or three HVRs selected from (a) HVR-L1 comprising the amino acid sequence of SEQ ID NO:86; (b) HVR-L2 comprising the amino acid sequence of SEQ ID NO:104; and (c) HVR-L3 comprising the amino acid sequence of SEQ ID NO:118.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:206 and SEQ ID NO:144, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:206 and a VL sequence of SEQ ID NO:144.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

In a further aspect, an anti-KLK5 antibody according to any of the above embodiments may incorporate any of the features, singly or in combination, as described below.

### Antibody 9H5 and Other Embodiments

In one aspect, an anti-KLK5 antibody is provided comprising at least one, two, three, four, five, or six HVRs selected from (a) HVR-H1 comprising the amino acid sequence of SEQ ID NO:22; (b) HVR-H2 comprising the amino acid sequence of SEQ ID NO:42; (c) HVR-H3 comprising the amino acid sequence of SEQ ID NO:69; (d) HVR-L1 comprising the amino acid sequence of SEQ ID NO:87; (e) HVR-L2 comprising the amino acid sequence of SEQ ID NO:105; and (f) HVR-L3 comprising the amino acid sequence of SEQ ID NO:119.

In one aspect, an anti-KLK5 antibody is provided comprising at least one, at least two, or all three VH HVR sequences selected from (a) HVR-H1 comprising the amino acid sequence of SEQ ID NO:22; (b) HVR-H2 comprising the amino acid sequence of SEQ ID NO:42; and (c) HVR-H3 comprising the amino acid sequence of SEQ ID NO:69. In one embodiment, the antibody comprises HVR-H3 comprising the amino acid sequence of SEQ ID NO:69. In another embodiment, the antibody comprises HVR-H3 comprising the amino acid sequence of SEQ ID NO:69 and HVR-L3 comprising the amino acid sequence of SEQ ID NO:119. In a further embodiment, the antibody comprises HVR-H3 comprising the amino acid sequence of SEQ ID NO:69, HVR-L3 comprising the amino acid sequence of SEQ ID NO:119, and HVR-H2 comprising the amino acid sequence of SEQ ID NO:42. In a further embodiment, the antibody comprises (a) HVR-H1 comprising the amino acid sequence of SEQ ID NO:22; (b) HVR-H2 comprising the amino acid sequence of SEQ ID NO:42; and (c) HVR-H3 comprising the amino acid sequence of SEQ ID NO:69.

In another aspect, an anti-KLK5 antibody is provided comprising at least one, at least two, or all three VL HVR sequences selected from (a) HVR-L1 comprising the amino acid sequence of SEQ ID NO:87; (b) HVR-L2 comprising the amino acid sequence of SEQ ID NO:105; and (c) HVR-L3 comprising the amino acid sequence of SEQ ID NO:119. In one embodiment, the antibody comprises (a) HVR-L1 comprising the amino acid sequence of SEQ ID NO:87; (b) HVR-L2 comprising the amino acid sequence of SEQ ID NO:105; and (c) HVR-L3 comprising the amino acid sequence of SEQ ID NO:119.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:22, (ii) HVR-H2 comprising the amino acid sequence of SEQ ID NO:42, and (iii) HVR-H3 comprising the amino acid sequence of SEQ ID NO:69; and (b) a VL domain comprising at least one, at least two, or all three VL HVR sequences selected from (i) HVR-L1 comprising the amino acid sequence of SEQ ID NO:87, (ii) HVR-L2 comprising the amino acid sequence of SEQ ID NO:105, and (c) HVR-L3 comprising the amino acid sequence of SEQ ID NO:119.

In another aspect, an anti-KLK5 antibody is provided comprising (a) HVR-H1 comprising the amino acid sequence of SEQ ID NO:22; (b) HVR-H2 comprising the amino acid sequence of SEQ ID NO:42; (c) HVR-H3 comprising the amino acid sequence of SEQ ID NO:69; (d) HVR-L1 comprising the amino acid sequence of SEQ ID NO:87; (e) HVR-L2 comprising the amino acid sequence of SEQ ID NO:105; and (f) HVR-L3 comprising the amino acid sequence of SEQ ID NO:119.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:221. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:221. In certain embodiments, substitutions, insertions, or deletions occur in regions outside the HVRs (i.e., in the FRs). Optionally, the anti-KLK5 antibody comprises the VH sequence in SEQ ID NO:221, including post-translational modifications of that sequence. In a particular embodiment, the VH comprises one, two or three HVRs selected from: (a) HVR-H1 comprising the amino acid sequence of SEQ ID NO:22, (b) HVR-H2 comprising the amino acid sequence of SEQ ID NO:42, and (c) HVR-H3 comprising the amino acid sequence of SEQ ID NO:69.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:149. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:149. In certain embodiments, the substitutions, insertions, or deletions occur in regions outside the HVRs (i.e., in the FRs). Optionally, the anti-KLK5 antibody comprises the VL sequence in SEQ ID NO:149, including post-translational modifications of that sequence. In a particular embodiment, the VL comprises one, two or three HVRs selected from (a) HVR-L1 comprising the amino acid sequence of SEQ ID NO:87; (b) HVR-L2 comprising the amino acid sequence of SEQ ID NO:105; and (c) HVR-L3 comprising the amino acid sequence of SEQ ID NO:119.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:221 and SEQ ID NO:149, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:221 and a VL sequence of SEQ ID NO:149. In certain embodiments, an antibody is provided that, when bound to KLK5 results in a thermodynamic epitope comprising one or more of the sequences selected from the group consisting of SEQ ID NO:316, SEQ ID NO:317, SEQ ID NO:318, and SEQ ID NO:319 as measured by hydrogen exchange mass spectrometry.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, the anti-KLK5 antibody comprises a VH domain comprising one or more heavy chain framework sequences selected from (a) a heavy chain frame work region 1 (HC-FR1) of SEQ ID NO:341, (b) a heavy chain frame work region 2 (HC-FR2) of SEQ ID NO:342, (c) a heavy chain frame work region 3 (HC-FR3) of SEQ ID NO:343, and (d) a heavy chain frame work region 4 (HC-FR4) of SEQ ID NO:344.

In one embodiment, the anti-KLK5 antibody comprises a VH domain comprising a HC-FR1 of SEQ ID NO:341. In one embodiment, the anti-KLK5 antibody comprises a VH domain comprising a HC-FR2 of SEQ ID NO:342. In one embodiment, the anti-KLK5 antibody comprises a VH domain comprising a HC-FR3 of SEQ ID NO:343. In one embodiment, the anti-KLK5 antibody comprises a VH domain comprising a HC-FR4 of SEQ ID NO:344.

In one embodiment, the anti-KLK5 antibody comprises a VL domain comprising one or more light chain framework sequences selected from (a) a light chain frame work region 1 (LC-FR1) of SEQ ID NO:337, (b) a light chain frame work region 2 (LC-FR2) of SEQ ID NO:338, (c) a light chain frame work region 3 (LC-FR3) of SEQ ID NO:339, and (d) a light chain frame work region 4 (LC-FR4) of SEQ ID NO:340.

In one embodiment, the anti-KLK5 antibody comprises a VL domain comprising a LC-FR1 of SEQ ID NO:337. In one embodiment, the anti-KLK5 antibody comprises a VL domain comprising a LC-FR2 of SEQ ID NO:338. In one embodiment, the anti-KLK5 antibody comprises a VL domain comprising a LC-FR3 of SEQ ID NO:339. In one embodiment, the anti-KLK5 antibody comprises a VL domain comprising a LC-FR4 of SEQ ID NO:340.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

Further provided herein is an antibody that specifically binds to human KLK5, wherein the antibody binds to an epitope on human KLK5 comprising one or more amino acid residues selected from the group consisting of Pro130, Ser131, Ala132, Gly133, Val162, Leu163, Ser164, Gln165, Lys166, Arg167, Glu169, Asp170, Ala171, Tyr172, Pro173, Arg174, Gln174A, Ile176, Asp177, and Lys233 according to standard protease numbering. In one embodiment, the antibody comprises (a) HVR-H1 comprising the amino acid sequence of SEQ ID NO:22; (b) HVR-H2 comprising the amino acid sequence of SEQ ID NO:42; (c) HVR-H3 comprising the amino acid sequence of SEQ ID NO:69; (d) HVR-L1 comprising the amino acid sequence of SEQ ID NO:87; (e) HVR-L2 comprising the amino acid sequence of SEQ ID NO:105; and (f) HVR-L3 comprising the amino acid sequence of SEQ ID NO:119. In one embodiment, the antibody comprises the VH and VL sequences in SEQ ID NO:221 and SEQ ID NO:149.

Further provided herein is an antibody when bound to human KLK5 results in a conformational change of human KLK5, wherein the conformational change allosterically results in the disruption of the substrate binding site and/or the active site of human KLK5. In one embodiment, the antibody comprises (a) HVR-H1 comprising the amino acid sequence of SEQ ID NO:22; (b) HVR-H2 comprising the amino acid sequence of SEQ ID NO:42; (c) HVR-H3 comprising the amino acid sequence of SEQ ID NO:69; (d) HVR-L1 comprising the amino acid sequence of SEQ ID NO:87; (e) HVR-L2 comprising the amino acid sequence of SEQ ID NO:105; and (f) HVR-L3 comprising the amino acid sequence of SEQ ID NO:119. In one embodiment, the antibody comprises the VH and VL sequences in SEQ ID NO:221 and SEQ ID NO:149.

In a further aspect, an anti-KLK5 antibody according to any of the above embodiments may incorporate any of the features, singly or in combination, as described below.

### Antibody 9F2 and Other Embodiments

In one aspect, an anti-KLK5 antibody is provided comprising at least one, two, three, four, five, or six HVRs selected from (a) HVR-H1 comprising the amino acid sequence of SEQ ID NO:22; (b) HVR-H2 comprising the amino acid sequence of SEQ ID NO:46; (c) HVR-H3 comprising the amino acid sequence of SEQ ID NO:69; (d) HVR-L1 comprising the amino acid sequence of SEQ ID NO:88; (e) HVR-L2 comprising the amino acid sequence of SEQ ID NO:105; and (f) HVR-L3 comprising the amino acid sequence of SEQ ID NO:120.

In one aspect, an anti-KLK5 antibody is provided comprising at least one, at least two, or all three VH HVR sequences selected from (a) HVR-H1 comprising the amino acid sequence of SEQ ID NO:22; (b) HVR-H2 comprising the amino acid sequence of SEQ ID NO:46; and (c) HVR-H3 comprising the amino acid sequence of SEQ ID NO:69. In one embodiment, the antibody comprises HVR-H3 comprising the amino acid sequence of SEQ ID NO:69. In another embodiment, the antibody comprises HVR-H3 comprising the amino acid sequence of SEQ ID NO:69 and HVR-L3 comprising the amino acid sequence of SEQ ID NO:120. In a further embodiment, the antibody comprises HVR-H3 comprising the amino acid sequence of SEQ ID NO:69, HVR-L3 comprising the amino acid sequence of SEQ ID NO:120, and HVR-H2 comprising the amino acid sequence of SEQ ID NO:46. In a further embodiment, the antibody comprises (a) HVR-H1 comprising the amino acid sequence of SEQ ID NO:22; (b) HVR-H2 comprising the amino acid sequence of SEQ ID NO:46; and (c) HVR-H3 comprising the amino acid sequence of SEQ ID NO:69.

In another aspect, an anti-KLK5 antibody is provided comprising at least one, at least two, or all three VL HVR sequences selected from (a) HVR-L1 comprising the amino acid sequence of SEQ ID NO:88; (b) HVR-L2 comprising the amino acid sequence of SEQ ID NO:105; and (c) HVR-L3 comprising the amino acid sequence of SEQ ID NO:120. In one embodiment, the antibody comprises (a) HVR-L1 comprising the amino acid sequence of SEQ ID NO:88; (b) HVR-L2 comprising the amino acid sequence of SEQ ID NO:105; and (c) HVR-L3 comprising the amino acid sequence of SEQ ID NO:120.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:22, (ii) HVR-H2 comprising the amino acid sequence of SEQ ID NO:46, and (iii) HVR-H3 comprising the amino acid sequence of SEQ ID NO:69; and (b) a VL domain comprising at least one, at least two, or all three VL HVR sequences selected from (i) HVR-L1 comprising the amino acid sequence of SEQ ID NO:88, (ii) HVR-L2 comprising the amino acid sequence of SEQ ID NO:105, and (c) HVR-L3 comprising the amino acid sequence of SEQ ID NO:120.

In another aspect, an anti-KLK5 antibody is provided comprising (a) HVR-H1 comprising the amino acid sequence of SEQ ID NO:22; (b) HVR-H2 comprising the amino acid sequence of SEQ ID NO:46; (c) HVR-H3 comprising the amino acid sequence of SEQ ID NO:69; (d) HVR-L1 comprising the amino acid sequence of SEQ ID NO:88; (e) HVR-L2 comprising the amino acid sequence of SEQ ID NO:105; and (f) HVR-L3 comprising the amino acid sequence of SEQ ID NO:120.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:226. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:226. In certain embodiments, substitutions, insertions, or deletions occur in regions outside the HVRs (i.e., in the FRs). Optionally, the anti-KLK5 antibody comprises the VH sequence in SEQ ID NO:226, including post-translational modifications of that sequence. In a particular embodiment, the VH comprises one, two or three HVRs selected from: (a) HVR-H1 comprising the amino acid sequence of SEQ ID NO:22, (b) HVR-H2 comprising the amino acid sequence of SEQ ID NO:46, and (c) HVR-H3 comprising the amino acid sequence of SEQ ID NO:69.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:152. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:152. In certain embodiments, the substitutions, insertions, or deletions occur in regions outside the HVRs (i.e., in the FRs). Optionally, the anti-KLK5 antibody comprises the VL sequence in SEQ ID NO:152, including post-translational modifications of that sequence. In a particular embodiment, the VL comprises one, two or three HVRs selected from (a) HVR-L1 comprising the amino acid sequence of SEQ ID NO:88; (b) HVR-L2 comprising the amino acid sequence of SEQ ID NO:105; and (c) HVR-L3 comprising the amino acid sequence of SEQ ID NO:120.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:226 and SEQ ID NO:152, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:226 and a VL sequence of SEQ ID NO:152.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

In a further aspect, an anti-KLK5 antibody according to any of the above embodiments may incorporate any of the features, singly or in combination, as described below.

### Antibody 10C8 and Other Embodiments

In one aspect, an anti-KLK5 antibody is provided comprising at least one, two, three, four, five, or six HVRs selected from (a) HVR-H1 comprising the amino acid sequence of SEQ ID NO:22; (b) HVR-H2 comprising the amino acid sequence of SEQ ID NO:42; (c) HVR-H3 comprising the amino acid sequence of SEQ ID NO:70; (d) HVR-L1 comprising the amino acid sequence of SEQ ID NO:89; (e) HVR-L2 comprising the amino acid sequence of SEQ ID NO:105; and (f) HVR-L3 comprising the amino acid sequence of SEQ ID NO:118.

In one aspect, an anti-KLK5 antibody is provided comprising at least one, at least two, or all three VH HVR sequences selected from (a) HVR-H1 comprising the amino acid sequence of SEQ ID NO:22; (b) HVR-H2 comprising the amino acid sequence of SEQ ID NO:42; and (c) HVR-H3 comprising the amino acid sequence of SEQ ID NO:70. In one embodiment, the antibody comprises HVR-H3 comprising the amino acid sequence of SEQ ID NO:70. In another embodiment, the antibody comprises HVR-H3 comprising the amino acid sequence of SEQ ID NO:70 and HVR-L3 comprising the amino acid sequence of SEQ ID NO:118. In a further embodiment, the antibody comprises HVR-H3 comprising the amino acid sequence of SEQ ID NO:70, HVR-L3 comprising the amino acid sequence of SEQ ID NO:118, and HVR-H2 comprising the amino acid sequence of SEQ ID NO:42. In a further embodiment, the antibody comprises (a) HVR-H1 comprising the amino acid sequence of SEQ ID NO:22; (b) HVR-H2 comprising the amino acid sequence of SEQ ID NO:42; and (c) HVR-H3 comprising the amino acid sequence of SEQ ID NO:70.

In another aspect, an anti-KLK5 antibody is provided comprising at least one, at least two, or all three VL HVR sequences selected from (a) HVR-L1 comprising the amino acid sequence of SEQ ID NO:89; (b) HVR-L2 comprising the amino acid sequence of SEQ ID NO:105; and (c) HVR-L3 comprising the amino acid sequence of SEQ ID NO:118. In one embodiment, the antibody comprises (a) HVR-L1 comprising the amino acid sequence of SEQ ID NO:89; (b) HVR-L2 comprising the amino acid sequence of SEQ ID NO:105; and (c) HVR-L3 comprising the amino acid sequence of SEQ ID NO:118.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:22, (ii) HVR-H2 comprising the amino acid sequence of SEQ ID NO:42, and (iii) HVR-H3 comprising the amino acid sequence of SEQ ID NO:70; and (b) a VL domain comprising at least one, at least two, or all three VL HVR sequences selected from (i) HVR-L1 comprising the amino acid sequence of SEQ ID NO:89, (ii) HVR-L2 comprising the amino acid sequence of SEQ ID NO:105, and (c) HVR-L3 comprising the amino acid sequence of SEQ ID NO:118.

In another aspect, an anti-KLK5 antibody is provided comprising (a) HVR-H1 comprising the amino acid sequence of SEQ ID NO:22; (b) HVR-H2 comprising the amino acid sequence of SEQ ID NO:42; (c) HVR-H3 comprising the amino acid sequence of SEQ ID NO:70; (d) HVR-L1 comprising the amino acid sequence of SEQ ID NO:89; (e) HVR-L2 comprising the amino acid sequence of SEQ ID NO:105; and (f) HVR-L3 comprising the amino acid sequence of SEQ ID NO:118.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:227. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:227. In certain embodiments, substitutions, insertions, or deletions occur in regions outside the HVRs (i.e., in the FRs). Optionally, the anti-KLK5 antibody comprises the VH sequence in SEQ ID NO:227, including post-translational modifications of that sequence. In a particular embodiment, the VH comprises one, two or three HVRs selected from: (a) HVR-H1 comprising the amino acid sequence of SEQ ID NO:22, (b) HVR-H2 comprising the amino acid sequence of SEQ ID NO:42, and (c) HVR-H3 comprising the amino acid sequence of SEQ ID NO:70.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:153. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:153. In certain embodiments, the substitutions, insertions, or deletions occur in regions outside the HVRs (i.e., in the FRs). Optionally, the anti-KLK5 antibody comprises the VL sequence in SEQ ID NO:153, including post-translational modifications of that sequence. In a particular embodiment, the VL comprises one, two or three HVRs selected from (a) HVR-L1 comprising the amino acid sequence of SEQ ID NO:89; (b) HVR-L2 comprising the amino acid sequence of SEQ ID NO:105; and (c) HVR-L3 comprising the amino acid sequence of SEQ ID NO:118.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:227 and SEQ ID NO:153, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:227 and a VL sequence of SEQ ID NO:153.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

In a further aspect, an anti-KLK5 antibody according to any of the above embodiments may incorporate any of the features, singly or in combination, as described below.

### Antibody 8F5 and Other Embodiments

In one aspect, an anti-KLK5 antibody is provided comprising at least one, two, three, four, five, or six HVRs selected from (a) HVR-H1 comprising the amino acid sequence of SEQ ID NO:23; (b) HVR-H2 comprising the amino acid sequence of SEQ ID NO:47; (c) HVR-H3 comprising the amino acid sequence of SEQ ID NO:71; (d) HVR-L1 comprising the amino acid sequence of SEQ ID NO:90; (e) HVR-L2 comprising the amino acid sequence of SEQ ID NO:106; and (f) HVR-L3 comprising the amino acid sequence of SEQ ID NO:121.

In one aspect, an anti-KLK5 antibody is provided comprising at least one, at least two, or all three VH HVR sequences selected from (a) HVR-H1 comprising the amino acid sequence of SEQ ID NO:23; (b) HVR-H2 comprising the amino acid sequence of SEQ ID NO:47; and (c) HVR-H3 comprising the amino acid sequence of SEQ ID NO:71. In one embodiment, the antibody comprises HVR-H3 comprising the amino acid sequence of SEQ ID NO:71. In another embodiment, the antibody comprises HVR-H3 comprising the amino acid sequence of SEQ ID NO:71 and HVR-L3 comprising the amino acid sequence of SEQ ID NO:121. In a further embodiment, the antibody comprises HVR-H3 comprising the amino acid sequence of SEQ ID NO:70, HVR-L3 comprising the amino acid sequence of SEQ ID NO:121, and HVR-H2 comprising the amino acid sequence of SEQ ID NO:47. In a further embodiment, the antibody comprises (a) HVR-H1 comprising the amino acid sequence of SEQ ID NO:23; (b) HVR-H2 comprising the amino acid sequence of SEQ ID NO:47; and (c) HVR-H3 comprising the amino acid sequence of SEQ ID NO:71.

In another aspect, an anti-KLK5 antibody is provided comprising at least one, at least two, or all three VL HVR sequences selected from (a) HVR-L1 comprising the amino acid sequence of SEQ ID NO:90; (b) HVR-L2 comprising the amino acid sequence of SEQ ID NO:106; and (c) HVR-L3 comprising the amino acid sequence of SEQ ID NO:121. In one embodiment, the antibody comprises (a) HVR-L1 comprising the amino acid sequence of SEQ ID NO:90; (b) HVR-L2 comprising the amino acid sequence of SEQ ID NO:106; and (c) HVR-L3 comprising the amino acid sequence of SEQ ID NO:121.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:23, (ii) HVR-H2 comprising the amino acid sequence of SEQ ID NO:47, and (iii) HVR-H3 comprising the amino acid sequence of SEQ ID NO:71; and (b) a VL domain comprising at least one, at least two, or all three VL HVR sequences selected from (i) HVR-L1 comprising the amino acid sequence of SEQ ID NO:90, (ii) HVR-L2 comprising the amino acid sequence of SEQ ID NO:106, and (c) HVR-L3 comprising the amino acid sequence of SEQ ID NO:121.

In another aspect, an anti-KLK5 antibody is provided comprising (a) HVR-H1 comprising the amino acid sequence of SEQ ID NO:23; (b) HVR-H2 comprising the amino acid sequence of SEQ ID NO:47; (c) HVR-H3 comprising the amino acid sequence of SEQ ID NO:71; (d) HVR-L1 comprising the amino acid sequence of SEQ ID NO:90; (e) HVR-L2 comprising the amino acid sequence of SEQ ID NO:106; and (f) HVR-L3 comprising the amino acid sequence of SEQ ID NO:121.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:228. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:228. In certain embodiments, substitutions, insertions, or deletions occur in regions outside the HVRs (i.e., in the FRs). Optionally, the anti-KLK5 antibody comprises the VH sequence in SEQ ID NO:228, including post-translational modifications of that sequence. In a particular embodiment, the VH comprises one, two or three HVRs selected from: (a) HVR-H1 comprising the amino acid sequence of SEQ ID NO:23, (b) HVR-H2 comprising the amino acid sequence of SEQ ID NO:47, and (c) HVR-H3 comprising the amino acid sequence of SEQ ID NO:71.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:154. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:154. In certain embodiments, the substitutions, insertions, or deletions occur in regions outside the HVRs (i.e., in the FRs). Optionally, the anti-KLK5 antibody comprises the VL sequence in SEQ ID NO:154, including post-translational modifications of that sequence. In a particular embodiment, the VL comprises one, two or three HVRs selected from (a) HVR-L1 comprising the amino acid sequence of SEQ ID NO:90; (b) HVR-L2 comprising the amino acid sequence of SEQ ID NO:106; and (c) HVR-L3 comprising the amino acid sequence of SEQ ID NO:121.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:228 and SEQ ID NO:154, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:228 and a VL sequence of SEQ ID NO:154.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

In a further aspect, an anti-KLK5 antibody according to any of the above embodiments may incorporate any of the features, singly or in combination, as described below.

### Antibody 3-3F5 and Other Embodiments

In one aspect, an anti-KLK5 antibody is provided comprising at least one, two, three, four, five, or six HVRs selected from (a) HVR-H1 comprising the amino acid sequence of SEQ ID NO:24; (b) HVR-H2 comprising the amino acid sequence of SEQ ID NO:52; (c) HVR-H3 comprising the amino acid sequence of SEQ ID NO:72; (d) HVR-L1 comprising the amino acid sequence of SEQ ID NO:91; (e) HVR-L2 comprising the amino acid sequence of SEQ ID NO:99; and (f) HVR-L3 comprising the amino acid sequence of SEQ ID NO:122.

In one aspect, an anti-KLK5 antibody is provided comprising at least one, at least two, or all three VH HVR sequences selected from (a) HVR-H1 comprising the amino acid sequence of SEQ ID NO:24; (b) HVR-H2 comprising the amino acid sequence of SEQ ID NO:52; and (c) HVR-H3 comprising the amino acid sequence of SEQ ID NO:72. In one embodiment, the antibody comprises HVR-H3 comprising the amino acid sequence of SEQ ID NO:72. In another embodiment, the antibody comprises HVR-H3 comprising the amino acid sequence of SEQ ID NO:72 and HVR-L3 comprising the amino acid sequence of SEQ ID NO:122. In a further embodiment, the antibody comprises HVR-H3 comprising the amino acid sequence of SEQ ID NO:72, HVR-L3 comprising the amino acid sequence of SEQ ID NO:122, and HVR-H2 comprising the amino acid sequence of SEQ ID NO:52. In a further embodiment, the antibody comprises (a) HVR-H1 comprising the amino acid sequence of SEQ ID NO:24; (b) HVR-H2 comprising the amino acid sequence of SEQ ID NO:52; and (c) HVR-H3 comprising the amino acid sequence of SEQ ID NO:72.

In another aspect, an anti-KLK5 antibody is provided comprising at least one, at least two, or all three VL HVR sequences selected from (a) HVR-L1 comprising the amino acid sequence of SEQ ID NO:91; (b) HVR-L2 comprising the amino acid sequence of SEQ ID NO:99; and (c) HVR-L3 comprising the amino acid sequence of SEQ ID NO:122. In one embodiment, the antibody comprises (a) HVR-L1 comprising the amino acid sequence of SEQ ID NO:91; (b) HVR-L2 comprising the amino acid sequence of SEQ ID NO:99; and (c) HVR-L3 comprising the amino acid sequence of SEQ ID NO:122.

In another aspect, an anti-KLK5 antibody is provided comprises (a) a VH domain comprising at least one, at least two, or all three VH HVR sequences selected from (i) HVR-H1 comprising the amino acid sequence of SEQ ID NO:24, (ii) HVR-H2 comprising the amino acid sequence of SEQ ID NO:52, and (iii) HVR-H3 comprising the amino acid sequence of SEQ ID NO:72; and (b) a VL domain comprising at least one, at least two, or all three VL HVR sequences selected from (i) HVR-L1 comprising the amino acid sequence of SEQ ID NO:91, (ii) HVR-L2 comprising the amino acid sequence of SEQ ID NO:99, and (c) HVR-L3 comprising the amino acid sequence of SEQ ID NO:122.

In another aspect, an anti-KLK5 antibody is provided comprising (a) HVR-H1 comprising the amino acid sequence of SEQ ID NO:24 (b) HVR-H2 comprising the amino acid sequence of SEQ ID NO:52; (c) HVR-H3 comprising the amino acid sequence of SEQ ID NO:72; (d) HVR-L1 comprising the amino acid sequence of SEQ ID NO:91; (e) HVR-L2 comprising the amino acid sequence of SEQ ID NO:99; and (f) HVR-L3 comprising the amino acid sequence of SEQ ID NO:122.

In another aspect, an anti-KLK5 antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:248. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:248. In certain embodiments, substitutions, insertions, or deletions occur in regions outside the HVRs (i.e., in the FRs). Optionally, the anti-KLK5 antibody comprises the VH sequence in SEQ ID NO:248, including post-translational modifications of that sequence. In a particular embodiment, the VH comprises one, two or three HVRs selected from: (a) HVR-H1 comprising the amino acid sequence of SEQ ID NO:24, (b) HVR-H2 comprising the amino acid sequence of SEQ ID NO:52, and (c) HVR-H3 comprising the amino acid sequence of SEQ ID NO:72.

In another aspect, an anti-KLK5 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:160. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-KLK5 antibody comprising that sequence retains the ability to bind to KLK5. In certain embodiments, a total of 1 to 10 amino acids have been substituted, inserted and/or deleted in SEQ ID NO:160. In certain embodiments, the substitutions, insertions, or deletions occur in regions outside the HVRs (i.e., in the FRs). Optionally, the anti-KLK5 antibody comprises the VL sequence in SEQ ID NO:160, including post-translational modifications of that sequence. In a particular embodiment, the VL comprises one, two or three HVRs selected from (a) HVR-L1 comprising the amino acid sequence of SEQ ID NO:91; (b) HVR-L2 comprising the amino acid sequence of SEQ ID NO:99; and (c) HVR-L3 comprising the amino acid sequence of SEQ ID NO:122.

In another aspect, an anti-KLK5 antibody is provided, wherein the antibody comprises a VH as in any of the embodiments provided above, and a VL as in any of the embodiments provided above. In one embodiment, the antibody comprises the VH and VL sequences in SEQ ID NO:248 and SEQ ID NO:160, respectively, including post-translational modifications of those sequences.

In a further aspect, an anti-KLK5 antibody is provided that binds to the same epitope as an anti-KLK5 antibody provided herein. For example, in certain embodiments, an antibody is provided that binds to the same epitope as an anti-KLK5 antibody comprising a VH sequence of SEQ ID NO:248 and a VL sequence of SEQ ID NO:160.

In a further aspect, an anti-KLK5 antibody according to any of the above embodiments is a monoclonal antibody, including a chimeric, humanized or human antibody. In one embodiment, an anti-KLK5 antibody is humanized. In one embodiment, an anti-KLK5 antibody comprises HVRs as in any of the above embodiments, and further comprises an acceptor human framework, e.g. a human immunoglobulin framework or a human consensus framework.

In one embodiment, the anti-KLK5 antibody comprises a VH domain comprising one or more heavy chain framework sequences selected from (a) a heavy chain frame work region 1 (HC-FR1) of SEQ ID NO:349, (b) a heavy chain frame work region 2 (HC-FR2) of SEQ ID NO:350, (c) a heavy chain frame work region 3 (HC-FR3) of SEQ ID NO:351, and (d) a heavy chain frame work region 4 (HC-FR4) of SEQ ID NO:352.

In one embodiment, the anti-KLK5 antibody comprises a VH domain comprising a HC-FR1 of SEQ ID NO:349. In one embodiment, the anti-KLK5 antibody comprises a VH domain comprising a HC-FR2 of SEQ ID NO:350. In one embodiment, the anti-KLK5 antibody comprises a VH domain comprising a HC-FR3 of SEQ ID NO:351. In one embodiment, the anti-KLK5 antibody comprises a VH domain comprising a HC-FR4 of SEQ ID NO:352.

In one embodiment, the anti-KLK5 antibody comprises a VL domain comprising one or more light chain framework sequences selected from (a) a light chain frame work region 1 (LC-FR1) of SEQ ID NO:345, (b) a light chain frame work region 2 (LC-FR2) of SEQ ID NO:346, (c) a light chain frame work region 3 (LC-FR3) of SEQ ID NO:347, and (d) a light chain frame work region 4 (LC-FR4) of SEQ ID NO:348.

In one embodiment, the anti-KLK5 antibody comprises a VL domain comprising a LC-FR1 of SEQ ID NO:345. In one embodiment, the anti-KLK5 antibody comprises a VL domain comprising a LC-FR2 of SEQ ID NO:346. In one embodiment, the anti-KLK5 antibody comprises a VL domain comprising a LC-FR3 of SEQ ID NO:347. In one embodiment, the anti-KLK5 antibody comprises a VL domain comprising a LC-FR4 of SEQ ID NO:348.

In one embodiment, an anti-KLK5 antibody is an antibody fragment, e.g., a Fv, Fab, Fab′, scFv, diabody, or F(ab′)₂ fragment. In another embodiment, the antibody is a full length antibody, e.g an intact IgG1 antibody or an intact IgG4 antibody or other antibody class or isotype as defined herein.

Further provided herein is an antibody that specifically binds to human KLK5, wherein the antibody binds to an epitope on human KLK5 comprising one or more amino acid residues selected from the group consisting of Ser131, Ala132, Gly133, Leu163, Ser164, Gln165, Lys166, Arg167, Glu169, Asp170, Ala171, Pro173, Arg174, Gly184, Asp185, Lys186, Ala186A, Arg188, Asn223, Arg224, and Pro225 according to standard protease numbering. In one embodiment, the antibody comprises (a) HVR-H1 comprising the amino acid sequence of SEQ ID NO:24 (b) HVR-H2 comprising the amino acid sequence of SEQ ID NO:52; (c) HVR-H3 comprising the amino acid sequence of SEQ ID NO:72; (d) HVR-L1 comprising the amino acid sequence of SEQ ID NO:91; (e) HVR-L2 comprising the amino acid sequence of SEQ ID NO:99; and (f) HVR-L3 comprising the amino acid sequence of SEQ ID NO:122. In one embodiment, the antibody comprises the VH and VL sequences in SEQ ID NO:248 and SEQ ID NO:160.

Further provided herein is an antibody when bound to human KLK5 results in a conformational change of human KLK5, wherein the conformational change allosterically results in the disruption of the substrate binding site and/or the active site of human KLK5. In one embodiment, the antibody comprises (a) HVR-H1 comprising the amino acid sequence of SEQ ID NO:24 (b) HVR-H2 comprising the amino acid sequence of SEQ ID NO:52; (c) HVR-H3 comprising the amino acid sequence of SEQ ID NO:72; (d) HVR-L1 comprising the amino acid sequence of SEQ ID NO:91; (e) HVR-L2 comprising the amino acid sequence of SEQ ID NO:99; and (f) HVR-L3 comprising the amino acid sequence of SEQ ID NO:122. In one embodiment, the antibody comprises the VH and VL sequences in SEQ ID NO:248 and SEQ ID NO:160.

In a further aspect, an anti-KLK5 antibody according to any of the above embodiments may incorporate any of the features, singly or in combination, as described below.

### 2. Antibody Affinity

In some embodiments of any of the anti-KLK5 antibodies, the anti-KLK5 antibody has a binding affinity (dissociation constant Kd) to KLK5 of ≤1 M, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (BIAcore, Inc., Piscataway, NJ) is performed at 25° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (−0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 3. Antibody Fragments

In some embodiments, the anti-KLK5 antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 4. Chimeric and Humanized Antibodies

In some embodiments, the anti-KLK5 antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 5. Human Antibodies

In some embodiments, the anti-KLK5 antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 6. Library-Derived Antibodies

Anti-KLK5 antibodies may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In some embodiments, the anti-KLK5 antibody provided herein is a multispecific antibody, e.g. a bispecific antibody. Multispecific antibodies are monoclonal antibodies that have binding specificities for at least two different sites. In certain embodiments, one of the binding specificities is for KLK5 and the other is for any other antigen. In certain embodiments, bispecific antibodies may bind to two different epitopes of KLK5. Bispecific antibodies may also be used to localize cytotoxic agents to cells which express KLK5. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to KLK5 as well as another, different antigen (see, US 2008/0069820, for example).

### 7. Antibody Variants

In some embodiments, amino acid sequence variants of the anti-KLK5 antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In some embodiments, anti-KLK5 antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be groped according to common side-chain properties:
(1) hydrophobic: Norleucine, Met, Ala, Val, Len, Ile;
(2) neutral hydrophilic: Cys, Ser, Thr, Asn, Gin;
(3) acidic: Asp, Gin;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### b) Glycosylation Variants

In certain embodiments, the anti-KLK5 antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In some embodiments, anti-KLK5 antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Anti-KLK5 antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In some embodiments, one or more amino acid modifications may be introduced into the Fc region of the anti-KLK5 antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc-RIII only, whereas monocytes express Fc-RI, Fc-RII and Fc-RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### d) Cysteine Engineered Antibody Variants

In some embodiments, it may be desirable to create cysteine engineered anti-KLK5 antibodies, e.g., THIOMAB™ antibodies in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In some embodiments, the anti-KLK5 antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In some embodiments, conjugates of the anti-KLK5 antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods and Compositions

Anti-KLK5 antibodies may be produced using recombinant methods and compositions, e.g., as described in U.S. Pat. No. 4,816,567. In one embodiment, isolated nucleic acid encoding an anti-KLK5 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-KLK5 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-KLK5 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.). After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### C. Assays

Anti-KLK5 antibodies provided herein may be identified, screened for, or characterized for their physical/chemical properties and/or biological activities by various assays known in the art.

### 1. Binding Assays and Other Assays

In one aspect, an antibody of the invention is tested for its antigen binding activity, e.g., by known methods such as ELISA, BIACore®, FACS, or Western blot, etc.

In another aspect, competition assays may be used to identify an antibody that competes with any of the antibodies described herein for binding to KLK5. In certain embodiments, such a competing antibody binds to the same epitope (e.g., a linear or a conformational epitope) that is bound by any of the antibodies described herein. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized KLK5 is incubated in a solution comprising a first labeled antibody that binds to KLK5 (e.g., any of the antibodies described herein) and a second unlabeled antibody that is being tested for its ability to compete with the first antibody for binding to KLK5. The second antibody may be present in a hybridoma supernatant. As a control, immobilized KLK5 is incubated in a solution comprising the first labeled antibody but not the second unlabeled antibody. After incubation under conditions permissive for binding of the first antibody to KLK5, excess unbound antibody is removed, and the amount of label associated with immobilized KLK5 is measured. If the amount of label associated with immobilized KLK5 is substantially reduced in the test sample relative to the control sample, then that indicates that the second antibody is competing with the first antibody for binding to KLK5. See Harlow and Lane (1988) Antibodies: A Laboratory Manual ch. 14 (Cold Spring Harbor Laboratory, Cold Spring Harbor, NY).

### 2. Activity Assays

In one aspect, assays are provided for identifying anti-KLK5 antibodies having biological activity. Biological activity may include, e.g. inhibition of KLK5. In one embodiment, the anti-KLK5 antibodies inhibit the serine protease activity of KLK5. Antibodies having such biological activity in vivo and/or in vitro are also provided.

In some embodiments, the anti-KLK5 antibodies are tested for such biological activity. In some embodiments, the biological activity is tested by one or more methods selected from the group consisting of a direct activity assay, fluorescent peptide assay, an LC/MS assay, and a Kᵢ₍ₐₚₚ₎ assay. In some embodiments, the biological activity is measured by one or more methods selected from the group consisting of a recombinant KLK5 direct activity assay, coupled pro-KLK1 fluorescent peptide assay, a coupled pro-KLK7 fluorescent peptide assay, a pro-KLK1 LC/MS assay, a pro-KLK7 LC/MS assay, and a Kᵢ₍ₐₚₚ₎ assay. In some embodiments, the IC₅₀ values are measured by the assays described herein above and in detail in the Examples below.

### D. Immunoconjugates

The invention also provides immunoconjugates comprising an anti-KLK5 antibody provided herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A.).

### E. Methods and Compositions for Diagnostics and Detection

In some embodiments, any of the anti-KLK5 antibodies provided herein is useful for detecting the presence of KLK5 in a biological sample. The term “detecting” as used herein encompasses quantitative or qualitative detection. In some embodiments, a biological sample comprises a cell or tissue, such as skin epidermis, lung parenchyma, bronchial sub-epithelium. In some embodiments, a biological sample comprises bronchial alveolar lavage.

In one embodiment, an anti-KLK5 antibody for use in a method of diagnosis or detection is provided. In a further aspect, a method of detecting the presence of KLK5 in a biological sample is provided. In certain embodiments, the method comprises contacting the biological sample with an anti-KLK5 antibody as described herein under conditions permissive for binding of the anti-KLK5 antibody to KLK5, and detecting whether a complex is formed between the anti-KLK5 antibody and KLK5. Such method may be an in vitro or in vivo method. In one embodiment, an anti-KLK5 antibody is used to select subjects eligible for therapy with an anti-KLK5 antibody, e.g. where KLK5 is a biomarker for selection of patients.

Exemplary disorders that may be diagnosed using an anti-KLK5 antibody of the invention include Netherton Syndrome, asthma, atopic dermatitis, psoriasis, and rosacea. In some embodiments, the disease is selected from the group consisting of Netherton Syndrome, asthma, atopic dermatitis, psoriasis, rosacea and eosinophilic esophagitis. In some embodiments, the asthma is selected from the group consisting of atopic asthma, allergic asthma, non-allergic asthma, exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, T helper lymphocyte type 2 (Th2) or type 2 (Th2) high, or Type 2 (T2)-driven asthma, eosinophilic asthma, periostin-high asthma, eosinophil-high asthma, Th2-low asthma or nonTh2-driven asthma, periostin-low asthma, and eosinophil-low asthma. In some embodiments, the asthma is TH2-low asthma.

In certain embodiments, labeled anti-KLK5 antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### F. Pharmaceutical Formulations

Pharmaceutical formulations of an anti-KLK5 antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### G. Therapeutic Methods and Compositions

Any of the anti-KLK5 antibodies provided herein may be used in therapeutic methods.

In one aspect, an anti-KLK5 antibody for use as a medicament is provided. In further aspects, an anti-KLK5 antibody is provided for use in treating a disease selected from the group consisting of Netherton Syndrome, asthma, atopic dermatitis, psoriasis, and rosacea. In some embodiments, the disease is selected from the group consisting of Netherton Syndrome, asthma, atopic dermatitis, psoriasis, rosacea and eosinophilic esophagitis. In some embodiments, the asthma is selected from the group consisting of atopic asthma, allergic asthma, non-allergic asthma, exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, T helper lymphocyte type 2 (Th2) or type 2 (Th2) high, or Type 2 (T2)-driven asthma, eosinophilic asthma, periostin-high asthma, eosinophil-high asthma, Th2-low asthma or nonTh2-driven asthma, periostin-low asthma, and eosinophil-low asthma. In some embodiments, the asthma is TH2-low asthma.

In certain embodiments, an anti-KLK5 antibody for use in a method of treatment is provided. In certain embodiments, the invention provides an anti-KLK5 antibody for use in a method of treating an individual having a disease, wherein the disease is selected from the group consisting of Netherton Syndrome, asthma, atopic dermatitis, psoriasis, and rosacea comprising administering to the individual an effective amount of the anti-KLK5 antibody. In one such embodiment, the method further comprises administering to the individual an effective amount of at least one additional therapeutic agent, e.g., as described below. In some embodiments, provided is an anti-KLK5 antibody for use in inhibiting the biological activity of KLK5. In some embodiments, provided is an anti-KLK5 antibody for use in a method of inhibiting the biological activity of KLK5 in an individual comprising administering to the individual an effective of the anti-KLK5 antibody to inhibit the biological activity of KLK5. An “individual” according to any of the above embodiments is preferably a human. In some embodiments, the disease is selected from the group consisting of Netherton Syndrome, asthma, atopic dermatitis, psoriasis, rosacea and eosinophilic esophagitis. In some embodiments, the asthma is selected from the group consisting of atopic asthma, allergic asthma, non-allergic asthma, exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, T helper lymphocyte type 2 (Th2) or type 2 (Th2) high, or Type 2 (T2)-driven asthma, eosinophilic asthma, periostin-high asthma, eosinophil-high asthma, Th2-low asthma or nonTh2-driven asthma, periostin-low asthma, and eosinophil-low asthma. In some embodiments, the asthma is TH2-low asthma.

In a further aspect, the invention provides for the use of an anti-KLK5 antibody in the manufacture or preparation of a medicament. In one embodiment, the medicament is for treatment a disease, wherein the disease is selected from the group consisting of Netherton Syndrome, asthma, atopic dermatitis, psoriasis, and rosacea. In a further embodiment, the medicament is for use in a method of treating a disease, wherein the disease is selected from the group consisting of Netherton Syndrome, asthma, atopic dermatitis, psoriasis, and rosacea comprising administering to an individual having said disease an effective amount of the medicament. In one such embodiment, the method further comprises administering to the individual an effective amount of at least one additional therapeutic agent, e.g., as described below. In a further embodiment, the medicament is for inhibiting the biological activity of KLK5. In a further embodiment, the medicament is for use in a method of inhibiting the biological activity of KLK5 in an individual comprising administering to the individual an amount effective of the medicament to inhibiting the biological activity of KLK5. An “individual” according to any of the above embodiments may be a human. In some embodiments, the disease is selected from the group consisting of Netherton Syndrome, asthma, atopic dermatitis, psoriasis, rosacea and eosinophilic esophagitis. In some embodiments, the asthma is selected from the group consisting of atopic asthma, allergic asthma, non-allergic asthma, exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, T helper lymphocyte type 2 (Th2) or type 2 (Th2) high, or Type 2 (T2)-driven asthma, eosinophilic asthma, periostin-high asthma, eosinophil-high asthma, Th2-low asthma or nonTh2-driven asthma, periostin-low asthma, and eosinophil-low asthma. In some embodiments, the asthma is TH2-low asthma.

In a further aspect, the invention provides a method for treating a disease, wherein the disease is selected from the group consisting of Netherton Syndrome, asthma, atopic dermatitis, psoriasis, and rosacea. In some embodiments, the disease is selected from the group consisting of Netherton Syndrome, asthma, atopic dermatitis, psoriasis, rosacea and eosinophilic esophagitis. In one embodiment, the method comprises administering to an individual having such Netherton Syndrome, asthma, atopic dermatitis, psoriasis, or rosacea an effective amount of an anti-KLK5 antibody. In one such embodiment, the method further comprises administering to the individual an effective amount of at least one additional therapeutic agent, as described below. An “individual” according to any of the above embodiments may be a human. In some embodiments, the asthma is selected from the group consisting of atopic asthma, allergic asthma, non-allergic asthma, exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, T helper lymphocyte type 2 (Th2) or type 2 (Th2) high, or Type 2 (T2)-driven asthma, eosinophilic asthma, periostin-high asthma, eosinophil-high asthma, Th2-low asthma or nonTh2-driven asthma, periostin-low asthma, and eosinophil-low asthma. In some embodiments, the asthma is TH2-low asthma.

In a further aspect, the invention provides a method for inhibiting the biological activity of KLK5 in an individual. In one embodiment, the method comprises administering to the individual an effective amount of an anti-KLK5 antibody to inhibiting the biological activity of KLK5. In one embodiment, an “individual” is a human.

In a further aspect, the invention provides pharmaceutical formulations comprising any of the anti-KLK5 antibodies provided herein, e.g., for use in any of the above therapeutic methods. In one embodiment, a pharmaceutical formulation comprises any of the anti-KLK5 antibodies provided herein and a pharmaceutically acceptable carrier. In another embodiment, a pharmaceutical formulation comprises any of the anti-KLK5 antibodies provided herein and at least one additional therapeutic agent, e.g., as described below.

Antibodies of the invention can be used either alone or in combination with other agents in a therapy. For instance, an antibody of the invention may be co-administered with at least one additional therapeutic agent. In some embodiments, the additional therapeutic agent is an IL-13 axis binding antagonist, an IL-5 axis binding antagonist, an IL-33 axis binding antagonist, an M1 prime antagonist, an IgE antagonist, a TRPA1 antagonist, a CRTH2 antagonist, a broncodilator or asthma symptom controller medication, an immunomodulator, a corticosteroid, a Th2 pathway inhibitor, a tyrosine kinase inhibitor, or a phosphodiesterase inhibitor. In some embodiments, the IL-13 axis binding antagonist is an anti-IL-13 antibody. In some embodiments, the anti-IL-13 antibody is lebrikizumab. In some embodiments, the IL-5 axis binding antagonist is an IL-5 binding antagonist or an IL-5 receptor binding antagonist. In some embodiments, the IL-33 axis binding antagonist is an IL-33 binding antagonist or an ST2 binding antagonist. In some embodiments, the IL-33 binding antagonist is an anti-IL-33 antibody. In some embodiments, the M1 prime antagonist is quilizumab.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the anti-KLK5 antibody and administration of an additional therapeutic agent occur within about one month, or within about one, two or three weeks, or within about one, two, three, four, five, or six days, of each other. Antibodies of the invention can also be used in combination with radiation therapy.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered.

It is understood that any of the above formulations or therapeutic methods may be carried out using an immunoconjugate of the invention in place of or in addition to an anti-KLK5 antibody.

### H. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-KLK5 antibody.

### III. Examples

The following are examples of methods and compositions of the invention. It is understood that various other embodiments may be practiced, given the general description provided above.

### Example 1—Material and Methods

### Generation of Anti-KLK5 Antibodies

New Zealand White rabbits were immunized with human KLK5 and single B cells were isolated using a modified protocol related to published literature, e.g. see Offner et al., PLoS ONE 9(2), 2014. This modified workflow included direct FACS sorting of IgG+ huKLK5+B cells into single wells. The B cell culture supernatants were assayed by ELISA for binding to human KLK5 and an irrelevant control protein. KLK5 specific B cells were lysed and immediately frozen in −80° C. for storage until molecular cloning. Variable regions (VH and VL) of each monoclonal antibody from rabbit B cells were cloned into expression vectors from extracted mRNA as previously described, e.g. see Offner et al., PLoS ONE 9(2), 2014. Individual recombinant rabbit antibodies were expressed in Expi293 cells and subsequently purified with protein A. Purified anti-KLK5 antibodies were then subjected to functional activity assays and kinetic screening.

Rats were immunized in a similar manner and hybridomas were generated using a modified fusion partner (e.g. see Price et al., J Immunol Methods 31; 343(1):28-41 (2009)). Various conditions were optimized to enable sorting of individual IgG+ huKLK5+ hybridomas into single wells followed by additional culturing after sorting. The resulting hybridoma supernatants were assayed by ELISA and positive samples were purified using protein A for subsequent functional and kinetic characterization.

### BIAcore™ Experiments

The binding affinity of the antibodies in this section was determined by BIAcore™ T200 machine. Briefly, BIAcore™ research grade CM5 chips were activated with 1-ethyl-3-(3-dimethylaminopropyl) carbodiimide (EDC) and N-hydroxysuccinimide (NHS) reagents according to the supplier's instructions. Goat anti-human Fc IgGs were coupled to the chips to achieve approximately 10,000 response units (RU) in each flow cell. Unreacted coupling groups were blocked with 1M ethanolamine. For kinetics measurements, antibodies were captured to achieve approximately 300 RU. Ten-fold serial dilutions of human KLK5 were injected in HBS-P buffer at 37° C. with a flow rate of 30 L/min. Association rates (ka) and dissociation rates (kd) were calculated using a 1:1 Langmuir binding model (BIAcore™ T200 Evaluation Software version 2.0). The equilibrium dissociation constant (KD) was calculated as the ratio kd/ka.

### Assays for Determination of KLK5 Inhibition

The inhibition of human KLK5 by anti-KLK5 antibodies was measured using a recombinant KLK5 direct activity assay. Recombinant human KLK5 (Genentech) was diluted to 5 nM in direct assay buffer (75 mM Tris (pH 8.0), 150 mM NaCl and 0.01% TWEEN® 20) and combined with anti-KLK5 antibodies in 384-well assay plate (384 Well Low Volume, Black, Round Bottom, Corning, Catalog No. 4514). Antibodies were supplied in either phosphate sample buffer (70 mM sodium phosphate (pH 6), 200 mM NaCl and 0.01% TWEEN® 20) or citrate/Tris sample buffer (10 mM citric acid, 30 mM Tris (pH 6) and 0.01% TWEEN® 20). Antibody dilutions were made in the appropriate sample buffer or in direct assay buffer. Plates were incubated for 30 minutes at ambient temperature. Fluorescent peptide substrate, Boc-VPR-AMC (Bachem, Part No. I-1120) was added directly to the assay plate. Final in-well concentrations were 50 μM Boc-VPR-AMC, 5 nM recombinant human KLK5, and 0.19-100 nM anti-KLK5 antibodies. Plates were examined every 102 s for 30-60 minute using a PHERAstar® Plus reader using a 340 nm excitation/460 nm emission module. The Reaction rate (expressed as RFU) was calculated by linear regression of readings in the linear range, typically beginning at 204 s and continuing until the end of the assay. Buffer alone and 100 nM final SPINK9.SRE.Fc (Genentech) were used as 100% and 0% activity controls, respectively. The IC₅₀ value of the anti-KLK5 antibodies were determined from a four-parameter fit for their respective curves.

The inhibition of human KLK5 by anti-KLK5 antibodies was measured using a coupled pro-KLK7 fluorescent peptide assay. Recombinant human KLK5 (Genentech) was diluted to 5 nM in pro-KLK7 citrate/Tris coupled buffer (50 mM Tris (pH 7.5), 150 mM NaCl and 0.01% TWEEN® 20) if antibody samples were in citrate/Tris sample buffer or pro-KLK7 phosphate coupled buffer (50 mM Tris (pH 8.0), 150 mM NaCl and 0.01% TWEEN® 20) if antibody samples were in phosphate sample buffer. Diluted KLK5 was then combined with anti-KLK5 antibodies in 384-well assay plate (384 Well Low Volume, Black, Round Bottom, Corning, Catalog No. 4514). Antibody dilutions were made as described for the direct KLK5 assay. Plates were incubated for 30 minutes at ambient temperature. Fluorescent peptide substrate, suc-LLVY-AMC (Bachem, Part No. I-1395) and pro-KLK7 (Genentech) were added directly to the assay plate and incubated at ambient temperature. Final in-well concentrations were 100 μM suc-LLVY-AMC, 125 nM pro-KLK7, 5 nM recombinant human KLK5, and 0.19-100 nM anti-KLK5 antibodies. After 24 hours, fluorescent readings were made every 102 s for 30-60 min and the RFU endpoint value was calculated by averaging the last 5 readings. Buffer alone and 100 nM final SPINK9.SRE.Fc (Genentech) were used as 100% and 0% activity controls, respectively. The IC₅₀ value of the anti-KLK5 antibodies were determined from a four-parameter fit for their respective curves.

IC₅₀ value of the anti-KLK5 antibodies was determined using a pro-KLK7 Assay by KLK5-derived cleavage peptide detection using LC/MS (pro-KLK7 LC/MS assay). The product peptide EEAQGDK from reaction between the enzyme KLK5 and substrate proKLK7 was detected by mass spectrometry coupled to liquid chromatography. All compounds were diluted with 50 mM ammonium bicarbonate buffer (Powder/Certified, Fisher Chemical, A643-500) with final concentrations in the assay at 5 nM KLK5 (Genentech) and antibodies ranging from 0.02 to 65 nM, diluted in 96-well plates (Bio-Rad, Hard-Shell 96-Well PCR Plates, low profile, thin wall, skirted, blue/clear #HSP9631). Plates were incubated at room temperature for 30 minutes. Afterward, 15 nM of substrate proKLK7 (Genentech) was added to the enzyme plus inhibitors. After 2 hours, the reaction was quenched using 0.5 μL of Formic Acid (99.5+%, Optima™ LC/MS Grade, Fisher Chemical, A117-10X1AMP). Peptide was detected using the combination of the following masses: Q1, 388.7 m/z and Q3, 319.0 m/z, in a QTRAP® 6500 LC-MS/MS mass spectrometer (Sciex, Framingham, MA). Quantitation of generated peptide was measured using a synthetic KLK7 peptide calibration curve. The IC₅₀ values were determined using Prism 6 Software (GraphPad Software, La Jolla, CA).

The inhibition of human KLK5 by anti-KLK5 antibodies was measured using a coupled pro-KLK1 fluorescent peptide assay. Recombinant human KLK5 (Genentech) was diluted to 0.5 nM in pro-KLK1 coupled assay buffer (100 mM Tris (pH 8.5) and 0.01% TWEEN® 20) and combined with anti-KLK5 antibodies in 384-well assay plate (384 Well Low Volume, Black, Round Bottom, Corning, Catalog No. 4514). Antibody dilutions were made as described for the direct KLK5 assay. Plates were incubated for 30 minutes at ambient temperature. Fluorescent peptide substrate, PFR-AMC (Bachem, Part No. I-1295) and pro-KLK1 (Genentech) were added directly to the assay plate and incubated at ambient temperature. Final in-well concentrations were 50 μM PFR-AMC, 31.25 nM pro-KLK1, 0.5 nM recombinant human KLK5, and 0.19-100 nM anti-KLK5 antibodies. Plates were examined every 102 s for 120 minute using a PHERAstar® Plus reader using a 340 nm excitation/460 nm emission module. The endpoint RFU values were calculated by averaging readings around a time dictated by the inflection point of a plot of RFU versus time for the no inhibitor controls. Buffer alone and 100 nM final SPINK9.SRE.Fc (Genentech) was used as 100% and 0% activity controls, respectively. The IC₅₀ values of the anti-KLK5 antibodies were determined from a four-parameter fit for their respective curves.

IC₅₀ values of the anti-KLK5 antibodies was determined using a pro-KLK1 Assay by KLK5-derived cleavage peptide detection using LC/MS (pro-KLK1 LC/MS Assay). The product peptide APPIQSR from reaction between the enzyme KLK5 and substrate proKLK1 was detected by mass spectrometry coupled to liquid chromatography. All compounds were diluted with 50 mM ammonium bicarbonate buffer (Powder/Certified, Fisher Chemical, A643-500) with final concentrations in the assay at 0.5 nM KLK5 (Genentech) and antibodies ranging from 0.01 to 29 nM, diluted in 96-well plates (Bio-Rad, Hard-Shell 96-Well PCR Plates, low profile, thin wall, skirted, blue/clear #HSP9631). Plates were incubated at room temperature for 60 minutes. Afterward, 300 nM of substrate proKLK1 (Genentech) was added to the enzyme plus inhibitors. After 20 minutes, the reaction was quenched using 0.5 μL of Formic Acid (99.5+%, Optima™ LC/MS Grade, Fisher Chemical, A117-10X1AMP). Peptide was detected using the combination of the following masses: Q1, 384.7 m/z and Q3, 600.3 m/z, in a QTRAP® 6500 LC-MS/MS mass spectrometer (Sciex, Framingham, MA). IC₅₀ value were determined using peak areas and Prism 6 Software (GraphPad Software, La Jolla, CA).

Kᵢ₍ₐₚₚ₎ assay using Z-VPR-pNA as substrate was performed at room temperature in a 96-well half-area plate (white with clear, flat bottom, Corning #3884). Inhibitor samples were diluted to 3× final concentration in assay buffer (100 mM Tris, pH 8.0, 100 mM NaCl, 0.01% TWEEN® 20). Buffer alone and 100 nM final SPINK9.SRE.Fc (Genentech) were used as 100% and 0% activity controls, respectively. Inhibitor or control samples (20 μL) were added to the plate followed by 20 μL KLK5 (Genentech) in assay buffer at final concentrations of 0.5, 0.25, 0.125, and 0.0625 nM. After 30 minutes, 20 μL of Z-VPR-pNA substrate (California Peptide, #876-08, prepared as a 30 mM stock solution in DMSO) in assay buffer was added at a final concentration of 300 μM. In some initial assays the final DMSO concentration was increased to 10% by making an initial dilution of the substrate to 3 mM in DMSO before diluting to 900 μM (i.e., 3× final) in assay buffer. After addition of substrate, the plate was read in a Versamax tunable microplate reader with measurements at 405 nm taken every 102 seconds for 3 hours. Reaction rates (expressed as AU/s) were calculated by linear regression in the range 4182-10710 seconds. Reaction rates were normalized to the values of the 0% and 100% activity controls and fit with a 4-parameter equation to calculate IC₅₀ values. In the case of a bivalent inhibitor, the raw IC₅₀ value was multiplied by two. The IC₅₀ value was then plotted versus KLK5 enzyme concentration, yielding the Ki(app) from the y-intercept value. In addition, if the inhibitor was potent enough, the combined purity of both enzyme and inhibitor could be judged by comparing the slope of the plot to the theoretical value of 0.5.

### Assays for Determination of Antibody Selectivity

KLK1 selectivity assay was performed at room temperature in a 384-well plate (black, low-volume round bottom, Corning #4514) with a final reaction volume of 15 μL. Inhibitor samples were diluted to 3× final concentration in assay buffer (75 mM Tris, pH 8.0, 150 mM NaCl, 0.01% TWEEN® 20). Reactions lacking inhibitor and reactions lacking enzyme were used as 100% and 0% activity controls, respectively. Inhibitor or control samples (5 μL) were added to the plate followed by 5 μL KLK1 (R&D Systems, 2337-SE, Lot NLY0315111, activated according to R&D systems protocol) in assay buffer at a final concentration of 3 nM. After 30 minutes, 5 μL of H-Pro-Phe-Arg-AMC acetate salt (Bachem I-1295, 10 mM stock solution in water) in assay buffer was added at a final concentration of 100 μM. After addition of substrate, the plate was read in PHERAstar® microplate reader using optic module FI 340 460 with the gain set to 85%. Measurements were taken every 102 seconds for 1 hour. Reaction rates (expressed as RFU/s) were calculated by linear regression in the range 204-918 seconds. Reaction rates were normalized to the values of the 0% and 100% activity controls and fit with a 4-parameter equation to calculate IC₅₀ values. In the case of bivalent inhibitors, the raw IC₅₀ value was multiplied by two.

KLK4 selectivity assay was performed at room temperature in a 384-well plate (black, low-volume round bottom, Corning #4514) with a final reaction volume of 15 μL. Inhibitor samples were diluted to 3× final concentration in assay buffer (75 mM Tris, pH 8.0, 150 mM NaCl, 0.01% TWEEN® 20). Reactions lacking inhibitor and reactions lacking enzyme were used as 100% and 0% activity controls, respectively. Inhibitor or control samples (5 μL) were added to the plate followed by 5 μL KLK4 (R&D Systems, 1719-SE, Lot MSY0116011, activated according to R&D systems protocol) in assay buffer at a final concentration of 2 nM. After 30 minutes, 5 μL of Boc-Val-Pro-Arg-AMC (Bachem I-1120, 31.3 mM stock solution in water) in assay buffer was added at a final concentration of 50 μM. After addition of substrate, the plate was read in PHERAstar® microplate reader using optic module FI 340 460 with the gain set to 85%. Measurements were taken every 102 seconds for 1 hour. Reaction rates (expressed as RFU/s) were calculated by linear regression in the range 510-3570 seconds. Reaction rates were normalized to the values of the 0% and 100% activity controls and fit with a 4-parameter equation to calculate IC₅₀ values. In the case of bivalent inhibitors, the raw IC₅₀ value was multiplied by two.

Trypsin selectivity assay was performed at room temperature in a 384-well plate (black, low-volume round bottom, Corning #4514) with a final reaction volume of 15 μL. Inhibitor samples were diluted to 3× final concentration in assay buffer (75 mM Tris, pH 8.0, 150 mM NaCl, 0.01% TWEEN® 20). Reactions lacking inhibitor and reactions lacking enzyme were used as 100% and 0% activity controls, respectively. Inhibitor or control samples (5 μL) were added to the plate followed by 5 μL trypsin (Sigma Aldrich T8003, Lot SLBM2321V, 42.0 μM stock solution in 1 mM HCl) in assay buffer at a final concentration of 0.25 nM. After 30 minutes, L of Boc-Val-Pro-Arg-AMC (Bachem I-1120, 31.3 mM stock solution in water) in assay buffer was added at a final concentration of 50 μM. After addition of substrate, the plate was read in PHERAstar® microplate reader using optic module FI 340 460 with the gain set to 85%. Measurements were taken every 102 seconds for 1 hour. Reaction rates (expressed as RFU/s) were calculated by linear regression in the range 204-3570 seconds. Reaction rates were normalized to the values of the 0% and 100% activity controls and fit with a 4-parameter equation to calculate IC₅₀ values. In the case of bivalent inhibitors, the raw IC₅₀ value was multiplied by two.

KLK7 selectivity assay was performed at room temperature in a 384-well plate (black, low-volume round bottom, Corning #4514) with a final reaction volume of 15 μL. Inhibitor samples were diluted to 3× final concentration in assay buffer (75 mM Tris, pH 8.0, 150 mM NaCl, 0.01% TWEEN® 20). Reactions lacking inhibitor and reactions lacking enzyme were used as 100% and 0% activity controls, respectively. Inhibitor or control samples (5 μL) were added to the plate followed by 5 μL KLK7 (Genentech) in assay buffer at a final concentration of 5 nM. After 30 min, 5 μL of Suc-Leu-Leu-Val-Tyr-AMC (Bachem I-1395, 25 mM stock solution in water) in assay buffer was added at a final concentration of 100 μM. After addition of substrate, the plate was read in PHERAstar® microplate reader using optic module FI 340 460 (AMC Module) with the gain set to 85%. Measurements were taken every 102 seconds for 1 hour 15 minute. Reaction rates (expressed as RFU/s) were calculated by linear regression in the range 2040-4488 seconds. Reaction rates were normalized to the values of the 0% and 100% activity controls and fit with a 4-parameter equation to calculate IC₅₀ values.

### PHERAstar® Experiments

Pre MCA PHERAstar® Module: After 30 min, 5 μL of Suc-Leu-Leu-Val-Tyr-AMC (Bachem I-1395, 25 mM stock solution in water) in assay buffer was added at a final concentration of 100 μM. After addition of substrate, the plate was read in PHERAstar® microplate reader using optic module FI 340 460 (AMC Module) with the gain set to 85%. Measurements were taken every 102 seconds for 1 hour 15 minute. Reaction rates (expressed as RFU/s) were calculated by linear regression in the range 2040-4488 seconds. Reaction rates were normalized to the values of the 0% and 100% activity controls and fit with a 4-parameter equation to calculate IC₅₀ values.

Post MCA PHERAstar® Module: After 30 min, 5 μL of Mca-RPKPVE-Nval-WRK(Dnp)-NH2 Fluorogenic MMP Substrate (R&D Systems ES002, 4.3 mM stock solution in DMSO) in assay buffer was added at a final concentration of 10 μM. After addition of substrate, the plate was read in PHERAstar® microplate reader using optic module FI 320 405 (MCA Module) with the gain set to 85%. Measurements were taken every 102 seconds for 1 hour 15 minute. Reaction rates (expressed as RFU/s) were calculated by linear regression in the range 204-1020 seconds. Reaction rates were normalized to the values of the 0% and 100% activity controls and fit with a 4-parameter equation to calculate IC₅₀ values.

### Wasatch Experiments

An array-based SPR imaging system (Carterra™, USA) was used to epitope bin a panel of 288 monoclonal antibodies. Purified antibodies were diluted at 10 μg/ml in 10 mM sodium acetate buffer pH 4.5. Using amine coupling, antibodies were directly immobilized onto a SPR sensorprism CMD 200M chip (XanTec Bioanalytics GmbH, Germany) using a Continuous Flow Microspotter (Carterra™, USA) to create an array of 288 antibodies. For analysis, the IBIS MX96 SPRi (Carterra™, USA) was used to evaluate analytes binding to the immobilized ligands. For kinetic analyses, human, cyno and murine KLK5 were injected for 3 minutes from 0 to 300 nM at 3-fold dilution followed by a dissociation period of 10 minutes. Human KLK1, KLK4, and KLK7 were injected at a single concentration of 500 nM to ensure the specificities of the antibodies. For epitope binning against SPINK9-Fc-SRE, human KLK5 was first injected for 4 minute at 100 nM and was followed by a second 4 minute injection of SPINK9-Fc-SRE at 10 g/ml. The surface was regenerated with 10 mM glycine pH1.5 between cycles. The experiment was performed at 25° C. in a running buffer of HBS-T buffer (0.01M HEPES pH 7.4, 0.15M NaCl, 0.05% surfactant P20). The kinetic data was processed using Scrubber 2.0 (BioLogic™ Software) and the epitope binning data was processed using Wasatch binning software tool (Carterra™ USA).

### Hydrogen Exchange Mass Spectrometry

Experimental starting material compositions are listed in Table 2. Two labeling solutions were prepared with ²H₂O (heavy water) having a pH of either 6.0 or 8.0, and consisting of a mixture of anhydrous monobasic sodium phosphate and dibasic sodium phosphate dehydrate combined in an appropriate ratio to achieve the stated deuterium activity at a total concentration 10 mM with 140 mM NaCl. Starting materials (Table 2) were diluted approximately 1:10 with labeling solutions using a leap robotics platform and incubated for various times before being diluted 1:1 with quench buffer (4M GdmCl, 0.5 M TCEP, 200 mM Citric acid), to a final pH of 2.5, and then prepared quickly for mass measurement. Quenched material was injected into an online flow system where it was digested using pepsin, buffer exchanged while bound to a trap column, separated by reversed-phase chromatography, and introduced into the gas phase by electrospray where individual peptides are assessed for the amount of carried deuterium.

TABLE 2

| | | Starting Material | |
|---|---|---|---|
| | Mab: | Exp. Composition | |
| Sample | KLK5 Ratio | [Mab] μM | [KLK5] μM |
| KLK5.10C5 | 0.70 | 23.10 | 33.00 |
| KLK5.10H3 | 0.65 | 20.98 | 33.00 |
| KLK5.9H5 | 0.55 | 18.07 | 33.00 |
| KLK5 | 0.00 | 0.00 | 33.00 |

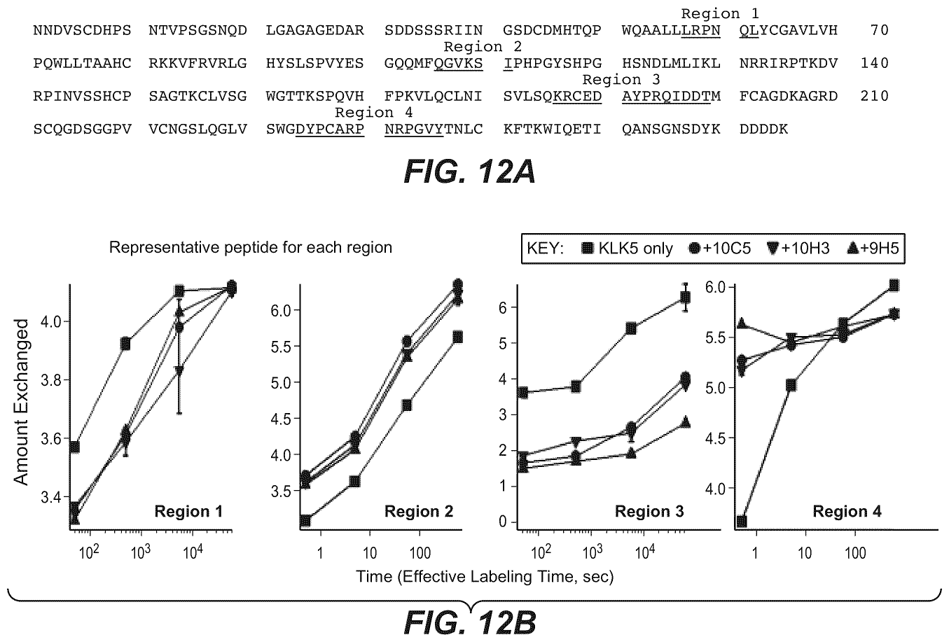

At either pH 6.0 or 8.0, samples were labeled for 0.5, 5.0, 56.0, and 600.0 minutes. Independent experimental replicates were included; on plots shown, error bars represent the range of measurements, whose average shown by markers in FIG. 12B. Feature abstraction and data analysis involved custom in-house software.

Effective labeling time at a reference pH was defined by both experimental pH and actual labeling time: t_{eff}=tₑₓₚ*10\hat{ }(pH_{ref}pHₑₓₚ). The reference pH in FIG. 12B is 6.0. To correct for a well-known proton activity offset that occurs as a result of ²H₂O, the following relationship connects pH measurements with pH values referred herein:

pH=pHₘₑₐₛᵤᵣₑ+([²H₂O/²H₂O+¹H₂O]*0.4).

The sequence and retention time of experimental peptides derived from KLK5 was determined in initial experiments where the same procedural workflow described above was followed but with a labeling buffer in ¹H₂O solvent. This provides a starting mass from which the amount of deuterium contained in each peptide during the labeling experiments may be determined; additionally, these experiments utilize tandem-MS for de novo sequencing by MS.

### IL-8 Secretion Assay

IL-8 is secreted by several cell types in response to inflammatory stimuli. KLK5 stimulates A459 cells, resulting in the secretion of IL-8. Adenocarcinomic human alveolar basal epithelial cells constitute the A549 cell-line. A549 cells were cultured with media containing RPMI, 10% FBS, L-glutamine, and supplemented with penicillin and streptomycin. Cells were plated into TC-treated 96-well plates (Corning, Cat. #3997) at 50,000 cells per well and starved of serum for 18 hours before KLK5 challenge. Human KLK5 was first incubated with KLK5 inhibitors or starvation media for 1 hour at room temperature prior to addition to plate (200 nM in-well concentration) for 24 hr at 37° C. in a humid environment in the presence of 5% CO₂. Endotoxin inhibitor (1 μM) was also added to each well. After incubation, the cell supernatants were collected and either analyzed immediately or stored at −20° C. Cell supernatants were serially diluted in sample diluent (PBS/0.5% BSA/0.05% polysorbate-20/5 mM EDTA/0.25% CHAPS/0.2% BGG/10 ppM Proclin) for analysis in an IL-8 sandwich ELISA using a standard protocol with mouse anti-human IL-8 monoclonal antibody for capture (R&D Systems, Cat #MAB208) and biotinylated mouse anti-human IL-8 monoclonal antibody for detection (R&D Systems, Cat #BAF208). The cell stimulation activity of KLK5 was analyzed by normalizing the IL-8 concentrations to 100% for KLK5 treatment alone and to 0% for starvation media alone treatment. IC₅₀ values were determined using a 4-parameter fit with GraphPad Prism software.

### KLK5 and Fab Expression, Purification and Crystallization

Recombinant human KLK5 residues I67-S293 (SEQ ID NO:328) was expressed in a Baculovirus expression system as a C-terminal fusion of ubiquitin with an Enterokinase cleavage site engineered between ubiquitin and KLK5. Sf9 cells were coexpressed with EndoH with 1 mg/mL of Kifunensine. Protein was purified on a HiTrap™ Heparin column. The column was washed with 25 mM TRIS pH 7.5 for 5 CV (column volumes), and then eluted with 0-750 mM NaCl over a gradient of 20 CV. Resultant protein pool was then further purified over S200 by SEC in 25 mM Tris pH 7.5, 300 mM NaCl. The Ubiquitin was then cleaved using Enterokinase, and sample further purified by SEC again. Mass spectrometry and SDS PAGE revealed a pure KLK5 sample, which was confirmed to be active. Fab fragments including heavy and light chains were expressed and purified as described. See Carter et al., Biotechnology 10(2), 163-167 (1992). KLK5 was then mixed with each of the Fabs separately, 10C5, 9H5, and 3-3F5, and the complex was purified by SEC in 25 mM HEPES pH 7.2, 100 mM NaCl. The crystallization conditions for the 3 Fab-KLK5 complexes were as follows: 3-3F5 complex—15% PEG 4K, 0.1 M MgCl2, 0.1 M Na Citrate pH 5.0; 10C5 Complex: 20% PEG4K, 0.2M Ammonium Sulfate, 25% Glycerol; 9H5 Complex: 15% PEG4K, 10% Isopropanol, 0.1M HEPES pH 7.5. Crystal for the 10C5 and 9H5 complexes were only obtained when the lysine residues in the protein complexes were methylated. See Walter et al., Structure, 14(11), 1617-1622 (2006).

### Data Collection and Structure Solution

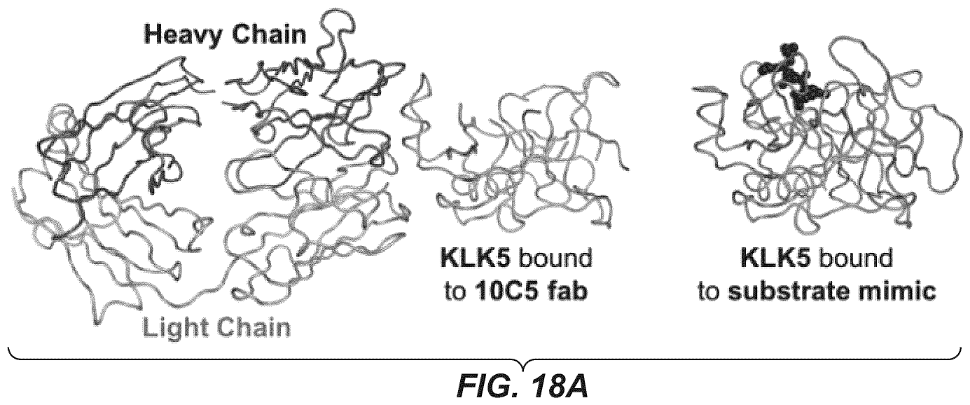

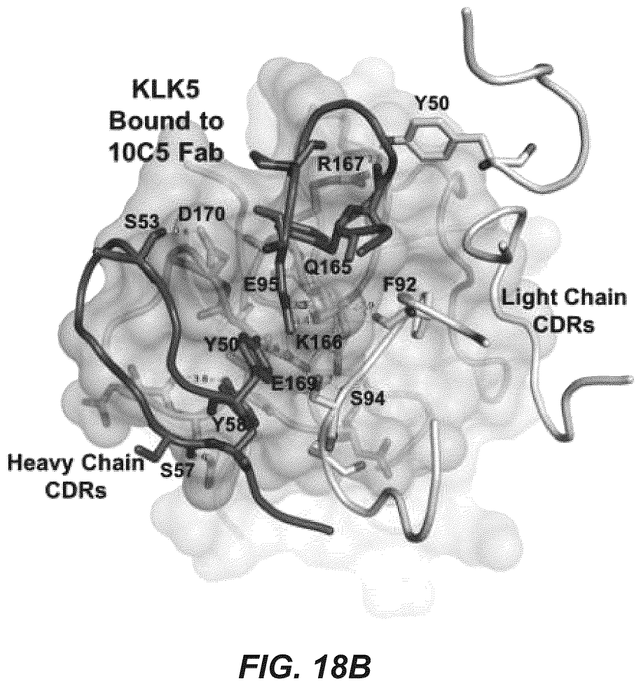

X-ray diffraction data was collected under cryo-cooled conditions at 100 Kelvin using various synchrotron X-ray radiation at the Advanced Light Source (Berkeley, CA) or Advanced Photon Source (Argonne, IL) according to standard methods. Diffraction images were processed and reduced using the data processing software XDS. See Kabsch W, Acta Crystallogr D Biol Crystallogr, 66(Pt 2), 125-132 (2010). Models were generated using the molecular replacement technique with the program PHASER. The structure of human TIGIT (see Debala et al., J Mol Biol, 373:1017-1031 (2007)) and Fab antibody model (See Nakamura et al., Cell Host Microbe, 14(1), 93-103 (2013)) were used as search models. The structures underwent iterative rounds of model adjustment using the program COOT and refinement using the Phenix.refine or BUSTER programs. Models were refined to acceptable R and R free values and Ramachandran statistics (calculated by Molprobity). The crystal structures of KLK5 complexed with each of the Fabs, 10C5, 9H5 and 3-3F5 as well as the details of interactions and epitopes can be found in FIGS. 18-20 and Tables 5-13.

### Example 2—Humanization of Anti-KLK5 Antibodies

By immunization of animals with human KLK5 as described herein above, as set of 540 monoclonal anti-KLK5 antibodies was obtained. As this set contained anti-KLK5 antibodies with highly variable characteristics, the antibodies were screened for desired characteristics, such as a certain IC₅₀ values and selectivity to human KLK5. This screening revealed a large number of antibodies with low affinity to human KLK5, no selectivity with respect to human KLK5, and/or insufficient functional activity. For example, some of the obtained anti-KLK5 antibodies were partial inhibitors, i.e. inhibition by ≤50% (e.g. clones 12B3, 1D10) or inhibition by ≤90% (e.g. clones 14C8, 14E12, 8E11). Some of the obtained anti-KLK5 antibodies inhibited human KLK5 only in one of the assays described herein (e.g. clones 9E3, 10D10). Only 13 anti-KLK5 antibodies were selected based on their characteristics (clones 8G10, 9B6, 2-3F4, 10C5, 2B11, 10H3, 9H3, 8B7, 9H5, 9F2, 10C8, 8F5, 3-3F5). The three anti-KLK5 antibodies with the highest inhibitory activities (clones 9H5, 10C5, 3-3F5) were then selected for humanization.

Rabbit monoclonal antibodies 9H5, 10C5, and 3-3F5 were humanized as described below. Residue numbers are according to Kabat et al., Sequences of proteins of immunological interest, 5th Ed., Public Health Service, National Institutes of Health, Bethesda, Md. (1991).

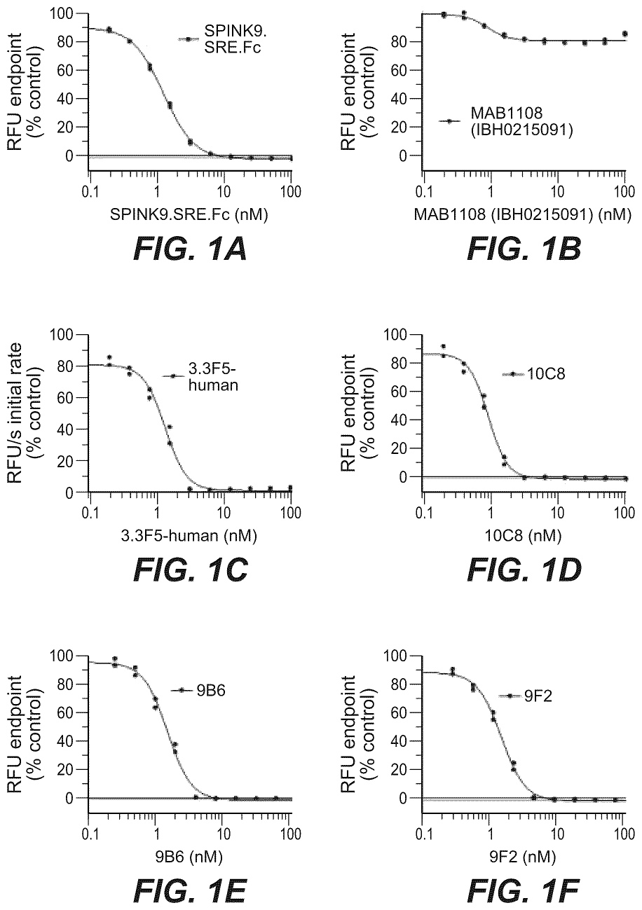

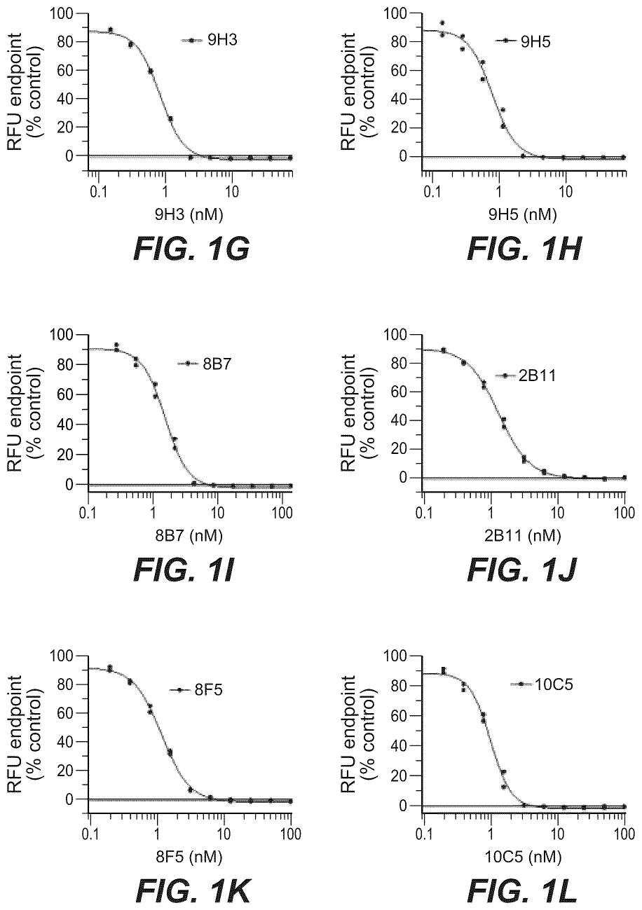

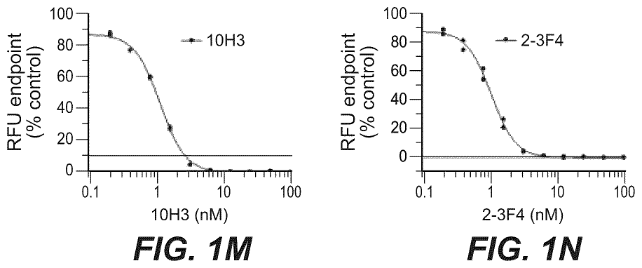

Variants constructed during the humanization of 9H5, 10C5, and 3-3F5 were assessed in the form of human IgG1. Hypervariable regions from each of the rabbit antibodies (namely positions 24-34 (L1), 50-56 (L2) and 89-97 (L3) in VL domain, and 26-35 (H1), 50-65 (H2) and 95-102 (H3) in VH domain) were grafted into various acceptor frameworks. Specifically, for 9H5, VL CDRs were grafted into KV1D-39*01 and VH CDRs were grafted into HV3-53*01. For 10C5, VL CDRs were grafted into KV1-8*01 and VH CDRs were grafted into HV3-64*01. For 3-3F5, VL CDRs were grafted into KV1-8*01 and VH CDRs were grafted into HV3-23*01. All VL and VH Vernier positions from rabbit antibodies were also grafted into their respective human germline frameworks. The grafts with all rabbit amino acids in Vernier positions are referred to as L1H1 (hu9H5.L1H1, hu10C5.L1H1 and hu3-3F5.L1H1) (FIGS. 14A and 14B-1 to 14B-3; 15A and 15B-1 to 15B-3; and 16A and 16B-1 to 16B-3).

The binding affinity of the antibodies in this section was determined using BIAcore™ T200 as described herein above. The binding affinity of humanized 9H5, 10C5 and 3-3F5 version L1H1 (hu9H5.L1H1, hu10C5.L1H1 and hu3-3F5.L1H1) antibodies were compared to their chimeric parental clones. Rabbit Vernier positions of version L1H1 antibodies were converted back to human residues to evaluate the contribution of each rabbit Vernier positions to binding affinity to hKLK5.

For 9H5, three additional light chains (L2: L1+Ala43, L3: L1+Tyr49, and L4: L1+Ala43+Tyr49 (CDR graft)) and sixteen additional heavy chains (H2: H1+Ala24, H3: H1+Trp47, H4: H1+Val48, H5: H1+Ser49, H6: H1+Phe67, H7: H1+Asn73, H8: H1+Leu78, H9: H1+Tyr91, H10: H1+Gln105, H11: no rabbit residues in Vernier positions (CDR graft), H12: H1+Asp61+Ser62+Val63+Gly65, H13: CDR graft+Asp61+Ser62+Val63+Gly65, H14: CDR graft+Tyr47+Gly49, H15: H14+Gln2, H16: H14+Asn72+Thr73+Asn74+Leu75, and H17: H14+Phe62) were made (FIGS. 15A and 15B-1 to 15B-3). Tyr47 and Gly49 on the heavy chain (H14) were determined to be the key rabbit Vernier residues based on binding affinity evaluation of the variant antibodies described above (data not shown). Chimeric 9H5 bound with a KD of 1.9E⁻¹⁰ M, while hu9H5.L4H14, bound with a KD of 6.9E⁻¹⁰ M.

For 10C5, four additional light chain variants L2-L5 (L2: L1+Ile2, L3: L1+Ala43, L4: L1+Ile2+Ala43 (CDR graft), L5: CDR graft+Ser77+Pro80, L6: CDR graft+Ser77+Pro80+Glu103+Val105+Val106) and twenty-seven additional heavy chain variants H2 to H28 (H2: H1+Ala24, H3: H1+Tyr47, H4: H1+Val48, H5: H1+Ser49, H6: H1+Phe67, H7: H1+Asn73, H8: H1+Leu78, H9: H1+Tyr91, H10: H1+Gln105, H11: H1+Asn61+Ser62+Val63+Gly65, H12: no rabbit residues in Vernier positions (CDR graft), H13: CDR graft+Asn61+Ser62+Val63+Gly65, H14: CDR graft+Trp47+Gly49, H15: H14+Gln2, H16: H14+Asn72+Leu73+Asn74+Thr75, H17: H14+Phe62, H18: H14+Asn72+Thr73+Asn74+Leu75, H19: H14+Asn72, H20: H14+Asn74, H21: H14+Asn72+Asn74, H22: H14+Ile48, H23: H14+Ser67, H24: H22+Ser67, H25: H14+Thr73, H26: H14+Thr78, H27: H25+Thr78, H28: H22+Ser67+Thr73+Thr78) were made (FIGS. 14A and 14B-1 to 14B-3). Trp47, Ile48, Gly49, Ser67, Thr73, and Val78 on the heavy chain (H28) were determined to be the key rabbit Vernier residues based on binding affinity evaluation of the variant antibodies described above (data not shown). Chimeric 10C5 bound with a KD of 1.65E⁻¹¹ M, while hu10C5.L5H28, bound with a KD of 5.70E⁻¹¹ M.

For 3-3F5, four additional light chain variants L2-L5 (L2: L1+Ile2, L3: L1+Ala43, L4: L1+Ile2+Ala43 (CDR graft), L5: CDR graft+Ser77+Pro80) and twenty-six additional heavy chain variants H2 to H27 (H2: H1+Ala24, H3: H1+Val48, H4: H1+Ser49, H5: H1+Phe67, H6: H1+Asn73, H7: H1+Leu78, H8: H1+Tyr91, H9: H1+Arg105, H10: H1+Phe58, H11: H1+Asp61+Ser62+Val63, H12: CDR graft+Phe58, H13: CDR graft+Phe58+Asp61+Ser62+Val63, H14: CDR graft+Gly49, H15: H14+Phe58, H16: H14+Gln2, H17: H14+Asn72+Thr73+Asn74+Leu75, H18: H14+Phe62, H19: H18+Val24, H20: H18+Ile48, H21: H18+Ser67, H22: H18+Ile48+Ser67, H23: H18+Thr73, H24: H18+Val78, H25: H18+Thr73+Val78, H26: H18+1148+Ser67+Thr73+Val78, H27: CDRs graft+Ala24+Ile48+Gly49+Phe58+Phe62+Ser67, H28: H19+Phe58+Thr73+Thr78) were made (FIGS. 16A and 16B-1 to 16B-3). Val24, Gly49, Thr73, and Val78 on the heavy chain (H28) were determined to be the key rabbit Vernier residues based on binding affinity evaluation of the variant antibodies described above (data not shown). Chimeric 3-3F5 bound with a KD of ≤1E⁻¹² M (KD is below the detection limit of 10E⁻⁶ s⁻¹ for the instrument), while hu3-3F5.L5H19 bound with a KD of 4.1E⁻¹² M and hu3-3F5.L5H25 bound with a KD of 9.1E⁻¹² M.

The hu9H5.L4H14, hu10C5.L5H28, hu3-3F5.L5H19, hu3-3F5.L5H25 and their chimeric counterparts were tested for their ability to bind human KLK5 as described herein above. Binding properties for the humanized antibodies are shown in Table 3.

TABLE 3

| Ligand | Sample | ka (1/Ms) | kd (1/s) | KD (M) |
|---|---|---|---|---|
| human KLK5 | 9H5 | 7.05E⁺⁵ | 1.37E⁻⁴ | 1.93E⁻¹⁰ |
| | hu9H5.L4H14 | 8.10E⁺⁵ | 5.62E⁻⁴ | 6.94E⁻¹⁰ |
| human KLK5 | 10C5 | 1.78E⁺⁷ | 2.92E⁻⁴ | 1.65E⁻¹¹ |
| | hu10C5.L5H28 | 1.90E⁺⁷ | 1.08E⁻³ | 5.70E⁻¹¹ |
| human KLK5 | 3-3F5 | 2.93E⁺⁷ | <1.00E⁻⁵ | <1.00E⁻¹² |
| | hu3-3F5.L5H19 | 4.84E⁺⁷ | 2.01E⁻⁴ | 4.14E⁻¹² |
| | hu3-3F5.L5H25 | 2.57E⁺⁷ | 2.35E⁻⁴ | 9.14E⁻¹² |

### Example 3—Evaluation of IC₅₀ Values and Specificity of Anti-KLK5 Antibodies

### Evaluation of KLK5 Inhibitor IC₅₀ Values Using Fluorescent Peptide Substrates

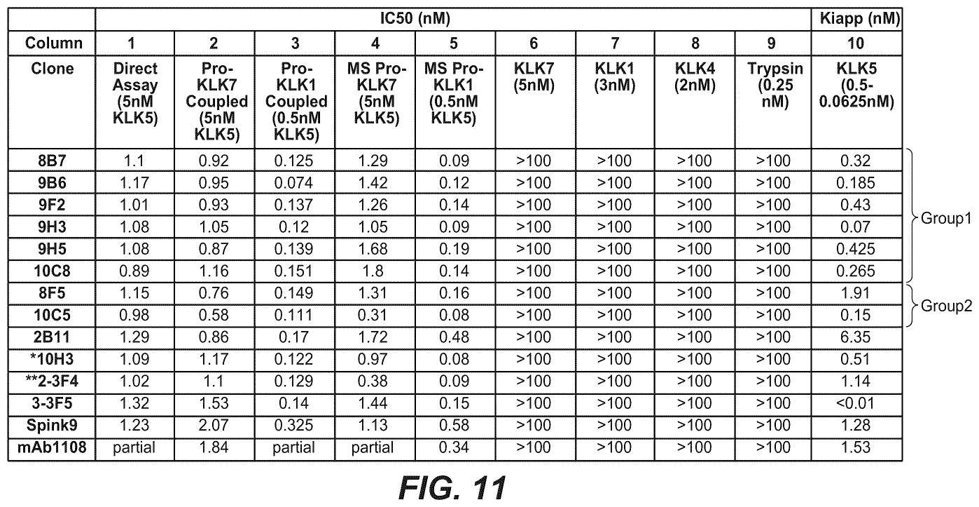

The ability of Spink9.SRE.Fc, mAb1108 or 12 selected antibodies to inhibit the proteolysis of the substrate, Boc-VPR-AMC, by human KLK5 was assessed using an enzymatic assay. In this assay the three-mer fluorogenic peptide substrate contains a highly fluorescent 7-amino4-methylcoumarin (AMC) group that is quenched by resonance energy transfer to the t-Butyloxycarbonyl (Boc). The cleavage of the peptide substrate by human KLK5 resulted in an increased fluorescent signal and the inhibition or absence of KLK5 resulted in a quenched fluorescent signal. The results were expressed as a percentage of maximum KLK5 activity (% control). The results of a single experiment run in duplicate are shown in FIG. 1. The calculated IC₅₀ value for Spink9.SRE.Fc (FIG. 1A) was 1.23 nM and the range of IC₅₀ values for the 12 selected antibodies (FIGS. 1 C-N) was 0.89 to 1.32 nM. Spink0.SRE.Fc as well as all 12 selected antibodies fully inhibited KLK5 activity, although mAb1108 (FIG. 1B) only demonstrated ˜20% inhibition of KLK5 activity. The IC₅₀ values from the curve fittings are presented in FIG. 11 (Column 1).

### Evaluation of KLK5 Inhibitor IC₅₀ Values Using Coupled Macromolecular Substrate Activity

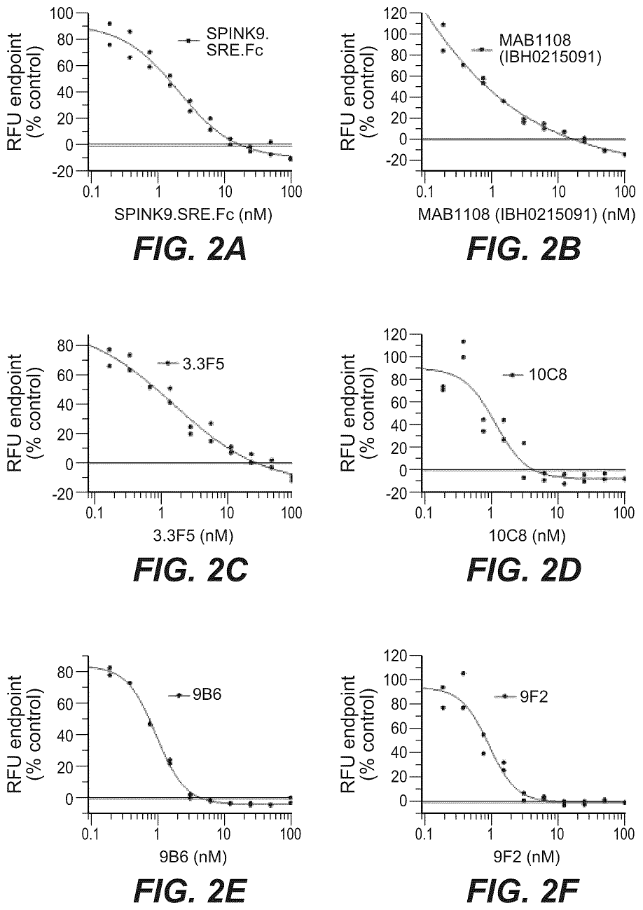

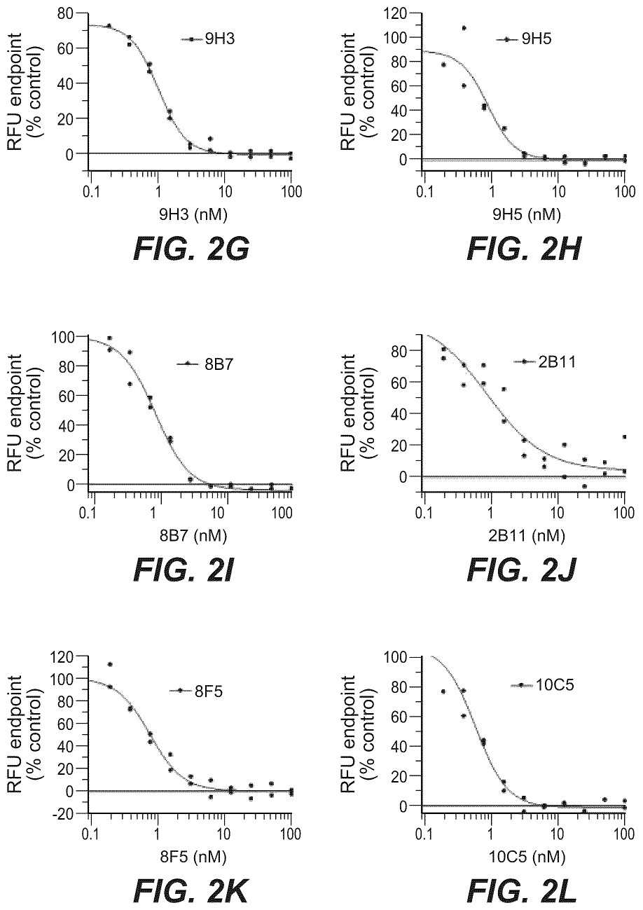

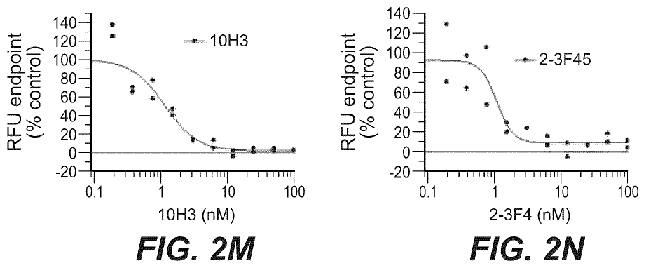

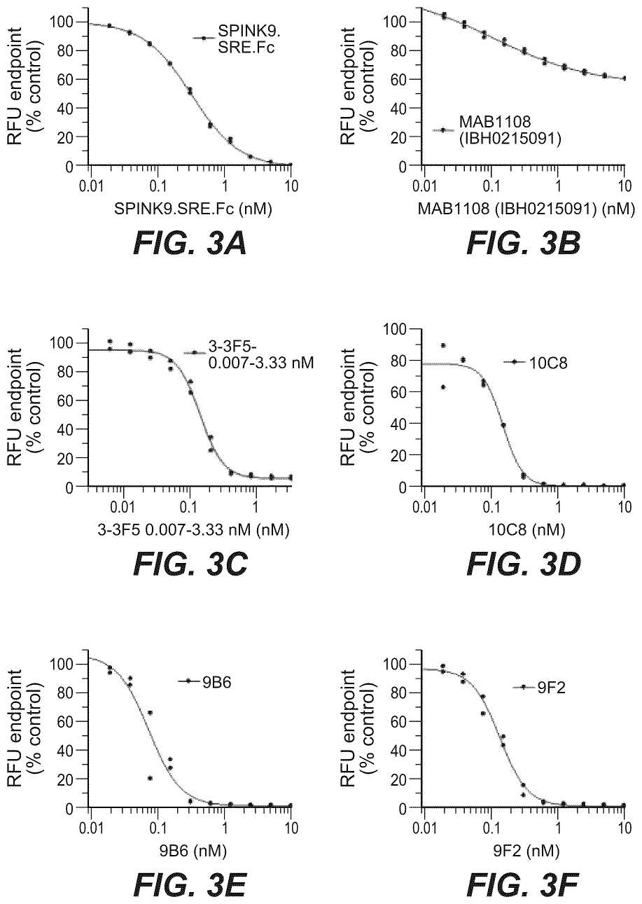

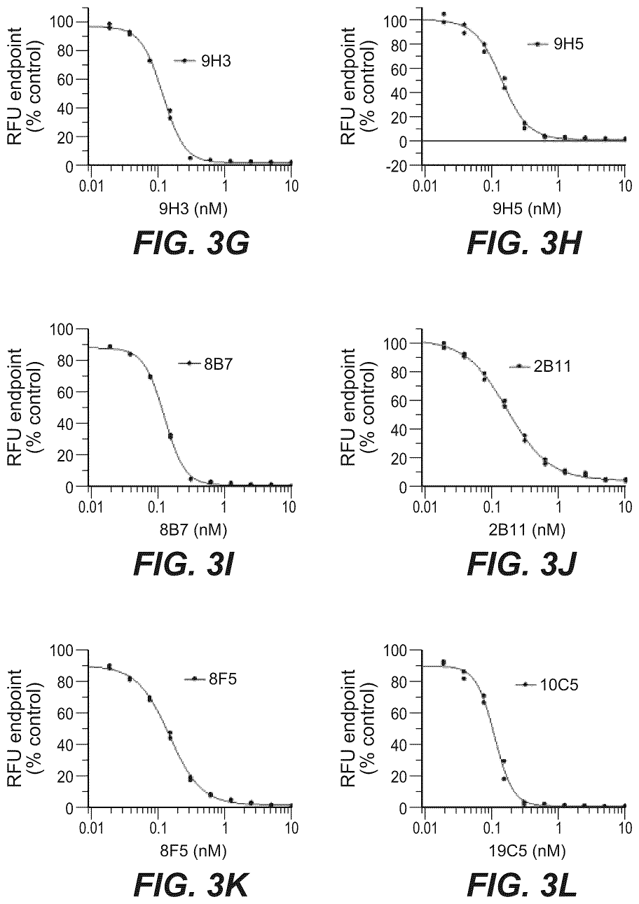

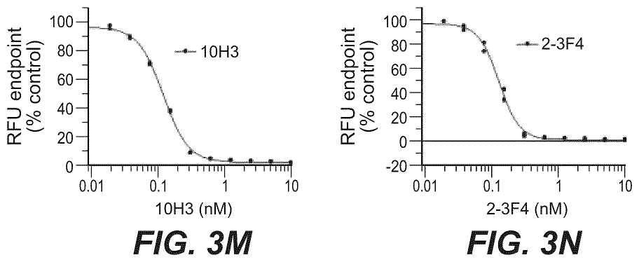

As demonstrated in FIG. 1, Spink9.SRE.Fc as well as the identified 12 anti-KLK5 antibodies are potent inhibitors of KLK5 activity as monitored using a peptide based substrate. To further evaluate the inhibitory profiles of these inhibitors, two assays were developed utilizing macromolecular substrates, pro-KLK7 (FIG. 2) and pro-KLK1 (FIG. 3), in combination with KLK specific fluorescent peptide substrates.

In FIG. 2, the ability of Spink9.SRE.Fc, mAb1108 or 12 selected anti-KLK5 antibodies to inhibit KLK5 mediated activation of pro-KLK7 was assessed using a coupled enzymatic assay. In this assay, human KLK5 is incubated with human pro-KLK7 resulting in cleavage, release of the KLK7 pro-domain and activation of KLK7. Activated human KLK7 can proteolyze a KLK7 specific substrate, Suc-LLVY-AMC, resulting in an increased fluorescent signal. The cleavage of pro-KLK7 by human KLK5 results in active KLK7 and an increased fluorescent signal whereas the inhibition or absence of KLK5 resulted in a quenched fluorescent signal. The results were expressed as a percentage of maximum human KLK5 activity (% control). The results of a single experiment run in duplicate are shown in FIG. 2.

Similar to data using the peptide-based substrate (FIG. 1), Spink9.SRE.Fc (FIG. 2A) was a potent inhibitor of KLK5 activation of pro-KLK7 with an IC₅₀ value of 2.07 nM. Additionally, mAb1108 (FIG. 2B) had an IC₅₀ value of 1.21 nM whereas the range of IC₅₀ values for the 12 selected anti-KLK5 antibodies (FIGS. 2 C-N) was 0.58 to 1.53 nM. In the KLK5 pro-KLK7 coupled assay all of inhibitors, Spink9.SRE.Fc as well as antibodies, fully inhibited KLK5 activity. The IC₅₀ values from the curve fittings are presented in FIG. 11 (Column 2).

In FIG. 3, the ability of Spink9.SRE.Fc, mAb1108 or 12 selected anti-KLK5 antibodies to inhibit KLK5 mediated activation of pro-KLK1 was assessed using a coupled enzymatic assay. In this assay, human KLK5 is incubated with human pro-KLK1 resulting in cleavage, release of the KLK7 pro-domain and activation of KLK1. Activated human KLK1 can proteolyze a KLK1 specific substrate, PFR-AMC, resulting in an increased fluorescent signal. The cleavage of pro-KLK1 by human KLK5 results in active KLK1 and an increased fluorescent signal whereas the inhibition or absence of KLK5 resulted in a quenched fluorescent signal. The results were expressed as a percentage of maximum human KLK5 activity (% control). The results of a single experiment run in duplicate are shown in FIG. 3. The IC₅₀ value for Spink9.SRE.Fc (FIG. 3A) was 0.325 nM and the range of IC₅₀ for the 12 selected anti-KLK5 antibodies (FIGS. 2 C-N) was 0.074 to 0.151 nM. Spink0.SRE.Fc as well as all 12 selected anti-KLK5 antibodies fully inhibited KLK5 activity, although mAb1108 (FIG. 1B) only demonstrated ˜40% inhibition of KLK5 activity. The IC₅₀ values from the curve fittings are presented in FIG. 11 (Column 3).

### KLK5-Derived Cleavage Peptide Detection by LC/MS for IC₅₀ Value Determination

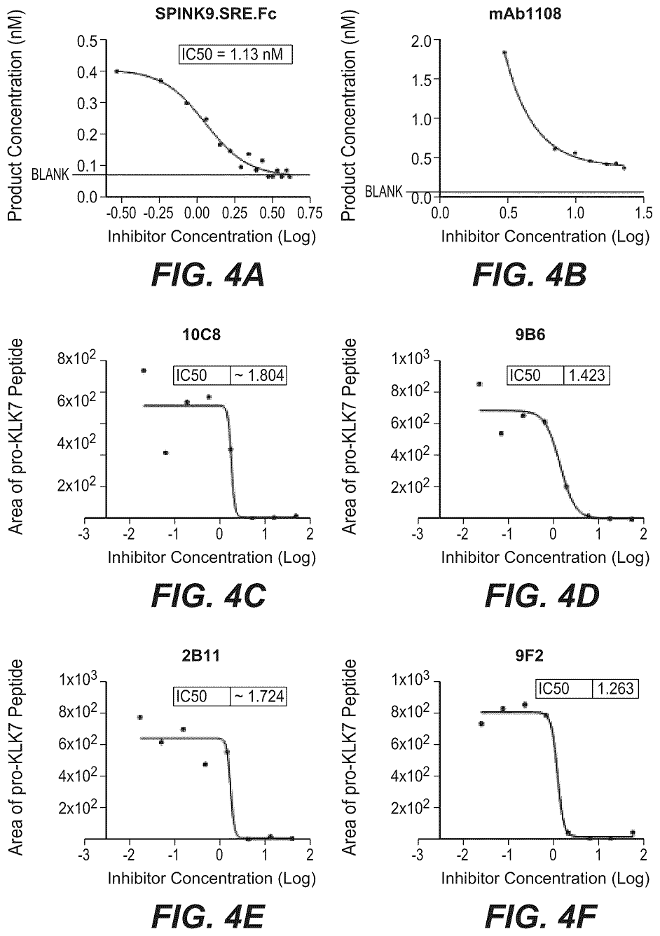

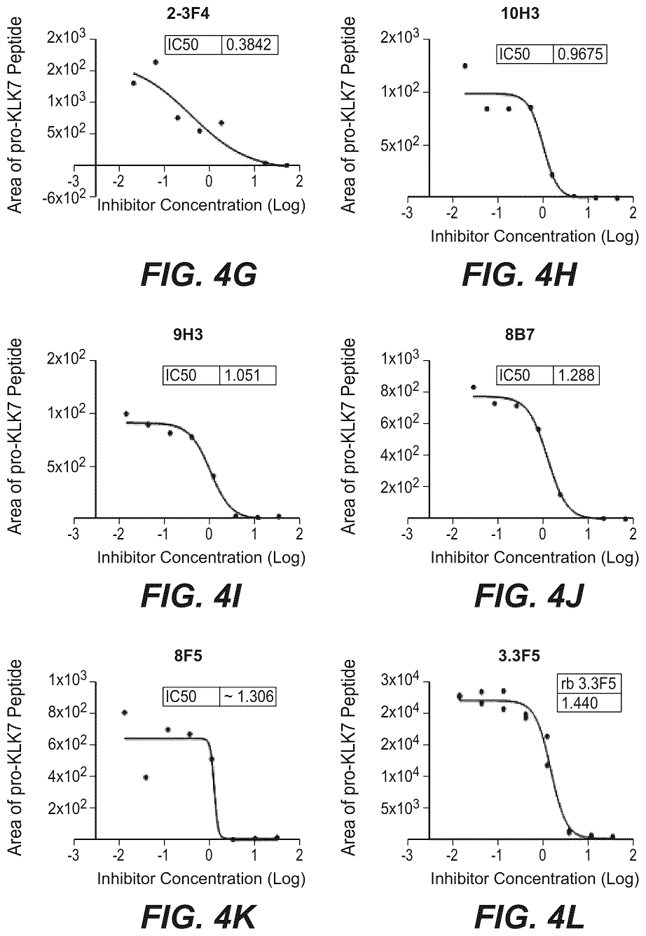

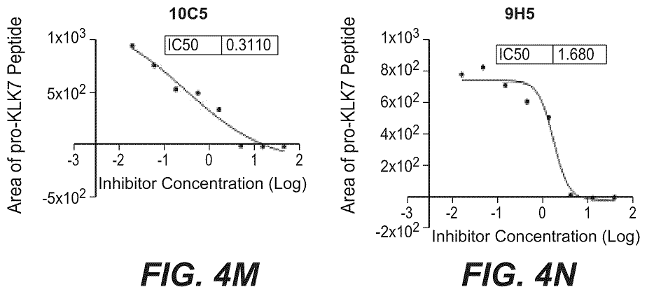

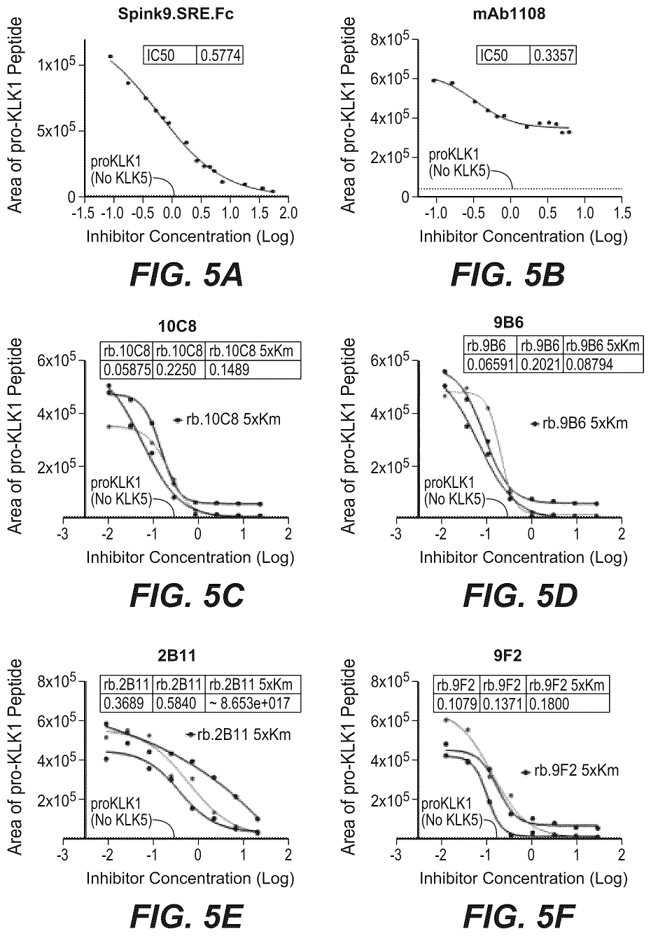

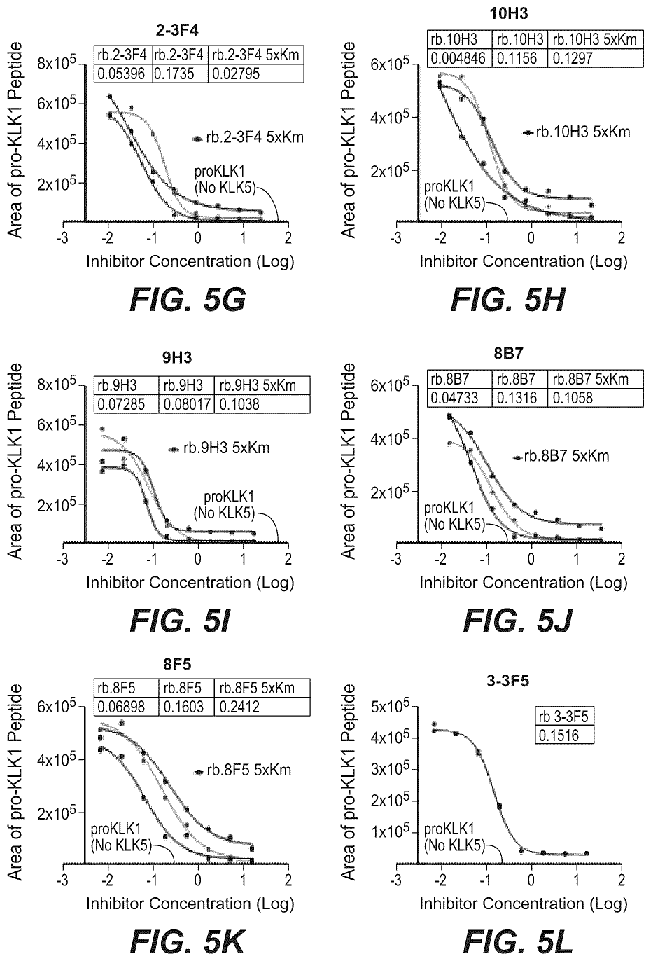

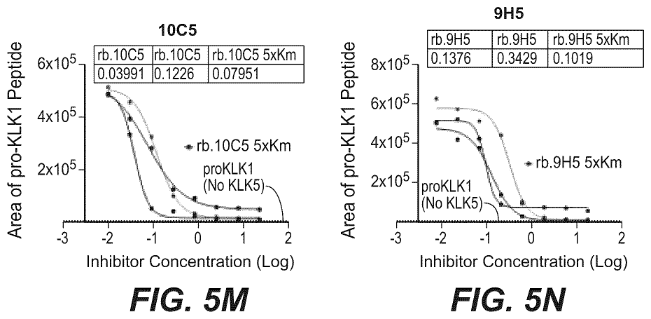

The ability of Spink9.SRE.Fc, mAb1108 or 12 selected anti-KLK5 antibodies to inhibit proteolysis of pro-KLK7 or pro-KLK1 by recombinant KLK5 was assessed using an LC/MS assay that monitors the KLK5-derived cleavage product peptides. For both assays, the cleavage of the pro-peptide KLK7 or KLK1 by human KLK5 results in a specific MS signal and the inhibition of KLK5 activity results in a measurable decrease of the peptide signal. The results of a single experiment for pro-KLK7 are shown in FIG. 4 and they are expressed as area of pro-peptide KLK7. The calculated IC₅₀ value for Spink9.SRE.Fc (FIG. 4A) was 1.13 nM, for MAb1108 (FIG. 4B) was 1.86 nM and the range of IC₅₀ values for the 12 selected anti-KLK5 antibodies (FIG. 4 C-N) was 0.31 to 1.72 nM. Spink9.SRE.Fc as well as all 12 selected anti-KLK5 antibodies fully inhibited KLK5 activity, and mAb1108 (FIG. 4B) demonstrated ˜80% inhibition of KLK5 activity. The IC₅₀ values from the curve fittings are presented in FIG. 11 (Column 4). FIG. 5 shows the results of three experiments for pro-KLK1 and they are expressed as area of pro-peptide KLK1. The IC₅₀ value for Spink9.SRE.Fc (FIG. 5A) was 0.58 nM, for MAb1108 (FIG. 5B) was 0.34 nM and the range of IC₅₀ values for the 12 selected anti-KLK5 antibodies (FIG. 5 C-N) was 0.08 to 0.48 nM. Spink9.SRE.Fc as well as all 12 selected anti-KLK5 antibodies fully inhibited KLK5 activity, although mAb1108 (FIG. 5B) demonstrated ˜40% inhibition of KLK5 activity. The IC₅₀ values from the curve fittings are presented in FIG. 11 (Column 5).

### Specificity of KLK5 Antibodies

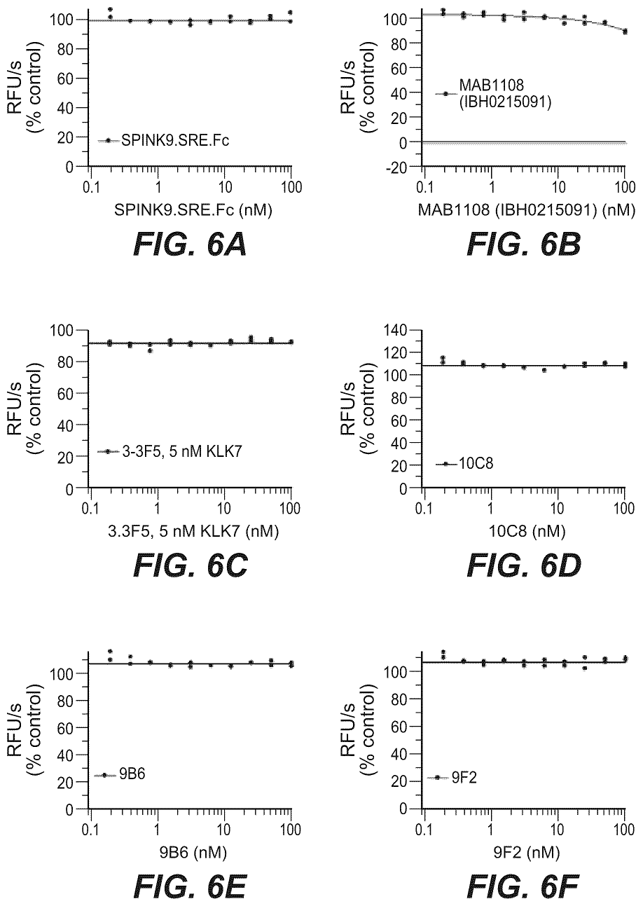

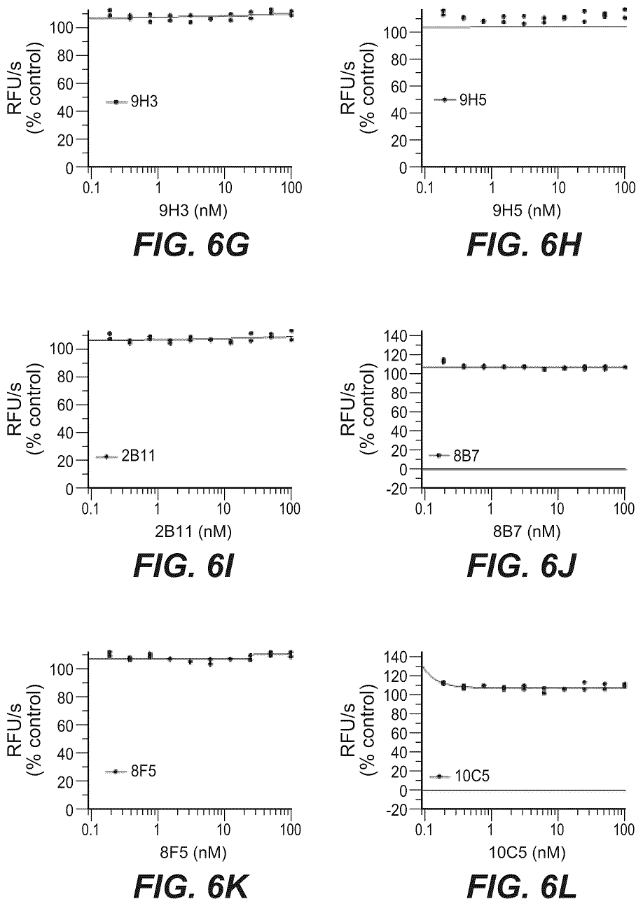

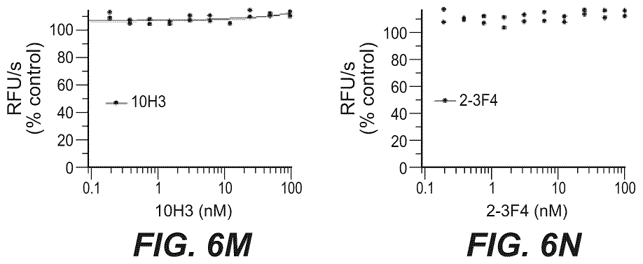

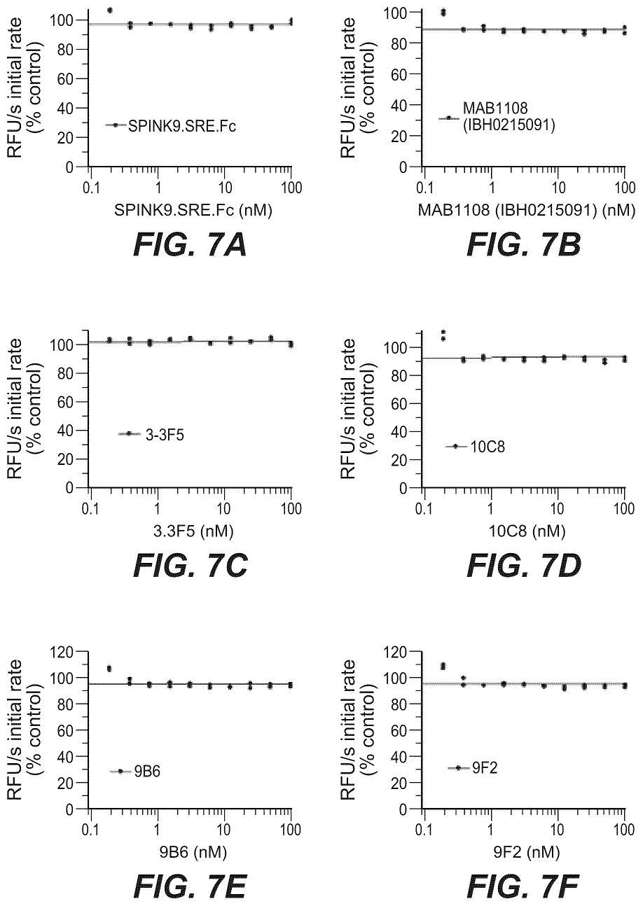

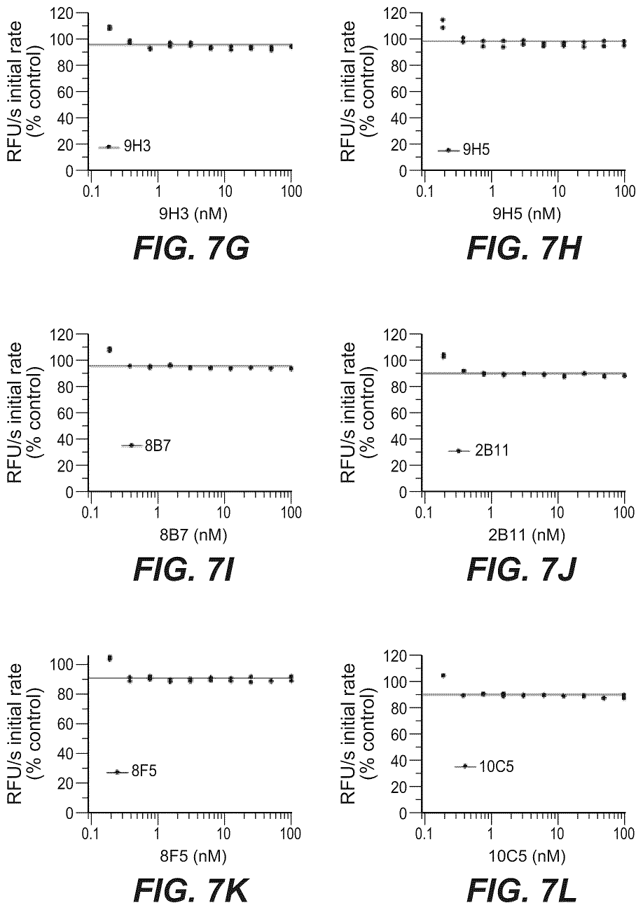

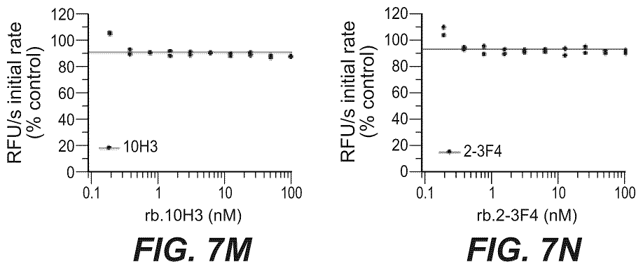

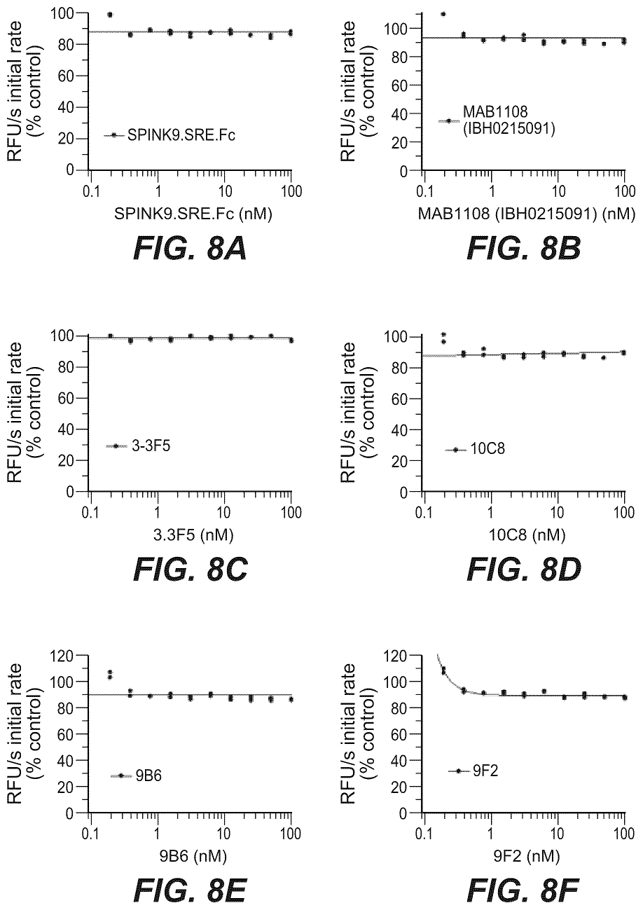

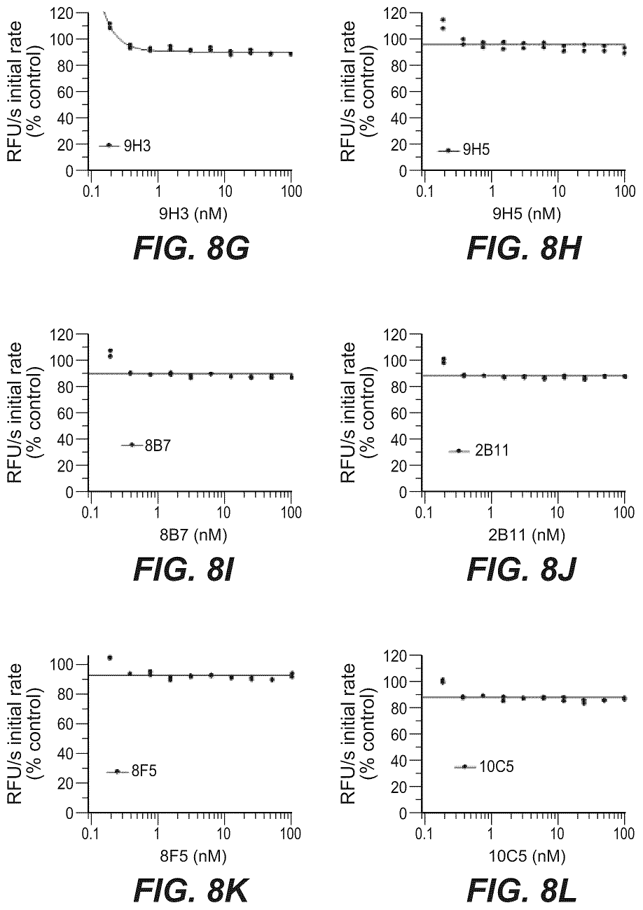

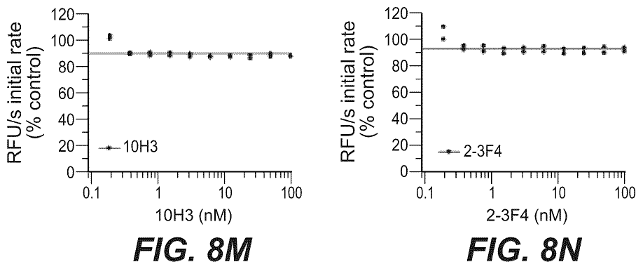

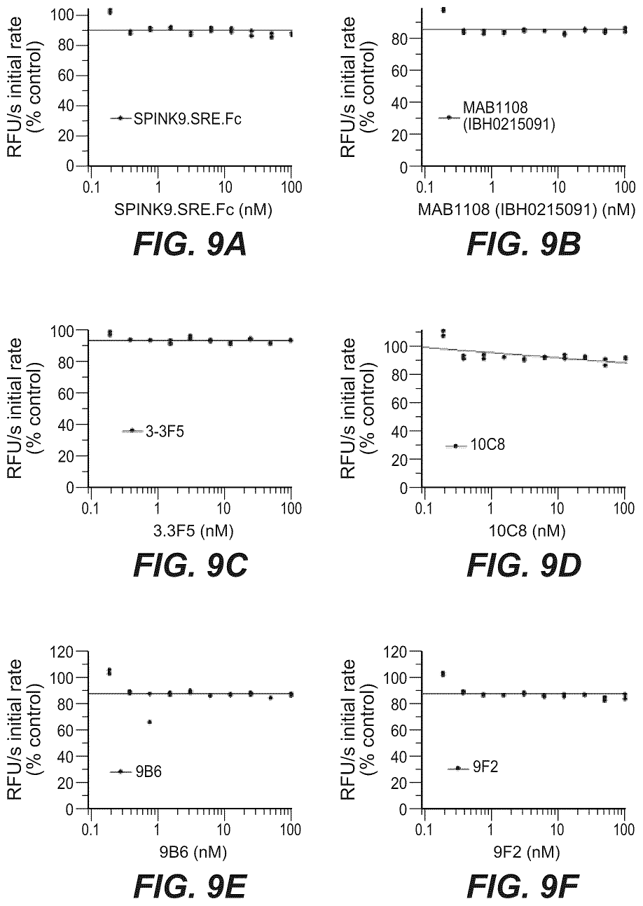

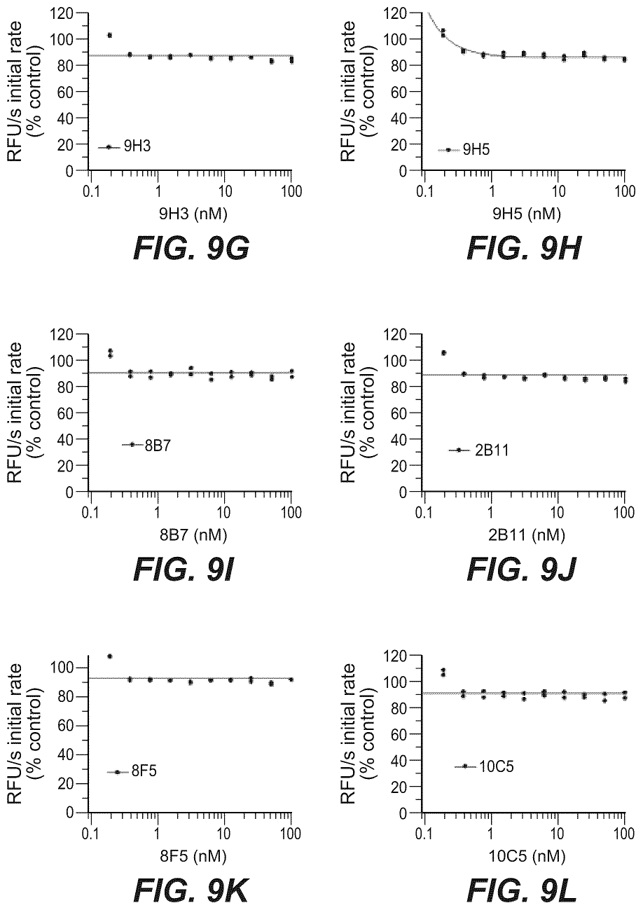

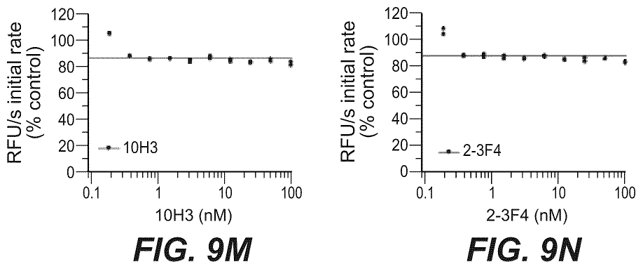

To evaluate the specificity the anti-KLK5 antibodies characterized in FIGS. 1-5, the 12 anti-KLK5 antibodies were assayed against activated human KLK7 (FIG. 6), human KLK1 (FIG. 7), human KLK4 (FIG. 8) and trypsin (FIG. 9) monitored by the cleavage of specific fluorescent peptide substrates. As these 12 anti-KLK5 antibodies were generated to selectively interact with KLK5, it was anticipated that these molecules should not inhibit other KLKs (FIGS. 6-9) or trypsin (FIG. 9).

In FIG. 6, the ability of Spink9.SRE.Fc, mAb1108 or 12 selected anti-KLK5 antibodies to inhibit the proteolysis of the substrate, Suc-LLVY-AMC, by human KLK7 was assessed using an enzymatic assay. The cleavage of the peptide substrate by human KLK7 resulted in an increased fluorescent signal and the inhibition or absence of KLK7 resulted in a quenched fluorescent signal. The results were expressed as a percentage of maximum KLK7 activity (% control). The results of a single experiment run in duplicate are shown in FIG. 6. Spink9.SRE.Fc (FIG. 6A) and mAb1108 (FIG. 6B) did not inhibit KLK7 up to 100 nM. As seen in FIG. 6, the 12 selected anti-KLK5 antibodies (FIGS. 6C-N) also do not inhibit KLK7 activity up to 100 nM. The IC₅₀ values from the curve fittings are presented in FIG. 11 (Column 6).

In FIG. 7, the ability of Spink9.SRE.Fc, mAb1108 or 12 selected anti-KLK5 antibodies to inhibit the proteolysis of the substrate, PFR-AMC, by human KLK1 was assessed using an enzymatic assay. The cleavage of the peptide substrate by human KLK1 resulted in an increased fluorescent signal and the inhibition or absence of KLK1 resulted in a quenched fluorescent signal. The results were expressed as a percentage of maximum KLK1 activity (% control). The results of a single experiment run in duplicate are shown in FIG. 7. Spink9.SRE.Fc (FIG. 7A) and mAb1108 (FIG. 7B) did not inhibit KLK1 up to 100 nM. As seen in FIG. 7, the 12 selected anti-KLK5 antibodies (FIGS. 7C-N) also do not inhibit KLK1 activity up to 100 nM. The IC₅₀ values from the curve fittings are presented in FIG. 11 (Column 7).

In FIG. 8, the ability of Spink9.SRE.Fc, mAb1108 or 12 selected anti-KLK5 antibodies to inhibit the proteolysis of the substrate, Boc-VPR-AMC, by human KLK4 was assessed using an enzymatic assay. The cleavage of the peptide substrate by human KLK4 resulted in an increased fluorescent signal and the inhibition or absence of KLK4 resulted in a quenched fluorescent signal. The results were expressed as a percentage of maximum KLK4 activity (% control). The results of a single experiment run in duplicate are shown in FIG. 8. Spink9.SRE.Fc (FIG. 8A) and mAb1108 (FIG. 8B) did not inhibit KLK4 up to 100 nM. As seen in FIG. 8, the 12 selected anti-KLK5 antibodies (FIGS. 8C-N) also do not inhibit KLK4 activity up to 100 nM. The IC₅₀ values from the curve fittings are presented in FIG. 11 (Column 8).

In FIG. 9, the ability of Spink9.SRE.Fc, mAb1108 or 12 selected antibodies to inhibit the proteolysis of the substrate, Boc-VPR-AMC, by bovine trypsin was assessed using an enzymatic assay. The cleavage of the peptide substrate by trypsin resulted in an increased fluorescent signal and the inhibition or absence of trypsin resulted in a quenched fluorescent signal. The results were expressed as a percentage of maximum trypsin activity (% control). The results of a single experiment run in duplicate are shown in FIG. 9. Spink9.SRE.Fc (FIG. 9A) and mAb1108 (FIG. 9B) did not inhibit trypsin up to 100 nM. As seen in FIG. 9, the 12 selected antibodies (FIG. 9C-N) also do not inhibit trypsin activity up to 100 nM. The IC₅₀ values from the curve fittings are presented in FIG. 11 (Column 9).

Taken together, these studies using KLK7 (FIG. 6 and Table 6), KLK1 (FIG. 7 and Table 7), KLK4 (FIG. 8 and Table 8) and trypsin (FIG. 9 and Table 9) show that Spink9.SRE.Fc, mAb1108 or the 12 selected antibodies specifically interact and inhibit only KLK5 activity.

Ranking of antibodies by Kiₐₚₚ

In characterizing the 12 selected antibodies in either the direct (FIG. 1) or coupled assays (FIGS. 2-5), it was observed that the selected antibodies have similar or greater inhibitory potency than Spink9.SRE.Fc for KLK5. However, several of the antibodies have similar IC₅₀ values in each of the assays (Tables 1-5) making it difficult to rank the potency of the antibodies.

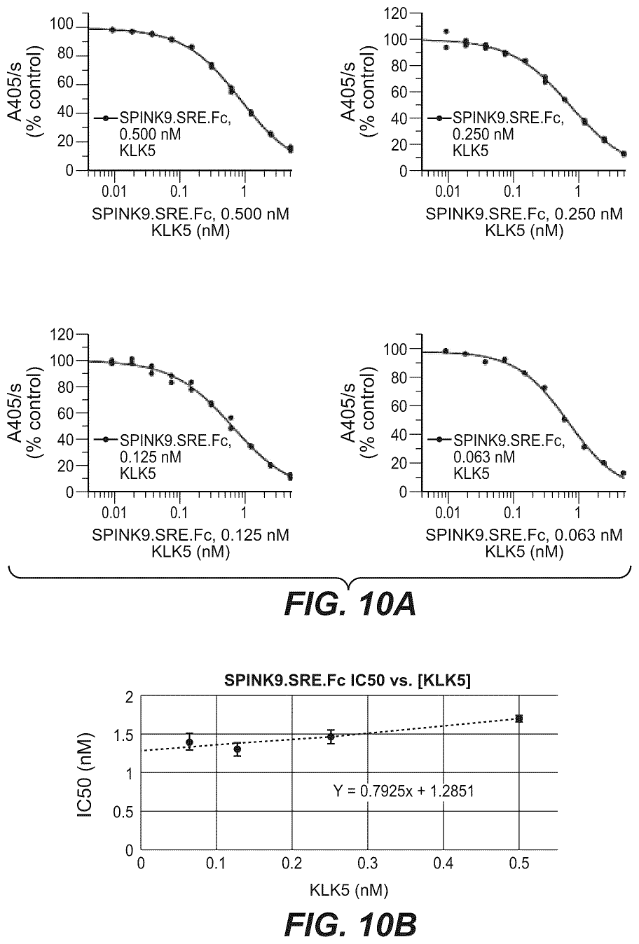

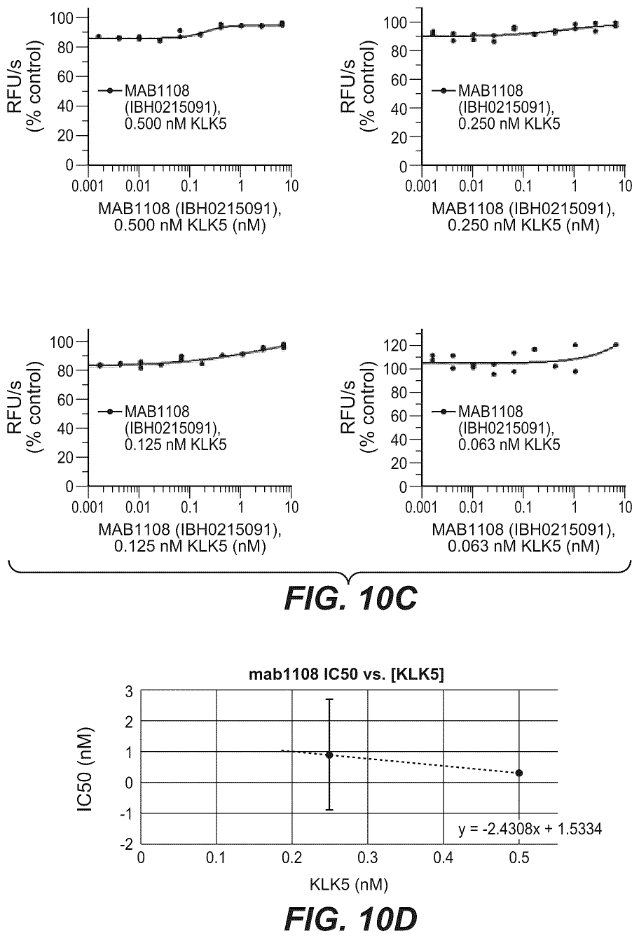

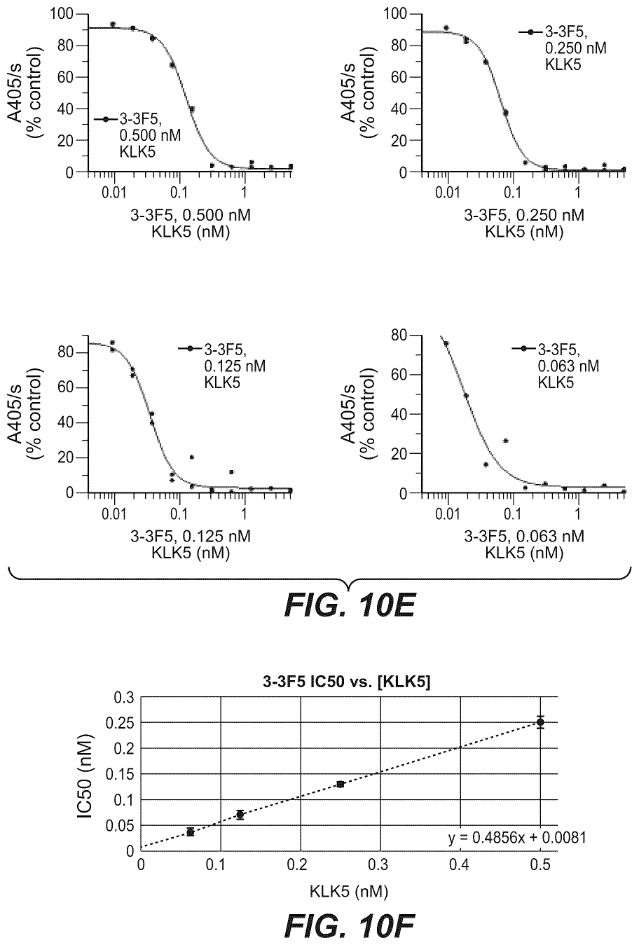

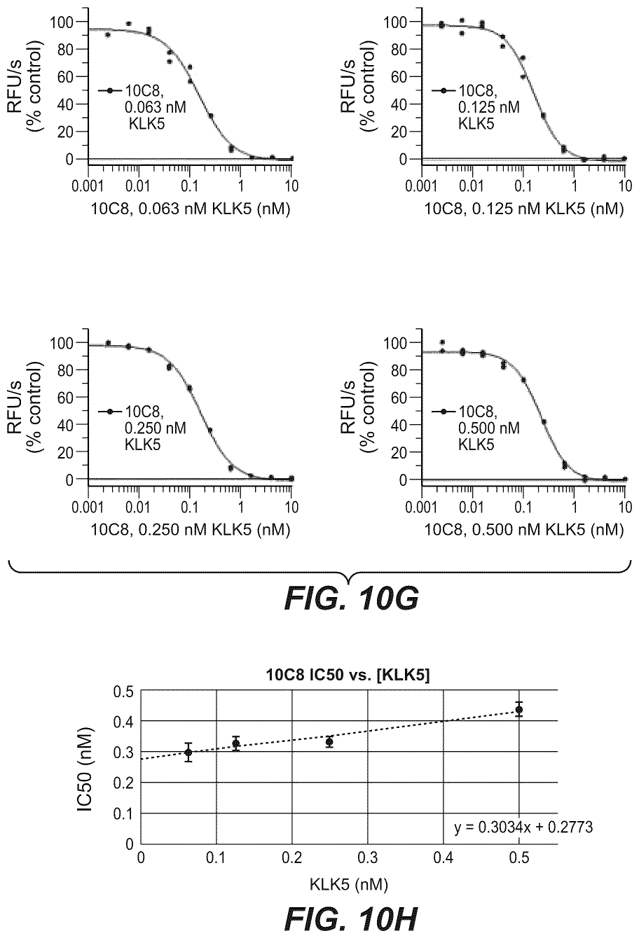

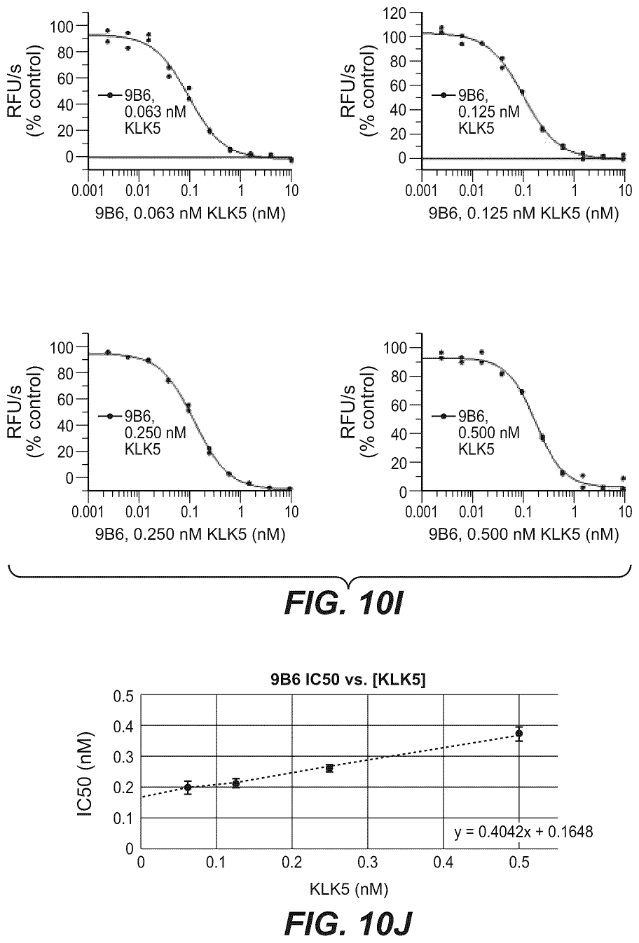

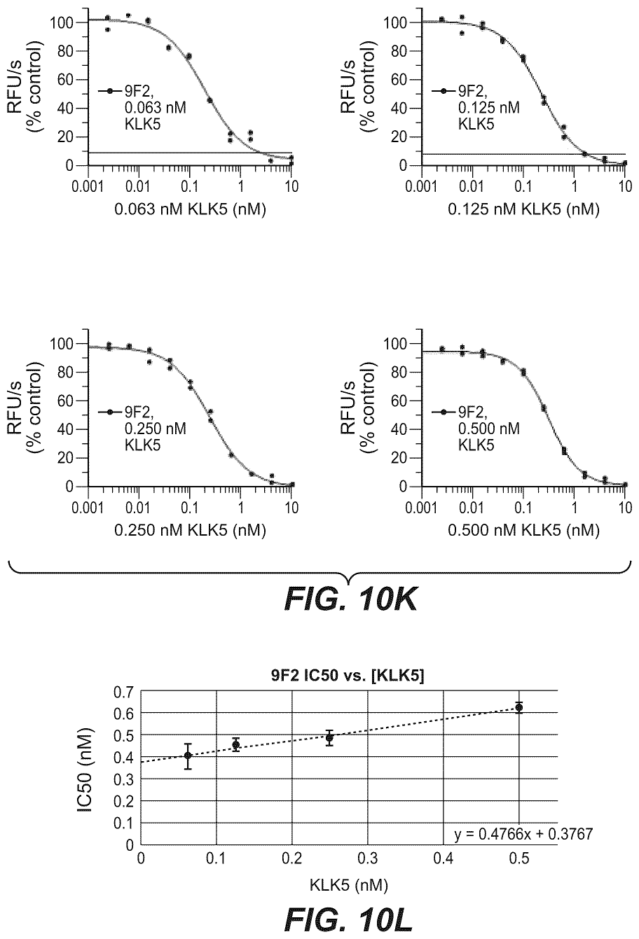

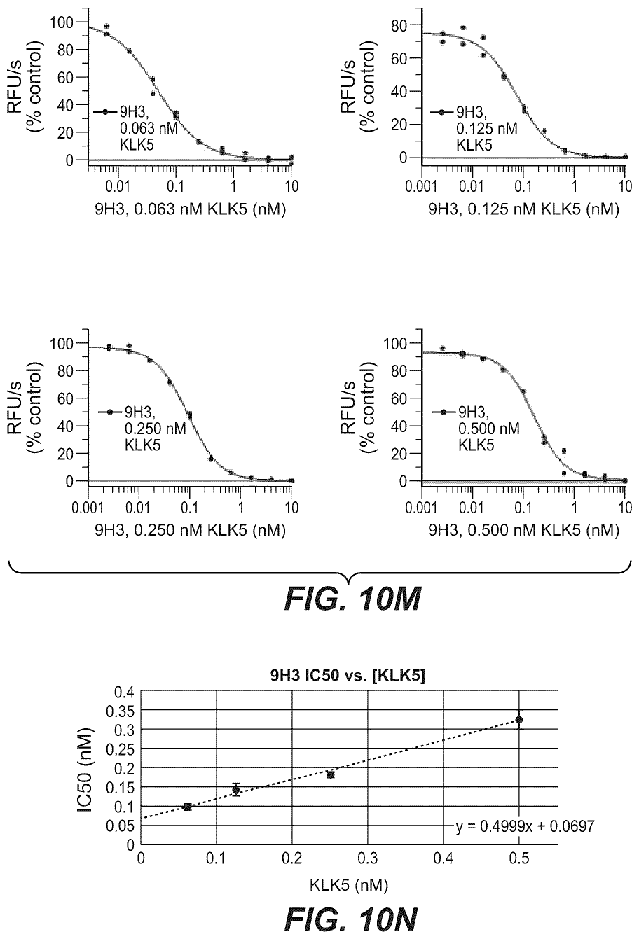

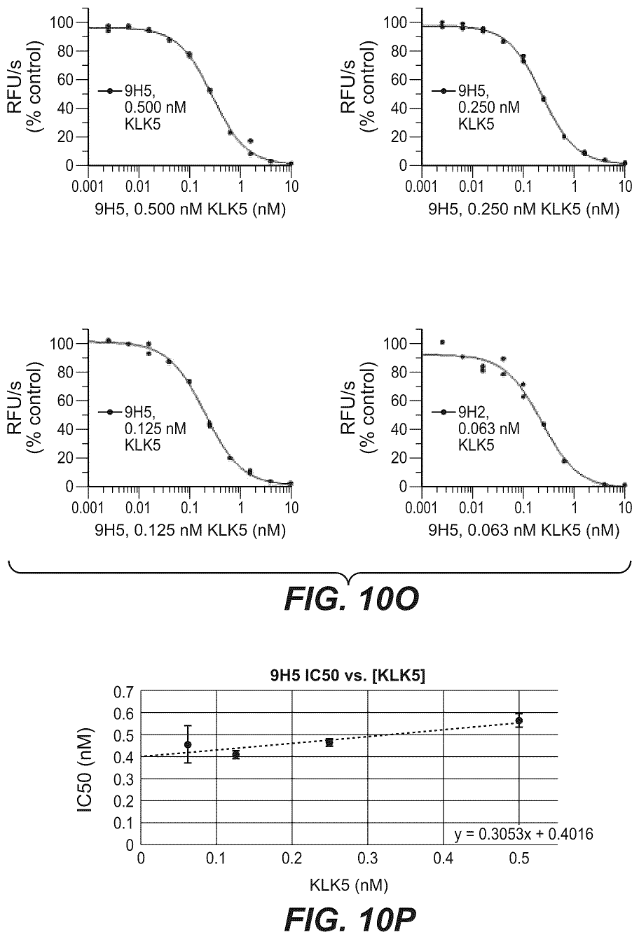

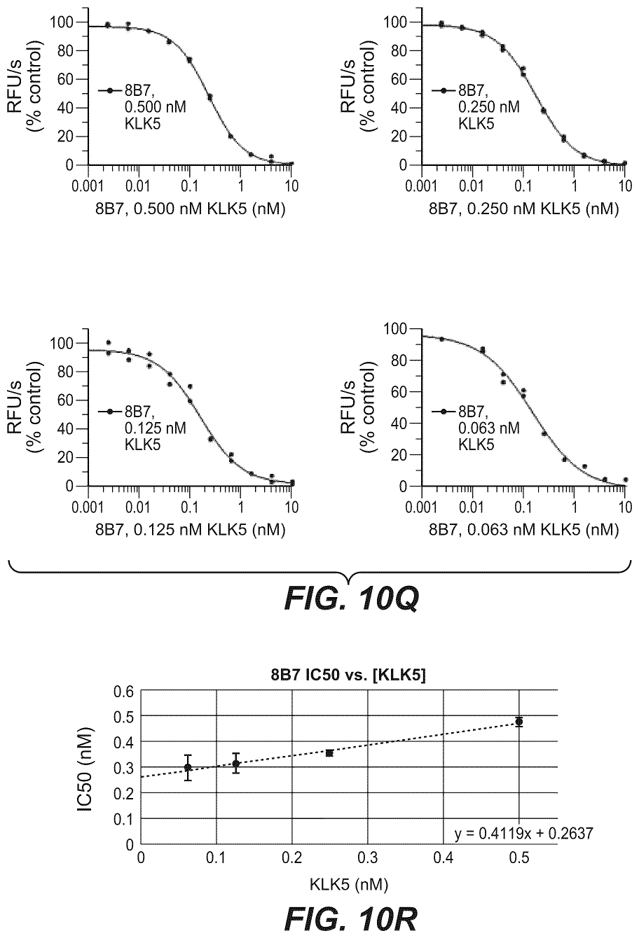

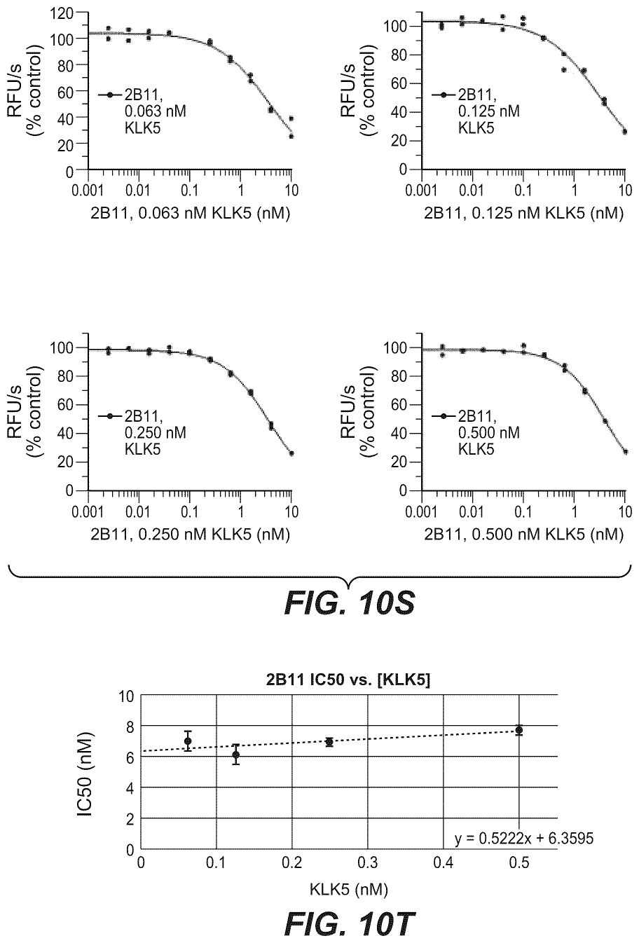

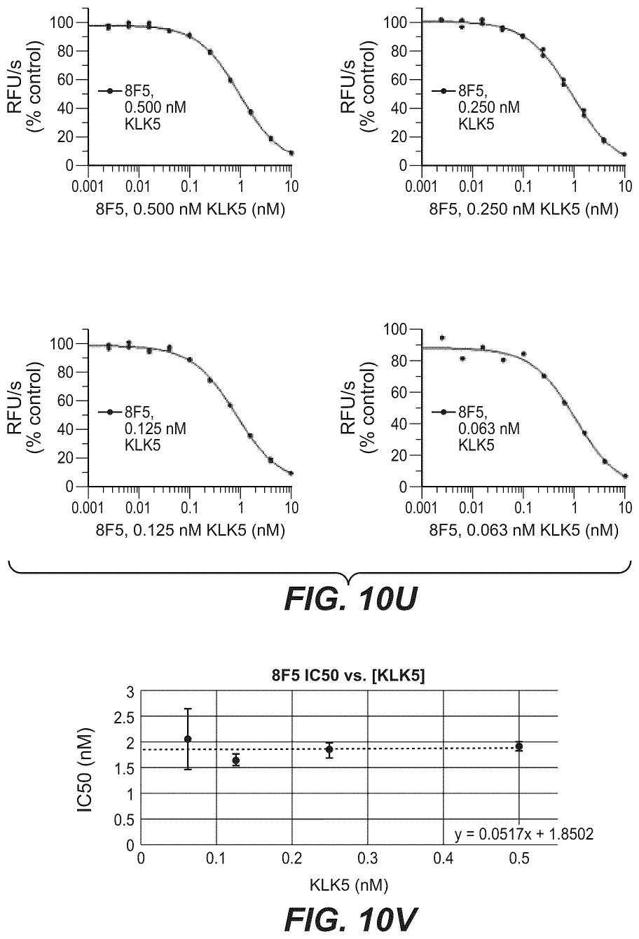

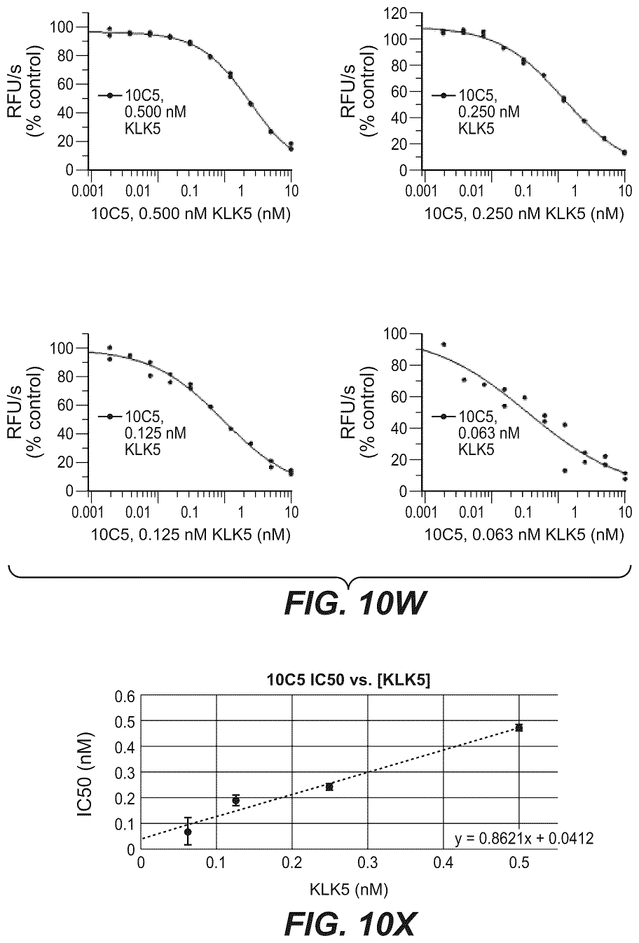

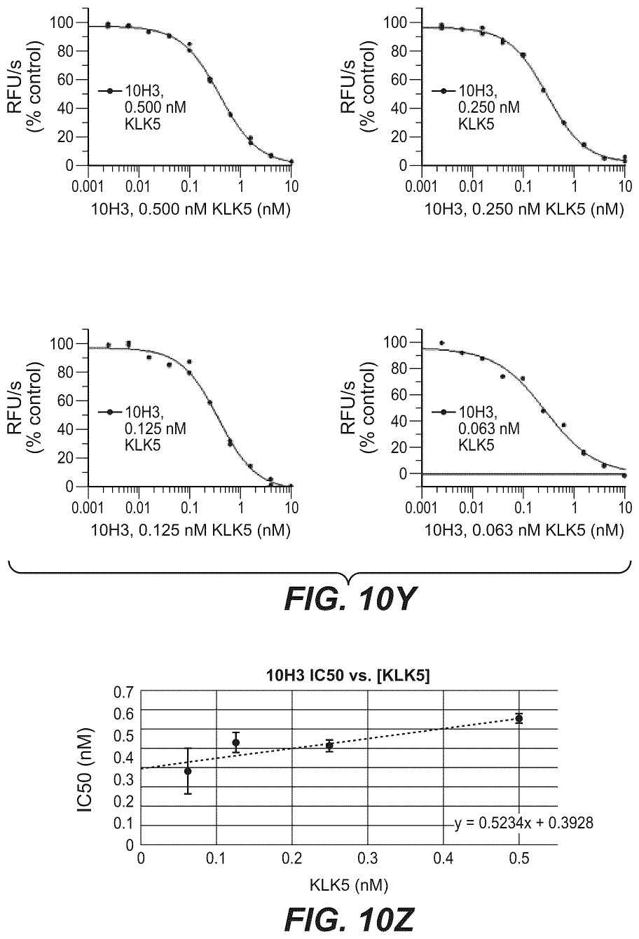

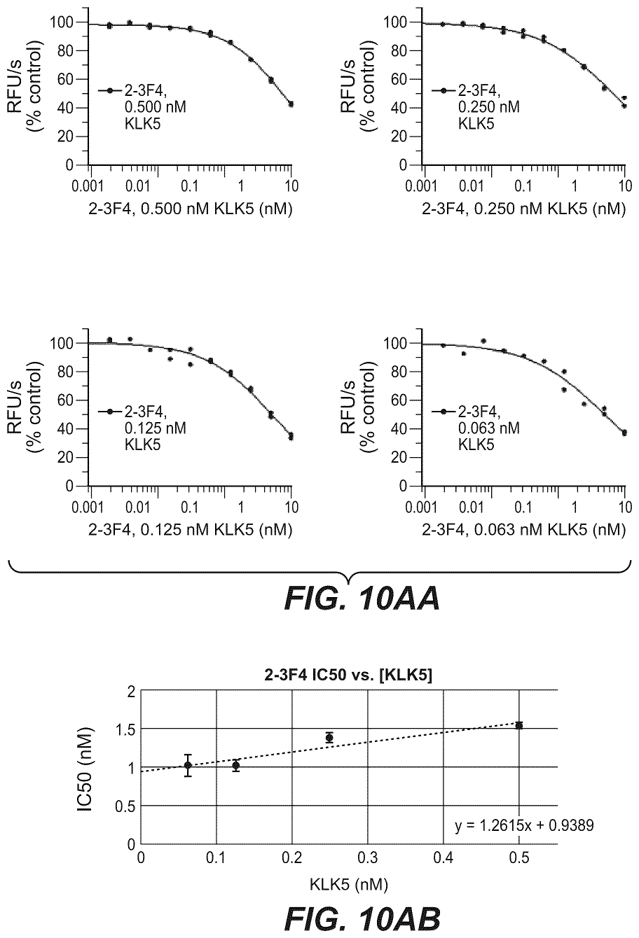

In FIG. 10, the ability of Spink9.SRE.Fc, mAb1108 or 12 selected antibodies to inhibit the proteolysis of the substrate, z-VPR-pNA, by human KLK5 at various concentrations was assessed using an enzymatic assay. In this assay the three-mer chromogenic peptide substrate contains a p-nitroanilide (pNA) group that is quenched by the terminal amino acid. The cleavage of the chromogenic peptide substrate by KLK5 resulted in an increased absorbance at 405 nm and the inhibition or absence of KLK5 resulted in a low absorbance 405 nm. The results were expressed as a percentage of maximum KLK5 activity (% control).

The IC₅₀ values of Spink9.SRE.Fc at various KLK5 concentrations (FIG. 10A) is determined and plotted as a function of KLK5 concentration (FIG. 10B) where the Kiₐₚₚ value is determined as the y-intercept. From this analysis, the Kiₐₚₚ value for Spink9.SRE.Fc is 1.28 nM. This analysis was also performed for mAb1108 (FIGS. 10 C-D) that has a Kiₐₚₚ value of 1.53 nM as well as the 12 selected antibodies (FIGS. 10 E-AB), which have a range of Kiₐₚₚ values of less than 0.01 nM to 6.35 nM. The Kiₐₚₚ values from this analysis are presented in FIG. 11 (Column 10).

### Example 4—Kinetic Analysis and Epitope Binning with Wasatch

The results of the epitope binning and off-rates against human KLK5 for a subset of the most potent antibodies are shown in Table 4. Variable levels of binding were observed to mouse and cyno KLK5. Importantly, no binding was observed to human KLK1, human KLK4, or human KLK7, confirming that these antibodies are specific. Most clones, except for 8E11 and 8G10, compete with SPINK9 for binding to human KLK5, suggesting that they either bind the same epitope (i.e., the active site) at SPINK9 or allosterically alter human KLK5 so that SPINK9 can no longer bind.

TABLE 4

| | SPINK9 | human |
|---|---|---|
| | competition | KLK5 |
| Clone ID | (Y/N) | kd (1/s) |
| 14C8 | Y | 3.76E⁻⁰⁴ |
| 14E12 | Y | 4.14E⁻⁰⁴ |
| 2B11 | Y | 5.74E⁻⁰⁴ |
| 8B7 | Y | 5.47E⁻⁰⁴ |
| 8E11 | N | 2.02E⁻⁰⁴ |
| 8F5 | Y | 3.16E⁻⁰⁴ |
| 8G10 | N | 2.45E⁻⁰⁴ |
| 9B6 | Y | 1.78E⁻⁰⁴ |
| 9F2 | Y | 3.39E⁻⁰⁴ |
| 9H3 | Y | 1.43E⁻⁰⁴ |
| 9H5 | Y | 3.67E⁻⁰⁴ |
| 10C5 | Y | 1.26E⁻⁰⁴ |
| 10C8 | Y | 6.16E⁻⁰⁵ |
| 10H3 | Y | 5.89E⁻⁰⁴ |
| 2.3F4 | Y | 1.84E⁻⁰⁴ |
| SPINK9-SRE-Fc | Y | 4.70E⁻⁰⁴ |

### Example 5—Epitope Mapping by Hydrogen Exchange Mass Spectrometry

Hydrogen exchange measurements as completed herein measure the exchange of protons bound to backbone amide residues with those bound to molecules of the solvent. When a protiated (¹H) sample is diluted into deuterated solvent, difference in mass between a proton and deuteron can be measured by mass spectrometry. Upon a series of increasing incubation times in deuterated solvent (²H₂O), rate of exchange can be measured. In this way, KLK 5 (SEQ ID NO:353) complexed to one of three anti-KLK5 antibodies (10C5, 10H3 and 9H5) over three separate experiments was measured and then compared to results obtained from performing the experiment on KLK5 alone. A differential analysis of mass differences was then performed and overlapping peptides utilized to narrow in on a common thermodynamic epitope shared between all three anti-KLK5 antibodies (10C5, 10H3 and 9H5) (FIG. 12 A). The term “thermodynamic epitope” refers to those portions of a protein whose backbone structural dynamics, or local free energy of unfolding is altered in response to a specific binding event such as becoming bound by an antibody. The structural epitope may be contained within the thermodynamic epitope.

Interpretation of data used the rule of N-2 whereby each peptide cannot carry deuterium on the first two sites due to back exchange. In addition, when there are many unique overlapping peptides, this information may be used to narrow in on those affected residues within each peptide. The four sequence regions of KLK5 identified to comprise a common thermodynamic epitope between are LRPNQL (SEQ ID NO: 316, FIG. 12 B, Region 1), QGVKSI (SEQ ID NO: 317, FIG. 12 B, Region 2), KRCEDAYPRQIDDT (SEQ ID NO: 318, FIG. 12 B, Region 3), and DYPCARPNRPGVY (SEQ ID NO: 319, FIG. 12 B, Region 4), a common structural epitope between all three antibodies tested is contained within these regions.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### Example 6—Evaluation of KLK5 Inhibitor IC50 Values in a Cell-Based IL-8 Secretion Assay

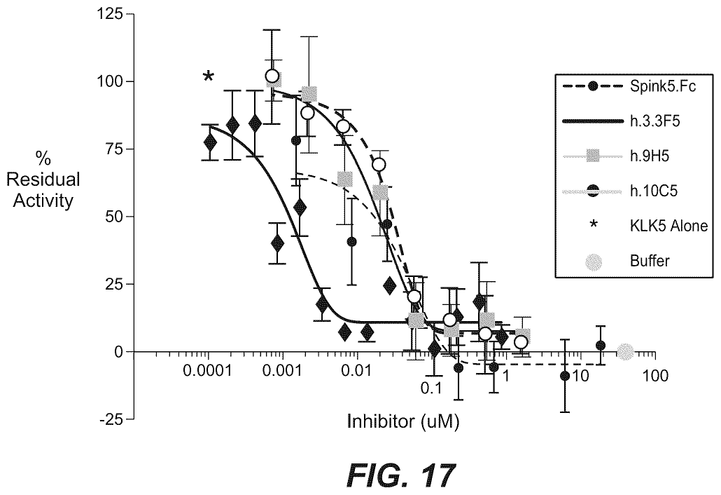

KLK5 stimulates A549 cells to secrete IL-8. The ability of Spink5.Fc, Spink9.SRE.Fc, or humanized anti-KLK5 antibodies 3-3F5, 9H5, or 10C5, to inhibit KLK5-induced IL-8 secretion was evaluated in the A549 cell-based IL-8 secretion assay. In this assay, serum-starved A549 cells were stimulated with 200 nM KLK5 for 24 hr (KLK5 alone or KLK5 pre-incubated with inhibitors for 1 hour). The IL-8 concentration was measured with a sandwich ELISA and IC50s of the inhibitors were determined. The results were expressed as a percentage of maximum KLK5 activity and the averages±standard deviations of at least three independent experiments are shown in FIG. 17. All inhibitors fully inhibited KLK5-induced IL-8 secretion to within 5% of the level of buffer alone (starvation media), which was within the error of the assay. All inhibitors showed potent inhibition of KLK5-induced IL-8 secretion and the average IC50s for at least three independent experiments were as follows: Spink5.Fc (21 nM); 10C5 (28 nM); 9H5 (15 nM); 3-3F5 (1.2 nM). The residual activity observed when KLK5 is incubated with 360 nM Spink9.SRE.Fc is 2% (data not shown).

### Example 7—Structure of 10C5, 9H5 and 3-3F5 Bound to Human KLK5

For crystal structure experiments described herein, human KLK5 residues I67-S293 (SEQ ID NO:328) were used. The numbering of the amino acid residues of human KLK5 used in this Example 7 and as shown in corresponding FIGS. 18-20 and Tables 5-13 is based on the standard numbering for proteases. See Debela et al., J Mol Biol, 373, 1017-1031 (2007). The numbering of the amino acid residues of the Fab fragments used in this Example 7 and as shown in corresponding FIGS. 18-20 and Tables 5-13 is based on Kabat.

### The Structure of 10C5 Bound to Human KLK5

The 10C5 Fab/KLK5 complex structure shows that the binding of 10C5 to KLK5 results in conformational changes that allosterically result in disruption of the substrate binding site as well as the active site of KLK5, including the catalytic triad, rendering the protease unable to bind substrates and losing activity. The most distinct element of KLK5 that is recognized by the 10C5 fab is the helix encompassing residues 163-174A. The loop/turn immediately following the helix, made up by the residues 173-174A is flipped up by the antibody binding which results in a steric clash with residues in the 220s loop as well as the 90s loop (standard protease convention), that are important for substrate binding as well as positioning of the catalytic triad in the active site that cleaves peptides (FIG. 18A).

The buried surface area between KLK5 and 10C5 Fab is ˜860 Å². In the 10C5 fab light chain, CDR-L1, residues Q24-S34 contact residues S131, A132, S164 and R167 in KLK5; Y50 from CDR-L2 contacts R167 in KLK5; CDR-L3 contacts A132, L163, S164, Q165, K166, E169, 1176, D177 and D178 in KLK5. In the 10C5 heavy chain, CDRH1, residues S31-T35 contact only D170 in KLK5; CDRH2, residues Y50-A63, contacts residues K166, E169, D170, A171, Y172, P173, R174, Q174A and 1176 in KLK5; CDRH3, E95-Y100c contacts residues S164, K166, R167 and D170 in KLK5 (Tables 5-7, FIG. 18B).

The sequences of the 10C5 Fab HC and LC used for the crystallization with human KLK5 is depicted in SEQ ID NO:322 and SEQ ID NO:323, respectively.

TABLE 5

| Interface | Interface | Interface | Interface |
|---|---|---|---|
| residues | residues | residues | residues |
| in 10C5 | in KLK5 | in 10C5 | in KLK5 |
| Heavy Chain | (Standard | Light Chain | (Standard |
| (Kabat | Protease | (Kabat | Protease |
| Numbering) | Numbering) | Numbering) | Numbering) |
| Ser 31 | Ser 164 | Glu 27 | Pro 130 |
| Gly 33 | Lys 166 | Ser 28 | Ser 131 |
| Tyr 50 | Arg 167 | Ser 30 | Ala 132 |
| Thr 52 | Glu 169 | Glu 32 | Val 162 |
| Ser 52A | Asp 170 | Tyr 50 | Leu 163 |
| Asn 53 | Ala 171 | Gly 91 | Ser 164 |
| Tyr 54 | Tyr 172 | Phe 92 | Gln 165 |
| Val 56 | Pro 173 | Gly 93 | Lys 166 |
| Ser 57 | Arg 174 | Ser 94 | Arg 167 |
| Tyr 58 | Gln 174A | Ser 95 | Glu 169 |
| Tyr 59 | Ile 176 | Val 97 | Gln 174A |
| Lys 64 | Arg 224 | | Ile 176 |
| Glu 95 | | | Asp 177 |
| Pro 97 | | | Asp 178 |
| Gly 100 | | | Lys 233 |
| Tyr 100A | | | |
| Tyr 100C | | | |

TABLE 6
Hydrogen Bonds

| | Bond | | | Bond | |
|---|---|---|---|---|---|
| Heavy Chain | Length | | Light Chain | Length | |
| Residue | (Angstrom) | KLK5 residue | Residue | (Angstrom) | KLK5 residue |
| Tyr 50 [OH] | 2.8 | Glu 169 [OE2] | Tyr 50 [OH] | 2.6 | Arg 167 [NH1] |
| Ser 52A [OG] | 2.6 | Asp 170 [OD1] | Phe 92 [O] | 2.9 | Gln 165 [N] |
| Ser 52A [N] | 3.4 | Asp 170 [OD2] | Phe 92 [O] | 3.2 | Lys 166 [N] |
| Tyr 58 [OH] | 3.8 | Arg 174 [O] | Ser 95 [OG] | 3.7 | Asp 178 [N] |
| Ser 57 [O] | 2.6 | Gln 174A [NE2] | Ser 94 [N] | 2.7 | Glu 169 [OE1] |
| Tyr 58 [OH] | 3.0 | Ile 176 [N] | | | |

TABLE 7
Salt Bridges

| | Bond | |
|---|---|---|
| Heavy Chain | Length | |
| Residue | (Angstrom) | KLK5 residue |
| Glu 95 [OE1] | 2.3 | Lys 166 [NZ] |
| Glu 95 [OE1] | 3.4 | Lys 166 [NZ] |

### The Structure of 9H5 Bound to Human KLK5

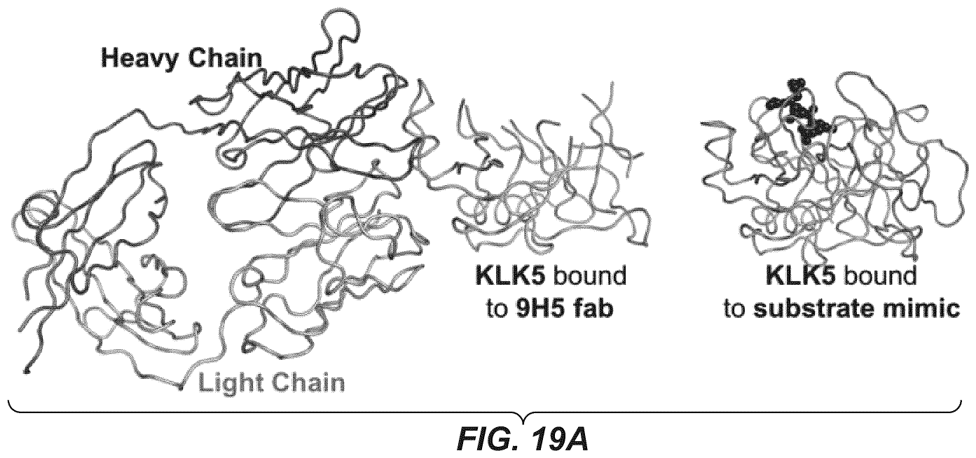

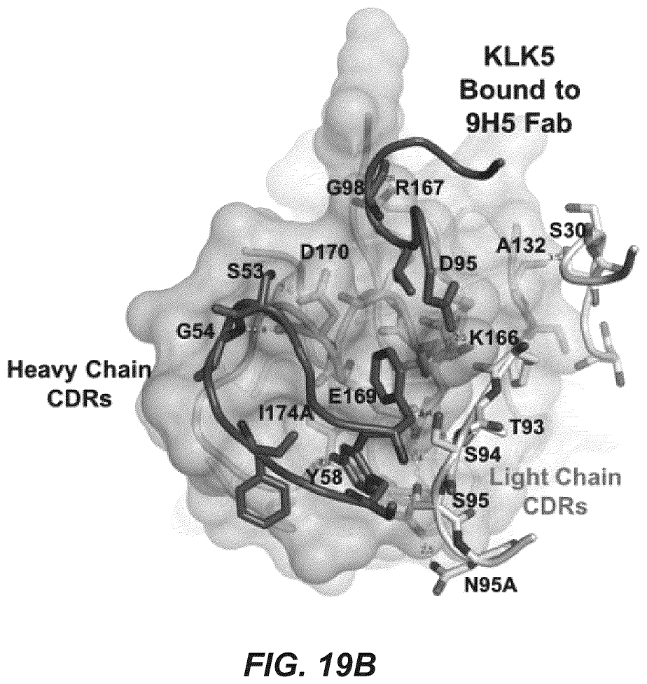

The 9H5 Fab/KLK5 complex structure shows that the binding of 9H5 to KLK5 results in conformational changes, similar to 10C5 fab, result in allosteric disruption of the substrate binding site as well as the active site of KLK5, including the catalytic triad, rendering the protease unable to bind substrates and losing activity. The most distinct element of KLK5 that is recognized by the 9H5 fab is the helix encompassing residues 163-174A. The loop/turn immediately following the helix, made up by the residues 173-174A is flipped up by the antibody binding which results in a steric clash with residues in the 220s loop as well as the 90s loop (standard protease convention), that are important for substrate binding as well as positioning of the catalytic triad in the active site that cleaves peptides. Overall, more of the KLK5 protein is disordered in the 9H5:KLK5 complex crystal structure, compared to 10C5 bound KLK5, and is likely a direct result of the Fab binding and the induced conformational changes (FIG. 19A).

The buried surface area between KLK5 and 91H5 Fab is 805 Å². In the 91H5 light chain, CDRL1, residues Q24-S34 contacts residues 5131, A132, G133, L163, 5164, R167 in KLK5; CDRL2, residues S50-56, does not contact any residues in KLK5; CDRL3, residues H89-T97 contact residues A132, S164, Q165, K166, E169, Q174A, 1176 and D177 in KLK5. In the 91H5 heavy chain, CDRH1, residues S31-35 do not contact KLK5; CDRH2, residues F50-A63 contacts residues K166, E169, D170, Y172, P173, R174, Q174A and 1176 in KLK5; CDRH3, residues D95-102 contacts residues K166, R167 and D170 in KLK5 (Tables 8-10, FIG. 19B).

The sequences of the 95 Fab HC and LC used for the crystallization with human KLK5 is depicted in SEQ ID NO:324 and SEQ ID NO:325, respectively.

TABLE 8

| Interface | Interface | Interface | Interface |
|---|---|---|---|
| residues | residues | residues | residues |
| in 9H5 | in KLK5 | in 9H5 | in KLK5 |
| Heavy Chain | (Standard | Light Chain | (Standard |
| (Kabat | Protease | (Kabat | Protease |
| Numbering) | Numbering) | Numbering) | Numbering) |
| Ser 31 | Ser 164 | Gln 27 | Pro 130 |
| Gly 33 | Lys 166 | Ser 28 | Ser 131 |
| Ser 35 | Arg 167 | Ser 30 | Ala 132 |
| Phe 50 | Glu 169 | Tyr 32 | Gly 133 |
| Gly 52 | Asp 170 | His 89 | Val 162 |
| Ser 53 | Ala 171 | Gln 90 | Leu 163 |
| Gly 54 | Tyr 172 | Asp 91 | Ser 164 |
| Phe 56 | Pro 173 | Tyr 92 | Gln 165 |
| Tyr 58 | Arg 174 | Thr 93 | Lys 166 |
| Lys 64 | Gln 174A | Ser 94 | Arg 167 |
| Asp 95 | Ile 176 | Ser 95 | Glu 169 |
| Val 97 | Asp 177 | Asn 95A | Gln 174A |
| Gly 98 | | Thr 97 | Ile 176 |
| Ser 100B | | | Asp 177 |
| Leu 100C | | | Lys 233 |

TABLE 9
Hydrogen Bonds

| | Bond | | | Bond | |
|---|---|---|---|---|---|
| Heavy Chain | Length | | Light Chain | Length | |
| Residue | (Angstrom) | KLK5 residue | Residue | (Angstrom) | KLK5 residue |
| Ser 53 [N] | 2.3 | Asp 170 [OD2] | Tyr 92 [O] | 3.2 | Gln 165 [N] |
| Ser 53 [N] | 3.5 | Asp 170 [O] | Tyr 92 [O] | 2.8 | Lys 166 [N] |
| Ser 53 [OG] | 2.2 | Asp 170 [OD2] | Ser 30 [OG] | 3.1 | Ala 132 [O] |
| Ser 53 [OG] | 3.7 | Asp 170 [O] | Thr 93 [OG1] | 2.9 | Glu 169 [OE1] |
| Gly 54 [N] | 3.5 | Asp 170 [OD2] | Ser 95 [OG] | 3.4 | Glu 169 [OE1] |
| Gly 54 [N] | 2.8 | Asp 170 [O] | Ser 94 [N] | 3.0 | Glu 169 [OE2] |
| Tyr 58 [OH] | 3.1 | Ile 176 [O] | Ser 95 [OG] | 3.6 | Ile 176 [O] |
| Gly 98 [O] | 3.6 | Arg 167 [NH2] | Asn 95A [ND2] | 2.5 | Asp 177 [OD1] |
| Tyr 58 [OH] | 2.7 | Ile 176 [N] | | | |

TABLE 10
Salt Bridges

| | Bond | |
|---|---|---|
| Heavy Chain | Length | |
| Residue | (Angstrom) | KLK5 residue |
| Asp 95 [OD1] | 2.5 | Lys 166 [NZ] |
| Asp 95 [OD2] | 3.7 | Lys 166 [NZ] |

### The Structure of 3F5.5 Fab Bound to Human KLK5

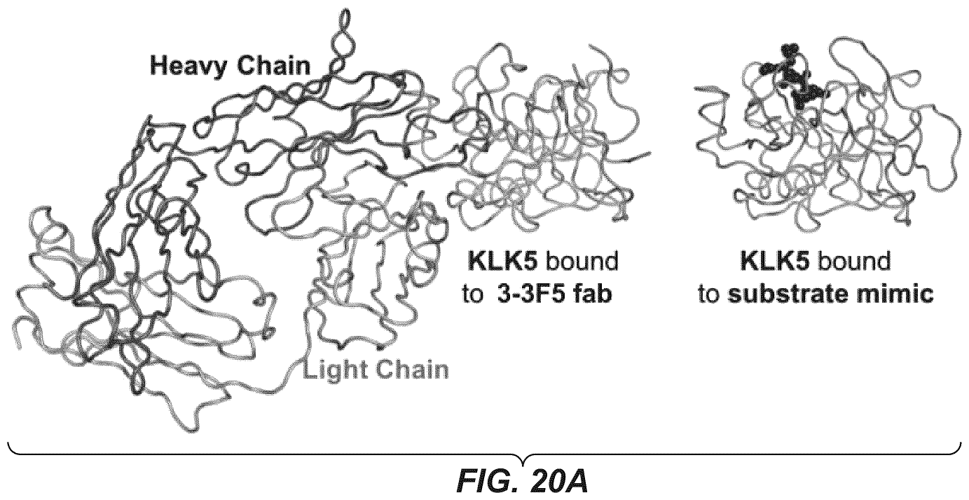

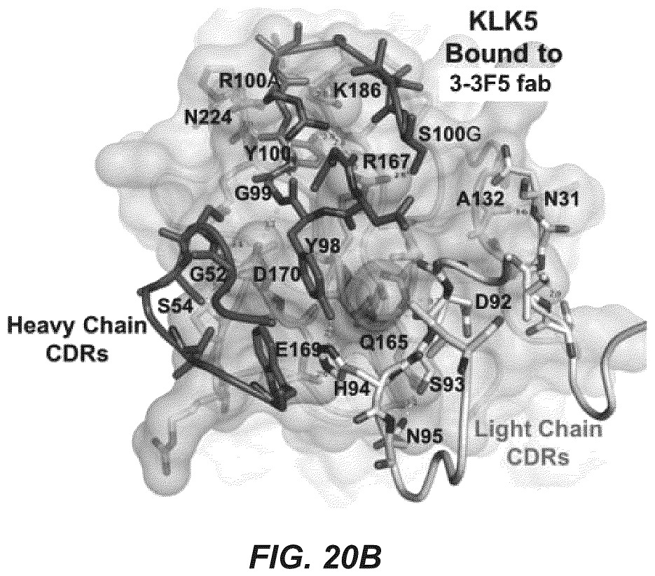

The 3-3F5 Fab/KLK5 complex structure shows that the binding of the 3-3F5 to KLK5 results in slight conformational changes, although not as drastic as observed with the 10C5 or the 9H5 complexes with KLK5. The substrate recognition pocket is altered, where the Asp 189 in KLK5, (which forms a salt bridge and thereby recognizing positively charged P1-residue (Arg/Lys) before proteolysis) becomes disordered. The catalytic triad in KLK5 appears intact, although the fab binding renders the catalytic pocket more open, resulting in KLK5 losing catalytic activity. The 140s loop and 70s loop are disordered in the crystal structure (FIG. 20A).

The buried surface area between KLK5 and 3F5.5 Fab is 956 A2. In the 3-3F5 light chain, CDRL1, residues Q24-A34 contact S131, A132 and G133 in KLK5; CDRL2, residues D50-S56 does not contact any residues in KLK5; CDRL3, residues Q89-I97 contact residues S164, Q165, K166 and E169 in KLK5. In the 3F5.5 heavy chain, CDRH1 residues D31-E46 does not interact with KLK5 at all, neither does CDRH2; CDRH3, residues D95-G100i contact residues L163, S164, K166, R167, D170, G184, D185, K186, A187, N224, R225 and P225 in KLK5 (Tables 11-13, FIG. 20B).

The sequences of the 3-3F5 Fab HC and LC used for the crystallization with human KLK5 is depicted in SEQ ID NO:326 and SEQ ID NO:327, respectively.

TABLE 11

| Interface | Interface | Interface | Interface |
|---|---|---|---|
| residues | residues | residues | residues |
| in 3-3F5 | in KLK5 | in 3-3F5 | in KLK5 |
| Heavy Chain | (Standard | Heavy Chain | (Standard |
| (Kabat | Protease | (Kabat | Protease |
| Numbering) | Numbering) | Numbering) | Numbering) |
| Asp 31 | Leu 163 | Ile2 | Ser 131 |
| Trp 47 | Ser 164 | Ser 28 | Ala 132 |
| Gly 52 | Lys 166 | Ile 29 | Gly 133 |
| Ser 53 | Arg 167 | Gly 30 | Ser 164 |
| Ser 54 | Glu 169 | Asn 31 | Gln 165 |
| Val 56 | Asp 170 | Ala 32 | Lys 166 |
| Trp 58 | Ala 171 | Asp 50 | Arg 167 |
| Asp 95 | Pro 173 | Gly 68 | Glu 169 |
| Arg 96 | Arg 174 | Gln 89 | Asp 170 |
| Asp 97 | Gly 184 | Gln 90 | |
| Tyr 98 | Asp 185 | Gly 91 | |
| Gly 99 | Lys 186 | Asp 92 | |
| Tyr 100 | Ala 186A | Ser 93 | |
| Arg 100A | Arg 188 | His 94 | |
| Ala 100B | Asn 223 | Asn 95 | |
| Asp 100C | Arg 224 | Ile 97 | |
| Ala 100E | Pro 225 | | |
| Thr 100F | | | |
| Ser 100G | | | |
| Met 100I | | | |

TABLE 12
Hydrogen Bonds

| | Bond | | | Bond | |
|---|---|---|---|---|---|
| Heavy Chain | Length | | Light Chain | Length | KLK5 |
| Residue | (Angstrom) | KLK5 residue | Residue | (Angstrom) | residue |
| Ser 53 [N] | 3.0 | Asp 170 [OD1] | Ser 28 [OG] | 2.9 | Ser 131 [OG] |
| Ser 54 [N] | 3.0 | Asp 170 [OD1] | Asp 92 [OD1] | 3.7 | Gln 165 [N] |
| Ser 54 [OG] | 2.8 | Asp 170 [OD1] | Asp 92 [O] | 3.1 | Gln 165 [N] |
| Gly 99 [N] | 3.1 | Asp 170 [OD2] | Asp 92 [O] | 3.0 | Lys 166 [N] |
| Ser 53 [N] | 3.1 | Asp 170 [OD2] | Asn 31 [N] | 3.6 | Ala 132 [O] |
| Ser 53 [OG] | 3.0 | Asp 170 [OD2] | Asn 95 [ND2] | 3.1 | Gln 165 [OE] |
| Ala 100B [N] | 2.8 | Asn 223 [OD1] | | | |
| Thr 100F [O] | 3.9 | Arg 167 [NH1] | | | |
| Arg 96 [O] | 2.8 | Arg 167 [NH1] | | | |
| Ser 100G [OG] | 2.8 | Arg 167 [NH2] | | | |
| Tyr 100 [OH] | 2.9 | Asp 185 [N] | | | |
| Tyr 100 [OH] | 3.7 | Lys 186 [N] | | | |
| Arg 100A [O] | 2.8 | Lys 186 [NZ] | | | |
| Tyr 100 [O] | 3.0 | Asn 223 [ND2] | | | |

TABLE 13
Salt Bridges

| | Bond | |
|---|---|---|
| Heavy Chain | Length | |
| Residue | (Angstrom) | KLK5 residue |
| His 94 [NE2] | 2.8 | Glu 169 [OE1] |
| His 94 [NE2] | 3.9 | Glu 169 [OE2] |

Table of Sequences

| | | SEQ |
|---|---|---|
| NAME | SEQUENCE | ID NQ |
| Human KLK5 | MATARPPWMWVLCALITALLLGVTEHVLANNDVSCDHPSNTVPSGSNQDLGAGAGE | 1 |
| Q9Y337 | DARSDDSSSRIINGSDCDMHTQPWQAALLLRPNQLYCGAVLVHPQWLLTAAHCRKK | |
| G55, D153 | VFRVRLGHYSLSPVYESGQQMFQGVKSIPHPGYSHPGHSNDLMLIKLNRRIRPTKD | |
| (incl. signal | VRPINVSSHCPSAGTKCLVSGWGTTKSPQVHFPKVLQCLNISVLSQKRCEDAYPRQ | |
| peptide | IDDTMFCAGDKAGRDSCQGDSGGPVVCNGSLQGLVSWGDYPCARPNRPGVYTNLCK | |
| (underlined)) | FTKWIQETIQANS | |
| Human KLK5 | VTEHVLANNDVSCDHPSNTVPSGSNQDLGAGAGEDARSDDSSSRIINGSDCDMHTQ | 2 |
| G55, D153 | PWQAALLLRPNQLYCGAVLVHPQWLLTAAHCRKKVFRVRLGHYSLSPVYESGQQMF | |
| (mature form) | QGVKSIPHPGYSHPGHSNDLMLIKLNRRIRPTKDVRPINVSSHCPSAGTKCLVSGW | |
| | GTTKSPQVHFPKVLQCLNISVLSQKRCEDAYPRQIDDTMFCAGDKAGRDSCQGDSG | |
| | GPVVCNGSLQGLVSWGDYPCARPNRPGVYTNLCKFTKWIQETIQANS | |
| Human KLK5 | MATARPPWMWVLCALITALLLGVTEHVLANNDVSCDHPSNTVPSGSNQDLGAGAGE | 3 |
| G55, N153 | DARSDDSSSRIINGSDCDMHTQPWQAALLLRPNQLYCGAVLVHPQWLLTAAHCRKK | |
| (incl. signal | VFRVRLGHYSLSPVYESGQQMFQGVKSIPHPGYSHPGHSNNLMLIKLNRRIRPTKD | |
| peptide | VRPINVSSHCPSAGTKCLVSGWGTTKSPQVHFPKVLQCLNISVLSQKRCEDAYPRQ | |
| (underlined)) | IDDTMFCAGDKAGRDSCQGDSGGPVVCNGSLQGLVSWGDYPCARPNRPGVYTNLCK | |
| | FTKWIQETIQANS | |
| Human KLK5 | VTEHVLANNDVSCDHPSNTVPSGSNQDLGAGAGEDARSDDSSSRIINGSDCDMHTQ | 4 |
| G55, N153 | PWQAALLLRPNQLYCGAVLVHPQWLLTAAHCRKKVFRVRLGHYSLSPVYESGQQMF | |
| (mature form) | QGVKSIPHPGYSHPGHSNNLMLIKLNRRIRPTKDVRPINVSSHCPSAGTKCLVSGW | |
| | GTTKSPQVHFPKVLQCLNISVLSQKRCEDAYPRQIDDTMFCAGDKAGRDSCQGDSG | |
| | GPVVCNGSLQGLVSWGDYPCARPNRPGVYTNLCKFTKWIQETIQANS | |
| Human KLK5 | MATARPPWMWVLCALITALLLGVTEHVLANNDVSCDHPSNTVPSGSNQDLGAGARE | 5 |
| R55, N153 | DARSDDSSSRIINGSDCDMHTQPWQAALLLRPNQLYCGAVLVHPQWLLTAAHCRKK | |
| (incl. signal | VFRVRLGHYSLSPVYESGQQMFQGVKSIPHPGYSHPGHSNNLMLIKLNRRIRPTKD | |
| peptide | VRPINVSSHCPSAGTKCLVSGWGTTKSPQVHFPKVLQCLNISVLSQKRCEDAYPRQ | |
| (underlined)) | IDDTMFCAGDKAGRDSCQGDSGGPVVCNGSLQGLVSWGDYPCARPNRPGVYTNLCK | |
| | FTKWIQETIQANS | |
| Human KLK5 | VTEHVLANNDVSCDHPSNTVPSGSNQDLGAGAREDARSDDSSSRIINGSDCDMHTQ | 6 |
| R55, N153 | PWQAALLLRPNQLYCGAVLVHPQWLLTAAHCRKKVFRVRLGHYSLSPVYESGQQMF | |
| (mature form) | QGVKSIPHPGYSHPGHSNNLMLIKLNRRIRPTKDVRPINVSSHCPSAGTKCLVSGW | |
| | GTTKSPQVHFPKVLQCLNISVLSQKRCEDAYPRQIDDTMFCAGDKAGRDSCQGDSG | |
| | GPVVCNGSLQGLVSWGDYPCARPNRPGVYTNLCKFTKWIQETIQANS | |
| Human KLK5 | MATARPPWMWVLCALITALLLGVTEHVLANNDVSCDHPSNTVPSGSNQDLGAGARE | 7 |
| R55, D153 | DARSDDSSSRIINGSDCDMHTQPWQAALLLRPNQLYCGAVLVHPQWLLTAAHCRKK | |
| (incl. signal | VFRVRLGHYSLSPVYESGQQMFQGVKSIPHPGYSHPGHSNDLMLIKLNRRIRPTKD | |
| peptide | VRPINVSSHCPSAGTKCLVSGWGTTKSPQVHFPKVLQCLNISVLSQKRCEDAYPRQ | |
| (underlined)) | IDDTMFCAGDKAGRDSCQGDSGGPVVCNGSLQGLVSWGDYPCARPNRPGVYTNLCK | |
| | FTKWIQETIQANS | |
| Human KLK5 | VTEHVLANNDVSCDHPSNTVPSGSNQDLGAGAREDARSDDSSSRIINGSDCDMHTQ | 8 |
| R55, D153 | PWQAALLLRPNQLYCGAVLVHPQWLLTAAHCRKKVFRVRLGHYSLSPVYESGQQMF | |
| (mature form) | QGVKSIPHPGYSHPGHSNDLMLIKLNRRIRPTKDVRPINVSSHCPSAGTKCLVSGW | |
| | GTTKSPQVHFPKVLQCLNISVLSQKRCEDAYPRQIDDTMFCAGDKAGRDSCQGDSG | |
| | GPVVCNGSLQGLVSWGDYPCARPNRPGVYTNLCKFTKWIQETIQANS | |
| Human SPINK5 | MKIATVSVLLPLALCLIQDAASKNEDQEMCHEFQAFMKNGKLFCPQDKKFFQSLDG | 9 |
| Q9NQ38 | IMFINKCATCKMILEKEAKSQKRARHLARAPKATAPTELNCDDFKKGERDGDFICP | |
| (incl. signal | DYYEAVCGTDGKTYDNRCALCAENAKTGSQIGVKSEGECKSSNPEQDVCSAFRPFV | |
| peptide | RDGRLGCTRENDPVLGPDGKTHGNKCAMCAELFLKEAENAKREGETRIRRNAEKDF | |
| (underlined)) | CKEYEKQVRNGRLFCTRESDPVRGPDGRMHGNKCALCAEIFKQRFSEENSKTDQNL | |
| | GKAEEKTKVKREIVKLCSQYQNQAKNGILFCTRENDPIRGPDGKMHGNLCSMCQAY | |
| | FQAENEEKKKAEARARNKRESGKATSYAELCSEYRKLVRNGKLACTRENDPIQGPD | |
| | GKVHGNTCSMCEVFFQAEEEEKKKKEGKSRNKRQSKSTASFEELCSEYRKSRKNGR | |
| | LFCTRENDPIQGPDGKMHGNTCSMCEAFFQQEERARAKAKREAAKEICSEFRDQVR | |
| | NGTLICTREHNPVRGPDGKMHGNKCAMCASVFKLEEEEKKNDKEEKGKVEAEKVKR | |
| | EAVQELCSEYRHYVRNGRLPCTRENDPIEGLDGKIHGNTCSMCEAFFQQEAKEKER | |
| | AEPRAKVKREAEKETCDEFRRLLQNGKLFCTRENDPVRGPDGKTHGNKCAMCKAVF | |
| | QKENEERKRKEEEDQRNAAGHGSSGGGGGNTQDECAEYREQMKNGRLSCTRESDPV | |
| | RDADGKSYNNQCTMCKAKLEREAERKNEYSRSRSNGTGSESGKDTCDEFRSQMKNG | |
| | KLICTRESDPVRGPDGKTHGNKCTMCKEKLEREAAEKKKKEDEDRSNTGERSNTGE | |
| | RSNDKEDLCREFRSMQRNGKLICTRENNPVRGPYGKMHINKCAMCQSIFDREANER | |
| | KKKDEEKSSSKPSNNAKDECSEFRNYIRNNELICPRENDPVHGADGKFYTNKCYMC | |
| | RAVFLTEALERAKLQEKPSHVRASQEEDSPDSFSSLDSEMCKDYRVLPRIGYLCPK | |
| | DLKPVCGDDGQTYNNPCMLCHENLIRQTNTHIRSTGKCEESSTPGTTAASMPPSDE | |
| Human SPINK5 | KNEDQEMCHEFQAFMKNGKLFCPQDKKFFQSLDGIMFINKCATCKMILEKEAKSQK | 10 |
| (mature form) | RARHLARAPKATAPTELNCDDFKKGERDGDFICPDYYEAVCGTDGKTYDNRCALCA | |
| | ENAKTGSQIGVKSEGECKSSNPEQDVCSAFRPFVRDGRLGCTRENDPVLGPDGKTH | |
| | GNKCAMCAELFLKEAENAKREGETRIRRNAEKDFCKEYEKQVRNGRLFCTRESDPV | |
| | RGPDGRMHGNKCALCAEIFKQRFSEENSKTDQNLGKAEEKTKVKREIVKLCSQYQN | |
| | QAKNGILFCTRENDPIRGPDGKMHGNLCSMCQAYFQAENEEKKKAEARARNKRESG | |
| | KATSYAELCSEYRKLVRNGKLACTRENDPIQGPDGKVHGNTCSMCEVFFQAEEEEK | |
| | KKKEGKSRNKRQSKSTASFEELCSEYRKSRKNGRLFCTRENDPIQGPDGKMHGNTC | |
| | SMCEAFFQQEERARAKAKREAAKEICSEFRDQVRNGTLICTREHNPVRGPDGKMHG | |
| | NKCAMCASVFKLEEEEKKNDKEEKGKVEAEKVKREAVQELCSEYRHYVRNGRLPCT | |
| | RENDPIEGLDGKIHGNTCSMCEAFFQQEAKEKERAEPRAKVKREAEKETCDEFRRL | |
| | LQNGKLFCTRENDPVRGPDGKTHGNKCAMCKAVFQKENEERKRKEEEDQRNAAGHG | |
| | SSGGGGGNTQDECAEYREQMKNGRLSCTRESDPVRDADGKSYNNQCTMCKAKLERE | |
| | AERKNEYSRSRSNGTGSESGKDTCDEFRSQMKNGKLICTRESDPVRGPDGKTHGNK | |
| | CTMCKEKLEREAAEKKKKEDEDRSNTGERSNTGERSNDKEDLCREFRSMQRNGKLI | |
| | CTRENNPVRGPYGKMHINKCAMCQSIFDREANERKKKDEEKSSSKPSNNAKDECSE | |
| | FRNYIRNNELICPRENDPVHGADGKFYTNKCYMCRAVELTEALERAKLQEKPSHVR | |
| | ASQEEDSPDSFSSLDSEMCKDYRVLPRIGYLCPKDLKPVCGDDGQTYNNPCMLCHE | |
| | NLIRQTNTHIRSTGKCEESSTPGTTAASMPPSDE | |

HVR-H1 Sequences

| | | |
|---|---|---|
| HVR-H1 | DYNMA | 11 |
| 14C8 | | |
| HVR-H1 | DYYMA | 12 |
| 14E12 | | |
| HVR-H1 | SSYWIC | 13 |
| 8E11 | | |
| HVR-H1 | TSYWIC | 14 |
| 8G10 | | |
| HVR-H1 | NYGVT | 15 |
| 9B6 | | |
| HVR-H1 | NYGVS | 16 |
| 2.3F4 | | |
| HVR-H1 | SYGVT | 17 |
| 10C5, | | |
| hu10C5-H1 to | | |
| hu10C5-H28 | | |
| HVR-H1 | NYGVS | 18 |
| 2B11 | | |
| HVR-H1 | TFAIN | 19 |
| 10H3 | | |
| HVR-H1 | GYGVS | 20 |
| 9H3 | | |
| HVR-H1 | NYGVT | 21 |
| 8B7 | | |
| HVR-H1 | SYGVS | 22 |
| 9F2, | | |
| 10C8, | | |
| 9H5, | | |
| hu9H5-H1 to | | |
| hu9H5-H17 | | |
| HVR-H1 | SYPIS | 23 |
| 8F5 | | |
| HVR-H1 | DYGVS | 24 |
| 3-3F5, | | |
| hu3-3F5-H1 to | | |
| hu3-3F5-H27 | | |
| HVR-H1 | NNYVNFVMC | 25 |
| 9E3 | | |
| HVR-H1 | DNYVMS | 26 |
| 10D10 | | |
| HVR-H1 | GGGIY | 27 |
| 12B3, | | |
| 1D10 | | |
| HVR-H1 | X₁YGVX₂ wherein X₁ is S or D, X₂ is T or S | 28 |
| 10C5, | | |
| hu10C5-H1 to | | |
| hu10C5-H28, | | |
| 9H5, | | |
| hu9H5-H1 to | | |
| hu9H5-H17, | | |
| 3-3F5, | | |
| hu3-3F5-H1 to | | |
| hu3-3F5-H27 | | |
| (consensus) | | |

HVR-H2 Sequences

| | | |
|---|---|---|
| HVR-H2 | TISYDAGRTYYRDSVKG | 29 |
| 14C8 | | |
| HVR-H2 | SISYDGDTTYYRDSVKG | 30 |
| 14E12 | | |
| HVR-H2 | CVYGLDVNIYYASWTK | 31 |
| 8E11 | | |
| HVR-H2 | CVYGLDVNIYYASWTE | 32 |
| 8G10 | | |
| HVR-H2 | FIGSGGSAYYASWAKS | 33 |
| 9B6, 9H3 | | |
| HVR-H2 | FIGYGGSTYYASWAKS | 34 |
| 2.3F4 | | |
| HVR-H2 | YITSNYGVSYYASWAKS | 35 |
| 10C5, | | |
| hu10C5-H1 to | | |
| hu10C5-H10, | | |
| hu10C5-H12, | | |
| hu10C5-H14 to | | |
| hu10C5-H16, | | |
| hu10C5-H18 to | | |
| hu10C5-H28 | | |
| HVR-H2 | YITSNYGVSYYANSVKG | 36 |
| hu10C5-H11 | | |
| hu10C5-H13 | | |
| HVR-H2 | YITSNYGVSYYASFAKS | 37 |
| hu10C5-H17 | | |
| HVR-H2 | YITSNYGVSYYAX₁X₂X₃KX₄ wherein X₁ is S or N, X₂ is W, S or | 38 |
| hu10C5-H1 to | F, X₃ is A or V, X₄ is S or G | |
| hu10C5-H28 | | |
| (consensus) | | |
| HVR-H2 | YIGSAGSTYYATWAKS | 39 |
| 2B11 | | |
| HVR-H2 | AIGRGGSAYYASWAKS | 40 |
| 10H3 | | |
| HVR-H2 | FIGSSGSAYYASWAKS | 41 |
| 8B7 | | |
| HVR-H2 | FIGSGGFAYYASWAKS | 42 |
| 10C8, 9H5, | | |
| hu9H5-H1 to | | |
| hu9H5-H10, | | |
| hu9H5-H12, | | |
| hu9H5-H14 to | | |
| hu9H5-H16 | | |
| HVR-H2 | FIGSGGFAYYADSVKG | 43 |
| hu9H5-H11, | | |
| hu9H5-H13 | | |
| HVR-H2 | FIGSGGFAYYASFAKS | 44 |
| hu9H5-H17 | | |
| HVR-H2 | FIGSGGFAYYAX₁X₂X₃KX₄ wherein X₁ is S or D, X₂ is W, S or | 45 |
| hu9H5-H1 to | F, X₃ is A or V, X₄ is S or G | |
| hu9H5-H17 | | |
| (consensus) | | |
| HVR-H2 | FIGSGGSPYYASWAKS | 46 |
| 9F2 | | |
| HVR-H2 | YITSEYGVAYYATWAES | 47 |
| 8F5 | | |
| HVR-H2 | AIGSSGVAWYANWAKG | 48 |
| 3-3F5, | | |
| hu3-3F5-H1 to | | |
| hu3-3F5-H9, | | |
| hu3-3F5-H14, | | |
| hu3-3F5-H16, | | |
| hu3-3F5-H17 | | |
| HVR-H2 | AIGSSGVAFYANWAKG | 49 |
| hu3-3F5-H10, | | |
| hu3-3F5-H12, | | |
| hu3-3F5-H15 | | |
| HVR-H2 | AIGSSGVAWYADSVKG | 50 |
| hu3-3F5-H11, | | |
| HVR-H2 | AIGSSGVAFYADSVKG | 51 |
| hu3-3F5-H13 | | |
| HVR-H2 | AIGSSGVAWYANFAKG | 52 |
| hu3-3F5-H18 | | |
| to | | |
| hu3-3F5-H26 | | |
| HVR-H2 | AIGSSGVAFYANFAKG | 53 |
| hu3-3F5-H27 | | |
| HVR-H2 | AIGSSGVAX₁YAX₂X₃X₄KG wherein X₁ is W or F, X₂ is N or D, X₃ | 54 |
| hu3-3F5-H1 to | is W, S or F, X₄ is A or V | |
| hu3-3F5-H27 | | |
| (consensus) | | |
| HVR-H2 | SIDPGDDSTDYASWAT | 55 |
| 9E3 | | |
| HVR-H2 | CIDPGDDSTYYASWAT | 56 |
| 10D10 | | |
| HVR-H2 | SIYPDHGSVDYANWVNG | 57 |
| 12B3 | | |
| HVR-H2 | YIYPDHGSADYATWVNG | 58 |
| 1D10 | | |
| HVR-H2 | X₁IGSX₂GX₃AX₄YAX₅X₆X₇KX₈ wherein X₁ is F or A, X₂ is G or S, | 59 |
| 9H5, | X₃ is F or V, X₄ is Y, W, or F, X₅ is S, D, or N, X₆ is W, | |
| hu9H5-H1 to | S, or F, X₇ is A or V, X₈ is S or G | |
| hu9H5-H17, | | |
| 3-3F5, | | |
| hu3-3F5-H1 to | | |
| hu3-3F5-H27 | | |
| (consensus) | | |

HVR-H3 Sequences

| | | |
|---|---|---|
| HVR-H3 | GIFNYGTDYFDY | 60 |
| 14C8 | | |
| HVR-H3 | DGTIPAGSWFAY | 61 |
| 14E12 | | |
| HVR-H3 | GGGSADFGFDL | 62 |
| 8E11, | | |
| 8G10 | | |
| HVR-H3 | DDVGGGKSLDI | 63 |
| 9B6 | | |
| HVR-H3 | LCGVDCADALDS | 64 |
| 2.3F4 | | |
| HVR-H3 | ENPDYGYAYDA | 65 |
| 10C5 | | |
| hu10C5-H1 to | | |
| hu10C5-H28 | | |
| HVR-H3 | AAYSAGSADAEDI | 66 |
| 2B11 | | |
| HVR-H3 | ENAGSGWGELDI | 67 |
| 10H3 | | |
| HVR-H3 | DNVGGDMSLDI | 68 |
| 9H3 | | |
| HVR-H3 | DDVGGGKSLDI | 69 |
| 8B7, | | |
| 9F2, | | |
| 9H5 | | |
| hu9H5-H1 to | | |
| hu9H5-H17 | | |
| HVR-H3 | DDVGGGRSLDI | 70 |
| 10C8 | | |
| HVR-H3 | ENPTYGYAYDA | 71 |
| 8F5 | | |
| HVR-H3 | DRDYGYRADDATSGMDL | 72 |
| 3-3F5, | | |
| hu3-3F5-H1 to | | |
| hu3-3F5-H27 | | |
| HVR-H3 | GDAGTSYSFNF | 73 |
| 9E3 | | |
| HVR-H3 | GDAAASYSFNF | 74 |
| 10D10 | | |
| HVR-H3 | ESGGSYYDL | 75 |
| 12B3 | | |
| HVR-H3 | ETGGSWYDL | 76 |
| 1D10 | | |

HVR-L1 Sequences

| | | |
|---|---|---|
| HVR-L1 | RASEDIYSGLA | 77 |
| 14C8 | | |
| HVR-L1 | LASKNIYRNLA | 78 |
| 14E12 | | |
| HVR-L1 | QASENIYSLLA | 79 |
| 8E11, | | |
| 8G10 | | |
| HVR-L1 | QASQNIGDYLS | 80 |
| 9B6 | | |
| HVR-L1 | QASEDIGSYCS | 81 |
| 2.3F4 | | |
| HVR-L1 | QASESISNELS | 82 |
| 10C5, | | |
| hu10C5-L1 to | | |
| hu10C5-L6 | | |
| HVR-L1 | QASQSISNYVA | 83 |
| 2B11 | | |
| HVR-L1 | QASESISSDLA | 84 |
| 10H3 | | |
| HVR-L1 | QASQNINNYLS | 85 |
| 9H3 | | |
| HVR-L1 | QASQSIGSYLS | 86 |
| 8B7 | | |
| HVR-L1 | QASQSISSYLS | 87 |
| 9H5, | | |
| hu9H5-L1 to | | |
| hu9H5-L4 | | |
| HVR-L1 | QASQSISNYLS | 88 |
| 9F2 | | |
| HVR-L1 | QASQSISTYLS | 89 |
| 10C8 | | |
| HVR-L1 | QASESIGNELS | 90 |
| 8F5 | | |
| HVR-L1 | QASESIGNALA | 91 |
| 3-3F5, | | |
| hu3-3F5-L1 to | | |
| hu3-3F5-L5 | | |
| HVR-L1 | QASESISRYLS | 92 |
| 9E3 | | |
| HVR-L1 | QASESISTYLS | 93 |
| 10D10 | | |
| HVR-L1 | QASQSISTYLA | 94 |
| 12B3 | | |
| HVR-L1 | QASQSISSYLA | 95 |
| 1D10 | | |
| HVR-L1 | QASX₁SIX₂X₃X₄LX₅ wherein X₁ is E or Q, X₂ is S or G, X₃ is | 96 |
| 10C5, | N or S, X₄ is E, Y or A, X₅ is S or A | |
| hu10C5-L1 to | | |
| hu10C5-L6, | | |
| 9H5, | | |
| hu9H5-L1 to | | |
| hu9H5-L4, | | |
| 3-3F5, | | |
| hu3-3F5-L1 to | | |
| hu3-3F5-L5 | | |
| (consensus) | | |

HVR-L2 Sequences

| | | |
|---|---|---|
| HVR-L2 | GATTLHD | 97 |
| 14C8 | | |
| HVR-L2 | DASRLQD | 98 |
| 14E12 | | |
| HVR-L2 | DASDLAS | 99 |
| 8E11, | | |
| 8G10, | | |
| 2.3F4, | | |
| 9E3, | | |
| 10D10, | | |
| 3-3F5, | | |
| hu3-3F5-L1 to | | |
| hu3-3F5-L5 | | |
| HVR-L2 | SASTLAS | 100 |
| 9B6 | | |
| HVR-L2 | YASTLAS | 101 |
| 10C5, | | |
| hu10C5-L1 to | | |
| hu10C5-L6 | | |
| HVR-L2 | RASTLAS | 102 |
| 2B11 | | |
| HVR-L2 | AASTLAS | 103 |
| 10H3, | | |
| 9H3 | | |
| HVR-L2 | DASNLAS | 104 |
| 8B7 | | |
| HVR-L2 | SASTLAS | 105 |
| 9F2 | | |
| 10C8 | | |
| 9H5, | | |
| hu9H5-L1 to | | |
| hu9H5-L4 | | |
| HVR-L2 | QASTLAS | 106 |
| 8F5 | | |
| HVR-L2 | KTSTLAS | 107 |
| 12B3 | | |
| HVR-L2 | KASTLAS | 108 |
| 1D10 | | |
| HVR-L2 | X₁ASX₂LAS wherein X₁ is Y, S or D, X₂ is T or D | 109 |
| 10C5, | | |
| hu10C5-L1 to | | |
| hu10C5-L6 | | |
| 9H5, | | |
| hu9H5-L1 to | | |
| hu9H5-L4, | | |
| 3-3F5, | | |
| hu3-3F5-L1 to | | |
| hu3-3F5-L5 | | |
| (consensus) | | |

HVR-L3 Sequences

| | | |
|---|---|---|
| HVR-L3 | HQGLSFPYT | 110 |
| 14C8 | | |
| HVR-L3 | QQYHDYPYT | 111 |
| 14E12 | | |
| HVR-L3 | QATAYGSSGNA | 112 |
| 8E11, | | |
| 8G10 | | |
| HVR-L3 | HQDYTSNDVENT | 113 |
| 9B6 | | |
| HVR-L3 | QQDYTGNNVDNT | 114 |
| 2.3F4 | | |
| HVR-L3 | AQGFGSSGVENV | 115 |
| 10C5, | | |
| hu10C5-L1 to | | |
| hu10C5-L6 | | |
| HVR-L3 | HQGYSSSNVDNI | 116 |
| 2B11 | | |
| HVR-L3 | QQGYTWNNVDNV | 117 |
| 10H3 | | |
| HVR-L3 | HQDYTSNNVDNT | 118 |
| 9H3, | | |
| 8B7, | | |
| 10C8 | | |
| HVR-L3 | HQDYTSSNVDNT | 119 |
| 9H5, | | |
| hu9H5-L1 to | | |
| hu9H5-L4 | | |
| HVR-L3 | HQDYTSNSVDNT | 120 |
| 9F2 | | |
| HVR-L3 | AQGFSSSGVENV | 121 |
| 8F5 | | |
| HVR-L3 | QQGDSHNNVDNI | 122 |
| 3-3F5, | | |
| hu3-3F5-L1 to | | |
| hu3-3F5-L5 | | |
| HVR-L3 | QQDYSRSNIVNS | 123 |
| 9E3 | | |
| HVR-L3 | QQDYSSSNIVNS | 124 |
| 10D10 | | |
| HVR-L3 | QQGYSGSSVENT | 125 |
| 12B3 | | |
| HVR-L3 | QQGYSGSNVENT | 126 |
| 1D10 | | |
| HVR-L3 | X₁QX₂X₃X₄X₅X₆X₇VX₈NX₉ wherein X₁ is A, H or Q, X₂ is G or D, | 127 |
| 10C5, | X₃ is F, Y or D, X₄ is G, T or S, X₅ is S or H, X₆ is S | |
| hu10C5-L1 to | or N, X₇ is G or N, X₈ is E or D, X₉ is V, T or I | |
| hu10C5-L6 | | |
| 9H5, | | |
| hu9H5-L1 to | | |
| hu9H5-L4, | | |
| 3-3F5, | | |
| hu3-3F5-L1 to | | |
| hu3-3F5-L5 | | |
| (consensus) | | |

VL Sequences (HVRs underlined)

| | | |
|---|---|---|
| VL | DIQMTQSPASLSASLGETVTIQCRASEDIYSGLAWYQQKPGKSPQLLIYGATTLHD | 128 |
| 14C8 | GVPSRFSGSGSGTQYSLKISSMHSEDEGIYFCHQGLSFPYTFGAGTKLELK | |
| VL | DIQMTQSPASLSASLGETVTIECLASKNIYRNLAWYQQKPGKSPQFLISDASRLQD | 129 |
| 14E12 | GVPSRFTGSDSGSQYSLKINSLQSEDVATYFCQQYHDYPYTFGAGTKLELK | |
| VL | DVVMTQTASPVSAAVGGTVTIKCQASENIYSLLAWYQQKPGQPPKVLIYDASDLAS | 130 |
| 8E11 | GVPSRFKGSGSGTQFTLTISDLECADAATYYCQATAYGSSGNAFGGGTEVVVK | |
| VL | DVVMTQTPASVSEPVGGTVTIKCQASENIYSLLAWYQQKPGQPPKVLIYDASDLAS | 131 |
| 8G10 | GVPSRFKGSGSGTQFTLTISDLECADAATYYCQATAYGSSGNAFGGGTEVVVK | |
| VL | NIVMTQTPASVEVAVGGTVVIKCQASQNIGDYLSWYQQKPGQRPKLLIYSASTLAS | 132 |
| 9B6 | GVPSRFKGSGSGTQFTLTISDLECADAATYYCHQDYTSNDVENTFGGGTEVVVK | |
| VL | AYYMTQTPASVEVAVGGTVTIKCQASEDIGSYCSWYQQKPGQPPKLLIYDASDLAS | 133 |
| 2.3F4 | GVPSRFKGSGSGTDFTLTISGVQCDDAATYYCQQDYTGNNVDNTFGGGSEVVVK | |
| VL | AYDMTQTPASLEAAVGGTVTINCQASESISNELSWYQQKPGQPPDLLIYYASTLAS | 134 |
| 10C5 | GVPSRFKGSGSGTEFTLTISDLECADAATYYCAQGFGSSGVENVFGGGTEVVVK | |
| VL | AYRMTQSPSSFSASTGDRVTITCQASESISNELSWYQQKPGKPPKLLIYYASTLAS | 135 |
| hu10C5-L1 | GVPSRFSGSGSGTDFTLTISCLQSEDFATYYCAQGFGSSGVENVFGGGTKVEIK | |
| VL | AIRMTQSPSSFSASTGDRVTITCQASESISNELSWYQQKPGKPPKLLIYYASTLAS | 136 |
| hu10C5-L2 | GVPSRFSGSGSGTDFTLTISCLQSEDFATYYCAQGFGSSGVENVFGGGTKVEIK | |
| VL | AYRMTQSPSSFSASTGDRVTITCQASESISNELSWYQQKPGKAPKLLIYYASTLAS | 137 |
| hu10C5-L3 | GVPSRFSGSGSGTDFTLTISCLQSEDFATYYCAQGFGSSGVENVFGGGTKVEIK | |
| VL | AIRMTQSPSSFSASTGDRVTITCQASESISNELSWYQQKPGKAPKLLIYYASTLAS | 138 |
| hu10C5-L4 | GVPSRFSGSGSGTDFTLTISCLQSEDFATYYCAQGFGSSGVENVFGGGTKVEIK | |
| VL | AIRMTQSPSSFSASTGDRVTITCQASESISNELSWYQQKPGKAPKLLIYYASTLAS | 139 |
| hu10C5-L5 | GVPSRFSGSGSGTDFTLTISSLQPEDFATYYCAQGFGSSGVENVFGGGTKVEIK | |
| VL | AX₁RMTQSPSSFSASTGDRVTITCQASESISNELSWYQQKPGKX₂PKLLIYYASTLA | 140 |
| 10C5, | SGVPSRFSGSGSGTDFTLTISX₃LQX₄EDFATYYCAQGFGSSGVENVFGGGTX₅VX₆ | |
| hu10C5-L1 to | X₇K | |
| hu10C5-L6 | wherein X₁ is Y or I, X₂ is P or A, X₃ is C or S, X₄ is S | |
| (consensus) | or P, X₅ is E or K, X₆ is E or V, X₇ is I or V | |
| VL | AYYMTQTPASVEAAVGGTVTIKCQASQSISNYVAWYQQKPGQPPKLLIYRASTLAS | 141 |
| 2B11 | GVSSRFSGSGSGTEFTLTISDLECADAATYYCHQGYSSSNVDNIFGGGTEVVVK | |
| VL | AYEMTQTPASVEVAVGGTVTINCQASESISSDLAWYQQKPGQRPKLLIYAASTLAS | 142 |
| 10H3 | GVPSRFKGSGSGTEFTLSISGVQCADAATYYCQQGYTWNNVDNVFGGGTEVVVK | |
| VL | DIVMTQTPASVSEPVGGTVTIKCQASQNINNYLSWYQQKPGQPPKQLIYAASTLAS | 143 |
| 9H3 | GVPSRFKGSGSGTQFTLTISDLECADAATYYCHQDYTSNNVDNTFGGGTEVVVK | |
| VL | NIVMTQTPASVEVAVGGTVTIKCQASQSIGSYLSWYQQKPGQPPKLLIYDASNLAS | 144 |
| 8B7 | GVPSRFKGSGSGTQFTLSISDLECADAATYYCHQDYTSNNVDNTFGGGTEVVIK | |
| VL | NIVMTQTPASVEAAVGGTVTINCQASQSISSYLSWYQQKSGQRPKLLIFSASTLAS | 145 |
| 9H5 | GVPSRFTGSGSGTQFTLTISDLQCADAATYYCHQDYTSSNVDNTFGGGTEVVVK | |
| VL | DIQMTQSPSSLSASVGDRVTITCQASQSISSYLSWYQQKPGKRPKLLIFSASTLAS | 146 |
| hu9H5-L1 | GVPSRFSGSGSGTDFTLTISSLQPEDFATYYCHQDYTSSNVDNTFGGGTKVEIK | |
| VL | DIQMTQSPSSLSASVGDRVTITCQASQSISSYLSWYQQKPGKAPKLLIFSASTLAS | 147 |
| hu9H5-L2 | GVPSRFSGSGSGTDFTLTISSLQPEDFATYYCHQDYTSSNVDNTFGGGTKVEIK | |
| VL | DIQMTQSPSSLSASVGDRVTITCQASQSISSYLSWYQQKPGKRPKLLIYSASTLAS | 148 |
| hu9H5-L3 | GVPSRFSGSGSGTDFTLTISSLQPEDFATYYCHQDYTSSNVDNTFGGGTKVEIK | |
| VL | DIQMTQSPSSLSASVGDRVTITCQASQSISSYLSWYQQKPGKAPKLLIYSASTLAS | 149 |
| hu9H5-L4 | GVPSRFSGSGSGTDFTLTISSLQPEDFATYYCHQDYTSSNVDNTFGGGTKVEIK | |
| VL | DIQMTQSPSSLSASVGDRVTITCQASQSISSYLSWYQQKPGKAPKLLIYSASTLAS | 150 |
| hu9H5-L5 | GVPSRFSGSGSGTDFTLTISSLQPEDFATYYCHQDYTSSNVDNTFGGGTEVVVK | |
| VL | DIQMTQSPSSLSASVGDRVTITCQASQSISSYLSWYQQKPGKX₁PKLLIX₂SASTLA | 151 |
| 9H5, | SGVPSRFSGSGSGTDFTLTISSLQPEDFATYYCHQDYTSSNVDNTFGGGTX₃VX₄X₅ | |
| hu9H5-L1 to | K | |
| hu9H5-L5 | wherein X₁ is R or A, X₂ is F or Y, X₃ is E or K, X₄ is E | |
| (consensus) | or V, X₅ is V or I | |
| VL | NIVMTQTPASVEVAVGGTVIIKCQASQSISNYLSWYHQKSGQRPRLLIYSASTLAS | 152 |
| 9F2 | GVPSRFKGSGSGTQFTLTISDLECADAATYYCHQDYTSNSVDNTFGGGTEVVVK | |
| VL | NIVMTQTPASVEVAMGGTVIIKCQASQSISTYLSWYQQKPGQPPKLLIYSASTLAS | 153 |
| 10C8 | GVSSRFEGSGSGTQFTLTISGVQCADAATYYCHQDYTSNNVDNTFGGGTEVVVK | |
| VL | AYDLTQTPASVEAAVGGTVTINCQASESIGNELSWYQQKSGQPPKLLIYQASTLAS | 154 |
| 8F5 | GVPSRFKGSGSGTDFTLTISDLECADAATYYCAQGFSSSGVENVFGGGTEVVVK | |
| VL | AYDMTQTPASVEAAVGGTVTIKCQASESIGNALAWYQQKPGQPPKLLIYDASDLAS | 155 |
| 3-3F5 | GVPSRFKGSGSGTQFTLTISGVECADAATYYCQQGDSHNNVDNIFGGGTEVVVK | |
| VL | AYRMTQSPSSFSASTGDRVTITCQASESIGNALAWYQQKPGKPPKLLIYDASDLAS | 156 |
| hu3-3F5-L1 | GVPSRFSGSGSGTDFTLTISCLQSEDFATYYCQQGDSHNNVDNIFGGGTKVEIK | |
| VL | AIRMTQSPSSFSASTGDRVTITCQASESIGNALAWYQQKPGKPPKLLIYDASDLAS | 157 |
| hu3-3F5-L2 | GVPSRFSGSGSGTDFTLTISCLQSEDFATYYCQQGDSHNNVDNIFGGGTKVEIK | |
| VL | AYRMTQSPSSFSASTGDRVTITCQASESIGNALAWYQQKPGKAPKLLIYDASDLAS | 158 |
| hu3-3F5-L3 | GVPSRFSGSGSGTDFTLTISCLQSEDFATYYCQQGDSHNNVDNIFGGGTKVEIK | |
| VL | AIRMTQSPSSFSASTGDRVTITCQASESIGNALAWYQQKPGKAPKLLIYDASDLAS | 159 |
| hu3-3F5-L4 | GVPSRFSGSGSGTDFTLTISCLQSEDFATYYCQQGDSHNNVDNIFGGGTKVEIK | |
| VL | AIRMTQSPSSFSASTGDRVTITCQASESIGNALAWYQQKPGKAPKLLIYDASDLAS | 160 |
| hu3-3F5-L5 | GVPSRFSGSGSGTDFTLTISSLQPEDFATYYCQQGDSHNNVDNIFGGGTKVEIK | |
| VL | AIRMTQSPSSFSASTGDRVTITCQASESIGNALAWYQQKPGKAPKLLIYDASDLAS | 161 |
| hu3-3F5-L6 | GVPSRFSGSGSGTDFTLTISSLQPEDFATYYCQQGDSHNNVDNIFGGGTEVVVK | |
| VL | AX₁RMTQSPSSFSASTGDRVTITCQASESIGNALAWYQQKPGKX₂PKLLIYDASDLA | 162 |
| 3-3F5, | SGVPSRFSGSGSGTDFTLTISX₃LQX₄EDFATYYCQQGDSHNNVDNIFGGGTX₅VX₆ | |
| hu3-3F5-L1 to | X₇K | |
| hu3-3F5-L6 | wherein X₁ is Y or I, X₂ is P or A, X₃ is C or S, X₄ is S | |
| (consensus) | or P | |
| | X₅ is E or K, X₆ is E or V, X₇ is V or I | |
| VL | AIEMTQTPSSASEPVGGTVTIKCQASESISRYLSWYQQKPGQPPKLLIYDASDLAS | 163 |
| 9E3 | GVSSRFKGSGSGTQFTLIISDVECADAATYYCQQDYSRSNIVNSFGGGTEVVVK | |
| VL | AYDMTQTPSSASEPVGGTVTIKCQASESISTYLSWYQQKPGQPPKLLIYDASDLAS | 164 |
| 10D10 | GVSSRFKGSGSGTQFTLTISDVECADAATYYCQQDYSSSNIVNSFGGGTEVVVK | |
| VL | AYDMTQTPASVEVAVGGTVTIKCQASQSISTYLAWYQQKPGQRPNLLIYKTSTLAS | 165 |
| 12B3 | GVPSRFRGSGSGTQFTLTISGVECADAATYYCQQGYSGSSVENTFGGGTEVVVK | |
| VL | AYDMTQTPVSVEAAVGGTVTIKCQASQSISSYLAWYQQKPGQPPKLLIYKASTLAS | 166 |
| 1D10 | GVSSRFKGSGSGTEFTLTISDLECADAATYYCQQGYSGSNVENTFGGGTEVVVK | |

VH Sequences (HVRs underlined)

| | | |
|---|---|---|
| VH | EVQLVESGGGLVQPGRSLKLSCTASGFTFSDYNMAWVRQAPKGGLEWVTTISYDAG | 167 |
| 14C8 | RTYYRDSVKGRFTISRDNAKRTLSLQMDSLRSEDTATYYCATGIFNYGTDYFDYWG | |
| | QGVMVTVSS | |
| VH | EVQLVESGGGLVRPGRSLRLSCAASGFTFSDYYMAWVRQAPTKGLEWVASISYDGD | 168 |
| 14E12 | TTYYRDSVKGRFTISRDNARSSLYLQMDSLRSDDTANYFCTTDGTIPAGSWFAYWG | |
| | QGTLVTVSS | |
| VH | QEQLEESGGDLVKPEGSLTLTCTASGFSFSSSYWICWVRQAPGKGLEWIACVYGLD | 169 |
| 8E11 | VNIYYASWTKGRFTISKTSSTTVTLQMTSLTAADTATYFCARGGGSADFGFDLWGP | |
| | GTLVTVSS | |
| VH | QSLEESGGDLVKPEGSLTLTCTASGFSFSTSYWICWVRQAPGKGLEWIACVYGLDV | 170 |
| 8G10 | NIYYASWTEGRFTISKTSSTTVTLQVTSLTAADTATYFCARGGGSADFGFDLWGPG | |
| | TLVTVSS | |
| VH | QSVKESEGGLFKPTDNLTLTCTVSGFSLSNYGVTWVRQAPGNGLEYIGFIGSGGSA | 171 |
| 9B6 | YYASWAKSRSTITRNTNLNTVTLKMTSLTAADTATYFCARDDVGGGKSLDIWGPGT | |
| | LVTVSS | |
| VH | QSVEESRGGLIKPTDTLTLTCTASGFSLSNYGVSWVRQAPGNGLEYIGFIGYGGST | 172 |
| 2.3F4 | YYASWAKSRSTITRNTNLNTVTLQMTSLTAADTATYFCARLCGVDCADALDSWGPG | |
| | TLVTVSS | |
| VH | QSLEESGGGLVKPTDTLTLTCTVSGFSLSSYGVTWVRQAPGRGLEWIGYITSNYGV | 173 |
| 10C5 | SYYASWAKSRSTITRNTNLNTVTLKMTSLTAADTATYFCARENPDYGYAYDAWGPG | |
| | TLVTVSV | |
| VH | EVQLVESGGGLVQPGGSLRLSCAVSGFSLSSYGVTWVRQAPGKGLEWIGYITSNYG | 174 |
| hu10C5-H1 | VSYYASWAKSRSTISRDTSKNTVYLQMGSLRAEDMAVYFCARENPDYGYAYDAWGP | |
| | GTTVTVSS | |
| VH | EVQLVESGGGLVQPGGSLRLSCAASGFSLSSYGVTWVRQAPGKGLEWIGYITSNYG | 175 |
| hu10C5-H2 | VSYYASWAKSRSTISRDTSKNTVYLQMGSLRAEDMAVYFCARENPDYGYAYDAWGP | |
| | GTTVTVSS | |
| VH | EVQLVESGGGLVQPGGSLRLSCAVSGFSLSSYGVTWVRQAPGKGLEYIGYITSNYG | 176 |
| hu10C5-H3 | VSYYASWAKSRSTISRDTSKNTVYLQMGSLRAEDMAVYFCARENPDYGYAYDAWGP | |
| | GTTVTVSS | |
| VH | EVQLVESGGGLVQPGGSLRLSCAVSGFSLSSYGVTWVRQAPGKGLEWVGYITSNYG | 177 |
| hu10C5-H4 | VSYYASWAKSRSTISRDTSKNTVYLQMGSLRAEDMAVYFCARENPDYGYAYDAWGP | |
| | GTTVTVSS | |
| VH | EVQLVESGGGLVQPGGSLRLSCAVSGFSLSSYGVTWVRQAPGKGLEWISYITSNYG | 178 |
| hu10C5-H5 | VSYYASWAKSRSTISRDTSKNTVYLQMGSLRAEDMAVYFCARENPDYGYAYDAWGP | |
| | GTTVTVSS | |
| VH | EVQLVESGGGLVQPGGSLRLSCAVSGFSLSSYGVTWVRQAPGKGLEWIGYITSNYG | 179 |
| hu10C5-H6 | VSYYASWAKSRFTISRDTSKNTVYLQMGSLRAEDMAVYFCARENPDYGYAYDAWGP | |
| | GTTVTVSS | |
| VH | EVQLVESGGGLVQPGGSLRLSCAVSGFSLSSYGVTWVRQAPGKGLEWIGYITSNYG | 180 |
| hu10C5-H7 | VSYYASWAKSRSTISRDNSKNTVYLQMGSLRAEDMAVYFCARENPDYGYAYDAWGP | |
| | GTTVTVSS | |
| VH | EVQLVESGGGLVQPGGSLRLSCAVSGFSLSSYGVTWVRQAPGKGLEWIGYITSNYG | 181 |
| hu10C5-H8 | VSYYASWAKSRSTISRDTSKNTLYLQMGSLRAEDMAVYFCARENPDYGYAYDAWGP | |
| | GTTVTVSS | |
| VH | EVQLVESGGGLVQPGGSLRLSCAVSGFSLSSYGVTWVRQAPGKGLEWIGYITSNYG | 182 |
| hu10C5-H9 | VSYYASWAKSRSTISRDTSKNTVYLQMGSLRAEDMAVYYCARENPDYGYAYDAWGP | |
| | GTTVTVSS | |
| VH | EVQLVESGGGLVQPGGSLRLSCAVSGFSLSSYGVTWVRQAPGKGLEWIGYITSNYG | 183 |
| hu10C5-H10 | VSYYASWAKSRSTISRDTSKNTVYLQMGSLRAEDMAVYFCARENPDYGYAYDAWGQ | |
| | GTTVTVSS | |
| VH | EVQLVESGGGLVQPGGSLRLSCAVSGFSLSSYGVTWVRQAPGKGLEWIGYITSNYG | 184 |
| hu10C5-H11 | VSYYANSVKGRSTISRDTSKNTVYLQMGSLRAEDMAVYFCARENPDYGYAYDAWGP | |
| | GTTVTVSS | |
| VH | EVQLVESGGGLVQPGGSLRLSCAASGFSLSSYGVTWVRQAPGKGLEYVSYITSNYG | 185 |
| hu10C5-H12 | VSYYASWAKSRFTISRDNSKNTLYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEYVSYITSNYG | 186 |
| hu10C5-H13 | VSYYANSVKGRFTISRDNSKNTLYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWVGYITSNYG | 187 |
| hu10C5-H14 | VSYYASWAKSRFTISRDNSKNTLYLQMGSLRAEDMAVYYCARENPDYGYAYDAWGQ | |
| | GTTVTVSS | |
| VH | EQQLVESGGGLVQPGGSLRLSCAASGFSLSSYGVTWVRQAPGKGLEWVGYITSNYG | 188 |
| hu10C5-H15 | VSYYASWAKSRFTISRDNSKNTLYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWVGYITSNYG | 189 |
| hu10C5-H16 | VSYYASWAKSRFTISRNLNTNTLYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWVGYITSNYG | 190 |
| hu10C5-H17 | VSYYASFAKSRFTISRDNSKNTLYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWVGYITSNYG | 191 |
| hu10C5-H18 | VSYYASWAKSRFTISRNTNLNTLYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWVGYITSNYG | 192 |
| hu10C5-H19 | VSYYASWAKSRFTISRNNSKNTLYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWVGYITSNYG | 193 |
| hu10C5-H20 | VSYYASWAKSRFTISRDNNKNTLYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWVGYITSNYG | 194 |
| hu10C5-H21 | VSYYASWAKSRFTISRNNNKNTLYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWIGYITSNYG | 195 |
| hu10C5-H22 | VSYYASWAKSRFTISRDNSKNTLYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWVGYITSNYG | 196 |
| hu10C5-H23 | VSYYASWAKSRSTISRDNSKNTLYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWIGYITSNYG | 197 |
| hu10C5-H24 | VSYYASWAKSRSTISRDNSKNTLYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWVGYITSNYG | 198 |
| hu10C5-H25 | VSYYASWAKSRFTISRDTSKNTLYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWVGYITSNYG | 199 |
| hu10C5-H26 | VSYYASWAKSRFTISRDNSKNTVYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWIGYITSNYG | 200 |
| hu10C5-H27 | VSYYASWAKSRSTISRDNSKNTVYLQMGSLRAEDMAVYYCARENPDYGYAYDAWGQ | |
| | GTTVTVSS | |
| VH | EVQLVESGGGLVQPGGSLRLSCAASGFSLSSYGVTWVRQAPGKGLEWIGYITSNYG | 201 |
| hu10C5-H28 | VSYYASWAKSRSTISRDTSKNTVYLQMGSLRAEDMAVYYCARENPDYGYAYDAWGQ | |
| | GTTVTVSS | |
| VH | EX₁QLVESGGGLVQPGGSLRLSCAX₂SGFSLSSYGVTWVRQAPGKGLEX₃X₄X₅YITS | 202 |
| 10C5, | NYGVSYYAX₆X₇X₈KX₉RX₁₀TISRX₁₁X₁₂X₁₃X₁₄NTX₁₅YLQMGSLRAEDMAVYX₁₆CA | |
| hu10C5-H1 to | RENPDYGYAYDAWGX₁₇GTTVTVSS | |
| hu10C5-H28 | wherein X₁ is V or Q, X₂ is V or A, X₃ is W or Y, X₄ is I | |
| (consensus) | or V, X₅ is G or S, X₆ is S or N, X₇ is W, S or F, X₈ is | |
| | A or V, X₉ is S or G, X₁₀ is S or F, X_{₁₁} is D or N, X₁₂ is | |
| | T, L or N, X₁₃ is S or N, X₁₄ is K, T or L, X₁₅ is V or L, | |
| | X₁₆ is F or Y, X₁₇ is P or Q | |
| VH | QSVKESEGGLFKPTDTLTLTCTVSGFSLINYGVSWVRQAPGKGLEWIGYIGSAGST | 203 |
| 2B11 | YYATWAKSRATITRNTNLNTVTLKMTSLTAADTATYFCARAAYSAGSADAEDIWGP | |
| | GTLVTVSS | |
| VH | QSVKESEGGLIKPTDTLTLTCTVSGFSLSTFAINWVRQAPGNGLEWIGAIGRGGSA | 204 |
| 10H3 | YYASWAKSRSTITKNTNLNTVTLKMTRPTAADTATYFCARENAGSGWGELDIWGPG | |
| | TLVTVSS | |
| VH | QSVKESEGGLFKPTDTLTLTCTVSGFSLSGYGVSWVRQAPGKGLEYIGFIGSGGSA | 205 |
| 9H3 | YYASWAKSRSTITRNTNLNTVTLKMTRLTAADTATYFCARDNVGGDMSLDIWGPGT | |
| | LVTVSS | |
| VH | QSVKESEGGLFKPTDNLTLTCTVSGFSLSNYGVTWVRQAPGNGLEYIGFIGSSGSA | 206 |
| 8B7 | YYASWAKSRSTITRNTNLNTVTLKMTSLTAADTATYFCARDDVGGGKSLDIWGPGT | |
| | LVTVSS | |
| VH | QSVKESEGGLFKPTDNLTLTCTVSGFSLSSYGVSWVRQAPGNGLEYIGFIGSGGFA | 207 |
| 9H5 | YYASWAKSRSTITRNTNLNTVTLKMTSLTAADTATYFCARDDVGGGKSLDIWGPGT | |
| | VVTVSV | |
| VH | EVQLVESGGGLIQPGGSLRLSCAVSGFSLSSYGVSWVRQAPGKGLEYIGFIGSGGF | 208 |
| hu9H5-H1 | AYYASWAKSRSTISRDTSKNTVYLQMNSLRAEDTAVYFCARDDVGGGKSLDIWGPG | |
| | TLVTVSS | |
| VH | EVQLVESGGGLIQPGGSLRLSCAASGFSLSSYGVSWVRQAPGKGLEYIGFIGSGGF | 209 |
| hu9H5-H2 | AYYASWAKSRSTISRDTSKNTVYLQMNSLRAEDTAVYFCARDDVGGGKSLDIWGPG | |
| | TLVTVSS | |
| VH | EVQLVESGGGLIQPGGSLRLSCAVSGFSLSSYGVSWVRQAPGKGLEWIGFIGSGGF | 210 |
| hu9H5-H3 | AYYASWAKSRSTISRDTSKNTVYLQMNSLRAEDTAVYFCARDDVGGGKSLDIWGPG | |
| | TLVTVSS | |
| VH | EVQLVESGGGLIQPGGSLRLSCAVSGFSLSSYGVSWVRQAPGKGLEYVGFIGSGGF | 211 |
| hu9H5-H4 | AYYASWAKSRSTISRDTSKNTVYLQMNSLRAEDTAVYFCARDDVGGGKSLDIWGPG | |
| | TLVTVSS | |
| VH | EVQLVESGGGLIQPGGSLRLSCAVSGFSLSSYGVSWVRQAPGKGLEYISFIGSGGF | 212 |
| hu9H5-H5 | AYYASWAKSRSTISRDTSKNTVYLQMNSLRAEDTAVYFCARDDVGGGKSLDIWGPG | |
| | TLVTVSS | |
| VH | EVQLVESGGGLIQPGGSLRLSCAVSGFSLSSYGVSWVRQAPGKGLEYIGFIGSGGF | 213 |
| hu9H5-H6 | AYYASWAKSRFTISRDTSKNTVYLQMNSLRAEDTAVYFCARDDVGGGKSLDIWGPG | |
| | TLVTVSS | |
| VH | EVQLVESGGGLIQPGGSLRLSCAVSGFSLSSYGVSWVRQAPGKGLEYIGFIGSGGF | 214 |
| hu9H5-H7 | AYYASWAKSRSTISRDNSKNTVYLQMNSLRAEDTAVYFCARDDVGGGKSLDIWGPG | |
| | TLVTVSS | |
| VH | EVQLVESGGGLIQPGGSLRLSCAVSGFSLSSYGVSWVRQAPGKGLEYIGFIGSGGF | 215 |
| hu9H5-H8 | AYYASWAKSRSTISRDTSKNTLYLQMNSLRAEDTAVYFCARDDVGGGKSLDIWGPG | |
| | TLVTVSS | |
| VH | EVQLVESGGGLIQPGGSLRLSCAVSGFSLSSYGVSWVRQAPGKGLEYIGFIGSGGF | 216 |
| hu9H5-H9 | AYYASWAKSRSTISRDTSKNTVYLQMNSLRAEDTAVYYCARDDVGGGKSLDIWGPG | |
| | TLVTVSS | |
| VH | EVQLVESGGGLIQPGGSLRLSCAVSGFSLSSYGVSWVRQAPGKGLEYIGFIGSGGF | 217 |
| hu9H5-H10 | AYYASWAKSRSTISRDTSKNTVYLQMNSLRAEDTAVYFCARDDVGGGKSLDIWGQG | |
| | TLVTVSS | |
| VH | EVQLVESGGGLIQPGGSLRLSCAVSGFSLSSYGVSWVRQAPGKGLEYIGFIGSGGF | 218 |
| hu9H5-H11 | AYYADSVKGRSTISRDTSKNTVYLQMNSLRAEDTAVYFCARDDVGGGKSLDIWGPG | |
| | TLVTVSS | |
| VH | EVQLVESGGGLIQPGGSLRLSCAASGFSLSSYGVSWVRQAPGKGLEWVSFIGSGGF | 219 |
| hu9H5-H12 | AYYASWAKSRFTISRDNSKNTLYLQMNSLRAEDTAVYYCARDDVGGGKSLDIWGQG | |
| | TLVTVSS | |
| VH | EVQLVESGGGLIQPGGSLRLSCAASGFSLSSYGVSWVRQAPGKGLEWVSFIGSGGF | 220 |
| hu9H5-H13 | AYYADSVKGRFTISRDNSKNTLYLQMNSLRAEDTAVYYCARDDVGGGKSLDIWGQG | |
| | TLVTVSS | |
| VH | EVQLVESGGGLIQPGGSLRLSCAASGFSLSSYGVSWVRQAPGKGLEYVGFIGSGGF | 221 |
| hu9H5-H14 | AYYASWAKSRFTISRDNSKNTLYLQMNSLRAEDTAVYYCARDDVGGGKSLDIWGQG | |
| | TLVTVSS | |
| VH | EQQLVESGGGLIQPGGSLRLSCAASGFSLSSYGVSWVRQAPGKGLEYVGFIGSGGF | 222 |
| hu9H5-H15 | AYYASWAKSRFTISRDNSKNTLYLQMNSLRAEDTAVYYCARDDVGGGKSLDIWGQG | |
| | TLVTVSS | |
| VH | EVQLVESGGGLIQPGGSLRLSCAASGFSLSSYGVSWVRQAPGKGLEYVGFIGSGGF | 223 |
| hu9H5-H16 | AYYASWAKSRFTISRNTNLNTLYLQMNSLRAEDTAVYYCARDDVGGGKSLDIWGQG | |
| | TLVTVSS | |
| VH | EVQLVESGGGLIQPGGSLRLSCAASGFSLSSYGVSWVRQAPGKGLEYVGFIGSGGF | 224 |
| hu9H5-H17 | AYYASFAKSRFTISRDNSKNTLYLQMNSLRAEDTAVYYCARDDVGGGKSLDIWGQG | |
| | TLVTVSS | |
| VH | EX₁QLVESGGGLIQPGGSLRLSCAX₂SGFSLSSYGVSWVRQAPGKGLEX₃X₄X₅FIGS | 225 |
| 9H5, | GGFAYYAX₆X₇X₈KX₉RX₁₀TISRX₁₁X₁₂X₁₃X₁₄NTX₁₅YLQMNSLRAEDTAVYX₁₆CAR | |
| hu9H5-H1 to | DDVGGGKSLDIWGX₁₇GTLVTVSS | |
| hu9H5-H17 | wherein X₁ is V or Q, X₂ is V or A, X₃ is Y or W, X₄ is I | |
| (consensus) | or V, X₅ is G or S, X₆ is S or D, X₇ is W, S or F, X₈ is | |
| | A or V, X₉ is S or G, X₁₀ is S or F, X₁₁ is D or N, X₁₂ is | |
| | T or N, X₁₃ is S or N, X₁₄ is K or L, X₁₅ is V or L, X₁₆ is | |
| | F or Y, X₁₇ is P or Q | |
| VH | QSVKESEGGLFKPTDNLTLTCTVSGFSLSSYGVSWVRQAPGNGLEYIGFIGSGGSP | 226 |
| 9F2 | YYASWAKSRSTITRNTNLNTVTLKMTSLTAADTATYFCARDDVGGGKSLDIWGPGT | |
| | LVTVSS | |
| VH | QSVKESEGGLFKPTDNLTLTCTVSGFSLSSYGVSWVRQAPGNGLEYIGFIGSGGFA | 227 |
| 10C8 | YYASWAKSRSTITRNTNLNTVTLKMTSLTAADTATYFCARDDVGGGRSLDIWGPGT | |
| | VVTVSS | |
| VH | QSVKESEGGLFKPTDTLTLTCTVSGFSLSSYPISWVRQAPGNGLEWIGYITSEYGV | 228 |
| 8F5 | AYYATWAESRSTITRNTNLNTVTLKMTSLTAADTATYFCVRENPTYGYAYDAWGPG | |
| | TLVTVSS | |
| VH | QSVKESEGGLFKPTDTLTLTCTVSGFSLNDYGVSWVRQAPGNGLEWIGAIGSSGVA | 229 |
| 3-3F5 | WYANWAKGRSTITRNTNLNTVTLKMASLTAADTATYFCARDRDYGYRADDATSGMD | |
| | LWGPGTLVTVSS | |
| VH | EVQLVESGGGLVQPGGSLRLSCAVSGFSLNDYGVSWVRQAPGKGLEWIGAIGSSGV | 230 |
| hu3-3F5-H1 | AWYANWAKGRSTISRHTSKNTVYLQMNSLRAEDTAVYFCARDRDYGYRADDATSGM | |
| | DLWGPGTLVTVSS | |
| VH | EVQLVESGGGLVQPGGSLRLSCAASGFSLNDYGVSWVRQAPGKGLEWIGAIGSSGV | 231 |
| hu3-3F5-H2 | AWYANWAKGRSTISRHTSKNTVYLQMNSLRAEDTAVYFCARDRDYGYRADDATSGM | |
| | DLWGPGTLVTVSS | |
| VH | EVQLVESGGGLVQPGGSLRLSCAVSGFSLNDYGVSWVRQAPGKGLEWVGAIGSSGV | 232 |
| hu3-3F5-H3 | AWYANWAKGRSTISRHTSKNTVYLQMNSLRAEDTAVYFCARDRDYGYRADDATSGM | |
| | DLWGPGTLVTVSS | |
| VH | EVQLVESGGGLVQPGGSLRLSCAVSGFSLNDYGVSWVRQAPGKGLEWISAIGSSGV | 233 |
| hu3-3F5-H4 | AWYANWAKGRSTISRHTSKNTVYLQMNSLRAEDTAVYFCARDRDYGYRADDATSGM | |
| | DLWGPGTLVTVSS | |
| VH | EVQLVESGGGLVQPGGSLRLSCAVSGFSLNDYGVSWVRQAPGKGLEWIGAIGSSGV | 234 |
| hu3-3F5-H5 | AWYANWAKGRFTISRHTSKNTVYLQMNSLRAEDTAVYFCARDRDYGYRADDATSGM | |
| | DLWGPGTLVTVSS | |
| VH | EVQLVESGGGLVQPGGSLRLSCAVSGFSLNDYGVSWVRQAPGKGLEWIGAIGSSGV | 235 |
| hu3-3F5-H6 | AWYANWAKGRSTISRHNSKNTVYLQMNSLRAEDTAVYFCARDRDYGYRADDATSGM | |
| | DLWGPGTLVTVSS | |
| VH | EVQLVESGGGLVQPGGSLRLSCAVSGFSLNDYGVSWVRQAPGKGLEWIGAIGSSGV | 236 |
| hu3-3F5-H7 | AWYANWAKGRSTISRHTSKNTLYLQMNSLRAEDTAVYFCARDRDYGYRADDATSGM | |
| | DLWGPGTLVTVSS | |
| VH | EVQLVESGGGLVQPGGSLRLSCAVSGFSLNDYGVSWVRQAPGKGLEWIGAIGSSGV | 237 |
| hu3-3F5-H8 | AWYANWAKGRSTISRHTSKNTVYLQMNSLRAEDTAVYYCARDRDYGYRADDATSGM | |
| | DLWGPGTLVTVSS | |
| VH | EVQLVESGGGLVQPGGSLRLSCAVSGFSLNDYGVSWVRQAPGKGLEWIGAIGSSGV | 238 |
| hu3-3F5-H9 | AWYANWAKGRSTISRHTSKNTVYLQMNSLRAEDTAVYFCARDRDYGYRADDATSGM | |
| | DLWGRGTLVTVSS | |
| VH | EVQLVESGGGLVQPGGSLRLSCAVSGFSLNDYGVSWVRQAPGKGLEWIGAIGSSGV | 239 |
| hu3-3F5-H10 | AFYANWAKGRSTISRHTSKNTVYLQMNSLRAEDTAVYFCARDRDYGYRADDATSGM | |
| | DLWGPGTLVTVSS | |
| VH | EVQLVESGGGLVQPGGSLRLSCAVSGFSLNDYGVSWVRQAPGKGLEWIGAIGSSGV | 240 |
| hu3-3F5-H11 | AWYADSVKGRSTISRHTSKNTVYLQMNSLRAEDTAVYFCARDRDYGYRADDATSGM | |
| | DLWGPGTLVTVSS | |
| VH | EVQLVESGGGLVQPGGSLRLSCAASGFSLNDYGVSWVRQAPGKGLEWVSAIGSSGV | 241 |
| hu3-3F5-H12 | AFYANWAKGRFTISRHNSKNTLYLQMNSLRAEDTAVYYCARDRDYGYRADDATSGM | |
| | DLWGRGTLVTVSS | |
| VH | EVQLVESGGGLVQPGGSLRLSCAASGFSLNDYGVSWVRQAPGKGLEWVSAIGSSGV | 242 |
| hu3-3F5-H13 | AFYADSVKGRFTISRHNSKNTLYLQMNSLRAEDTAVYYCARDRDYGYRADDATSGM | |
| | DLWGRGTLVTVSS | |
| VH | EVQLLESGGGLVQPGGSLRLSCAASGFSLNDYGVSWVRQAPGKGLEWVGAIGSSGV | 243 |
| hu3-3F5-H14 | AWYANWAKGRFTISRDNSKNTLYLQMNSLRAEDTAVYYCARDRDYGYRADDATSGM | |
| | DLWGRGTLVTVSS | |
| VH | EVQLLESGGGLVQPGGSLRLSCAASGFSLNDYGVSWVRQAPGKGLEWVGAIGSSGV | 244 |
| hu3-3F5-H15 | AFYANWAKGRFTISRDNSKNTLYLQMNSLRAEDTAVYYCARDRDYGYRADDATSGM | |
| | DLWGRGTLVTVSS | |
| VH | EQQLLESGGGLVQPGGSLRLSCAASGFSLNDYGVSWVRQAPGKGLEWVGAIGSSGV | 245 |
| hu3-3F5-H16 | AWYANWAKGRFTISRDNSKNTLYLQMNSLRAEDTAVYYCARDRDYGYRADDATSGM | |
| | DLWGRGTLVTVSS | |
| VH | EVQLLESGGGLVQPGGSLRLSCAASGFSLNDYGVSWVRQAPGKGLEWVGAIGSSGV | 246 |
| hu3-3F5-H17 | AWYANWAKGRFTISRNTNLNTLYLQMNSLRAEDTAVYYCARDRDYGYRADDATSGM | |
| | DLWGRGTLVTVSS | |
| VH | EVQLLESGGGLVQPGGSLRLSCAASGFSLNDYGVSWVRQAPGKGLEWVGAIGSSGV | 247 |
| hu3-3F5-H18 | AWYANFAKGRFTISRDNSKNTLYLQMNSLRAEDTAVYYCARDRDYGYRADDATSGM | |
| | DLWGRGTLVTVSS | |
| VH | EVQLLESGGGLVQPGGSLRLSCAVSGFSLNDYGVSWVRQAPGKGLEWVGAIGSSGV | 248 |
| hu3-3F5-H19 | AWYANFAKGRFTISRDNSKNTLYLQMNSLRAEDTAVYYCARDRDYGYRADDATSGM | |
| | DLWGRGTLVTVSS | |
| VH | EVQLLESGGGLVQPGGSLRLSCAASGFSLNDYGVSWVRQAPGKGLEWIGAIGSSGV | 249 |
| hu3-3F5-H20 | AWYANFAKGRFTISRDNSKNTLYLQMNSLRAEDTAVYYCARDRDYGYRADDATSGM | |
| | DLWGRGTLVTVSS | |
| VH | EVQLLESGGGLVQPGGSLRLSCAASGFSLNDYGVSWVRQAPGKGLEWVGAIGSSGV | 250 |
| hu3-3F5-H21 | AWYANFAKGRSTISRDNSKNTLYLQMNSLRAEDTAVYYCARDRDYGYRADDATSGM | |
| | DLWGRGTLVTVSS | |
| VH | EVQLLESGGGLVQPGGSLRLSCAASGFSLNDYGVSWVRQAPGKGLEWIGAIGSSGV | 251 |
| hu3-3F5-H22 | AWYANFAKGRSTISRDNSKNTLYLQMNSLRAEDTAVYYCARDRDYGYRADDATSGM | |
| | DLWGRGTLVTVSS | |
| VH | EVQLLESGGGLVQPGGSLRLSCAASGFSLNDYGVSWVRQAPGKGLEWVGAIGSSGV | 252 |
| hu3-3F5-H23 | AWYANFAKGRFTISRDTSKNTLYLQMNSLRAEDTAVYYCARDRDYGYRADDATSGM | |
| | DLWGRGTLVTVSS | |
| VH | EVQLLESGGGLVQPGGSLRLSCAASGFSLNDYGVSWVRQAPGKGLEWVGAIGSSGV | 253 |
| hu3-3F5-H24 | AWYANFAKGRFTISRDNSKNTVYLQMNSLRAEDTAVYYCARDRDYGYRADDATSGM | |
| | DLWGRGTLVTVSS | |
| VH | EVQLLESGGGLVQPGGSLRLSCAASGFSLNDYGVSWVRQAPGKGLEWVGAIGSSGV | 254 |
| hu3-3F5-H25 | AWYANFAKGRFTISRDTSKNTVYLQMNSLRAEDTAVYYCARDRDYGYRADDATSGM | |
| | DLWGRGTLVTVSS | |
| VH | EVQLLESGGGLVQPGGSLRLSCAASGFSLNDYGVSWVRQAPGKGLEWIGAIGSSGV | 255 |
| hu3-3F5-H26 | AWYANFAKGRSTISRDTSKNTVYLQMNSLRAEDTAVYYCARDRDYGYRADDATSGM | |
| | DLWGRGTLVTVSS | |
| VH | EVQLLESGGGLVQPGGSLRLSCAASGFSLNDYGVSWVRQAPGKGLEWIGAIGSSGV | 256 |
| hu3-3F5-H27 | AFYANFAKGRSTISRDTSKNTVYLQMNSLRAEDTAVYYCARDRDYGYRADDATSGM | |
| | DLWGRGTLVTVSS | |
| VH | EX₁QLX₂ESGGGLVQPGGSLRLSCAX₃SGFSLNDYGVSWVRQAPGKGLEWX₄X₅AIGS | 257 |
| 3-3F5, | SGVAX₆YAX₇X₈X₉KGRX₁₀TISRX₁₁X₁₂X₁₃X₁₄NTX₁₅YLQMNSLRAEDTAVYX₁₆CAR | |
| hu3-3F5-H1 to | DRDYGYRADDATSGMDLWGX₁₇GTLVTVSS | |
| hu3-3F5-H27 | wherein X₁ is V or Q, X₂ is V or L, X₃ is V or A, X₄ is I | |
| (consensus) | or V, X₅ is G or S, X₆ is W or F, X₇ is N or D, X₈ is W, S | |
| | or F, X₉ is A or V, X₁₀ is S or F, X₁₁ is H, N or D, X₁₂ is | |
| | T or N, X₁₃ is S or N, X₁₄ is K or L, X₁₅ is V or L, X₁₆ is | |
| | F or Y, X₁₇ is P or R | |
| VH | QEQLEESGGGLVKPGASLTLTCTVSGFSLTNNYVNFVMCWVRQAPGKGLEWIASID | 258 |
| 9E3 | PGDDSTDYASWATGRFTISKASSTTVTLQVTSLTAADTATYFCARGDAGTSYSFNF | |
| | WGPGTLVTVS | |
| VH | QEQLVESGGGLVKPGASLTLTCTASGFSLTDNYVMSWVRQAPGKGLEWIACIDPGD | 259 |
| 10D10 | DSTYYASWATGRFTISRASSTTVTLQVTSLTAADTATYFCARGDAAASYSFNFWGP | |
| | GTLVTVSS | |
| VH | QSLEESGGGLVKPGGTLTLTCKASGIDFNGGGIYWVRQAPGKGLEWIASIYPDHGS | 260 |
| 12B3 | VDYANWVNGRFTISLDNAQNTVFLQLTSLTVADTATYFCARESGGSYYDLWGPGTL | |
| | VTVSS | |
| VH | QSLEESGGGLVKPGGTLTLTCTASGFDFNGGGIYWVRQAPGKGLEWIAYIYPDHGS | 261 |
| 1D10 | ADYATWVNGRFTISLDNAQNTVFLQMTSLTVADTATYFCARETGGSWYDLWGPGTL | |
| | VTVSS | |

LC Sequences

| | | |
|---|---|---|
| LC | DIQMTQSPASLSASLGETVTIQCRASEDIYSGLAWYQQKPGKSPQLLIYGATTLHD | 262 |
| 14C8 | GVPSRFSGSGSGTQYSLKISSMHSEDEGIYFCHQGLSFPYTFGAGTKLELKRADAA | |
| | PTVSIFPPSSEQLTSGGASVVCFLNNFYPKDINVKWKIDGSERQNGVLNSWTDQDS | |
| | KDSTYSMSSTLTLTKDEYERHNSYTCEATHKTSTSPIVKSFNRNEC | |
| LC | DIQMTQSPASLSASLGETVTIECLASKNIYRNLAWYQQKPGKSPQFLISDASRLQD | 263 |
| 14E12 | GVPSRFTGSDSGSQYSLKINSLQSEDVATYFCQQYHDYPYTFGAGTKLELKRADAA | |
| | PTVSIFPPSSEQLTSGGASVVCFLNNFYPKDINVKWKIDGSERQNGVLNSWTDQDS | |
| | KDSTYSMSSTLTLTKDEYERHNSYTCEATHKTSTSPIVKSFNRNEC | |
| LC | DVVMTQTASPVSAAVGGTVTIKCQASENIYSLLAWYQQKPGQPPKVLIYDASDLAS | 264 |
| 8E11 | GVPSRFKGSGSGTQFTLTISDLECADAATYYCQATAYGSSGNAFGGGTEVVVKRAD | |
| | AAPTVSIFPPSSEQLTSGGASVVCFLNNFYPKDINVKWKIDGSERQNGVLNSWTDQ | |
| | DSKDSTYSMSSTLTLTKDEYERHNSYTCEATHKTSTSPIVKSFNRNEC | |
| LC | DVVMTQTPASVSEPVGGTVTIKCQASENIYSLLAWYQQKPGQPPKVLIYDASDLAS | 265 |
| 8G10 | GVPSRFKGSGSGTQFTLTISDLECADAATYYCQATAYGSSGNAFGGGTEVVVKRAD | |
| | AAPTVSIFPPSSEQLTSGGASVVCFLNNFYPKDINVKWKIDGSERQNGVLNSWTDQ | |
| | DSKDSTYSMSSTLTLTKDEYERHNSYTCEATHKTSTSPIVKSFNRNEC | |
| LC | NIVMTQTPASVEVAVGGTVVIKCQASQNIGDYLSWYQQKPGQRPKLLIYSASTLAS | 266 |
| 9B6 | GVPSRFKGSGSGTQFTLTISDLECADAATYYCHQDYTSNDVENTFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | AYYMTQTPASVEVAVGGTVTIKCQASEDIGSYCSWYQQKPGQPPKLLIYDASDLAS | 267 |
| 2.3F4 | GVPSRFKGSGSGTDFTLTISGVQCDDAATYYCQQDYTGNNVDNTFGGGSEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | AYDMTQTPASLEAAVGGTVTINCQASESISNELSWYQQKPGQPPDLLIYYASTLAS | 268 |
| 10C5 | GVPSRFKGSGSGTEFTLTISDLECADAATYYCAQGFGSSGVENVFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | AIRMTQSPSSFSASTGDRVTITCQASESISNELSWYQQKPGKAPKLLIYYASTLAS | 269 |
| 10C5-L5 | GVPSRFSGSGSGTDFTLTISSLQPEDFATYYCAQGFGSSGVENVFGGGTKVEIKRT | |
| huKappa | VAAPSVFIFPPSDEQLKSGTASVVCLLNNFYPREAKVQWKVDNALQSGNSQESVTE | |
| | QDSKDSTYSLSSTLTLSKADYEKHKVYACEVTHQGLSSPVTKSFNRGEC | |
| LC | AYYMTQTPASVEAAVGGTVTIKCQASQSISNYVAWYQQKPGQPPKLLIYRASTLAS | 270 |
| 2B11 | GVSSRFSGSGSGTEFTLTISDLECADAATYYCHQGYSSSNVDNIFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | AYEMTQTPASVEVAVGGTVTINCQASESISSDLAWYQQKPGQRPKLLIYAASTLAS | 271 |
| 10H3 | GVPSRFKGSGSGTEFTLSISGVQCADAATYYCQQGYTWNNVDNVFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | DIVMTQTPASVSEPVGGTVTIKCQASQNINNYLSWYQQKPGQPPKQLIYAASTLAS | 272 |
| 9H3 | GVPSRFKGSGSGTQFTLTISDLECADAATYYCHQDYTSNNVDNTFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | NIVMTQTPASVEVAVGGTVTIKCQASQSIGSYLSWYQQKPGQPPKLLIYDASNLAS | 273 |
| 8B7 | GVPSRFKGSGSGTQFTLSISDLECADAATYYCHQDYTSNNVDNTFGGGTEVVIKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | NIVMTQTPASVEAAVGGTVTINCQASQSISSYLSWYQQKSGQRPKLLIFSASTLAS | 274 |
| 9H5 | GVPSRFTGSGSGTQFTLTISDLQCADAATYYCHQDYTSSNVDNTFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | DIQMTQSPSSLSASVGDRVTITCQASQSISSYLSWYQQKPGKAPKLLIYSASTLAS | 275 |
| 9H5-L4 | GVPSRFSGSGSGTDFTLTISSLQPEDFATYYCHQDYTSSNVDNTFGGGTKVEIKRT | |
| huKappa | VAAPSVFIFPPSDEQLKSGTASVVCLLNNFYPREAKVQWKVDNALQSGNSQESVTE | |
| | QDSKDSTYSLSSTLTLSKADYEKHKVYACEVTHQGLSSPVTKSFNRGEC | |
| LC | NIVMTQTPASVEVAVGGTVIIKCQASQSISNYLSWYHQKSGQRPRLLIYSASTLAS | 276 |
| 9F2 | GVPSRFKGSGSGTQFTLTISDLECADAATYYCHQDYTSNSVDNTFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | NIVMTQTPASVEVAMGGTVIIKCQASQSISTYLSWYQQKPGQPPKLLIYSASTLAS | 277 |
| 10C8 | GVSSRFEGSGSGTQFTLTISGVQCADAATYYCHQDYTSNNVDNTFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | AYDLTQTPASVEAAVGGTVTINCQASESIGNELSWYQQKSGQPPKLLIYQASTLAS | 278 |
| 8F5 | GVPSRFKGSGSGTDFTLTISDLECADAATYYCAQGFSSSGVENVFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | AYDMTQTPASVEAAVGGTVTIKCQASESIGNALAWYQQKPGQPPKLLIYDASDLAS | 279 |
| 3-3F5 | GVPSRFKGSGSGTQFTLTISGVECADAATYYCQQGDSHNNVDNIFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | AIRMTQSPSSFSASTGDRVTITCQASESIGNALAWYQQKPGKAPKLLIYDASDLAS | 280 |
| 3-3F5-L5 | GVPSRFSGSGSGTDFTLTISSLQPEDFATYYCQQGDSHNNVDNIFGGGTKVEIKRT | |
| huKappa | VAAPSVFIFPPSDEQLKSGTASVVCLLNNFYPREAKVQWKVDNALQSGNSQESVTE | |
| | QDSKDSTYSLSSTLTLSKADYEKHKVYACEVTHQGLSSPVTKSFNRGEC | |
| LC | AIEMTQTPSSASEPVGGTVTIKCQASESISRYLSWYQQKPGQPPKLLIYDASDLAS | 281 |
| 9E3 | GVSSRFKGSGSGTQFTLIISDVECADAATYYCQQDYSRSNIVNSFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | AYDMTQTPSSASEPVGGTVTIKCQASESISTYLSWYQQKPGQPPKLLIYDASDLAS | 282 |
| 10D10 | GVSSRFKGSGSGTQFTLTISDVECADAATYYCQQDYSSSNIVNSFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | AYDMTQTPASVEVAVGGTVTIKCQASQSISTYLAWYQQKPGQRPNLLIYKTSTLAS | 283 |
| 12B3 | GVPSRFRGSGSGTQFTLTISGVECADAATYYCQQGYSGSSVENTFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |
| LC | AYDMTQTPVSVEAAVGGTVTIKCQASQSISSYLAWYQQKPGQPPKLLIYKASTLAS | 284 |
| 1D10 | GVSSRFKGSGSGTEFTLTISDLECADAATYYCQQGYSGSNVENTFGGGTEVVVKRA | |
| | DAAPTVSIFPPSSEQLTSGGASVVCFLNNFYPKDINVKWKIDGSERQNGVLNSWTD | |
| | QDSKDSTYSMSSTLTLTKDEYERHNSYTCEATHKTSTSPIVKSFNRNEC | |

HC Sequences

| | | |
|---|---|---|
| HC | EVQLVESGGGLVQPGRSLKLSCTASGFTFSDYNMAWVRQAPKGGLEWVTTISYDAG | 285 |
| 14C8 | RTYYRDSVKGRFTISRDNAKRTLSLQMDSLRSEDTATYYCATGIFNYGTDYFDYWG | |
| | QGVMVTVSSASTKGPSVYPLAPVCGDTTGSSVTLGCLVKGYFPEPVTLTWNSGSLS | |
| | SGVHTFPAVLQSDLYTLSSSVTVTSSTWPSQSITCNVAHPASSTKVDKKIEPRGPT | |
| | IKPCPPCKCPAPNLLGGPSVFIFPPKIKDVLMISLSPIVTCVVVDVSEDDPDVQIS | |
| | WFVNNVEVHTAQTQTHREDYNSTLRVVSALPIQHQDWMSGKEFKCKVNNKDLPAPI | |
| | ERTISKPKGSVRAPQVYVLPPPEEEMTKKQVTLTCMVTDFMPEDIYVEWTNNGKTE | |
| | LNYKNTEPVLDSDGSYFMYSKLRVEKKNWVERNSYSCSVVHEGLHNHHTTKSFSRT | |
| | PGK | |
| HC | EVQLVESGGGLVRPGRSLRLSCAASGFTFSDYYMAWVRQAPTKGLEWVASISYDGD | 286 |
| 14E12 | TTYYRDSVKGRFTISRDNARSSLYLQMDSLRSDDTANYFCTTDGTIPAGSWFAYWG | |
| | QGTLVTVSSASTKGPSVYPLAPVCGDTTGSSVTLGCLVKGYFPEPVTLTWNSGSLS | |
| | SGVHTFPAVLQSDLYTLSSSVTVTSSTWPSQSITCNVAHPASSTKVDKKIEPRGPT | |
| | IKPCPPCKCPAPNLLGGPSVFIFPPKIKDVLMISLSPIVTCVVVDVSEDDPDVQIS | |
| | WFVNNVEVHTAQTQTHREDYNSTLRVVSALPIQHQDWMSGKEFKCKVNNKDLPAPI | |
| | ERTISKPKGSVRAPQVYVLPPPEEEMTKKQVTLTCMVTDFMPEDIYVEWTNNGKTE | |
| | LNYKNTEPVLDSDGSYFMYSKLRVEKKNWVERNSYSCSVVHEGLHNHHTTKSFSRT | |
| | PGK | |
| HC | QEQLEESGGDLVKPEGSLTLTCTASGFSFSSSYWICWVRQAPGKGLEWIACVYGLD | 287 |
| 8E11 | VNIYYASWTKGRFTISKTSSTTVTLQMTSLTAADTATYFCARGGGSADFGFDLWGP | |
| | GTLVTVSSASTKGPSVYPLAPVCGDTTGSSVTLGCLVKGYFPEPVTLTWNSGSLSS | |
| | GVHTFPAVLQSDLYTLSSSVTVTSSTWPSQSITCNVAHPASSTKVDKKIEPRGPTI | |
| | KPCPPCKCPAPNLLGGPSVFIFPPKIKDVLMISLSPIVTCVVVDVSEDDPDVQISW | |
| | FVNNVEVHTAQTQTHREDYNSTLRVVSALPIQHQDWMSGKEFKCKVNNKDLPAPIE | |
| | RTISKPKGSVRAPQVYVLPPPEEEMTKKQVTLTCMVTDFMPEDIYVEWTNNGKTEL | |
| | NYKNTEPVLDSDGSYFMYSKLRVEKKNWVERNSYSCSVVHEGLHNHHTTKSFSRTP | |
| | GK | |
| HC | QSLEESGGDLVKPEGSLTLTCTASGFSFSTSYWICWVRQAPGKGLEWIACVYGLDV | 288 |
| 8G10 | NIYYASWTEGRFTISKTSSTTVTLQVTSLTAADTATYFCARGGGSADFGFDLWGPG | |
| | TLVTVSSASTKGPSVYPLAPVCGDTTGSSVTLGCLVKGYFPEPVTLTWNSGSLSSG | |
| | VHTFPAVLQSDLYTLSSSVTVTSSTWPSQSITCNVAHPASSTKVDKKIEPRGPTIK | |
| | PCPPCKCPAPNLLGGPSVFIFPPKIKDVLMISLSPIVTCVVVDVSEDDPDVQISWF | |
| | VNNVEVHTAQTQTHREDYNSTLRVVSALPIQHQDWMSGKEFKCKVNNKDLPAPIER | |
| | TISKPKGSVRAPQVYVLPPPEEEMTKKQVTLTCMVTDFMPEDIYVEWTNNGKTELN | |
| | YKNTEPVLDSDGSYFMYSKLRVEKKNWVERNSYSCSVVHEGLHNHHTTKSFSRTPG | |
| | K | |
| HC | QSVKESEGGLFKPTDNLTLTCTVSGFSLSNYGVTWVRQAPGNGLEYIGFIGSGGSA | 289 |
| 9B6 | YYASWAKSRSTITRNTNLNTVTLKMTSLTAADTATYFCARDDVGGGKSLDIWGPGT | |
| | LVTVSSASTKGPSVYPLAPVCGDTTGSSVTLGCLVKGYFPEPVTLTWNSGSLSSGV | |
| | HTFPAVLQSDLYTLSSSVTVTSSTWPSQSITCNVAHPASSTKVDKKIEPRGPTIKP | |
| | CPPCKCPAPNLLGGPSVFIFPPKIKDVLMISLSPIVTCVVVDVSEDDPDVQISWFV | |
| | NNVEVHTAQTQTHREDYNSTLRVVSALPIQHQDWMSGKEFKCKVNNKDLPAPIERT | |
| | ISKPKGSVRAPQVYVLPPPEEEMTKKQVTLTCMVTDFMPEDIYVEWTNNGKTELNY | |
| | KNTEPVLDSDGSYFMYSKLRVEKKNWVERNSYSCSVVHEGLHNHHTTKSFSRTPGK | |
| HC | QSVEESRGGLIKPTDTLTLTCTASGFSLSNYGVSWVRQAPGNGLEYIGFIGYGGST | 290 |
| 2.3F4 | YYASWAKSRSTITRNTNLNTVTLQMTSLTAADTATYFCARLCGVDCADALDSWGPG | |
| | TLVTVSSASTKGPSVYPLAPVCGDTTGSSVTLGCLVKGYFPEPVTLTWNSGSLSSG | |
| | VHTFPAVLQSDLYTLSSSVTVTSSTWPSQSITCNVAHPASSTKVDKKIEPRGPTIK | |
| | PCPPCKCPAPNLLGGPSVFIFPPKIKDVLMISLSPIVTCVVVDVSEDDPDVQISWF | |
| | VNNVEVHTAQTQTHREDYNSTLRVVSALPIQHQDWMSGKEFKCKVNNKDLPAPIER | |
| | TISKPKGSVRAPQVYVLPPPEEEMTKKQVTLTCMVTDFMPEDIYVEWTNNGKTELN | |
| | YKNTEPVLDSDGSYFMYSKLRVEKKNWVERNSYSCSVVHEGLHNHHTTKSFSRTPG | |
| | K | |
| HC | QSLEESGGGLVKPTDTLTLTCTVSGFSLSSYGVTWVRQAPGRGLEWIGYITSNYGV | 291 |
| 10C5 | SYYASWAKSRSTITRNTNLNTVTLKMTSLTAADTATYFCARENPDYGYAYDAWGPG | |
| | TLVTVSSASTKGPSVYPLAPVCGDTTGSSVTLGCLVKGYFPEPVTLTWNSGSLSSG | |
| | VHTFPAVLQSDLYTLSSSVTVTSSTWPSQSITCNVAHPASSTKVDKKIEPRGPTIK | |
| | PCPPCKCPAPNLLGGPSVFIFPPKIKDVLMISLSPIVTCVVVDVSEDDPDVQISWF | |
| | VNNVEVHTAQTQTHREDYNSTLRVVSALPIQHQDWMSGKEFKCKVNNKDLPAPIER | |
| | TISKPKGSVRAPQVYVLPPPEEEMTKKQVTLTCMVTDFMPEDIYVEWTNNGKTELN | |
| | YKNTEPVLDSDGSYFMYSKLRVEKKNWVERNSYSCSVVHEGLHNHHTTKSFSRTPG | |
| | K | |
| HC | EVQLVESGGGLVQPGGSLRLSCAASGFSLSSYGVTWVRQAPGKGLEWIGYITSNYG | 292 |
| 10C5-H28 | VSYYASWAKSRSTISRDTSKNTVYLQMGSLRAEDMAVYYCARENPDYGYAYDAWGQ | |
| hIgG1 | GTTVTVSSASTKGPSVFPLAPSSKSTSGGTAALGCLVKDYFPEPVTVSWNSGALTS | |
| N297G | GVHTFPAVLQSSGLYSLSSVVTVPSSSLGTQTYICNVNHKPSNTKVDKKVEPKSCD | |
| | KTHTCPPCPAPELLGGPSVFLFPPKPKDTLMISRTPEVTCVVVDVSHEDPEVKFNW | |
| | YVDGVEVHNAKTKPREEQYGSTYRVVSVLTVLHQDWLNGKEYKCKVSNKALPAPIE | |
| | KTISKAKGQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYTQKSLSLSP | |
| | GK | |
| HC | EVQLVESGGGLVQPGGSLRLSCAASGFSLSSYGVTWVRQAPGKGLEWIGYITSNYG | 293 |
| 10C5-H28 | VSYYASWAKSRSTISRDTSKNTVYLQMGSLRAEDMAVYYCARENPDYGYAYDAWGQ | |
| hIgG4 | GTTVTVSSASTKGPSVFPLAPCSRSTSESTAALGCLVKDYFPEPVTVSWNSGALTS | |
| S228P | GVHTFPAVLQSSGLYSLSSVVTVPSSSLGTKTYTCNVDHKPSNTKVDKRVESKYGP | |
| | PCPPCPAPEFLGGPSVFLFPPKPKDTLMISRTPEVTCVVVDVSQEDPEVQFNWYVD | |
| | GVEVHNAKTKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESNGQPENNYK | |
| | TTPPVLDSDGSFFLYSRLTVDKSRWQEGNVFSCSVMHEALHNHYTQKSLSLSLGK | |
| HC | QSVKESEGGLFKPTDTLTLTCTVSGFSLINYGVSWVRQAPGKGLEWIGYIGSAGST | 294 |
| 2B11 | YYATWAKSRATITRNTNLNTVTLKMTSLTAADTATYFCARAAYSAGSADAEDIWGP | |
| | GTLVTVSSASTKGPSVYPLAPVCGDTTGSSVTLGCLVKGYFPEPVTLTWNSGSLSS | |
| | GVHTFPAVLQSDLYTLSSSVTVTSSTWPSQSITCNVAHPASSTKVDKKIEPRGPTI | |
| | KPCPPCKCPAPNLLGGPSVFIFPPKIKDVLMISLSPIVTCVVVDVSEDDPDVQISW | |
| | FVNNVEVHTAQTQTHREDYNSTLRVVSALPIQHQDWMSGKEFKCKVNNKDLPAPIE | |
| | RTISKPKGSVRAPQVYVLPPPEEEMTKKQVTLTCMVTDFMPEDIYVEWTNNGKTEL | |
| | NYKNTEPVLDSDGSYFMYSKLRVEKKNWVERNSYSCSVVHEGLHNHHTTKSFSRTP | |
| | GK | |
| HC | QSVKESEGGLIKPTDTLTLTCTVSGFSLSTFAINWVRQAPGNGLEWIGAIGRGGSA | 295 |
| 10H3 | YYASWAKSRSTITKNTNLNTVTLKMTRPTAADTATYFCARENAGSGWGELDIWGPG | |
| | TLVTVSSASTKGPSVYPLAPVCGDTTGSSVTLGCLVKGYFPEPVTLTWNSGSLSSG | |
| | VHTFPAVLQSDLYTLSSSVTVTSSTWPSQSITCNVAHPASSTKVDKKIEPRGPTIK | |
| | PCPPCKCPAPNLLGGPSVFIFPPKIKDVLMISLSPIVTCVVVDVSEDDPDVQISWF | |
| | VNNVEVHTAQTQTHREDYNSTLRVVSALPIQHQDWMSGKEFKCKVNNKDLPAPIER | |
| | TISKPKGSVRAPQVYVLPPPEEEMTKKQVTLTCMVTDFMPEDIYVEWTNNGKTELN | |
| | YKNTEPVLDSDGSYFMYSKLRVEKKNWVERNSYSCSVVHEGLHNHHTTKSFSRTPG | |
| | K | |
| HC | QSVKESEGGLFKPTDTLTLTCTVSGFSLSGYGVSWVRQAPGKGLEYIGFIGSGGSA | 296 |
| 9H3 | YYASWAKSRSTITRNTNLNTVTLKMTRLTAADTATYFCARDNVGGDMSLDIWGPGT | |
| | LVTVSSASTKGPSVYPLAPVCGDTTGSSVTLGCLVKGYFPEPVTLTWNSGSLSSGV | |
| | HTFPAVLQSDLYTLSSSVTVTSSTWPSQSITCNVAHPASSTKVDKKIEPRGPTIKP | |
| | CPPCKCPAPNLLGGPSVFIFPPKIKDVLMISLSPIVTCVVVDVSEDDPDVQISWFV | |
| | NNVEVHTAQTQTHREDYNSTLRVVSALPIQHQDWMSGKEFKCKVNNKDLPAPIERT | |
| | ISKPKGSVRAPQVYVLPPPEEEMTKKQVTLTCMVTDFMPEDIYVEWTNNGKTELNY | |
| | KNTEPVLDSDGSYFMYSKLRVEKKNWVERNSYSCSVVHEGLHNHHTTKSFSRTPGK | |
| HC | QSVKESEGGLFKPTDNLTLTCTVSGFSLSNYGVTWVRQAPGNGLEYIGFIGSSGSA | 297 |
| 8B7 | YYASWAKSRSTITRNTNLNTVTLKMTSLTAADTATYFCARDDVGGGKSLDIWGPGT | |
| | LVTVSSASTKGPSVYPLAPVCGDTTGSSVTLGCLVKGYFPEPVTLTWNSGSLSSGV | |
| | HTFPAVLQSDLYTLSSSVTVTSSTWPSQSITCNVAHPASSTKVDKKIEPRGPTIKP | |
| | CPPCKCPAPNLLGGPSVFIFPPKIKDVLMISLSPIVTCVVVDVSEDDPDVQISWFV | |
| | NNVEVHTAQTQTHREDYNSTLRVVSALPIQHQDWMSGKEFKCKVNNKDLPAPIERT | |
| | ISKPKGSVRAPQVYVLPPPEEEMTKKQVTLTCMVTDFMPEDIYVEWTNNGKTELNY | |
| | KNTEPVLDSDGSYFMYSKLRVEKKNWVERNSYSCSVVHEGLHNHHTTKSFSRTPGK | |
| HC | QSVKESEGGLFKPTDNLTLTCTVSGFSLSSYGVSWVRQAPGNGLEYIGFIGSGGFA | 298 |
| 9H5 | YYASWAKSRSTITRNTNLNTVTLKMTSLTAADTATYFCARDDVGGGKSLDIWGPGT | |
| | VVTVSSASTKGPSVYPLAPVCGDTTGSSVTLGCLVKGYFPEPVTLTWNSGSLSSGV | |
| | HTFPAVLQSDLYTLSSSVTVTSSTWPSQSITCNVAHPASSTKVDKKIEPRGPTIKP | |
| | CPPCKCPAPNLLGGPSVFIFPPKIKDVLMISLSPIVTCVVVDVSEDDPDVQISWFV | |
| | NNVEVHTAQTQTHREDYNSTLRVVSALPIQHQDWMSGKEFKCKVNNKDLPAPIERT | |
| | ISKPKGSVRAPQVYVLPPPEEEMTKKQVTLTCMVTDFMPEDIYVEWTNNGKTELNY | |
| | KNTEPVLDSDGSYFMYSKLRVEKKNWVERNSYSCSVVHEGLHNHHTTKSFSRTPGK | |
| HC | EVQLVESGGGLIQPGGSLRLSCAASGFSLSSYGVSWVRQAPGKGLEYVGFIGSGGF | 299 |
| 9H5-H14 | AYYASWAKSRFTISRDNSKNTLYLQMNSLRAEDTAVYYCARDDVGGGKSLDIWGQG | |
| hIgG1 | TLVTVSSASTKGPSVFPLAPSSKSTSGGTAALGCLVKDYFPEPVTVSWNSGALTSG | |
| N297G | VHTFPAVLQSSGLYSLSSVVTVPSSSLGTQTYICNVNHKPSNTKVDKKVEPKSCDK | |
| | THTCPPCPAPELLGGPSVFLFPPKPKDTLMISRTPEVTCVVVDVSHEDPEVKFNWY | |
| | VDGVEVHNAKTKPREEQYGSTYRVVSVLTVLHQDWLNGKEYKCKVSNKALPAPIEK | |
| | TISKAKGQPREPQVYTLPPSREEMTKNQVSLTCLVKGFYPSDIAVEWESNGQPENN | |
| | YKTTPPVLDSDGSFFLYSKLTVDKSRWQQGNVFSCSVMHEALHNHYTQKSLSLSPG | |
| | K | |
| HC | EVQLVESGGGLIQPGGSLRLSCAASGFSLSSYGVSWVRQAPGKGLEYVGFIGSGGF | 300 |
| 9H5-H14 | AYYASWAKSRFTISRDNSKNTLYLQMNSLRAEDTAVYYCARDDVGGGKSLDIWGQG | |
| hIgG4 | TLVTVSSASTKGPSVFPLAPCSRSTSESTAALGCLVKDYFPEPVTVSWNSGALTSG | |
| S228P | VHTFPAVLQSSGLYSLSSVVTVPSSSLGTKTYTCNVDHKPSNTKVDKRVESKYGPP | |
| | CPPCPAPEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTIS | |
| | KAKGQPREPQVYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQKSLSLSLGK | |
| HC | QSVKESEGGLFKPTDNLTLTCTVSGFSLSSYGVSWVRQAPGNGLEYIGFIGSGGSP | 301 |
| 9F2 | YYASWAKSRSTITRNTNLNTVTLKMTSLTAADTATYFCARDDVGGGKSLDIWGPGT | |
| | LVTVSSASTKGPSVYPLAPVCGDTTGSSVTLGCLVKGYFPEPVTLTWNSGSLSSGV | |
| | HTFPAVLQSDLYTLSSSVTVTSSTWPSQSITCNVAHPASSTKVDKKIEPRGPTIKP | |
| | CPPCKCPAPNLLGGPSVFIFPPKIKDVLMISLSPIVTCVVVDVSEDDPDVQISWFV | |
| | NNVEVHTAQTQTHREDYNSTLRVVSALPIQHQDWMSGKEFKCKVNNKDLPAPIERT | |
| | ISKPKGSVRAPQVYVLPPPEEEMTKKQVTLTCMVTDFMPEDIYVEWTNNGKTELNY | |
| | KNTEPVLDSDGSYFMYSKLRVEKKNWVERNSYSCSVVHEGLHNHHTTKSFSRTPGK | |
| HC | QSVKESEGGLFKPTDNLTLTCTVSGFSLSSYGVSWVRQAPGNGLEYIGFIGSGGFA | 302 |
| 10C8 | YYASWAKSRSTITRNTNLNTVTLKMTSLTAADTATYFCARDDVGGGRSLDIWGPGT | |
| | VVTVSSASTKGPSVYPLAPVCGDTTGSSVTLGCLVKGYFPEPVTLTWNSGSLSSGV | |
| | HTFPAVLQSDLYTLSSSVTVTSSTWPSQSITCNVAHPASSTKVDKKIEPRGPTIKP | |
| | CPPCKCPAPNLLGGPSVFIFPPKIKDVLMISLSPIVTCVVVDVSEDDPDVQISWFV | |
| | NNVEVHTAQTQTHREDYNSTLRVVSALPIQHQDWMSGKEFKCKVNNKDLPAPIERT | |
| | ISKPKGSVRAPQVYVLPPPEEEMTKKQVTLTCMVTDFMPEDIYVEWTNNGKTELNY | |
| | KNTEPVLDSDGSYFMYSKLRVEKKNWVERNSYSCSVVHEGLHNHHTTKSFSRTPGK | |
| HC | QSVKESEGGLFKPTDTLTLTCTVSGFSLSSYPISWVRQAPGNGLEWIGYITSEYGV | 303 |
| 8F5 | AYYATWAESRSTITRNTNLNTVTLKMTSLTAADTATYFCVRENPTYGYAYDAWGPG | |
| | TLVTVSSASTKGPSVYPLAPVCGDTTGSSVTLGCLVKGYFPEPVTLTWNSGSLSSG | |
| | VHTFPAVLQSDLYTLSSSVTVTSSTWPSQSITCNVAHPASSTKVDKKIEPRGPTIK | |
| | PCPPCKCPAPNLLGGPSVFIFPPKIKDVLMISLSPIVTCVVVDVSEDDPDVQISWF | |
| | VNNVEVHTAQTQTHREDYNSTLRVVSALPIQHQDWMSGKEFKCKVNNKDLPAPIER | |
| | TISKPKGSVRAPQVYVLPPPEEEMTKKQVTLTCMVTDFMPEDIYVEWTNNGKTELN | |
| | YKNTEPVLDSDGSYFMYSKLRVEKKNWVERNSYSCSVVHEGLHNHHTTKSFSRTPG | |
| | K | |
| HC | QSVKESEGGLFKPTDTLTLTCTVSGFSLNDYGVSWVRQAPGNGLEWIGAIGSSGVA | 304 |
| 3-3F5 | WYANWAKGRSTITRNTNLNTVTLKMASLTAADTATYFCARDRDYGYRADDATSGMD | |
| | LWGPGTLVTVSSASTKGPSVYPLAPVCGDTTGSSVTLGCLVKGYFPEPVTLTWNSG | |
| | SLSSGVHTFPAVLQSDLYTLSSSVTVTSSTWPSQSITCNVAHPASSTKVDKKIEPR | |
| | GPTIKPCPPCKCPAPNLLGGPSVFIFPPKIKDVLMISLSPIVTCVVVDVSEDDPDV | |
| | QISWFVNNVEVHTAQTQTHREDYNSTLRVVSALPIQHQDWMSGKEFKCKVNNKDLP | |
| | APIERTISKPKGSVRAPQVYVLPPPEEEMTKKQVTLTCMVTDFMPEDIYVEWTNNG | |
| | KTELNYKNTEPVLDSDGSYFMYSKLRVEKKNWVERNSYSCSVVHEGLHNHHTTKSF | |
| | SRTPGK | |
| HC | EVQLLESGGGLVQPGGSLRLSCAVSGFSLNDYGVSWVRQAPGKGLEWVGAIGSSGV | 305 |
| 3-3F5-H19 | AWYANFAKGRFTISRDNSKNTLYLQMNSLRAEDTAVYYCARDRDYGYRADDATSGM | |
| hIgG1 | DLWGRGTLVTVSSASTKGPSVFPLAPSSKSTSGGTAALGCLVKDYFPEPVTVSWNS | |
| N297G | GALTSGVHTFPAVLQSSGLYSLSSVVTVPSSSLGTQTYICNVNHKPSNTKVDKKVE | |
| | PKSCDKTHTCPPCPAPELLGGPSVFLFPPKPKDTLMISRTPEVTCVVVDVSHEDPE | |
| | VKFNWYVDGVEVHNAKTKPREEQYGSTYRVVSVLTVLHQDWLNGKEYKCKVSNKAL | |
| | PAPIEKTISKAKGQPREPQVYTLPPSREEMTKNQVSLTCLVKGFYPSDIAVEWESN | |
| | GQPENNYKTTPPVLDSDGSFFLYSKLTVDKSRWQQGNVFSCSVMHEALHNHYTQKS | |
| | LSLSPGK | |
| HC | EVQLLESGGGLVQPGGSLRLSCAVSGFSLNDYGVSWVRQAPGKGLEWVGAIGSSGV | 306 |
| 3-3F5-H19 | AWYANFAKGRFTISRDNSKNTLYLQMNSLRAEDTAVYYCARDRDYGYRADDATSGM | |
| hIgG4 | DLWGRGTLVTVSSASTKGPSVFPLAPCSRSTSESTAALGCLVKDYFPEPVTVSWNS | |
| S228P | GALTSGVHTFPAVLQSSGLYSLSSVVTVPSSSLGTKTYTCNVDHKPSNTKVDKRVE | |
| | SKYGPPCPPCPAPEFLGGPSVFLFPPKPKDTLMISRTPEVTCVVVDVSQEDPEVQF | |
| | NWYVDGVEVHNAKTKPREEQFNSTYRVVSVLTVLHQDWLNGKEYKCKVSNKGLPSS | |
| | IEKTISKAKGQPREPQVYTLPPSQEEMTKNQVSLTCLVKGFYPSDIAVEWESNGQP | |
| | ENNYKTTPPVLDSDGSFFLYSRLTVDKSRWQEGNVFSCSVMHEALHNHYTQKSLSL | |
| | SLGK | |
| HC | QEQLEESGGGLVKPGASLTLTCTVSGFSLTNNYVNFVMCWVRQAPGKGLEWIASID | 307 |
| 9E3 | PGDDSTDYASWATGRFTISKASSTTVTLQVTSLTAADTATYFCARGDAGTSYSFNF | |
| | WGPGTLVTVSSASTKGPSVYPLAPVCGDTTGSSVTLGCLVKGYFPEPVTLTWNSGS | |
| | LSSGVHTFPAVLQSDLYTLSSSVTVTSSTWPSQSITCNVAHPASSTKVDKKIEPRG | |
| | PTIKPCPPCKCPAPNLLGGPSVFIFPPKIKDVLMISLSPIVTCVVVDVSEDDPDVQ | |
| | ISWFVNNVEVHTAQTQTHREDYNSTLRVVSALPIQHQDWMSGKEFKCKVNNKDLPA | |
| | PIERTISKPKGSVRAPQVYVLPPPEEEMTKKQVTLTCMVTDFMPEDIYVEWTNNGK | |
| | TELNYKNTEPVLDSDGSYFMYSKLRVEKKNWVERNSYSCSVVHEGLHNHHTTKSFS | |
| | RTPGK | |
| HC | QEQLVESGGGLVKPGASLTLTCTASGFSLTDNYVMSWVRQAPGKGLEWIACIDPGD | 308 |
| 10D10 | DSTYYASWATGRFTISRASSTTVTLQVTSLTAADTATYFCARGDAAASYSFNFWGP | |
| | GTLVTVSSASTKGPSVYPLAPVCGDTTGSSVTLGCLVKGYFPEPVTLTWNSGSLSS | |
| | GVHTFPAVLQSDLYTLSSSVTVTSSTWPSQSITCNVAHPASSTKVDKKIEPRGPTI | |
| | KPCPPCKCPAPNLLGGPSVFIFPPKIKDVLMISLSPIVTCVVVDVSEDDPDVQISW | |
| | FVNNVEVHTAQTQTHREDYNSTLRVVSALPIQHQDWMSGKEFKCKVNNKDLPAPIE | |
| | RTISKPKGSVRAPQVYVLPPPEEEMTKKQVTLTCMVTDFMPEDIYVEWTNNGKTEL | |
| | NYKNTEPVLDSDGSYFMYSKLRVEKKNWVERNSYSCSVVHEGLHNHHTTKSFSRTP | |
| | GK | |
| HC | QSLEESGGGLVKPGGTLTLTCKASGIDFNGGGIYWVRQAPGKGLEWIASIYPDHGS | 309 |
| 12B3 | VDYANWVNGRFTISLDNAQNTVFLQLTSLTVADTATYFCARESGGSYYDLWGPGTL | |
| | VTVSSASTKGPSVYPLAPVCGDTTGSSVTLGCLVKGYFPEPVTLTWNSGSLSSGVH | |
| | TFPAVLQSDLYTLSSSVTVTSSTWPSQSITCNVAHPASSTKVDKKIEPRGPTIKPC | |
| | PPCKCPAPNLLGGPSVFIFPPKIKDVLMISLSPIVTCVVVDVSEDDPDVQISWFVN | |
| | NVEVHTAQTQTHREDYNSTLRVVSALPIQHQDWMSGKEFKCKVNNKDLPAPIERTI | |
| | SKPKGSVRAPQVYVLPPPEEEMTKKQVTLTCMVTDFMPEDIYVEWTNNGKTELNYK | |
| | NTEPVLDSDGSYFMYSKLRVEKKNWVERNSYSCSVVHEGLHNHHTTKSFSRTPGK | |
| HC | QSLEESGGGLVKPGGTLTLTCTASGFDFNGGGIYWVRQAPGKGLEWIAYIYPDHGS | 310 |
| 1D10 | ADYATWVNGRFTISLDNAQNTVFLQMTSLTVADTATYFCARETGGSWYDLWGPGTL | |
| | VTVSSASTKGPSVYPLAPVCGDTTGSSVTLGCLVKGYFPEPVTLTWNSGSLSSGVH | |
| | TFPAVLQSDLYTLSSSVTVTSSTWPSQSITCNVAHPASSTKVDKKIEPRGPTIKPC | |
| | PPCKCPAPNLLGGPSVFIFPPKIKDVLMISLSPIVTCVVVDVSEDDPDVQISWFVN | |
| | NVEVHTAQTQTHREDYNSTLRVVSALPIQHQDWMSGKEFKCKVNNKDLPAPIERTI | |
| | SKPKGSVRAPQVYVLPPPEEEMTKKQVTLTCMVTDFMPEDIYVEWTNNGKTELNYK | |
| | NTEPVLDSDGSYFMYSKLRVEKKNWVERNSYSCSVVHEGLHNHHTTKSFSRTPGK | |

Constant Regions

| | | |
|---|---|---|
| huKappa | RTVAAPSVFIFPPSDEQLKSGTASVVCLLNNFYPREAKVQWKVDNALQSGNSQESV | 311 |
| | TEQDSKDSTYSLSSTLTLSKADYEKHKVYACEVTHQGLSSPVTKSFNRGEC | |
| hIgG1 | ASTKGPSVFPLAPSSKSTSGGTAALGCLVKDYFPEPVTVSWNSGALTSGVHTFPAV | 312 |
| N297 | LQSSGLYSLSSVVTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIEKTISKAKG | |
| | QPREPQVYTLPPSREEMTKNQVSLTCLVKGFYPSDIAVEWESNGQPENNYKTTPPV | |
| | LDSDGSFFLYSKLTVDKSRWQQGNVFSCSVMHEALHNHYTQKSLSLSPGK | |
| hIgG1 | ASTKGPSVFPLAPSSKSTSGGTAALGCLVKDYFPEPVTVSWNSGALTSGVHTFPAV | 313 |
| G297 | LQSSGLYSLSSVVTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKFNWYVDGVEVH | |
| | NAKTKPREEQYGSTYRVVSVLTVLHQDWLNGKEYKCKVSNKALPAPIEKTISKAKG | |
| | QPREPQVYTLPPSREEMTKNQVSLTCLVKGFYPSDIAVEWESNGQPENNYKTTPPV | |
| | LDSDGSFFLYSKLTVDKSRWQQGNVFSCSVMHEALHNHYTQKSLSLSPGK | |
| hIgG4 | ASTKGPSVFPLAPCSRSTSESTAALGCLVKDYFPEPVTVSWNSGALTSGVHTFPAV | 314 |
| S228 | LQSSGLYSLSSVVTVPSSSLGTKTYTCNVDHKPSNTKVDKRVESKYGPPCPSCPAP | |
| | EFLGGPSVFLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKTTPPVLDS | |
| | DGSFFLYSRLTVDKSRWQEGNVFSCSVMHEALHNHYTQKSLSLSLGK | |
| hIgG4 | ASTKGPSVFPLAPCSRSTSESTAALGCLVKDYFPEPVTVSWNSGALTSGVHTFPAV | 315 |
| P228 | LQSSGLYSLSSVVTVPSSSLGTKTYTCNVDHKPSNTKVDKRVESKYGPPCPPCPAP | |
| | EFLGGPSVFLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKTTPPVLDS | |
| | DGSFFLYSRLTVDKSRWQEGNVFSCSVMHEALHNHYTQKSLSLSLGK | |

Miscellaneous Sequences

| | | |
|---|---|---|
| KLK5 thermo- | LRPNQL | 316 |
| dynamic | | |
| epitope | | |
| Region 1 | | |
| KLK5 thermo- | QGVKSI | 317 |
| dynamic | | |
| epitope | | |
| Region 2 | | |
| KLK5 thermo- | KRCEDAYPRQIDDT | 318 |
| dynamic | | |
| epitope | | |
| Region 3 | | |
| KLK5 thermo- | DYPCARPNRPGVY | 319 |
| dynamic | | |
| epitope | | |
| Region 4 | | |
| Hu SPINK9 | IESAKQTKQMVDCSHYKKLPPGQQRFCHREYDPICGSDGKTYKNDCFFCSK | 320 |
| (I20-C86. | VKKTDGTLKFVHFGKCGNSVTDKTHTCPPCPAPELLGGPSVFLFPPKPKDT | |
| C22S.H48R. | LMISRTPEVTCVVVDVSHEDPEVKFNWYVDGVEVHNAKTKPREEQYNSTYR | |
| M49E) ; Fc | VVSVLTVLHQDWLNGKEYKCKVSNKALPAPIEKTISKAKGQPREPQVYTLP | |
| human IgG1 | PSREEMTKNQVSLTCLVKGFYPSDIAVEWESNGQPENNYKTTPPVLDSDGS | |
| E356.M358 | FFLYSKLTVDKSRWQQGNVFSCSVMHEALHNHYTQKSLSLSPGK | |
| VL | AIRMTQSPSSFSASTGDRVTITCQASESISNELSWYQQKPGKAPKLLIYYASTLAS | 321 |
| hu10C5-L6 | GVPSRFSGSGSGTDFTLTISSLQPEDFATYYCAQGFGSSGVENVFGGGTEVVVK | |
| Fab HC | EVQLVESGGGLVQPGGSLRLSCAASGFSLSSYGVTWVRQAPGKGLEWIGYITSNYG | 322 |
| 10C5 | VSYYASWAKSRSTISRDTSKNTVYLQMGSLRAEDMAVYYCARENPDYGYAYDAWGQ | |
| Humanized | GTTVTVSSASTKGPSVFPLAPSSKSTSGGTAALGCLVKDYFPEPVTVSWNSGALTS | |
| | GVHTFPAVLQSSGLYSLSSVVTVPSSSLGTQTYICNVNHKPSNTKVDKKVEPKSCD | |
| Fab LC | AIRMTQSPSSFSASTGDRVTITCQASESISNELSWYQQKPGKAPKLLIYYASTLAS | 323 |
| 10C5 | GVPSRFSGSGSGTDFTLTISSLQPEDFATYYCAQGFGSSGVENVFGGGTKVEIKRT | |
| Humanized | VAAPSVFIFPPSDEQLKSGTASVVCLLNNFYPREAKVQWKVDNALQSGNSQESVTE | |
| | QDSKDSTYSLSSTLTLSKADYEKHKVYACEVTHQGLSSPVTKSFNRGEC | |
| Fab HC | EVQLVESGGGLIQPGGSLRLSCAASGFSLSSYGVSWVRQAPGKGLEYVGFIGSGGF | 324 |
| 9H5 | AYYASWAKSRFTISRDNSKNTLYLQMNSLRAEDTAVYYCARDDVGGGKSLDIWGQG | |
| Humanized | TLVTVSSASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKKVEPKSCD | |
| Fab LC | DIQMTQSPSSLSASVGDRVTITCQASQSISSYLSWYQQKPGKAPKLLIYSASTLAS | 325 |
| 9H5 | GVPSRFSGSGSGTDFTLTISSLQPEDFATYYCHQDYTSSNVDNTFGGGTKVEIKRT | |
| Humanized | VAAPSVFIFPPSDEQLKSGTASVVCLLNNFYPREAKVQWKVDNALQSGNSQESVTE | |
| | QDSKDSTYSLSSTLTLSKADYEKHKVYACEVTHQGLSSPVTKSFNRGEC | |
| Fab HC | EVQLLESGGGLVQPGGSLRLSCAVSGFSLNDYGVSWVRQAPGKGLEWVGAIGSSGV | 326 |
| 3-3F5 | AWYANFAKGRFTISRDNSKNTLYLQMNSLRAEDTAVYYCARDRDYGYRADDATSGM | |
| Humanized | DLWGRGTLVTVSSASTKGPSVFPLAPSSKSTSGGTAALGCLVKDYFPEPVTVSWNS | |
| | GALTSGVHTFPAVLQSSGLYSLSSVVTVPSSSLGTQTYICNVNHKPSNT | |
| | KVDKKVEPKSCD | |
| Fab LC | AIRMTQSPSSFSASTGDRVTITCQASESIGNALAWYQQKPGKAPKLLIYDASDLAS | 327 |
| 3-3F5 | GVPSRFSGSGSGTDFTLTISSLQPEDFATYYCQQGDSHNNVDNIFGGGTKVEIKRT | |
| Humanized | VAAPSVFIFPPSDEQLKSGTASVVCLLNNFYPREAKVQWKVDNALQSGNSQESVTE | |
| | QDSKDSTYSLSSTLTLSKADYEKHKVYACEVTHQGLSSPVTKSFNRGEC | |
| Human KLK5 | IINGSDCDMHTQPWQAALLLRPNQLYCGAVLVHPQWLLTAAHCRKKVFRVRLGHYS | 328 |
| Crystal | LSPVYESGQQMFQGVKSIPHPGYSHPGHSNDLMLIKLNRRIRPTKDVRPINVSSHC | |
| Structure | PSAGTKCLVSGWGTTKSPQVHFPKVLQCLNISVLSQKRCEDAYPRQIDDTMFCAGD | |
| Experiments | KAGRDSCQGDSGGPVVCNGSLQGLVSWGDYPCARPNRPGVYTNLCKFTKWIQETIQ | |
| | ANS | |

Framework Sequences

| | | |
|---|---|---|
| FR-L1 | AX₁RMTQSPSSFSASTGDRVTITC | 329 |
| 10C5 | wherein X₁ is Y or H | |
| (consensus) | | |
| FR-L2 | WYQQKPGKX₁PKLLIY | 330 |
| 10C5 | wherein X₁ is P or A | |
| (consensus) | | |
| FR-L3 | GVPSRFSGSGSGTDFTLTISX₁LQX₂EDFATYYC | 331 |
| 10C5 | wherein X₁ is C or S, X₂ is S or P | |
| (consensus) | | |
| FR-L4 | FGGGTX₁VX₂X₃K | 332 |
| 10C5 | wherein X₁ is E or K, X₂ is E or V, X₃ is I or V | |
| (consensus) | | |
| FR-H1 | EX₁QLVESGGGLVQPGGSLRLSCAX₂SGFSLS | 333 |
| 10C5 | wherein X₁ is V or Q, X₂ is V or A | |
| (consensus) | | |
| FR-H2 | WVRQAPGKGLEX₁X₂X₃ | 334 |
| 10C5 | wherein X₁ is W or Y, X₂ is I or V, X₃ is G or S | |
| (consensus) | | |
| FR-H3 | RX₁TISRX₂X₃X₄X₅NTX₆YLQMGSLRAEDMAVYX₇CAR | 335 |
| 10C5 | wherein X₁ is S or F, X₂ is D or N, X₃ is T, L or N, X₄ | |
| (consensus) | is S or N, X₅ is K, T or L, X₆ is V or L, X₇ is F or Y | |
| FR-H4 | WGX₁GTTVTVSS | 336 |
| 10C5 | wherein X₁ is P or Q | |
| (consensus) | | |
| FR-L1 | DIQMTQSPSSLSASVGDRVTITC | 337 |
| 9H5 | | |
| FR-L2 | WYQQKPGKX₁PKLLIX₂ | 338 |
| 9H5 | wherein X₁ is R or A, X₂ is F or Y | |
| (consensus) | | |
| FR-L3 | GVPSRFSGSGSGTDFTLTISSLQPEDFATYYC | 339 |
| 9H5 | | |
| FR-L4 | FGGGTX₃VX₄X₅K | 340 |
| 9H5 | wherein X₃ is E or K, X₄ is E or V, X₅ is V or I | |
| (consensus) | | |
| FR-H1 | EX₁QLVESGGGLIQPGGSLRLSCAX₂SGFSLS | 341 |
| 9H5 | wherein X₁ is V or Q, X₂ is V or A | |
| (consensus) | | |
| FR-H2 | WVRQAPGKGLEX₁X₂X₃ | 342 |
| 9H5 | wherein X₁ is Y or W, X₂ is I or V, X₃ is G or S | |
| (consensus) | | |
| FR-H3 | RX₁TISRX₂X₃X₄X₅NTX₆YLQMNSLRAEDTAVYX₇CAR | 343 |
| 9H5 | wherein X₁ is S or F, X₂ is D or N, X₃ is T or N, X₄ is S | |
| (consensus) | or N, X₅ is K or L, X₆ is V or L, X₇ is F or Y | |
| FR-H4 | WGX₁GTLVTVSS | 344 |
| 9H5 | wherein X₁ is P or Q | |
| (consensus) | | |
| FR-L1 | AX₁RMTQSPSSFSASTGDRVTITC | 345 |
| 3-3F5 | wherein X₁ is Y or I | |
| (consensus) | | |
| FR-L2 | WYQQKPGKX₁PKLLIY | 346 |
| 3-3F5 | wherein X₁ is P or A | |
| (consensus) | | |
| FR-L3 | GVPSRFSGSGSGTDFTLTISX₁LQX₂EDFATYYC | 347 |
| 3-3F5 | wherein X₁ is C or S, X₂ is S or P | |
| (consensus) | | |
| FR-L4 | FGGGTX₁VX₂X₃K | 348 |
| 3-3F5 | wherein X₁ is E or K, X₂ is E or V, X₃ is V or I | |
| (consensus) | | |
| FR-H1 | EX₁QLX₂ESGGGLVQPGGSLRLSCAX₃SGFSLN | 349 |
| 3-3F5 | wherein X₁ is V or Q, X₂ is V or L, X₃ is V or A | |
| (consensus) | | |
| FR-H2 | WVRQAPGKGLEWX₁X₂ | 350 |
| 3-3F5 | wherein X₁ is I or V, X₂ is G or S | |
| (consensus) | | |
| FR-H3 | RX₁TISRX₂X₃X₄X₅NTX₆YLQMNSLRAEDTAVYX₇CAR | 351 |
| 3-3F5 | wherein X₁ is S or F, X₂ is H, N or D, X₃ is T or N, X₄ is | |
| (consensus) | S or N, X₅ is K or L, X₆ is V or L, X₇ is F or Y | |
| FR-H4 | WGX₁GTLVTVSS | 352 |
| 3-3F5 | wherein X₁ is P or R | |
| (consensus) | | |
| Human KLK5 | NNDVSCDHPSNTVPSGSNQDLGAGAGEDARSDDSSSRIINGSDCDMHTQPWQAALL | 353 |
| HDX | LRPNQLYCGAVLVHPQWLLTAAHCRKKVFRVRLGHYSLSPVYESGQQMFQGVKSIP | |
| Experiments | HPGYSHPGHSNDLMLIKLNRRIRPTKDVRPINVSSHCPSAGTKCLVSGWGTTKSPQ | |
| | VHFPKVLQCLNISVLSQKRCEDAYPRQIDDTMFCAGDKAGRDSCQGDSGGPVVCNG | |
| | SLQGLVSWGDYPCARPNRPGVYTNLCKFTKWIQETIQANSGNSDYKDDDDK | |

1.-6. (canceled)

7. An isolated antibody that binds to KLK5, wherein the antibody comprises:
(i) (a) HVR-H1 comprising the amino acid sequence of SEQ ID NO:14; (b) HVR-H2 comprising the amino acid sequence of SEQ ID NO:32; (c) HVR-H3 comprising the amino acid sequence of SEQ ID NO:62; (d) HVR-L1 comprising the amino acid sequence of SEQ ID NO:79; (e) HVR-L2 comprising the amino acid sequence of SEQ ID NO:99; and (f) HVR-L3 comprising the amino acid sequence of SEQ ID NO:112;
(ii) (a) HVR-H1 comprising the amino acid sequence of SEQ ID NO:15; (b) HVR-H2 comprising the amino acid sequence of SEQ ID NO:33; (c) HVR-H3 comprising the amino acid sequence of SEQ ID NO:63; (d) HVR-L1 comprising the amino acid sequence of SEQ ID NO:80; (e) HVR-L2 comprising the amino acid sequence of SEQ ID NO:100; and (f) HVR-L3 comprising the amino acid sequence of SEQ ID NO:113;
(iii) (a) HVR-H1 comprising the amino acid sequence of SEQ ID NO:16; (b) HVR-H2 comprising the amino acid sequence of SEQ ID NO:34; (c) HVR-H3 comprising the amino acid sequence of SEQ ID NO:64; (d) HVR-L1 comprising the amino acid sequence of SEQ ID NO:81; (e) HVR-L2 comprising the amino acid sequence of SEQ ID NO:99; and (f) HVR-L3 comprising the amino acid sequence of SEQ ID NO:114;
(iv) (a) HVR-H1 comprising the amino acid sequence of SEQ ID NO:18; (b) HVR-H2 comprising the amino acid sequence of SEQ ID NO:39; (c) HVR-H3 comprising the amino acid sequence of SEQ ID NO:66; (d) HVR-L1 comprising the amino acid sequence of SEQ ID NO:83; (e) HVR-L2 comprising the amino acid sequence of SEQ ID NO:102; and (f) HVR-L3 comprising the amino acid sequence of SEQ ID NO:116;
(v) (a) HVR-H1 comprising the amino acid sequence of SEQ ID NO:19; (b) HVR-H2 comprising the amino acid sequence of SEQ ID NO:40; (c) HVR-H3 comprising the amino acid sequence of SEQ ID NO:67; (d) HVR-L1 comprising the amino acid sequence of SEQ ID NO:84; (e) HVR-L2 comprising the amino acid sequence of SEQ ID NO:103; and (f) HVR-L3 comprising the amino acid sequence of SEQ ID NO:117;
(vi) (a) HVR-H1 comprising the amino acid sequence of SEQ ID NO:20; (b) HVR-H2 comprising the amino acid sequence of SEQ ID NO:33; (c) HVR-H3 comprising the amino acid sequence of SEQ ID NO:68; (d) HVR-L1 comprising the amino acid sequence of SEQ ID NO:85; (e) HVR-L2 comprising the amino acid sequence of SEQ ID NO:103; and (f) HVR-L3 comprising the amino acid sequence of SEQ ID NO:118;
(vii) (a) HVR-H1 comprising the amino acid sequence of SEQ ID NO:21; (b) HVR-H2 comprising the amino acid sequence of SEQ ID NO:41; (c) HVR-H3 comprising the amino acid sequence of SEQ ID NO:69; (d) HVR-L1 comprising the amino acid sequence of SEQ ID NO:86; (e) HVR-L2 comprising the amino acid sequence of SEQ ID NO:104; and (f) HVR-L3 comprising the amino acid sequence of SEQ ID NO:118;
(viii) (a) HVR-H1 comprising the amino acid sequence of SEQ ID NO:22; (b) HVR-H2 comprising the amino acid sequence of SEQ ID NO:46; (c) HVR-H3 comprising the amino acid sequence of SEQ ID NO:69; (d) HVR-L1 comprising the amino acid sequence of SEQ ID NO:88; (e) HVR-L2 comprising the amino acid sequence of SEQ ID NO:105; and (f) HVR-L3 comprising the amino acid sequence of SEQ ID NO:120;
(ix) (a) HVR-H1 comprising the amino acid sequence of SEQ ID NO:22; (b) HVR-H2 comprising the amino acid sequence of SEQ ID NO:42; (c) HVR-H3 comprising the amino acid sequence of SEQ ID NO:70; (d) HVR-L1 comprising the amino acid sequence of SEQ ID NO:89; (e) HVR-L2 comprising the amino acid sequence of SEQ ID NO:105; and (f) HVR-L3 comprising the amino acid sequence of SEQ ID NO:118; or
(x) (a) HVR-H1 comprising the amino acid sequence of SEQ ID NO:23; (b) HVR-H2 comprising the amino acid sequence of SEQ ID NO:47; (c) HVR-H3 comprising the amino acid sequence of SEQ ID NO:71; (d) HVR-L1 comprising the amino acid sequence of SEQ ID NO:90; (e) HVR-L2 comprising the amino acid sequence of SEQ ID NO:106; and (f) HVR-L3 comprising the amino acid sequence of SEQ ID NO:121.

8.-9. (canceled)

10. The isolated antibody of claim 7, wherein the antibody comprises (a) a VH sequence having at least 95% sequence identity to the amino acid sequence of SEQ ID NO:170 and a VL sequence having at least 95% sequence identity to the amino acid sequence of SEQ ID NO:131; (b) a VH sequence having at least 95% sequence identity to the amino acid sequence of SEQ ID NO:171 and a VL sequence having at least 95% sequence identity to the amino acid sequence of SEQ ID NO:132; (c) a VH sequence having at least 95% sequence identity to the amino acid sequence of SEQ ID NO:172 and a VL sequence having at least 95% sequence identity to the amino acid sequence of SEQ ID NO:133; (d) a VH sequence having at least 95% sequence identity to the amino acid sequence of SEQ ID NO:203 and a VL sequence having at least 95% sequence identity to the amino acid sequence of SEQ ID NO:141; (e) a VH sequence having at least 95% sequence identity to the amino acid sequence of SEQ ID NO:204 and a VL sequence having at least 95% sequence identity to the amino acid sequence of SEQ ID NO:142; (f) a VH sequence having at least 95% sequence identity to the amino acid sequence of SEQ ID NO:205 and a VL sequence having at least 95% sequence identity to the amino acid sequence of SEQ ID NO:143; (g) a VH sequence having at least 95% sequence identity to the amino acid sequence of SEQ ID NO:206 and a VL sequence having at least 95% sequence identity to the amino acid sequence of SEQ ID NO:144; (h) a VH sequence having at least 95% sequence identity to the amino acid sequence of SEQ ID NO:226 and a VL sequence having at least 95% sequence identity to the amino acid sequence of SEQ ID NO:152; (i) a VH sequence having at least 95% sequence identity to the amino acid sequence of SEQ ID NO:227 and a VL sequence having at least 95% sequence identity to the amino acid sequence of SEQ ID NO:153; or (j) a VH sequence having at least 95% sequence identity to the amino acid sequence of SEQ ID NO:228 and a VL sequence having at least 95% sequence identity to the amino acid sequence of SEQ ID NO:154.

11. The antibody of claim 7, wherein the antibody is an IgG1 or IgG4.

12. The antibody of claim 7, wherein the antibody inhibits the biological activity of KLK5 by at least 50% as measured by one or more methods selected from the group consisting of a recombinant KLK5 direct activity assay, coupled pro-KLK1 fluorescent peptide assay, a coupled pro-KLK7 fluorescent peptide assay, a pro-KLK1 LC/MS assay, a pro-KLK7 LC/MS assay, and a Kᵢ₍ₐₚₚ₎ assay.

13. The antibody of claim 12, wherein the biological activity is the serine protease activity of KLK5.

14. The antibody of claim 7, wherein the antibody is a monoclonal antibody.

15. The antibody of claim 7, wherein the antibody is a human, humanized, or chimeric antibody.

16. The antibody of claim 7, wherein the antibody is an antibody fragment that binds KLK5.

17.-19. (canceled)

20. An isolated nucleic acid encoding the antibody of claim 7.

21. A host cell comprising the nucleic acid of claim 20.

22. A method of producing an antibody comprising culturing the host cell of claim 21 so that the antibody is produced.

23. An immunoconjugate comprising the antibody of claim 7.

24. A pharmaceutical formulation comprising the antibody of claim 7 and a pharmaceutically acceptable carrier.

25.-34. (canceled)

35. A method of treating an individual having a disease, wherein the disease is selected from the group consisting of Netherton Syndrome, asthma, atopic dermatitis, psoriasis, eosinophilic esophagitis and rosacea comprising administering to the individual an effective amount of the antibody of claim 7.

36. The method of claim 35, wherein the asthma is selected from the group consisting of atopic asthma, allergic asthma, non-allergic asthma, exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, T helper lymphocyte type 2 (Th2) or type 2 (Th2) high, or Type 2 (T2)-driven asthma, eosinophilic asthma, periostin-high asthma, eosinophil-high asthma, Th2-low asthma or nonTh2-driven asthma, periostin-low asthma, and eosinophil-low asthma.

37. The method of claim 36, wherein the asthma is Th2-low asthma.

38. A method of inhibiting the biological activity of KLK5 in an individual comprising administering to the individual an effective amount of the antibody of claim 7 to inhibit the biological activity of KLK5.

39.-43. (canceled)

44. The isolated antibody of claim 10, wherein the antibody comprises (a) a VH sequence of SEQ ID NO:170 and a VL sequence of SEQ ID NO:131; (b) a VH sequence of SEQ ID NO:171 and a VL sequence of SEQ ID NO:132; (c) a VH sequence of SEQ ID NO:172 and a VL sequence of SEQ ID NO:133; (d) a VH sequence of SEQ ID NO:203 and a VL sequence of SEQ ID NO:141; (e) a VH sequence of SEQ ID NO:204 and a VL sequence of SEQ ID NO:142; (f) a VH sequence of SEQ ID NO:205 and a VL sequence of SEQ ID NO:143; (g) a VH sequence of SEQ ID NO:206 and a VL sequence of SEQ ID NO:144; (h) a VH sequence of SEQ ID NO:226 and a VL sequence of SEQ ID NO:152; (i) a VH sequence of SEQ ID NO:227 and a VL sequence of SEQ ID NO:153; or (j) a VH sequence of SEQ ID NO:228 and a VL sequence of SEQ ID NO:154.


SEQUENCE LISTING (356 sequences)

SEQ ID NO: 1 - protein, 293 aa, Homo sapiens
MATARPPWMWVLCALITALLLGVTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 2 - protein, 271 aa, Homo sapiens
VTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 3 - protein, 293 aa, Homo sapiens [sequence omitted]

SEQ ID NO: 4 - protein, 271 aa, Homo sapiens
VTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNNLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 5 - protein, 293 aa, Homo sapiens
MATARPPWMWVLCALITALLLGVTEHVLANNDVSCDHPSNTVPSGSNQDLGAGAREDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNNLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 6 - protein, 271 aa, Homo sapiens
VTEHVLANNDVSCDHPSNTVPSGSNQDLGAGAREDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNNLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 7 - protein, 293 aa, Homo sapiens
MATARPPWMWVLCALITALLLGVTEHVLANNDVSCDHPSNTVPSGSNQDLGAGAREDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 8 - protein, 271 aa, Homo sapiens
VTEHVLANNDVSCDHPSNTVPSGSNQDLGAGAREDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 9 - protein, 1064 aa, Homo sapiens
MKIATVSVLLPLALCLIQDAASKNEDQEMCHEFQAFMKNGKLFCPQDKKFFQSLDGIMFINKCATCKMILEKEAKSQKRARHLARAPKATAPTELNCDDFKKGERDGDFICPDYYEAVCGTDGKTYDNRCALCAENAKTGSQIGVKSEGECKSSNPEQDVCSAFRPFVRDGRLGCTRENDPVLGPDGKTHGNKCAMCAELFLKEAENAKREGETRIRRNAEKDFCKEYEKQVRNGRLFCTRESDPVRGPDGRMHGNKCALCAEIFKQRFSEENSKTDQNLGKAEEKTKVKREIVKLCSQYQNQAKNGILFCTRENDPIRGPDGKMHGNLCSMCQAYFQAENEEKKKAEARARNKRESGKATSYAELCSEYRKLVRNGKLACTRENDPIQGPDGKVHGNTCSMCEVFFQAEEEEKKKKEGKSRNKRQSKSTASFEELCSEYRKSRKNGRLFCTRENDPIQGPDGKMHGNTCSMCEAFFQQEERARAKAKREAAKEICSEFRDQVRNGTLICTREHNPVRGPDGKMHGNKCAMCASVFKLEEEEKKNDKEEKGKVEAEKVKREAVQELCSEYRHYVRNGRLPCTRENDPIEGLDGKIHGNTCSMCEAFFQQEAKEKERAEPRAKVKREAEKETCDEFRRLLQNGKLFCTRENDPVRGPDGKTHGNKCAMCKAVFQKENEERKRKEEEDQRNAAGHGSSGGGGGNTQDECAEYREQMKNGRLSCTRESDPVRDADGKSYNNQCTMCKAKLEREAERKNEYSRSRSNGTGSESGKDTCDEFRSQMKNGKLICTRESDPVRGPDGKTHGNKCTMCKEKLEREAAEKKKKEDEDRSNTGERSNTGERSNDKEDLCREFRSMQRNGKLICTRENNPVRGPYGKMHINKCAMCQSIFDREANERKKKDEEKSSSKPSNNAKDECSEFRNYIRNNELICPRENDPVHGADGKFYTNKCYMCRAVFLTEALERAKLQEKPSHVRASQEEDSPDSFSSLDSEMCKDYRVLPRIGYLCPKDLKPVCGDDGQTYNNPCMLCHENLIRQTNTHIRSTGKCEESSTPGTTAASMPPSDE

SEQ ID NO: 10 - protein, 1042 aa, Homo sapiens
KNEDQEMCHEFQAFMKNGKLFCPQDKKFFQSLDGIMFINKCATCKMILEKEAKSQKRARHLARAPKATAPTELNCDDFKKGERDGDFICPDYYEAVCGTDGKTYDNRCALCAENAKTGSQIGVKSEGECKSSNPEQDVCSAFRPFVRDGRLGCTRENDPVLGPDGKTHGNKCAMCAELFLKEAENAKREGETRIRRNAEKDFCKEYEKQVRNGRLFCTRESDPVRGPDGRMHGNKCALCAEIFKQRFSEENSKTDQNLGKAEEKTKVKREIVKLCSQYQNQAKNGILFCTRENDPIRGPDGKMHGNLCSMCQAYFQAENEEKKKAEARARNKRESGKATSYAELCSEYRKLVRNGKLACTRENDPIQGPDGKVHGNTCSMCEVFFQAEEEEKKKKEGKSRNKRQSKSTASFEELCSEYRKSRKNGRLFCTRENDPIQGPDGKMHGNTCSMCEAFFQQEERARAKAKREAAKEICSEFRDQVRNGTLICTREHNPVRGPDGKMHGNKCAMCASVFKLEEEEKKNDKEEKGKVEAEKVKREAVQELCSEYRHYVRNGRLPCTRENDPIEGLDGKIHGNTCSMCEAFFQQEAKEKERAEPRAKVKREAEKETCDEFRRLLQNGKLFCTRENDPVRGPDGKTHGNKCAMCKAVFQKENEERKRKEEEDQRNAAGHGSSGGGGGNTQDECAEYREQMKNGRLSCTRESDPVRDADGKSYNNQCTMCKAKLEREAERKNEYSRSRSNGTGSESGKDTCDEFRSQMKNGKLICTRESDPVRGPDGKTHGNKCTMCKEKLEREAAEKKKKEDEDRSNTGERSNTGERSNDKEDLCREFRSMQRNGKLICTRENNPVRGPYGKMHINKCAMCQSIFDREANERKKKDEEKSSSKPSNNAKDECSEFRNYIRNNELICPRENDPVHGADGKFYTNKCYMCRAVFLTEALERAKLQEKPSHVRASQEEDSPDSFSSLDSEMCKDYRVLPRIGYLCPKDLKPVCGDDGQTYNNPCMLCHENLIRQTNTHIRSTGKCEESSTPGTTAASMPPSDE

SEQ ID NO: 11 - protein, 5 aa, synthetic construct
  REGION 1..5: note = Description of Artificial Sequence: Synthetic peptide
DYNMA

SEQ ID NO: 12 - protein, 5 aa, synthetic construct
  REGION 1..5: note = Description of Artificial Sequence: Synthetic peptide
DYYMA

SEQ ID NO: 13 - protein, 6 aa, synthetic construct
  REGION 1..6: note = Description of Artificial Sequence: Synthetic peptide
SSYWIC

SEQ ID NO: 14 - protein, 6 aa, synthetic construct
  REGION 1..6: note = Description of Artificial Sequence: Synthetic peptide
TSYWIC

SEQ ID NO: 15 - protein, 5 aa, synthetic construct
  REGION 1..5: note = Description of Artificial Sequence: Synthetic peptide
NYGVT

SEQ ID NO: 16 - protein, 5 aa, synthetic construct
  REGION 1..5: note = Description of Artificial Sequence: Synthetic peptide
NYGVS

SEQ ID NO: 17 - protein, 5 aa, synthetic construct
  REGION 1..5: note = Description of Artificial Sequence: Synthetic peptide
SYGVT

SEQ ID NO: 18 - protein, 5 aa, synthetic construct
  REGION 1..5: note = Description of Artificial Sequence: Synthetic peptide
NYGVS

SEQ ID NO: 19 - protein, 5 aa, synthetic construct
  REGION 1..5: note = Description of Artificial Sequence: Synthetic peptide
TFAIN

SEQ ID NO: 20 - protein, 5 aa, synthetic construct
  REGION 1..5: note = Description of Artificial Sequence: Synthetic peptide
GYGVS

SEQ ID NO: 21 - protein, 5 aa, synthetic construct
  REGION 1..5: note = Description of Artificial Sequence: Synthetic peptide
NYGVT

SEQ ID NO: 22 - protein, 5 aa, synthetic construct
  REGION 1..5: note = Description of Artificial Sequence: Synthetic peptide
SYGVS

SEQ ID NO: 23 - protein, 5 aa, synthetic construct
  REGION 1..5: note = Description of Artificial Sequence: Synthetic peptide
SYPIS

SEQ ID NO: 24 - protein, 5 aa, synthetic construct
  REGION 1..5: note = Description of Artificial Sequence: Synthetic peptide
DYGVS

SEQ ID NO: 25 - protein, 9 aa, synthetic construct
  REGION 1..9: note = Description of Artificial Sequence: Synthetic peptide
NNYVNFVMC

SEQ ID NO: 26 - protein, 6 aa, synthetic construct
  REGION 1..6: note = Description of Artificial Sequence: Synthetic peptide
DNYVMS

SEQ ID NO: 27 - protein, 5 aa, synthetic construct
  REGION 1..5: note = Description of Artificial Sequence: Synthetic peptide
GGGIY

SEQ ID NO: 28 - skipped

SEQ ID NO: 29 - protein, 17 aa, synthetic construct
  REGION 1..17: note = Description of Artificial Sequence: Synthetic peptide
TISYDAGRTYYRDSVKG

SEQ ID NO: 30 - protein, 17 aa, synthetic construct
  REGION 1..17: note = Description of Artificial Sequence: Synthetic peptide
SISYDGDTTYYRDSVKG

SEQ ID NO: 31 - protein, 16 aa, synthetic construct
  REGION 1..16: note = Description of Artificial Sequence: Synthetic peptide
CVYGLDVNIYYASWTK

SEQ ID NO: 32 - protein, 16 aa, synthetic construct
  REGION 1..16: note = Description of Artificial Sequence: Synthetic peptide
CVYGLDVNIYYASWTE

SEQ ID NO: 33 - protein, 16 aa, synthetic construct
  REGION 1..16: note = Description of Artificial Sequence: Synthetic peptide
FIGSGGSAYYASWAKS

SEQ ID NO: 34 - protein, 16 aa, synthetic construct
  REGION 1..16: note = Description of Artificial Sequence: Synthetic peptide
FIGYGGSTYYASWAKS

SEQ ID NO: 35 - protein, 17 aa, synthetic construct
  REGION 1..17: note = Description of Artificial Sequence: Synthetic peptide
YITSNYGVSYYASWAKS

SEQ ID NO: 36 - protein, 17 aa, synthetic construct
  REGION 1..17: note = Description of Artificial Sequence: Synthetic peptide
YITSNYGVSYYANSVKG

SEQ ID NO: 37 - protein, 17 aa, synthetic construct
  REGION 1..17: note = Description of Artificial Sequence: Synthetic peptide
YITSNYGVSYYASFAKS

SEQ ID NO: 38 - protein, 17 aa, synthetic construct
  REGION 1..17: note = Description of Artificial Sequence: Synthetic peptide
  MOD_RES 13: note = Ser or Asn
  MOD_RES 14: note = Trp, Ser or Phe
  MOD_RES 15: note = Ala or Val
  MOD_RES 17: note = Ser or Gly
YITSNYGVSYYAXXXKX

SEQ ID NO: 39 - protein, 16 aa, synthetic construct
  REGION 1..16: note = Description of Artificial Sequence: Synthetic peptide
YIGSAGSTYYATWAKS

SEQ ID NO: 40 - protein, 16 aa, synthetic construct
  REGION 1..16: note = Description of Artificial Sequence: Synthetic peptide
AIGRGGSAYYASWAKS

SEQ ID NO: 41 - protein, 16 aa, synthetic construct
  REGION 1..16: note = Description of Artificial Sequence: Synthetic peptide
FIGSSGSAYYASWAKS

SEQ ID NO: 42 - protein, 16 aa, synthetic construct
  REGION 1..16: note = Description of Artificial Sequence: Synthetic peptide
FIGSGGFAYYASWAKS

SEQ ID NO: 43 - protein, 16 aa, synthetic construct
  REGION 1..16: note = Description of Artificial Sequence: Synthetic peptide
FIGSGGFAYYADSVKG

SEQ ID NO: 44 - protein, 16 aa, synthetic construct
  REGION 1..16: note = Description of Artificial Sequence: Synthetic peptide
FIGSGGFAYYASFAKS

SEQ ID NO: 45 - protein, 16 aa, synthetic construct
  REGION 1..16: note = Description of Artificial Sequence: Synthetic peptide
  MOD_RES 12: note = Ser or Asp
  MOD_RES 13: note = Trp, Ser or Phe
  MOD_RES 14: note = Ala or Val
  MOD_RES 16: note = Ser or Gly
FIGSGGFAYYAXXXKX

SEQ ID NO: 46 - protein, 16 aa, synthetic construct
  REGION 1..16: note = Description of Artificial Sequence: Synthetic peptide
FIGSGGSPYYASWAKS

SEQ ID NO: 47 - protein, 17 aa, synthetic construct
  REGION 1..17: note = Description of Artificial Sequence: Synthetic peptide
YITSEYGVAYYATWAES

SEQ ID NO: 48 - protein, 16 aa, synthetic construct
  REGION 1..16: note = Description of Artificial Sequence: Synthetic peptide
AIGSSGVAWYANWAKG

SEQ ID NO: 49 - protein, 16 aa, synthetic construct
  REGION 1..16: note = Description of Artificial Sequence: Synthetic peptide
AIGSSGVAFYANWAKG

SEQ ID NO: 50 - protein, 16 aa, synthetic construct
  REGION 1..16: note = Description of Artificial Sequence: Synthetic peptide
AIGSSGVAWYADSVKG

SEQ ID NO: 51 - protein, 16 aa, synthetic construct
  REGION 1..16: note = Description of Artificial Sequence: Synthetic peptide
AIGSSGVAFYADSVKG

SEQ ID NO: 52 - protein, 16 aa, synthetic construct
  REGION 1..16: note = Description of Artificial Sequence: Synthetic peptide
AIGSSGVAWYANFAKG

SEQ ID NO: 53 - protein, 16 aa, synthetic construct
  REGION 1..16: note = Description of Artificial Sequence: Synthetic peptide
AIGSSGVAFYANFAKG

SEQ ID NO: 54 - protein, 16 aa, synthetic construct
  REGION 1..16: note = Description of Artificial Sequence: Synthetic peptide
  MOD_RES 9: note = Trp or Phe
  MOD_RES 12: note = Asn or Asp
  MOD_RES 13: note = Trp, Ser or Phe
  MOD_RES 14: note = Ala or Val
AIGSSGVAXYAXXXKG

SEQ ID NO: 55 - protein, 16 aa, synthetic construct
  REGION 1..16: note = Description of Artificial Sequence: Synthetic peptide
SIDPGDDSTDYASWAT

SEQ ID NO: 56 - protein, 16 aa, synthetic construct
  REGION 1..16: note = Description of Artificial Sequence: Synthetic peptide
CIDPGDDSTYYASWAT

SEQ ID NO: 57 - protein, 17 aa, synthetic construct
  REGION 1..17: note = Description of Artificial Sequence: Synthetic peptide
SIYPDHGSVDYANWVNG

SEQ ID NO: 58 - protein, 17 aa, synthetic construct
  REGION 1..17: note = Description of Artificial Sequence: Synthetic peptide
YIYPDHGSADYATWVNG

SEQ ID NO: 59 - protein, 16 aa, synthetic construct
  REGION 1..16: note = Description of Artificial Sequence: Synthetic peptide
  MOD_RES 1: note = Phe or Ala
  MOD_RES 5: note = Gly or Ser
  MOD_RES 7: note = Phe or Val
  MOD_RES 9: note = Tyr, Trp or Phe
  MOD_RES 12: note = Ser, Asp or Asn
  MOD_RES 13: note = Trp, Ser or Phe
  MOD_RES 14: note = Ala or Val
  MOD_RES 16: note = Ser or Gly
XIGSXGXAXYAXXXKX

SEQ ID NO: 60 - protein, 12 aa, synthetic construct
  REGION 1..12: note = Description of Artificial Sequence: Synthetic peptide
GIFNYGTDYFDY

SEQ ID NO: 61 - protein, 12 aa, synthetic construct
  REGION 1..12: note = Description of Artificial Sequence: Synthetic peptide
DGTIPAGSWFAY

SEQ ID NO: 62 - protein, 11 aa, synthetic construct
  REGION 1..11: note = Description of Artificial Sequence: Synthetic peptide
GGGSADFGFDL

SEQ ID NO: 63 - protein, 11 aa, synthetic construct
  REGION 1..11: note = Description of Artificial Sequence: Synthetic peptide
DDVGGGKSLDI

SEQ ID NO: 64 - protein, 12 aa, synthetic construct
  REGION 1..12: note = Description of Artificial Sequence: Synthetic peptide
LCGVDCADALDS

SEQ ID NO: 65 - protein, 11 aa, synthetic construct
  REGION 1..11: note = Description of Artificial Sequence: Synthetic peptide
ENPDYGYAYDA

SEQ ID NO: 66 - protein, 13 aa, synthetic construct
  REGION 1..13: note = Description of Artificial Sequence: Synthetic peptide
AAYSAGSADAEDI

SEQ ID NO: 67 - protein, 12 aa, synthetic construct
  REGION 1..12: note = Description of Artificial Sequence: Synthetic peptide
ENAGSGWGELDI

SEQ ID NO: 68 - protein, 11 aa, synthetic construct
  REGION 1..11: note = Description of Artificial Sequence: Synthetic peptide
DNVGGDMSLDI

SEQ ID NO: 69 - protein, 11 aa, synthetic construct
  REGION 1..11: note = Description of Artificial Sequence: Synthetic peptide
DDVGGGKSLDI

SEQ ID NO: 70 - protein, 11 aa, synthetic construct
  REGION 1..11: note = Description of Artificial Sequence: Synthetic peptide
DDVGGGRSLDI

SEQ ID NO: 71 - protein, 11 aa, synthetic construct
  REGION 1..11: note = Description of Artificial Sequence: Synthetic peptide
ENPTYGYAYDA

SEQ ID NO: 72 - protein, 17 aa, synthetic construct
  REGION 1..17: note = Description of Artificial Sequence: Synthetic peptide
DRDYGYRADDATSGMDL

SEQ ID NO: 73 - protein, 11 aa, synthetic construct
  REGION 1..11: note = Description of Artificial Sequence: Synthetic peptide
GDAGTSYSFNF

SEQ ID NO: 74 - protein, 11 aa, synthetic construct
  REGION 1..11: note = Description of Artificial Sequence: Synthetic peptide
GDAAASYSFNF

SEQ ID NO: 75 - protein, 9 aa, synthetic construct
  REGION 1..9: note = Description of Artificial Sequence: Synthetic peptide
ESGGSYYDL

SEQ ID NO: 76 - protein, 9 aa, synthetic construct
  REGION 1..9: note = Description of Artificial Sequence: Synthetic peptide
ETGGSWYDL

SEQ ID NO: 77 - protein, 11 aa, synthetic construct
  REGION 1..11: note = Description of Artificial Sequence: Synthetic peptide
RASEDIYSGLA

SEQ ID NO: 78 - protein, 11 aa, synthetic construct
  REGION 1..11: note = Description of Artificial Sequence: Synthetic peptide
LASKNIYRNLA

SEQ ID NO: 79 - protein, 11 aa, synthetic construct
  REGION 1..11: note = Description of Artificial Sequence: Synthetic peptide
QASENIYSLLA

SEQ ID NO: 80 - protein, 11 aa, synthetic construct
  REGION 1..11: note = Description of Artificial Sequence: Synthetic peptide
QASQNIGDYLS

SEQ ID NO: 81 - protein, 11 aa, synthetic construct
  REGION 1..11: note = Description of Artificial Sequence: Synthetic peptide
QASEDIGSYCS

SEQ ID NO: 82 - protein, 11 aa, synthetic construct
  REGION 1..11: note = Description of Artificial Sequence: Synthetic peptide
QASESISNELS

SEQ ID NO: 83 - protein, 11 aa, synthetic construct
  REGION 1..11: note = Description of Artificial Sequence: Synthetic peptide
QASQSISNYVA

SEQ ID NO: 84 - protein, 11 aa, synthetic construct
  REGION 1..11: note = Description of Artificial Sequence: Synthetic peptide
QASESISSDLA

SEQ ID NO: 85 - protein, 11 aa, synthetic construct
  REGION 1..11: note = Description of Artificial Sequence: Synthetic peptide
QASQNINNYLS

SEQ ID NO: 86 - protein, 11 aa, synthetic construct
  REGION 1..11: note = Description of Artificial Sequence: Synthetic peptide
QASQSIGSYLS

SEQ ID NO: 87 - protein, 11 aa, synthetic construct
  REGION 1..11: note = Description of Artificial Sequence: Synthetic peptide
QASQSISSYLS

SEQ ID NO: 88 - protein, 11 aa, synthetic construct
  REGION 1..11: note = Description of Artificial Sequence: Synthetic peptide
QASQSISNYLS

SEQ ID NO: 89 - protein, 11 aa, synthetic construct
  REGION 1..11: note = Description of Artificial Sequence: Synthetic peptide
QASQSISTYLS

SEQ ID NO: 90 - protein, 11 aa, synthetic construct
  REGION 1..11: note = Description of Artificial Sequence: Synthetic peptide
QASESIGNELS

SEQ ID NO: 91 - protein, 11 aa, synthetic construct
  REGION 1..11: note = Description of Artificial Sequence: Synthetic peptide
QASESIGNALA

SEQ ID NO: 92 - protein, 11 aa, synthetic construct
  REGION 1..11: note = Description of Artificial Sequence: Synthetic peptide
QASESISRYLS

SEQ ID NO: 93 - protein, 11 aa, synthetic construct
  REGION 1..11: note = Description of Artificial Sequence: Synthetic peptide
QASESISTYLS

SEQ ID NO: 94 - protein, 11 aa, synthetic construct
  REGION 1..11: note = Description of Artificial Sequence: Synthetic peptide
QASQSISTYLA

SEQ ID NO: 95 - protein, 11 aa, synthetic construct
  REGION 1..11: note = Description of Artificial Sequence: Synthetic peptide
QASQSISSYLA

SEQ ID NO: 96 - protein, 11 aa, synthetic construct
  REGION 1..11: note = Description of Artificial Sequence: Synthetic peptide
  MOD_RES 4: note = Glu or Gln
  MOD_RES 7: note = Ser or Gly
  MOD_RES 8: note = Asn or Ser
  MOD_RES 9: note = Glu, Tyr or Ala
  MOD_RES 11: note = Ser or Ala
QASXSIXXXLX

SEQ ID NO: 97 - protein, 7 aa, synthetic construct
  REGION 1..7: note = Description of Artificial Sequence: Synthetic peptide
GATTLHD

SEQ ID NO: 98 - protein, 7 aa, synthetic construct
  REGION 1..7: note = Description of Artificial Sequence: Synthetic peptide
DASRLQD

SEQ ID NO: 99 - protein, 7 aa, synthetic construct
  REGION 1..7: note = Description of Artificial Sequence: Synthetic peptide
DASDLAS

SEQ ID NO: 100 - protein, 7 aa, synthetic construct
  REGION 1..7: note = Description of Artificial Sequence: Synthetic peptide
SASTLAS

SEQ ID NO: 101 - protein, 7 aa, synthetic construct
  REGION 1..7: note = Description of Artificial Sequence: Synthetic peptide
YASTLAS

SEQ ID NO: 102 - protein, 7 aa, synthetic construct
  REGION 1..7: note = Description of Artificial Sequence: Synthetic peptide
RASTLAS

SEQ ID NO: 103 - protein, 7 aa, synthetic construct
  REGION 1..7: note = Description of Artificial Sequence: Synthetic peptide
AASTLAS

SEQ ID NO: 104 - protein, 7 aa, synthetic construct
  REGION 1..7: note = Description of Artificial Sequence: Synthetic peptide
DASNLAS

SEQ ID NO: 105 - protein, 7 aa, synthetic construct
  REGION 1..7: note = Description of Artificial Sequence: Synthetic peptide
SASTLAS

SEQ ID NO: 106 - protein, 7 aa, synthetic construct
  REGION 1..7: note = Description of Artificial Sequence: Synthetic peptide
QASTLAS

SEQ ID NO: 107 - protein, 7 aa, synthetic construct
  REGION 1..7: note = Description of Artificial Sequence: Synthetic peptide
KTSTLAS

SEQ ID NO: 108 - protein, 7 aa, synthetic construct
  REGION 1..7: note = Description of Artificial Sequence: Synthetic peptide
KASTLAS

SEQ ID NO: 109 - protein, 7 aa, synthetic construct
  REGION 1..7: note = Description of Artificial Sequence: Synthetic peptide
  MOD_RES 1: note = Tyr, Ser or Asp
  MOD_RES 4: note = Thr or Asp
XASXLAS

SEQ ID NO: 110 - protein, 9 aa, synthetic construct
  REGION 1..9: note = Description of Artificial Sequence: Synthetic peptide
HQGLSFPYT

SEQ ID NO: 111 - protein, 9 aa, synthetic construct
  REGION 1..9: note = Description of Artificial Sequence: Synthetic peptide
QQYHDYPYT

SEQ ID NO: 112 - protein, 11 aa, synthetic construct
  REGION 1..11: note = Description of Artificial Sequence: Synthetic peptide
QATAYGSSGNA

SEQ ID NO: 113 - protein, 12 aa, synthetic construct
  REGION 1..12: note = Description of Artificial Sequence: Synthetic peptide
HQDYTSNDVENT

SEQ ID NO: 114 - protein, 12 aa, synthetic construct
  REGION 1..12: note = Description of Artificial Sequence: Synthetic peptide
QQDYTGNNVDNT

SEQ ID NO: 115 - protein, 12 aa, synthetic construct
  REGION 1..12: note = Description of Artificial Sequence: Synthetic peptide
AQGFGSSGVENV

SEQ ID NO: 116 - protein, 12 aa, synthetic construct
  REGION 1..12: note = Description of Artificial Sequence: Synthetic peptide
HQGYSSSNVDNI

SEQ ID NO: 117 - protein, 12 aa, synthetic construct
  REGION 1..12: note = Description of Artificial Sequence: Synthetic peptide
QQGYTWNNVDNV

SEQ ID NO: 118 - protein, 12 aa, synthetic construct
  REGION 1..12: note = Description of Artificial Sequence: Synthetic peptide
HQDYTSNNVDNT

SEQ ID NO: 119 - protein, 12 aa, synthetic construct
  REGION 1..12: note = Description of Artificial Sequence: Synthetic peptide
HQDYTSSNVDNT

SEQ ID NO: 120 - protein, 12 aa, synthetic construct
  REGION 1..12: note = Description of Artificial Sequence: Synthetic peptide
HQDYTSNSVDNT

SEQ ID NO: 121 - protein, 12 aa, synthetic construct
  REGION 1..12: note = Description of Artificial Sequence: Synthetic peptide
AQGFSSSGVENV

SEQ ID NO: 122 - protein, 12 aa, synthetic construct
  REGION 1..12: note = Description of Artificial Sequence: Synthetic peptide
QQGDSHNNVDNI

SEQ ID NO: 123 - protein, 12 aa, synthetic construct
  REGION 1..12: note = Description of Artificial Sequence: Synthetic peptide
QQDYSRSNIVNS

SEQ ID NO: 124 - protein, 12 aa, synthetic construct
  REGION 1..12: note = Description of Artificial Sequence: Synthetic peptide
QQDYSSSNIVNS

SEQ ID NO: 125 - protein, 12 aa, synthetic construct
  REGION 1..12: note = Description of Artificial Sequence: Synthetic peptide
QQGYSGSSVENT

SEQ ID NO: 126 - protein, 12 aa, synthetic construct
  REGION 1..12: note = Description of Artificial Sequence: Synthetic peptide
QQGYSGSNVENT

SEQ ID NO: 127 - skipped

SEQ ID NO: 128 - protein, 107 aa, synthetic construct
  REGION 1..107: note = Description of Artificial Sequence: Synthetic polypeptide
DIQMTQSPASLSASLGETVTIQCRASEDIYSGLAWYQQKPGKSPQLLIYGATTLHDGVPSRFSGSGSGTQYSLKISSMHSEDEGIYFCHQGLSFPYTFGAGTKLELK

SEQ ID NO: 129 - protein, 107 aa, synthetic construct
  REGION 1..107: note = Description of Artificial Sequence: Synthetic polypeptide
DIQMTQSPASLSASLGETVTIECLASKNIYRNLAWYQQKPGKSPQFLISDASRLQDGVPSRFTGSDSGSQYSLKINSLQSEDVATYFCQQYHDYPYTFGAGTKLELK

SEQ ID NO: 130 - protein, 109 aa, synthetic construct
  REGION 1..109: note = Description of Artificial Sequence: Synthetic polypeptide
DVVMTQTASPVSAAVGGTVTIKCQASENIYSLLAWYQQKPGQPPKVLIYDASDLASGVPSRFKGSGSGTQFTLTISDLECADAATYYCQATAYGSSGNAFGGGTEVVVK

SEQ ID NO: 131 - protein, 109 aa, synthetic construct
  REGION 1..109: note = Description of Artificial Sequence: Synthetic polypeptide
DVVMTQTPASVSEPVGGTVTIKCQASENIYSLLAWYQQKPGQPPKVLIYDASDLASGVPSRFKGSGSGTQFTLTISDLECADAATYYCQATAYGSSGNAFGGGTEVVVK

SEQ ID NO: 132 - protein, 110 aa, synthetic construct
  REGION 1..110: note = Description of Artificial Sequence: Synthetic polypeptide
NIVMTQTPASVEVAVGGTVVIKCQASQNIGDYLSWYQQKPGQRPKLLIYSASTLASGVPSRFKGSGSGTQFTLTISDLECADAATYYCHQDYTSNDVENTFGGGTEVVVK

SEQ ID NO: 133 - protein, 110 aa, synthetic construct
  REGION 1..110: note = Description of Artificial Sequence: Synthetic polypeptide
AYYMTQTPASVEVAVGGTVTIKCQASEDIGSYCSWYQQKPGQPPKLLIYDASDLASGVPSRFKGSGSGTDFTLTISGVQCDDAATYYCQQDYTGNNVDNTFGGGSEVVVK

SEQ ID NO: 134 - protein, 110 aa, synthetic construct
  REGION 1..110: note = Description of Artificial Sequence: Synthetic polypeptide
AYDMTQTPASLEAAVGGTVTINCQASESISNELSWYQQKPGQPPDLLIYYASTLASGVPSRFKGSGSGTEFTLTISDLECADAATYYCAQGFGSSGVENVFGGGTEVVVK

SEQ ID NO: 135 - protein, 110 aa, synthetic construct
  REGION 1..110: note = Description of Artificial Sequence: Synthetic polypeptide
AYRMTQSPSSFSASTGDRVTITCQASESISNELSWYQQKPGKPPKLLIYYASTLASGVPSRFSGSGSGTDFTLTISCLQSEDFATYYCAQGFGSSGVENVFGGGTKVEIK

SEQ ID NO: 136 - protein, 110 aa, synthetic construct
  REGION 1..110: note = Description of Artificial Sequence: Synthetic polypeptide
AIRMTQSPSSFSASTGDRVTITCQASESISNELSWYQQKPGKPPKLLIYYASTLASGVPSRFSGSGSGTDFTLTISCLQSEDFATYYCAQGFGSSGVENVFGGGTKVEIK

SEQ ID NO: 137 - protein, 110 aa, synthetic construct
  REGION 1..110: note = Description of Artificial Sequence: Synthetic polypeptide
AYRMTQSPSSFSASTGDRVTITCQASESISNELSWYQQKPGKAPKLLIYYASTLASGVPSRFSGSGSGTDFTLTISCLQSEDFATYYCAQGFGSSGVENVFGGGTKVEIK

SEQ ID NO: 138 - protein, 110 aa, synthetic construct
  REGION 1..110: note = Description of Artificial Sequence: Synthetic polypeptide
AIRMTQSPSSFSASTGDRVTITCQASESISNELSWYQQKPGKAPKLLIYYASTLASGVPSRFSGSGSGTDFTLTISCLQSEDFATYYCAQGFGSSGVENVFGGGTKVEIK

SEQ ID NO: 139 - protein, 110 aa, synthetic construct
  REGION 1..110: note = Description of Artificial Sequence: Synthetic polypeptide
AIRMTQSPSSFSASTGDRVTITCQASESISNELSWYQQKPGKAPKLLIYYASTLASGVPSRFSGSGSGTDFTLTISSLQPEDFATYYCAQGFGSSGVENVFGGGTKVEIK

SEQ ID NO: 140 - protein, 110 aa, synthetic construct
  REGION 1..110: note = Description of Artificial Sequence: Synthetic polypeptide
  MOD_RES 2: note = Tyr or Ile
  MOD_RES 43: note = Pro or Ala
  MOD_RES 77: note = Cys or Ser
  MOD_RES 80: note = Ser or Pro
  MOD_RES 106: note = Glu or Lys
  MOD_RES 108: note = Glu or Val
  MOD_RES 109: note = Ile or Val
AXRMTQSPSSFSASTGDRVTITCQASESISNELSWYQQKPGKXPKLLIYYASTLASGVPSRFSGSGSGTDFTLTISXLQXEDFATYYCAQGFGSSGVENVFGGGTXVXXK

SEQ ID NO: 141 - protein, 110 aa, synthetic construct
  REGION 1..110: note = Description of Artificial Sequence: Synthetic polypeptide
AYYMTQTPASVEAAVGGTVTIKCQASQSISNYVAWYQQKPGQPPKLLIYRASTLASGVSSRFSGSGSGTEFTLTISDLECADAATYYCHQGYSSSNVDNIFGGGTEVVVK

SEQ ID NO: 142 - protein, 110 aa, synthetic construct
  REGION 1..110: note = Description of Artificial Sequence: Synthetic polypeptide
AYEMTQTPASVEVAVGGTVTINCQASESISSDLAWYQQKPGQRPKLLIYAASTLASGVPSRFKGSGSGTEFTLSISGVQCADAATYYCQQGYTWNNVDNVFGGGTEVVVK

SEQ ID NO: 143 - protein, 110 aa, synthetic construct
  REGION 1..110: note = Description of Artificial Sequence: Synthetic polypeptide
DIVMTQTPASVSEPVGGTVTIKCQASQNINNYLSWYQQKPGQPPKQLIYAASTLASGVPSRFKGSGSGTQFTLTISDLECADAATYYCHQDYTSNNVDNTFGGGTEVVVK

SEQ ID NO: 144 - protein, 110 aa, synthetic construct
  REGION 1..110: note = Description of Artificial Sequence: Synthetic polypeptide
NIVMTQTPASVEVAVGGTVTIKCQASQSIGSYLSWYQQKPGQPPKLLIYDASNLASGVPSRFKGSGSGTQFTLSISDLECADAATYYCHQDYTSNNVDNTFGGGTEVVIK

SEQ ID NO: 145 - protein, 110 aa, synthetic construct
  REGION 1..110: note = Description of Artificial Sequence: Synthetic polypeptide
NIVMTQTPASVEAAVGGTVTINCQASQSISSYLSWYQQKSGQRPKLLIFSASTLASGVPSRFTGSGSGTQFTLTISDLQCADAATYYCHQDYTSSNVDNTFGGGTEVVVK

SEQ ID NO: 146 - protein, 110 aa, synthetic construct
  REGION 1..110: note = Description of Artificial Sequence: Synthetic polypeptide
DIQMTQSPSSLSASVGDRVTITCQASQSISSYLSWYQQKPGKRPKLLIFSASTLASGVPSRFSGSGSGTDFTLTISSLQPEDFATYYCHQDYTSSNVDNTFGGGTKVEIK

SEQ ID NO: 147 - protein, 110 aa, synthetic construct
  REGION 1..110: note = Description of Artificial Sequence: Synthetic polypeptide
DIQMTQSPSSLSASVGDRVTITCQASQSISSYLSWYQQKPGKAPKLLIFSASTLASGVPSRFSGSGSGTDFTLTISSLQPEDFATYYCHQDYTSSNVDNTFGGGTKVEIK

SEQ ID NO: 148 - protein, 110 aa, synthetic construct
  REGION 1..110: note = Description of Artificial Sequence: Synthetic polypeptide
DIQMTQSPSSLSASVGDRVTITCQASQSISSYLSWYQQKPGKRPKLLIYSASTLASGVPSRFSGSGSGTDFTLTISSLQPEDFATYYCHQDYTSSNVDNTFGGGTKVEIK

SEQ ID NO: 149 - protein, 110 aa, synthetic construct
  REGION 1..110: note = Description of Artificial Sequence: Synthetic polypeptide
DIQMTQSPSSLSASVGDRVTITCQASQSISSYLSWYQQKPGKAPKLLIYSASTLASGVPSRFSGSGSGTDFTLTISSLQPEDFATYYCHQDYTSSNVDNTFGGGTKVEIK

SEQ ID NO: 150 - protein, 110 aa, synthetic construct
  REGION 1..110: note = Description of Artificial Sequence: Synthetic polypeptide
DIQMTQSPSSLSASVGDRVTITCQASQSISSYLSWYQQKPGKAPKLLIYSASTLASGVPSRFSGSGSGTDFTLTISSLQPEDFATYYCHQDYTSSNVDNTFGGGTEVVVK

SEQ ID NO: 151 - protein, 110 aa, synthetic construct
  REGION 1..110: note = Description of Artificial Sequence: Synthetic polypeptide
  MOD_RES 43: note = Arg or Ala
  MOD_RES 49: note = Phe or Tyr
  MOD_RES 106: note = Glu or Lys
  MOD_RES 108: note = Glu or Val
  MOD_RES 109: note = Val or Ile
DIQMTQSPSSLSASVGDRVTITCQASQSISSYLSWYQQKPGKXPKLLIXSASTLASGVPSRFSGSGSGTDFTLTISSLQPEDFATYYCHQDYTSSNVDNTFGGGTXVXXK

SEQ ID NO: 152 - protein, 110 aa, synthetic construct
  REGION 1..110: note = Description of Artificial Sequence: Synthetic polypeptide
NIVMTQTPASVEVAVGGTVIIKCQASQSISNYLSWYHQKSGQRPRLLIYSASTLASGVPSRFKGSGSGTQFTLTISDLECADAATYYCHQDYTSNSVDNTFGGGTEVVVK

SEQ ID NO: 153 - protein, 110 aa, synthetic construct
  REGION 1..110: note = Description of Artificial Sequence: Synthetic polypeptide
NIVMTQTPASVEVAMGGTVIIKCQASQSISTYLSWYQQKPGQPPKLLIYSASTLASGVSSRFEGSGSGTQFTLTISGVQCADAATYYCHQDYTSNNVDNTFGGGTEVVVK

SEQ ID NO: 154 - protein, 110 aa, synthetic construct
  REGION 1..110: note = Description of Artificial Sequence: Synthetic polypeptide
AYDLTQTPASVEAAVGGTVTINCQASESIGNELSWYQQKSGQPPKLLIYQASTLASGVPSRFKGSGSGTDFTLTISDLECADAATYYCAQGFSSSGVENVFGGGTEVVVK

SEQ ID NO: 155 - protein, 110 aa, synthetic construct
  REGION 1..110: note = Description of Artificial Sequence: Synthetic polypeptide
AYDMTQTPASVEAAVGGTVTIKCQASESIGNALAWYQQKPGQPPKLLIYDASDLASGVPSRFKGSGSGTQFTLTISGVECADAATYYCQQGDSHNNVDNIFGGGTEVVVK

SEQ ID NO: 156 - protein, 110 aa, synthetic construct
  REGION 1..110: note = Description of Artificial Sequence: Synthetic polypeptide
AYRMTQSPSSFSASTGDRVTITCQASESIGNALAWYQQKPGKPPKLLIYDASDLASGVPSRFSGSGSGTDFTLTISCLQSEDFATYYCQQGDSHNNVDNIFGGGTKVEIK

SEQ ID NO: 157 - protein, 110 aa, synthetic construct
  REGION 1..110: note = Description of Artificial Sequence: Synthetic polypeptide
AIRMTQSPSSFSASTGDRVTITCQASESIGNALAWYQQKPGKPPKLLIYDASDLASGVPSRFSGSGSGTDFTLTISCLQSEDFATYYCQQGDSHNNVDNIFGGGTKVEIK

SEQ ID NO: 158 - protein, 110 aa, synthetic construct
  REGION 1..110: note = Description of Artificial Sequence: Synthetic polypeptide
AYRMTQSPSSFSASTGDRVTITCQASESIGNALAWYQQKPGKAPKLLIYDASDLASGVPSRFSGSGSGTDFTLTISCLQSEDFATYYCQQGDSHNNVDNIFGGGTKVEIK

SEQ ID NO: 159 - protein, 110 aa, synthetic construct
  REGION 1..110: note = Description of Artificial Sequence: Synthetic polypeptide
AIRMTQSPSSFSASTGDRVTITCQASESIGNALAWYQQKPGKAPKLLIYDASDLASGVPSRFSGSGSGTDFTLTISCLQSEDFATYYCQQGDSHNNVDNIFGGGTKVEIK

SEQ ID NO: 160 - protein, 110 aa, synthetic construct
  REGION 1..110: note = Description of Artificial Sequence: Synthetic polypeptide
AIRMTQSPSSFSASTGDRVTITCQASESIGNALAWYQQKPGKAPKLLIYDASDLASGVPSRFSGSGSGTDFTLTISSLQPEDFATYYCQQGDSHNNVDNIFGGGTKVEIK

SEQ ID NO: 161 - protein, 110 aa, synthetic construct
  REGION 1..110: note = Description of Artificial Sequence: Synthetic polypeptide
AIRMTQSPSSFSASTGDRVTITCQASESIGNALAWYQQKPGKAPKLLIYDASDLASGVPSRFSGSGSGTDFTLTISSLQPEDFATYYCQQGDSHNNVDNIFGGGTEVVVK

SEQ ID NO: 162 - protein, 110 aa, synthetic construct
  REGION 1..110: note = Description of Artificial Sequence: Synthetic polypeptide
  MOD_RES 2: note = Tyr or Ile
  MOD_RES 43: note = Pro or Ala
  MOD_RES 77: note = Cys or Ser
  MOD_RES 80: note = Ser or Pro
  MOD_RES 106: note = Glu or Lys
  MOD_RES 108: note = Glu or Val
  MOD_RES 109: note = Val or Ile
AXRMTQSPSSFSASTGDRVTITCQASESIGNALAWYQQKPGKXPKLLIYDASDLASGVPSRFSGSGSGTDFTLTISXLQXEDFATYYCQQGDSHNNVDNIFGGGTXVXXK

SEQ ID NO: 163 - protein, 110 aa, synthetic construct
  REGION 1..110: note = Description of Artificial Sequence: Synthetic polypeptide
AIEMTQTPSSASEPVGGTVTIKCQASESISRYLSWYQQKPGQPPKLLIYDASDLASGVSSRFKGSGSGTQFTLIISDVECADAATYYCQQDYSRSNIVNSFGGGTEVVVK

SEQ ID NO: 164 - protein, 110 aa, synthetic construct
  REGION 1..110: note = Description of Artificial Sequence: Synthetic polypeptide
AYDMTQTPSSASEPVGGTVTIKCQASESISTYLSWYQQKPGQPPKLLIYDASDLASGVSSRFKGSGSGTQFTLTISDVECADAATYYCQQDYSSSNIVNSFGGGTEVVVK

SEQ ID NO: 165 - protein, 110 aa, synthetic construct
  REGION 1..110: note = Description of Artificial Sequence: Synthetic polypeptide
AYDMTQTPASVEVAVGGTVTIKCQASQSISTYLAWYQQKPGQRPNLLIYKTSTLASGVPSRFRGSGSGTQFTLTISGVECADAATYYCQQGYSGSSVENTFGGGTEVVVK

SEQ ID NO: 166 - protein, 110 aa, synthetic construct
  REGION 1..110: note = Description of Artificial Sequence: Synthetic polypeptide
AYDMTQTPVSVEAAVGGTVTIKCQASQSISSYLAWYQQKPGQPPKLLIYKASTLASGVSSRFKGSGSGTEFTLTISDLECADAATYYCQQGYSGSNVENTFGGGTEVVVK

SEQ ID NO: 167 - protein, 121 aa, synthetic construct
  REGION 1..121: note = Description of Artificial Sequence: Synthetic polypeptide
EVQLVESGGGLVQPGRSLKLSCTASGFTFSDYNMAWVRQAPKGGLEWVTTISYDAGRTYYRDSVKGRFTISRDNAKRTLSLQMDSLRSEDTATYYCATGIFNYGTDYFDYWGQGVMVTVSS

SEQ ID NO: 168 - protein, 121 aa, synthetic construct
  REGION 1..121: note = Description of Artificial Sequence: Synthetic polypeptide
EVQLVESGGGLVRPGRSLRLSCAASGFTFSDYYMAWVRQAPTKGLEWVASISYDGDTTYYRDSVKGRFTISRDNARSSLYLQMDSLRSDDTANYFCTTDGTIPAGSWFAYWGQGTLVTVSS

SEQ ID NO: 169 - protein, 120 aa, synthetic construct
  REGION 1..120: note = Description of Artificial Sequence: Synthetic polypeptide
QEQLEESGGDLVKPEGSLTLTCTASGFSFSSSYWICWVRQAPGKGLEWIACVYGLDVNIYYASWTKGRFTISKTSSTTVTLQMTSLTAADTATYFCARGGGSADFGFDLWGPGTLVTVSS

SEQ ID NO: 170 - protein, 119 aa, synthetic construct
  REGION 1..119: note = Description of Artificial Sequence: Synthetic polypeptide
QSLEESGGDLVKPEGSLTLTCTASGFSFSTSYWICWVRQAPGKGLEWIACVYGLDVNIYYASWTEGRFTISKTSSTTVTLQVTSLTAADTATYFCARGGGSADFGFDLWGPGTLVTVSS

SEQ ID NO: 171 - protein, 118 aa, synthetic construct
  REGION 1..118: note = Description of Artificial Sequence: Synthetic polypeptide
QSVKESEGGLFKPTDNLTLTCTVSGFSLSNYGVTWVRQAPGNGLEYIGFIGSGGSAYYASWAKSRSTITRNTNLNTVTLKMTSLTAADTATYFCARDDVGGGKSLDIWGPGTLVTVSS

SEQ ID NO: 172 - protein, 119 aa, synthetic construct
  REGION 1..119: note = Description of Artificial Sequence: Synthetic polypeptide
QSVEESRGGLIKPTDTLTLTCTASGFSLSNYGVSWVRQAPGNGLEYIGFIGYGGSTYYASWAKSRSTITRNTNLNTVTLQMTSLTAADTATYFCARLCGVDCADALDSWGPGTLVTVSS

SEQ ID NO: 173 - protein, 119 aa, synthetic construct
  REGION 1..119: note = Description of Artificial Sequence: Synthetic polypeptide
QSLEESGGGLVKPTDTLTLTCTVSGFSLSSYGVTWVRQAPGRGLEWIGYITSNYGVSYYASWAKSRSTITRNTNLNTVTLKMTSLTAADTATYFCARENPDYGYAYDAWGPGTLVTVSV

SEQ ID NO: 174 - protein, 120 aa, synthetic construct
  REGION 1..120: note = Description of Artificial Sequence: Synthetic polypeptide
EVQLVESGGGLVQPGGSLRLSCAVSGFSLSSYGVTWVRQAPGKGLEWIGYITSNYGVSYYASWAKSRSTISRDTSKNTVYLQMGSLRAEDMAVYFCARENPDYGYAYDAWGPGTTVTVSS

SEQ ID NO: 175 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWIGYITSNYGVSYYASWAKSRSTISRDTSKNTVYLQMGSLRAEDMAVYFCARENPDYGYAYDAWGPGTTVTVSS

SEQ ID NO: 176 - protein, 120 aa, synthetic construct
  REGION 1..120: note = Description of Artificial Sequence: Synthetic polypeptide
EVQLVESGGGLVQPGGSLRLSCAVSGFSLSSYGVTWVRQAPGKGLEYIGYITSNYGVSYYASWAKSRSTISRDTSKNTVYLQMGSLRAEDMAVYFCARENPDYGYAYDAWGPGTTVTVSS

SEQ ID NO: 177 - protein, 120 aa, synthetic construct
  REGION 1..120: note = Description of Artificial Sequence: Synthetic polypeptide
EVQLVESGGGLVQPGGSLRLSCAVSGFSLSSYGVTWVRQAPGKGLEWVGYITSNYGVSYYASWAKSRSTISRDTSKNTVYLQMGSLRAEDMAVYFCARENPDYGYAYDAWGPGTTVTVSS

SEQ ID NO: 178 - protein, 120 aa, synthetic construct
  REGION 1..120: note = Description of Artificial Sequence: Synthetic polypeptide
EVQLVESGGGLVQPGGSLRLSCAVSGFSLSSYGVTWVRQAPGKGLEWISYITSNYGVSYYASWAKSRSTISRDTSKNTVYLQMGSLRAEDMAVYFCARENPDYGYAYDAWGPGTTVTVSS

SEQ ID NO: 179 - protein, 120 aa, synthetic construct
  REGION 1..120: note = Description of Artificial Sequence: Synthetic polypeptide
EVQLVESGGGLVQPGGSLRLSCAVSGFSLSSYGVTWVRQAPGKGLEWIGYITSNYGVSYYASWAKSRFTISRDTSKNTVYLQMGSLRAEDMAVYFCARENPDYGYAYDAWGPGTTVTVSS

SEQ ID NO: 180 - protein, 120 aa, synthetic construct
  REGION 1..120: note = Description of Artificial Sequence: Synthetic polypeptide
EVQLVESGGGLVQPGGSLRLSCAVSGFSLSSYGVTWVRQAPGKGLEWIGYITSNYGVSYYASWAKSRSTISRDNSKNTVYLQMGSLRAEDMAVYFCARENPDYGYAYDAWGPGTTVTVSS

SEQ ID NO: 181 - protein, 120 aa, synthetic construct
  REGION 1..120: note = Description of Artificial Sequence: Synthetic polypeptide
EVQLVESGGGLVQPGGSLRLSCAVSGFSLSSYGVTWVRQAPGKGLEWIGYITSNYGVSYYASWAKSRSTISRDTSKNTLYLQMGSLRAEDMAVYFCARENPDYGYAYDAWGPGTTVTVSS

SEQ ID NO: 182 - protein, 120 aa, synthetic construct
  REGION 1..120: note = Description of Artificial Sequence: Synthetic polypeptide
EVQLVESGGGLVQPGGSLRLSCAVSGFSLSSYGVTWVRQAPGKGLEWIGYITSNYGVSYYASWAKSRSTISRDTSKNTVYLQMGSLRAEDMAVYYCARENPDYGYAYDAWGPGTTVTVSS

SEQ ID NO: 183 - protein, 120 aa, synthetic construct
  REGION 1..120: note = Description of Artificial Sequence: Synthetic polypeptide
EVQLVESGGGLVQPGGSLRLSCAVSGFSLSSYGVTWVRQAPGKGLEWIGYITSNYGVSYYASWAKSRSTISRDTSKNTVYLQMGSLRAEDMAVYFCARENPDYGYAYDAWGQGTTVTVSS

SEQ ID NO: 184 - protein, 120 aa, synthetic construct
  REGION 1..120: note = Description of Artificial Sequence: Synthetic polypeptide
EVQLVESGGGLVQPGGSLRLSCAVSGFSLSSYGVTWVRQAPGKGLEWIGYITSNYGVSYYANSVKGRSTISRDTSKNTVYLQMGSLRAEDMAVYFCARENPDYGYAYDAWGPGTTVTVSS

SEQ ID NO: 185 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEYVSYITSNYGVSYYASWAKSRFTISRDNSKNTLYLQMGSLRAEDMAVYYCARENPDYGYAYDAWGQGTTVTVSS

SEQ ID NO: 186 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEYVSYITSNYGVSYYANSVKGRFTISRDNSKNTLYLQMGSLRAEDMAVYYCARENPDYGYAYDAWGQGTTVTVSS

SEQ ID NO: 187 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWVGYITSNYGVSYYASWAKSRFTISRDNSKNTLYLQMGSLRAEDMAVYYCARENPDYGYAYDAWGQGTTVTVSS

SEQ ID NO: 188 - protein, 120 aa, synthetic construct
  REGION 1..120: note = Description of Artificial Sequence: Synthetic polypeptide
EQQLVESGGGLVQPGGSLRLSCAASGFSLSSYGVTWVRQAPGKGLEWVGYITSNYGVSYYASWAKSRFTISRDNSKNTLYLQMGSLRAEDMAVYYCARENPDYGYAYDAWGQGTTVTVSS

SEQ ID NO: 189 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWVGYITSNYGVSYYASWAKSRFTISRNLNTNTLYLQMGSLRAEDMAVYYCARENPDYGYAYDAWGQGTTVTVSS

SEQ ID NO: 190 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWVGYITSNYGVSYYASFAKSRFTISRDNSKNTLYLQMGSLRAEDMAVYYCARENPDYGYAYDAWGQGTTVTVSS

SEQ ID NO: 191 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWVGYITSNYGVSYYASWAKSRFTISRNTNLNTLYLQMGSLRAEDMAVYYCARENPDYGYAYDAWGQGTTVTVSS

SEQ ID NO: 192 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWVGYITSNYGVSYYASWAKSRFTISRNNSKNTLYLQMGSLRAEDMAVYYCARENPDYGYAYDAWGQGTTVTVSS

SEQ ID NO: 193 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWVGYITSNYGVSYYASWAKSRFTISRDNNKNTLYLQMGSLRAEDMAVYYCARENPDYGYAYDAWGQGTTVTVSS

SEQ ID NO: 194 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWVGYITSNYGVSYYASWAKSRFTISRNNNKNTLYLQMGSLRAEDMAVYYCARENPDYGYAYDAWGQGTTVTVSS

SEQ ID NO: 195 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWIGYITSNYGVSYYASWAKSRFTISRDNSKNTLYLQMGSLRAEDMAVYYCARENPDYGYAYDAWGQGTTVTVSS

SEQ ID NO: 196 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWVGYITSNYGVSYYASWAKSRSTISRDNSKNTLYLQMGSLRAEDMAVYYCARENPDYGYAYDAWGQGTTVTVSS

SEQ ID NO: 197 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWIGYITSNYGVSYYASWAKSRSTISRDNSKNTLYLQMGSLRAEDMAVYYCARENPDYGYAYDAWGQGTTVTVSS

SEQ ID NO: 198 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWVGYITSNYGVSYYASWAKSRFTISRDTSKNTLYLQMGSLRAEDMAVYYCARENPDYGYAYDAWGQGTTVTVSS

SEQ ID NO: 199 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWVGYITSNYGVSYYASWAKSRFTISRDNSKNTVYLQMGSLRAEDMAVYYCARENPDYGYAYDAWGQGTTVTVSS

SEQ ID NO: 200 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWIGYITSNYGVSYYASWAKSRSTISRDNSKNTVYLQMGSLRAEDMAVYYCARENPDYGYAYDAWGQGTTVTVSS

SEQ ID NO: 201 - protein, 120 aa, synthetic construct
  REGION 1..120: note = Description of Artificial Sequence: Synthetic polypeptide
EVQLVESGGGLVQPGGSLRLSCAASGFSLSSYGVTWVRQAPGKGLEWIGYITSNYGVSYYASWAKSRSTISRDTSKNTVYLQMGSLRAEDMAVYYCARENPDYGYAYDAWGQGTTVTVSS

SEQ ID NO: 202 - protein, 120 aa, synthetic construct
  REGION 1..120: note = Description of Artificial Sequence: Synthetic polypeptide
  VARIANT 2
  VARIANT 24
  VARIANT 47
  VARIANT 48
  VARIANT 49
  VARIANT 62
  VARIANT 63
  VARIANT 64
  VARIANT 66
  VARIANT 68
  VARIANT 73
  VARIANT 74
  VARIANT 75
  VARIANT 76
  VARIANT 79
  VARIANT 95
  VARIANT 112
EXQLVESGGGLVQPGGSLRLSCAXSGFSLSSYGVTWVRQAPGKGLEXXXYITSNYGVSYYAXXXKXRXTISRXXXXNTXYLQMGSLRAEDMAVYXCARENPDYGYAYDAWGXGTTVTVSS

SEQ ID NO: 203 - protein, 120 aa, synthetic construct
  REGION 1..120: note = Description of Artificial Sequence: Synthetic polypeptide
QSVKESEGGLFKPTDTLTLTCTVSGFSLINYGVSWVRQAPGKGLEWIGYIGSAGSTYYATWAKSRATITRNTNLNTVTLKMTSLTAADTATYFCARAAYSAGSADAEDIWGPGTLVTVSS

SEQ ID NO: 204 - protein, 119 aa, synthetic construct
  REGION 1..119: note = Description of Artificial Sequence: Synthetic polypeptide
QSVKESEGGLIKPTDTLTLTCTVSGFSLSTFAINWVRQAPGNGLEWIGAIGRGGSAYYASWAKSRSTITKNTNLNTVTLKMTRPTAADTATYFCARENAGSGWGELDIWGPGTLVTVSS

SEQ ID NO: 205 - protein, 118 aa, synthetic construct
  REGION 1..118: note = Description of Artificial Sequence: Synthetic polypeptide
QSVKESEGGLFKPTDTLTLTCTVSGFSLSGYGVSWVRQAPGKGLEYIGFIGSGGSAYYASWAKSRSTITRNTNLNTVTLKMTRLTAADTATYFCARDNVGGDMSLDIWGPGTLVTVSS

SEQ ID NO: 206 - protein, 118 aa, synthetic construct
  REGION 1..118: note = Description of Artificial Sequence: Synthetic polypeptide
QSVKESEGGLFKPTDNLTLTCTVSGFSLSNYGVTWVRQAPGNGLEYIGFIGSSGSAYYASWAKSRSTITRNTNLNTVTLKMTSLTAADTATYFCARDDVGGGKSLDIWGPGTLVTVSS

SEQ ID NO: 207 - protein, 118 aa, synthetic construct
  REGION 1..118: note = Description of Artificial Sequence: Synthetic polypeptide
QSVKESEGGLFKPTDNLTLTCTVSGFSLSSYGVSWVRQAPGNGLEYIGFIGSGGFAYYASWAKSRSTITRNTNLNTVTLKMTSLTAADTATYFCARDDVGGGKSLDIWGPGTVVTVSV

SEQ ID NO: 208 - protein, 119 aa, synthetic construct
  REGION 1..119: note = Description of Artificial Sequence: Synthetic polypeptide
EVQLVESGGGLIQPGGSLRLSCAVSGFSLSSYGVSWVRQAPGKGLEYIGFIGSGGFAYYASWAKSRSTISRDTSKNTVYLQMNSLRAEDTAVYFCARDDVGGGKSLDIWGPGTLVTVSS

SEQ ID NO: 209 - protein, 119 aa, synthetic construct
  REGION 1..119: note = Description of Artificial Sequence: Synthetic polypeptide
EVQLVESGGGLIQPGGSLRLSCAASGFSLSSYGVSWVRQAPGKGLEYIGFIGSGGFAYYASWAKSRSTISRDTSKNTVYLQMNSLRAEDTAVYFCARDDVGGGKSLDIWGPGTLVTVSS

SEQ ID NO: 210 - protein, 119 aa, synthetic construct
  REGION 1..119: note = Description of Artificial Sequence: Synthetic polypeptide
EVQLVESGGGLIQPGGSLRLSCAVSGFSLSSYGVSWVRQAPGKGLEWIGFIGSGGFAYYASWAKSRSTISRDTSKNTVYLQMNSLRAEDTAVYFCARDDVGGGKSLDIWGPGTLVTVSS

SEQ ID NO: 211 - protein, 119 aa, synthetic construct
  REGION 1..119: note = Description of Artificial Sequence: Synthetic polypeptide
EVQLVESGGGLIQPGGSLRLSCAVSGFSLSSYGVSWVRQAPGKGLEYVGFIGSGGFAYYASWAKSRSTISRDTSKNTVYLQMNSLRAEDTAVYFCARDDVGGGKSLDIWGPGTLVTVSS

SEQ ID NO: 212 - protein, 119 aa, synthetic construct
  REGION 1..119: note = Description of Artificial Sequence: Synthetic polypeptide
EVQLVESGGGLIQPGGSLRLSCAVSGFSLSSYGVSWVRQAPGKGLEYISFIGSGGFAYYASWAKSRSTISRDTSKNTVYLQMNSLRAEDTAVYFCARDDVGGGKSLDIWGPGTLVTVSS

SEQ ID NO: 213 - protein, 119 aa, synthetic construct
  REGION 1..119: note = Description of Artificial Sequence: Synthetic polypeptide
EVQLVESGGGLIQPGGSLRLSCAVSGFSLSSYGVSWVRQAPGKGLEYIGFIGSGGFAYYASWAKSRFTISRDTSKNTVYLQMNSLRAEDTAVYFCARDDVGGGKSLDIWGPGTLVTVSS

SEQ ID NO: 214 - protein, 119 aa, synthetic construct
  REGION 1..119: note = Description of Artificial Sequence: Synthetic polypeptide
EVQLVESGGGLIQPGGSLRLSCAVSGFSLSSYGVSWVRQAPGKGLEYIGFIGSGGFAYYASWAKSRSTISRDNSKNTVYLQMNSLRAEDTAVYFCARDDVGGGKSLDIWGPGTLVTVSS

SEQ ID NO: 215 - protein, 119 aa, synthetic construct
  REGION 1..119: note = Description of Artificial Sequence: Synthetic polypeptide
EVQLVESGGGLIQPGGSLRLSCAVSGFSLSSYGVSWVRQAPGKGLEYIGFIGSGGFAYYASWAKSRSTISRDTSKNTLYLQMNSLRAEDTAVYFCARDDVGGGKSLDIWGPGTLVTVSS

SEQ ID NO: 216 - protein, 119 aa, synthetic construct
  REGION 1..119: note = Description of Artificial Sequence: Synthetic polypeptide
EVQLVESGGGLIQPGGSLRLSCAVSGFSLSSYGVSWVRQAPGKGLEYIGFIGSGGFAYYASWAKSRSTISRDTSKNTVYLQMNSLRAEDTAVYYCARDDVGGGKSLDIWGPGTLVTVSS

SEQ ID NO: 217 - protein, 119 aa, synthetic construct
  REGION 1..119: note = Description of Artificial Sequence: Synthetic polypeptide
EVQLVESGGGLIQPGGSLRLSCAVSGFSLSSYGVSWVRQAPGKGLEYIGFIGSGGFAYYASWAKSRSTISRDTSKNTVYLQMNSLRAEDTAVYFCARDDVGGGKSLDIWGQGTLVTVSS

SEQ ID NO: 218 - protein, 119 aa, synthetic construct
  REGION 1..119: note = Description of Artificial Sequence: Synthetic polypeptide
EVQLVESGGGLIQPGGSLRLSCAVSGFSLSSYGVSWVRQAPGKGLEYIGFIGSGGFAYYADSVKGRSTISRDTSKNTVYLQMNSLRAEDTAVYFCARDDVGGGKSLDIWGPGTLVTVSS

SEQ ID NO: 219 - protein, 119 aa, synthetic construct
  REGION 1..119: note = Description of Artificial Sequence: Synthetic polypeptide
EVQLVESGGGLIQPGGSLRLSCAASGFSLSSYGVSWVRQAPGKGLEWVSFIGSGGFAYYASWAKSRFTISRDNSKNTLYLQMNSLRAEDTAVYYCARDDVGGGKSLDIWGQGTLVTVSS

SEQ ID NO: 220 - protein, 119 aa, synthetic construct
  REGION 1..119: note = Description of Artificial Sequence: Synthetic polypeptide
EVQLVESGGGLIQPGGSLRLSCAASGFSLSSYGVSWVRQAPGKGLEWVSFIGSGGFAYYADSVKGRFTISRDNSKNTLYLQMNSLRAEDTAVYYCARDDVGGGKSLDIWGQGTLVTVSS

SEQ ID NO: 221 - protein, 119 aa, synthetic construct
  REGION 1..119: note = Description of Artificial Sequence: Synthetic polypeptide
EVQLVESGGGLIQPGGSLRLSCAASGFSLSSYGVSWVRQAPGKGLEYVGFIGSGGFAYYASWAKSRFTISRDNSKNTLYLQMNSLRAEDTAVYYCARDDVGGGKSLDIWGQGTLVTVSS

SEQ ID NO: 222 - protein, 119 aa, synthetic construct
  REGION 1..119: note = Description of Artificial Sequence: Synthetic polypeptide
EQQLVESGGGLIQPGGSLRLSCAASGFSLSSYGVSWVRQAPGKGLEYVGFIGSGGFAYYASWAKSRFTISRDNSKNTLYLQMNSLRAEDTAVYYCARDDVGGGKSLDIWGQGTLVTVSS

SEQ ID NO: 223 - protein, 119 aa, synthetic construct
  REGION 1..119: note = Description of Artificial Sequence: Synthetic polypeptide
EVQLVESGGGLIQPGGSLRLSCAASGFSLSSYGVSWVRQAPGKGLEYVGFIGSGGFAYYASWAKSRFTISRNTNLNTLYLQMNSLRAEDTAVYYCARDDVGGGKSLDIWGQGTLVTVSS

SEQ ID NO: 224 - protein, 119 aa, synthetic construct
  REGION 1..119: note = Description of Artificial Sequence: Synthetic polypeptide
EVQLVESGGGLIQPGGSLRLSCAASGFSLSSYGVSWVRQAPGKGLEYVGFIGSGGFAYYASFAKSRFTISRDNSKNTLYLQMNSLRAEDTAVYYCARDDVGGGKSLDIWGQGTLVTVSS

SEQ ID NO: 225 - protein, 119 aa, synthetic construct
  REGION 1..119: note = Description of Artificial Sequence: Synthetic polypeptide
  VARIANT 2
  VARIANT 24
  VARIANT 47
  VARIANT 48
  VARIANT 49
  VARIANT 61
  VARIANT 62
  VARIANT 63
  VARIANT 65
  VARIANT 67
  VARIANT 72
  VARIANT 73
  VARIANT 74
  VARIANT 75
  VARIANT 78
  VARIANT 94
  VARIANT 111
EXQLVESGGGLIQPGGSLRLSCAXSGFSLSSYGVSWVRQAPGKGLEXXXFIGSGGFAYYAXXXKXRXTISRXXXXNTXYLQMNSLRAEDTAVYXCARDDVGGGKSLDIWGXGTLVTVSS

SEQ ID NO: 226 - protein, 118 aa, synthetic construct
  REGION 1..118: note = Description of Artificial Sequence: Synthetic polypeptide
QSVKESEGGLFKPTDNLTLTCTVSGFSLSSYGVSWVRQAPGNGLEYIGFIGSGGSPYYASWAKSRSTITRNTNLNTVTLKMTSLTAADTATYFCARDDVGGGKSLDIWGPGTLVTVSS

SEQ ID NO: 227 - protein, 118 aa, synthetic construct
  REGION 1..118: note = Description of Artificial Sequence: Synthetic polypeptide
QSVKESEGGLFKPTDNLTLTCTVSGFSLSSYGVSWVRQAPGNGLEYIGFIGSGGFAYYASWAKSRSTITRNTNLNTVTLKMTSLTAADTATYFCARDDVGGGRSLDIWGPGTVVTVSS

SEQ ID NO: 228 - protein, 119 aa, synthetic construct
  REGION 1..119: note = Description of Artificial Sequence: Synthetic polypeptide
QSVKESEGGLFKPTDTLTLTCTVSGFSLSSYPISWVRQAPGNGLEWIGYITSEYGVAYYATWAESRSTITRNTNLNTVTLKMTSLTAADTATYFCVRENPTYGYAYDAWGPGTLVTVSS

SEQ ID NO: 229 - protein, 124 aa, synthetic construct
  REGION 1..124: note = Description of Artificial Sequence: Synthetic polypeptide
QSVKESEGGLFKPTDTLTLTCTVSGFSLNDYGVSWVRQAPGNGLEWIGAIGSSGVAWYANWAKGRSTITRNTNLNTVTLKMASLTAADTATYFCARDRDYGYRADDATSGMDLWGPGTLVTVSS

SEQ ID NO: 230 - protein, 125 aa, synthetic construct
  REGION 1..125: note = Description of Artificial Sequence: Synthetic polypeptide
EVQLVESGGGLVQPGGSLRLSCAVSGFSLNDYGVSWVRQAPGKGLEWIGAIGSSGVAWYANWAKGRSTISRHTSKNTVYLQMNSLRAEDTAVYFCARDRDYGYRADDATSGMDLWGPGTLVTVSS

SEQ ID NO: 231 - protein, 125 aa, synthetic construct
  REGION 1..125: note = Description of Artificial Sequence: Synthetic polypeptide
EVQLVESGGGLVQPGGSLRLSCAASGFSLNDYGVSWVRQAPGKGLEWIGAIGSSGVAWYANWAKGRSTISRHTSKNTVYLQMNSLRAEDTAVYFCARDRDYGYRADDATSGMDLWGPGTLVTVSS

SEQ ID NO: 232 - protein, 125 aa, synthetic construct
  REGION 1..125: note = Description of Artificial Sequence: Synthetic polypeptide
EVQLVESGGGLVQPGGSLRLSCAVSGFSLNDYGVSWVRQAPGKGLEWVGAIGSSGVAWYANWAKGRSTISRHTSKNTVYLQMNSLRAEDTAVYFCARDRDYGYRADDATSGMDLWGPGTLVTVSS

SEQ ID NO: 233 - protein, 125 aa, synthetic construct
  REGION 1..125: note = Description of Artificial Sequence: Synthetic polypeptide
EVQLVESGGGLVQPGGSLRLSCAVSGFSLNDYGVSWVRQAPGKGLEWISAIGSSGVAWYANWAKGRSTISRHTSKNTVYLQMNSLRAEDTAVYFCARDRDYGYRADDATSGMDLWGPGTLVTVSS

SEQ ID NO: 234 - protein, 125 aa, synthetic construct
  REGION 1..125: note = Description of Artificial Sequence: Synthetic polypeptide
EVQLVESGGGLVQPGGSLRLSCAVSGFSLNDYGVSWVRQAPGKGLEWIGAIGSSGVAWYANWAKGRFTISRHTSKNTVYLQMNSLRAEDTAVYFCARDRDYGYRADDATSGMDLWGPGTLVTVSS

SEQ ID NO: 235 - protein, 125 aa, synthetic construct
  REGION 1..125: note = Description of Artificial Sequence: Synthetic polypeptide
EVQLVESGGGLVQPGGSLRLSCAVSGFSLNDYGVSWVRQAPGKGLEWIGAIGSSGVAWYANWAKGRSTISRHNSKNTVYLQMNSLRAEDTAVYFCARDRDYGYRADDATSGMDLWGPGTLVTVSS

SEQ ID NO: 236 - protein, 125 aa, synthetic construct
  REGION 1..125: note = Description of Artificial Sequence: Synthetic polypeptide
EVQLVESGGGLVQPGGSLRLSCAVSGFSLNDYGVSWVRQAPGKGLEWIGAIGSSGVAWYANWAKGRSTISRHTSKNTLYLQMNSLRAEDTAVYFCARDRDYGYRADDATSGMDLWGPGTLVTVSS

SEQ ID NO: 237 - protein, 125 aa, synthetic construct
  REGION 1..125: note = Description of Artificial Sequence: Synthetic polypeptide
EVQLVESGGGLVQPGGSLRLSCAVSGFSLNDYGVSWVRQAPGKGLEWIGAIGSSGVAWYANWAKGRSTISRHTSKNTVYLQMNSLRAEDTAVYYCARDRDYGYRADDATSGMDLWGPGTLVTVSS

SEQ ID NO: 238 - protein, 125 aa, synthetic construct
  REGION 1..125: note = Description of Artificial Sequence: Synthetic polypeptide
EVQLVESGGGLVQPGGSLRLSCAVSGFSLNDYGVSWVRQAPGKGLEWIGAIGSSGVAWYANWAKGRSTISRHTSKNTVYLQMNSLRAEDTAVYFCARDRDYGYRADDATSGMDLWGRGTLVTVSS

SEQ ID NO: 239 - protein, 125 aa, synthetic construct
  REGION 1..125: note = Description of Artificial Sequence: Synthetic polypeptide
EVQLVESGGGLVQPGGSLRLSCAVSGFSLNDYGVSWVRQAPGKGLEWIGAIGSSGVAFYANWAKGRSTISRHTSKNTVYLQMNSLRAEDTAVYFCARDRDYGYRADDATSGMDLWGPGTLVTVSS

SEQ ID NO: 240 - protein, 125 aa, synthetic construct
  REGION 1..125: note = Description of Artificial Sequence: Synthetic polypeptide
EVQLVESGGGLVQPGGSLRLSCAVSGFSLNDYGVSWVRQAPGKGLEWIGAIGSSGVAWYADSVKGRSTISRHTSKNTVYLQMNSLRAEDTAVYFCARDRDYGYRADDATSGMDLWGPGTLVTVSS

SEQ ID NO: 241 - protein, 125 aa, synthetic construct
  REGION 1..125: note = Description of Artificial Sequence: Synthetic polypeptide
EVQLVESGGGLVQPGGSLRLSCAASGFSLNDYGVSWVRQAPGKGLEWVSAIGSSGVAFYANWAKGRFTISRHNSKNTLYLQMNSLRAEDTAVYYCARDRDYGYRADDATSGMDLWGRGTLVTVSS

SEQ ID NO: 242 - protein, 125 aa, synthetic construct
  REGION 1..125: note = Description of Artificial Sequence: Synthetic polypeptide
EVQLVESGGGLVQPGGSLRLSCAASGFSLNDYGVSWVRQAPGKGLEWVSAIGSSGVAFYADSVKGRFTISRHNSKNTLYLQMNSLRAEDTAVYYCARDRDYGYRADDATSGMDLWGRGTLVTVSS

SEQ ID NO: 243 - protein, 125 aa, synthetic construct
  REGION 1..125: note = Description of Artificial Sequence: Synthetic polypeptide
EVQLLESGGGLVQPGGSLRLSCAASGFSLNDYGVSWVRQAPGKGLEWVGAIGSSGVAWYANWAKGRFTISRDNSKNTLYLQMNSLRAEDTAVYYCARDRDYGYRADDATSGMDLWGRGTLVTVSS

SEQ ID NO: 244 - protein, 125 aa, synthetic construct
  REGION 1..125: note = Description of Artificial Sequence: Synthetic polypeptide
EVQLLESGGGLVQPGGSLRLSCAASGFSLNDYGVSWVRQAPGKGLEWVGAIGSSGVAFYANWAKGRFTISRDNSKNTLYLQMNSLRAEDTAVYYCARDRDYGYRADDATSGMDLWGRGTLVTVSS

SEQ ID NO: 245 - protein, 125 aa, synthetic construct
  REGION 1..125: note = Description of Artificial Sequence: Synthetic polypeptide
EQQLLESGGGLVQPGGSLRLSCAASGFSLNDYGVSWVRQAPGKGLEWVGAIGSSGVAWYANWAKGRFTISRDNSKNTLYLQMNSLRAEDTAVYYCARDRDYGYRADDATSGMDLWGRGTLVTVSS

SEQ ID NO: 246 - protein, 125 aa, synthetic construct
  REGION 1..125: note = Description of Artificial Sequence: Synthetic polypeptide
EVQLLESGGGLVQPGGSLRLSCAASGFSLNDYGVSWVRQAPGKGLEWVGAIGSSGVAWYANWAKGRFTISRNTNLNTLYLQMNSLRAEDTAVYYCARDRDYGYRADDATSGMDLWGRGTLVTVSS

SEQ ID NO: 247 - protein, 125 aa, synthetic construct
  REGION 1..125: note = Description of Artificial Sequence: Synthetic polypeptide
EVQLLESGGGLVQPGGSLRLSCAASGFSLNDYGVSWVRQAPGKGLEWVGAIGSSGVAWYANFAKGRFTISRDNSKNTLYLQMNSLRAEDTAVYYCARDRDYGYRADDATSGMDLWGRGTLVTVSS

SEQ ID NO: 248 - protein, 125 aa, synthetic construct
  REGION 1..125: note = Description of Artificial Sequence: Synthetic polypeptide
EVQLLESGGGLVQPGGSLRLSCAVSGFSLNDYGVSWVRQAPGKGLEWVGAIGSSGVAWYANFAKGRFTISRDNSKNTLYLQMNSLRAEDTAVYYCARDRDYGYRADDATSGMDLWGRGTLVTVSS

SEQ ID NO: 249 - protein, 125 aa, synthetic construct
  REGION 1..125: note = Description of Artificial Sequence: Synthetic polypeptide
EVQLLESGGGLVQPGGSLRLSCAASGFSLNDYGVSWVRQAPGKGLEWIGAIGSSGVAWYANFAKGRFTISRDNSKNTLYLQMNSLRAEDTAVYYCARDRDYGYRADDATSGMDLWGRGTLVTVSS

SEQ ID NO: 250 - protein, 125 aa, synthetic construct
  REGION 1..125: note = Description of Artificial Sequence: Synthetic polypeptide
EVQLLESGGGLVQPGGSLRLSCAASGFSLNDYGVSWVRQAPGKGLEWVGAIGSSGVAWYANFAKGRSTISRDNSKNTLYLQMNSLRAEDTAVYYCARDRDYGYRADDATSGMDLWGRGTLVTVSS

SEQ ID NO: 251 - protein, 125 aa, synthetic construct
  REGION 1..125: note = Description of Artificial Sequence: Synthetic polypeptide
EVQLLESGGGLVQPGGSLRLSCAASGFSLNDYGVSWVRQAPGKGLEWIGAIGSSGVAWYANFAKGRSTISRDNSKNTLYLQMNSLRAEDTAVYYCARDRDYGYRADDATSGMDLWGRGTLVTVSS

SEQ ID NO: 252 - protein, 125 aa, synthetic construct
  REGION 1..125: note = Description of Artificial Sequence: Synthetic polypeptide
EVQLLESGGGLVQPGGSLRLSCAASGFSLNDYGVSWVRQAPGKGLEWVGAIGSSGVAWYANFAKGRFTISRDTSKNTLYLQMNSLRAEDTAVYYCARDRDYGYRADDATSGMDLWGRGTLVTVSS

SEQ ID NO: 253 - protein, 125 aa, synthetic construct
  REGION 1..125: note = Description of Artificial Sequence: Synthetic polypeptide
EVQLLESGGGLVQPGGSLRLSCAASGFSLNDYGVSWVRQAPGKGLEWVGAIGSSGVAWYANFAKGRFTISRDNSKNTVYLQMNSLRAEDTAVYYCARDRDYGYRADDATSGMDLWGRGTLVTVSS

SEQ ID NO: 254 - protein, 125 aa, synthetic construct
  REGION 1..125: note = Description of Artificial Sequence: Synthetic polypeptide
EVQLLESGGGLVQPGGSLRLSCAASGFSLNDYGVSWVRQAPGKGLEWVGAIGSSGVAWYANFAKGRFTISRDTSKNTVYLQMNSLRAEDTAVYYCARDRDYGYRADDATSGMDLWGRGTLVTVSS

SEQ ID NO: 255 - protein, 125 aa, synthetic construct
  REGION 1..125: note = Description of Artificial Sequence: Synthetic polypeptide
EVQLLESGGGLVQPGGSLRLSCAASGFSLNDYGVSWVRQAPGKGLEWIGAIGSSGVAWYANFAKGRSTISRDTSKNTVYLQMNSLRAEDTAVYYCARDRDYGYRADDATSGMDLWGRGTLVTVSS

SEQ ID NO: 256 - protein, 125 aa, synthetic construct
  REGION 1..125: note = Description of Artificial Sequence: Synthetic polypeptide
EVQLLESGGGLVQPGGSLRLSCAASGFSLNDYGVSWVRQAPGKGLEWIGAIGSSGVAFYANFAKGRSTISRDTSKNTVYLQMNSLRAEDTAVYYCARDRDYGYRADDATSGMDLWGRGTLVTVSS

SEQ ID NO: 257 - protein, 125 aa, synthetic construct
  REGION 1..125: note = Description of Artificial Sequence: Synthetic polypeptide
  VARIANT 2
  VARIANT 5
  VARIANT 24
  VARIANT 48
  VARIANT 49
  VARIANT 58
  VARIANT 61
  VARIANT 62
  VARIANT 63
  VARIANT 67
  VARIANT 72
  VARIANT 73
  VARIANT 74
  VARIANT 75
  VARIANT 78
  VARIANT 94
  VARIANT 117
EXQLXESGGGLVQPGGSLRLSCAXSGFSLNDYGVSWVRQAPGKGLEWXXAIGSSGVAXYAXXXKGRXTISRXXXXNTXYLQMNSLRAEDTAVYXCARDRDYGYRADDATSGMDLWGXGTLVTVSS

SEQ ID NO: 258 - protein, 122 aa, synthetic construct
  REGION 1..122: note = Description of Artificial Sequence: Synthetic polypeptide
QEQLEESGGGLVKPGASLTLTCTVSGFSLTNNYVNFVMCWVRQAPGKGLEWIASIDPGDDSTDYASWATGRFTISKASSTTVTLQVTSLTAADTATYFCARGDAGTSYSFNFWGPGTLVTVS

SEQ ID NO: 259 - protein, 120 aa, synthetic construct
  REGION 1..120: note = Description of Artificial Sequence: Synthetic polypeptide
QEQLVESGGGLVKPGASLTLTCTASGFSLTDNYVMSWVRQAPGKGLEWIACIDPGDDSTYYASWATGRFTISRASSTTVTLQVTSLTAADTATYFCARGDAAASYSFNFWGPGTLVTVSS

SEQ ID NO: 260 - protein, 117 aa, synthetic construct
  REGION 1..117: note = Description of Artificial Sequence: Synthetic polypeptide
QSLEESGGGLVKPGGTLTLTCKASGIDFNGGGIYWVRQAPGKGLEWIASIYPDHGSVDYANWVNGRFTISLDNAQNTVFLQLTSLTVADTATYFCARESGGSYYDLWGPGTLVTVSS

SEQ ID NO: 261 - protein, 117 aa, synthetic construct
  REGION 1..117: note = Description of Artificial Sequence: Synthetic polypeptide
QSLEESGGGLVKPGGTLTLTCTASGFDFNGGGIYWVRQAPGKGLEWIAYIYPDHGSADYATWVNGRFTISLDNAQNTVFLQMTSLTVADTATYFCARETGGSWYDLWGPGTLVTVSS

SEQ ID NO: 262 - protein, 214 aa, synthetic construct
  REGION 1..214: note = Description of Artificial Sequence: Synthetic polypeptide
DIQMTQSPASLSASLGETVTIQCRASEDIYSGLAWYQQKPGKSPQLLIYGATTLHDGVPSRFSGSGSGTQYSLKISSMHSEDEGIYFCHQGLSFPYTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 263 - protein, 214 aa, synthetic construct
  REGION 1..214: note = Description of Artificial Sequence: Synthetic polypeptide
DIQMTQSPASLSASLGETVTIECLASKNIYRNLAWYQQKPGKSPQFLISDASRLQDGVPSRFTGSDSGSQYSLKINSLQSEDVATYFCQQYHDYPYTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 264 - protein, 216 aa, synthetic construct
  REGION 1..216: note = Description of Artificial Sequence: Synthetic polypeptide
DVVMTQTASPVSAAVGGTVTIKCQASENIYSLLAWYQQKPGQPPKVLIYDASDLASGVPSRFKGSGSGTQFTLTISDLECADAATYYCQATAYGSSGNAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 265 - protein, 216 aa, synthetic construct
  REGION 1..216: note = Description of Artificial Sequence: Synthetic polypeptide
DVVMTQTPASVSEPVGGTVTIKCQASENIYSLLAWYQQKPGQPPKVLIYDASDLASGVPSRFKGSGSGTQFTLTISDLECADAATYYCQATAYGSSGNAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 266 - protein, 217 aa, synthetic construct
  REGION 1..217: note = Description of Artificial Sequence: Synthetic polypeptide
NIVMTQTPASVEVAVGGTVVIKCQASQNIGDYLSWYQQKPGQRPKLLIYSASTLASGVPSRFKGSGSGTQFTLTISDLECADAATYYCHQDYTSNDVENTFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 267 - protein, 217 aa, synthetic construct
  REGION 1..217: note = Description of Artificial Sequence: Synthetic polypeptide
AYYMTQTPASVEVAVGGTVTIKCQASEDIGSYCSWYQQKPGQPPKLLIYDASDLASGVPSRFKGSGSGTDFTLTISGVQCDDAATYYCQQDYTGNNVDNTFGGGSEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 268 - protein, 217 aa, synthetic construct
  REGION 1..217: note = Description of Artificial Sequence: Synthetic polypeptide
AYDMTQTPASLEAAVGGTVTINCQASESISNELSWYQQKPGQPPDLLIYYASTLASGVPSRFKGSGSGTEFTLTISDLECADAATYYCAQGFGSSGVENVFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 269 - protein, 217 aa, synthetic construct
  REGION 1..217: note = Description of Artificial Sequence: Synthetic polypeptide
AIRMTQSPSSFSASTGDRVTITCQASESISNELSWYQQKPGKAPKLLIYYASTLASGVPSRFSGSGSGTDFTLTISSLQPEDFATYYCAQGFGSSGVENVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 270 - protein, 217 aa, synthetic construct
  REGION 1..217: note = Description of Artificial Sequence: Synthetic polypeptide
AYYMTQTPASVEAAVGGTVTIKCQASQSISNYVAWYQQKPGQPPKLLIYRASTLASGVSSRFSGSGSGTEFTLTISDLECADAATYYCHQGYSSSNVDNIFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 271 - protein, 217 aa, synthetic construct
  REGION 1..217: note = Description of Artificial Sequence: Synthetic polypeptide
AYEMTQTPASVEVAVGGTVTINCQASESISSDLAWYQQKPGQRPKLLIYAASTLASGVPSRFKGSGSGTEFTLSISGVQCADAATYYCQQGYTWNNVDNVFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 272 - protein, 217 aa, synthetic construct
  REGION 1..217: note = Description of Artificial Sequence: Synthetic polypeptide
DIVMTQTPASVSEPVGGTVTIKCQASQNINNYLSWYQQKPGQPPKQLIYAASTLASGVPSRFKGSGSGTQFTLTISDLECADAATYYCHQDYTSNNVDNTFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 273 - protein, 217 aa, synthetic construct
  REGION 1..217: note = Description of Artificial Sequence: Synthetic polypeptide
NIVMTQTPASVEVAVGGTVTIKCQASQSIGSYLSWYQQKPGQPPKLLIYDASNLASGVPSRFKGSGSGTQFTLSISDLECADAATYYCHQDYTSNNVDNTFGGGTEVVIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 274 - protein, 217 aa, synthetic construct
  REGION 1..217: note = Description of Artificial Sequence: Synthetic polypeptide
NIVMTQTPASVEAAVGGTVTINCQASQSISSYLSWYQQKSGQRPKLLIFSASTLASGVPSRFTGSGSGTQFTLTISDLQCADAATYYCHQDYTSSNVDNTFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 275 - protein, 217 aa, synthetic construct
  REGION 1..217: note = Description of Artificial Sequence: Synthetic polypeptide
DIQMTQSPSSLSASVGDRVTITCQASQSISSYLSWYQQKPGKAPKLLIYSASTLASGVPSRFSGSGSGTDFTLTISSLQPEDFATYYCHQDYTSSNVDNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 276 - protein, 217 aa, synthetic construct
  REGION 1..217: note = Description of Artificial Sequence: Synthetic polypeptide
NIVMTQTPASVEVAVGGTVIIKCQASQSISNYLSWYHQKSGQRPRLLIYSASTLASGVPSRFKGSGSGTQFTLTISDLECADAATYYCHQDYTSNSVDNTFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 277 - protein, 217 aa, synthetic construct
  REGION 1..217: note = Description of Artificial Sequence: Synthetic polypeptide
NIVMTQTPASVEVAMGGTVIIKCQASQSISTYLSWYQQKPGQPPKLLIYSASTLASGVSSRFEGSGSGTQFTLTISGVQCADAATYYCHQDYTSNNVDNTFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 278 - protein, 217 aa, synthetic construct
  REGION 1..217: note = Description of Artificial Sequence: Synthetic polypeptide
AYDLTQTPASVEAAVGGTVTINCQASESIGNELSWYQQKSGQPPKLLIYQASTLASGVPSRFKGSGSGTDFTLTISDLECADAATYYCAQGFSSSGVENVFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 279 - protein, 217 aa, synthetic construct
  REGION 1..217: note = Description of Artificial Sequence: Synthetic polypeptide
AYDMTQTPASVEAAVGGTVTIKCQASESIGNALAWYQQKPGQPPKLLIYDASDLASGVPSRFKGSGSGTQFTLTISGVECADAATYYCQQGDSHNNVDNIFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 280 - protein, 217 aa, synthetic construct
  REGION 1..217: note = Description of Artificial Sequence: Synthetic polypeptide
AIRMTQSPSSFSASTGDRVTITCQASESIGNALAWYQQKPGKAPKLLIYDASDLASGVPSRFSGSGSGTDFTLTISSLQPEDFATYYCQQGDSHNNVDNIFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 281 - protein, 217 aa, synthetic construct
  REGION 1..217: note = Description of Artificial Sequence: Synthetic polypeptide
AIEMTQTPSSASEPVGGTVTIKCQASESISRYLSWYQQKPGQPPKLLIYDASDLASGVSSRFKGSGSGTQFTLIISDVECADAATYYCQQDYSRSNIVNSFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 282 - protein, 217 aa, synthetic construct
  REGION 1..217: note = Description of Artificial Sequence: Synthetic polypeptide
AYDMTQTPSSASEPVGGTVTIKCQASESISTYLSWYQQKPGQPPKLLIYDASDLASGVSSRFKGSGSGTQFTLTISDVECADAATYYCQQDYSSSNIVNSFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 283 - protein, 217 aa, synthetic construct
  REGION 1..217: note = Description of Artificial Sequence: Synthetic polypeptide
AYDMTQTPASVEVAVGGTVTIKCQASQSISTYLAWYQQKPGQRPNLLIYKTSTLASGVPSRFRGSGSGTQFTLTISGVECADAATYYCQQGYSGSSVENTFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 284 - protein, 217 aa, synthetic construct
  REGION 1..217: note = Description of Artificial Sequence: Synthetic polypeptide
AYDMTQTPVSVEAAVGGTVTIKCQASQSISSYLAWYQQKPGQPPKLLIYKASTLASGVSSRFKGSGSGTEFTLTISDLECADAATYYCQQGYSGSNVENTFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 285 - protein, 451 aa, synthetic construct
  REGION 1..451: note = Description of Artificial Sequence: Synthetic polypeptide
EVQLVESGGGLVQPGRSLKLSCTASGFTFSDYNMAWVRQAPKGGLEWVTTISYDAGRTYYRDSVKGRFTISRDNAKRTLSLQMDSLRSEDTATYYCATGIFNYGTDYFDYWGQGVMVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 286 - protein, 451 aa, synthetic construct
  REGION 1..451: note = Description of Artificial Sequence: Synthetic polypeptide
EVQLVESGGGLVRPGRSLRLSCAASGFTFSDYYMAWVRQAPTKGLEWVASISYDGDTTYYRDSVKGRFTISRDNARSSLYLQMDSLRSDDTANYFCTTDGTIPAGSWFAYWGQGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 287 - protein, 450 aa, synthetic construct
  REGION 1..450: note = Description of Artificial Sequence: Synthetic polypeptide
QEQLEESGGDLVKPEGSLTLTCTASGFSFSSSYWICWVRQAPGKGLEWIACVYGLDVNIYYASWTKGRFTISKTSSTTVTLQMTSLTAADTATYFCARGGGSADFGFDLWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 288 - protein, 449 aa, synthetic construct
  REGION 1..449: note = Description of Artificial Sequence: Synthetic polypeptide
QSLEESGGDLVKPEGSLTLTCTASGFSFSTSYWICWVRQAPGKGLEWIACVYGLDVNIYYASWTEGRFTISKTSSTTVTLQVTSLTAADTATYFCARGGGSADFGFDLWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 289 - protein, 448 aa, synthetic construct
  REGION 1..448: note = Description of Artificial Sequence: Synthetic polypeptide
QSVKESEGGLFKPTDNLTLTCTVSGFSLSNYGVTWVRQAPGNGLEYIGFIGSGGSAYYASWAKSRSTITRNTNLNTVTLKMTSLTAADTATYFCARDDVGGGKSLDIWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 290 - protein, 449 aa, synthetic construct
  REGION 1..449: note = Description of Artificial Sequence: Synthetic polypeptide
QSVEESRGGLIKPTDTLTLTCTASGFSLSNYGVSWVRQAPGNGLEYIGFIGYGGSTYYASWAKSRSTITRNTNLNTVTLQMTSLTAADTATYFCARLCGVDCADALDSWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 291 - protein, 449 aa, synthetic construct
  REGION 1..449: note = Description of Artificial Sequence: Synthetic polypeptide
QSLEESGGGLVKPTDTLTLTCTVSGFSLSSYGVTWVRQAPGRGLEWIGYITSNYGVSYYASWAKSRSTITRNTNLNTVTLKMTSLTAADTATYFCARENPDYGYAYDAWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 292 - protein, 450 aa, synthetic construct
  REGION 1..450: note = Description of Artificial Sequence: Synthetic polypeptide
EVQLVESGGGLVQPGGSLRLSCAASGFSLSSYGVTWVRQAPGKGLEWIGYITSNYGVSYYASWAKSRSTISRDTSKNTVYLQMGSLRAEDMAVYYCARENPDYGYAYDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 293 - protein, 447 aa, synthetic construct
  REGION 1..447: note = Description of Artificial Sequence: Synthetic polypeptide
EVQLVESGGGLVQPGGSLRLSCAASGFSLSSYGVTWVRQAPGKGLEWIGYITSNYGVSYYASWAKSRSTISRDTSKNTVYLQMGSLRAEDMAVYYCARENPDYGYAYDAWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 294 - protein, 450 aa, synthetic construct
  REGION 1..450: note = Description of Artificial Sequence: Synthetic polypeptide
QSVKESEGGLFKPTDTLTLTCTVSGFSLINYGVSWVRQAPGKGLEWIGYIGSAGSTYYATWAKSRATITRNTNLNTVTLKMTSLTAADTATYFCARAAYSAGSADAEDIWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 295 - protein, 449 aa, synthetic construct
  REGION 1..449: note = Description of Artificial Sequence: Synthetic polypeptide
QSVKESEGGLIKPTDTLTLTCTVSGFSLSTFAINWVRQAPGNGLEWIGAIGRGGSAYYASWAKSRSTITKNTNLNTVTLKMTRPTAADTATYFCARENAGSGWGELDIWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 296 - protein, 448 aa, synthetic construct
  REGION 1..448: note = Description of Artificial Sequence: Synthetic polypeptide
QSVKESEGGLFKPTDTLTLTCTVSGFSLSGYGVSWVRQAPGKGLEYIGFIGSGGSAYYASWAKSRSTITRNTNLNTVTLKMTRLTAADTATYFCARDNVGGDMSLDIWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 297 - protein, 448 aa, synthetic construct
  REGION 1..448: note = Description of Artificial Sequence: Synthetic polypeptide
QSVKESEGGLFKPTDNLTLTCTVSGFSLSNYGVTWVRQAPGNGLEYIGFIGSSGSAYYASWAKSRSTITRNTNLNTVTLKMTSLTAADTATYFCARDDVGGGKSLDIWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 298 - protein, 448 aa, synthetic construct
  REGION 1..448: note = Description of Artificial Sequence: Synthetic polypeptide
QSVKESEGGLFKPTDNLTLTCTVSGFSLSSYGVSWVRQAPGNGLEYIGFIGSGGFAYYASWAKSRSTITRNTNLNTVTLKMTSLTAADTATYFCARDDVGGGKSLDIWGPGTVVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 299 - protein, 449 aa, synthetic construct
  REGION 1..449: note = Description of Artificial Sequence: Synthetic polypeptide
EVQLVESGGGLIQPGGSLRLSCAASGFSLSSYGVSWVRQAPGKGLEYVGFIGSGGFAYYASWAKSRFTISRDNSKNTLYLQMNSLRAEDTAVYYCARDDVGGGKSLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 300 - protein, 446 aa, synthetic construct
  REGION 1..446: note = Description of Artificial Sequence: Synthetic polypeptide
EVQLVESGGGLIQPGGSLRLSCAASGFSLSSYGVSWVRQAPGKGLEYVGFIGSGGFAYYASWAKSRFTISRDNSKNTLYLQMNSLRAEDTAVYYCARDDVGGGKSLDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 301 - protein, 448 aa, synthetic construct
  REGION 1..448: note = Description of Artificial Sequence: Synthetic polypeptide
QSVKESEGGLFKPTDNLTLTCTVSGFSLSSYGVSWVRQAPGNGLEYIGFIGSGGSPYYASWAKSRSTITRNTNLNTVTLKMTSLTAADTATYFCARDDVGGGKSLDIWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 302 - protein, 448 aa, synthetic construct
  REGION 1..448: note = Description of Artificial Sequence: Synthetic polypeptide
QSVKESEGGLFKPTDNLTLTCTVSGFSLSSYGVSWVRQAPGNGLEYIGFIGSGGFAYYASWAKSRSTITRNTNLNTVTLKMTSLTAADTATYFCARDDVGGGRSLDIWGPGTVVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 303 - protein, 449 aa, synthetic construct
  REGION 1..449: note = Description of Artificial Sequence: Synthetic polypeptide
QSVKESEGGLFKPTDTLTLTCTVSGFSLSSYPISWVRQAPGNGLEWIGYITSEYGVAYYATWAESRSTITRNTNLNTVTLKMTSLTAADTATYFCVRENPTYGYAYDAWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 304 - protein, 454 aa, synthetic construct
  REGION 1..454: note = Description of Artificial Sequence: Synthetic polypeptide
QSVKESEGGLFKPTDTLTLTCTVSGFSLNDYGVSWVRQAPGNGLEWIGAIGSSGVAWYANWAKGRSTITRNTNLNTVTLKMASLTAADTATYFCARDRDYGYRADDATSGMDLWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 305 - protein, 455 aa, synthetic construct
  REGION 1..455: note = Description of Artificial Sequence: Synthetic polypeptide
EVQLLESGGGLVQPGGSLRLSCAVSGFSLNDYGVSWVRQAPGKGLEWVGAIGSSGVAWYANFAKGRFTISRDNSKNTLYLQMNSLRAEDTAVYYCARDRDYGYRADDATSGMDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - protein, 452 aa, synthetic construct
  REGION 1..452: note = Description of Artificial Sequence: Synthetic polypeptide
EVQLLESGGGLVQPGGSLRLSCAVSGFSLNDYGVSWVRQAPGKGLEWVGAIGSSGVAWYANFAKGRFTISRDNSKNTLYLQMNSLRAEDTAVYYCARDRDYGYRADDATSGMDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 307 - protein, 453 aa, synthetic construct
  REGION 1..453: note = Description of Artificial Sequence: Synthetic polypeptide
QEQLEESGGGLVKPGASLTLTCTVSGFSLTNNYVNFVMCWVRQAPGKGLEWIASIDPGDDSTDYASWATGRFTISKASSTTVTLQVTSLTAADTATYFCARGDAGTSYSFNFWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 308 - protein, 450 aa, synthetic construct
  REGION 1..450: note = Description of Artificial Sequence: Synthetic polypeptide
QEQLVESGGGLVKPGASLTLTCTASGFSLTDNYVMSWVRQAPGKGLEWIACIDPGDDSTYYASWATGRFTISRASSTTVTLQVTSLTAADTATYFCARGDAAASYSFNFWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 309 - protein, 447 aa, synthetic construct
  REGION 1..447: note = Description of Artificial Sequence: Synthetic polypeptide
QSLEESGGGLVKPGGTLTLTCKASGIDFNGGGIYWVRQAPGKGLEWIASIYPDHGSVDYANWVNGRFTISLDNAQNTVFLQLTSLTVADTATYFCARESGGSYYDLWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 310 - protein, 447 aa, synthetic construct
  REGION 1..447: note = Description of Artificial Sequence: Synthetic polypeptide
QSLEESGGGLVKPGGTLTLTCTASGFDFNGGGIYWVRQAPGKGLEWIAYIYPDHGSADYATWVNGRFTISLDNAQNTVFLQMTSLTVADTATYFCARETGGSWYDLWGPGTLVTVSSASTKGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 311 - protein, 107 aa, synthetic construct [sequence omitted]

SEQ ID NO: 312 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 327 aa, synthetic construct
  REGION 1..327: note = Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 315 - protein, 327 aa, synthetic construct
  REGION 1..327: note = Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 316 - protein, 6 aa, synthetic construct
  REGION 1..6: note = Description of Artificial Sequence: Synthetic peptide
LRPNQL

SEQ ID NO: 317 - protein, 6 aa, synthetic construct
  REGION 1..6: note = Description of Artificial Sequence: Synthetic peptide
QGVKSI

SEQ ID NO: 318 - protein, 14 aa, synthetic construct
  REGION 1..14: note = Description of Artificial Sequence: Synthetic peptide
KRCEDAYPRQIDDT

SEQ ID NO: 319 - protein, 13 aa, synthetic construct
  REGION 1..13: note = Description of Artificial Sequence: Synthetic peptide
DYPCARPNRPGVY

SEQ ID NO: 320 - protein, 299 aa, synthetic construct
  REGION 1..299: note = Description of Artificial Sequence: Synthetic polypeptide
IESAKQTKQMVDCSHYKKLPPGQQRFCHREYDPICGSDGKTYKNDCFFCSKVKKTDGTLKFVHFGKCGNSVTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 110 aa, synthetic construct
  REGION 1..110: note = Description of Artificial Sequence: Synthetic polypeptide
AIRMTQSPSSFSASTGDRVTITCQASESISNELSWYQQKPGKAPKLLIYYASTLASGVPSRFSGSGSGTDFTLTISSLQPEDFATYYCAQGFGSSGVENVFGGGTEVVVK

SEQ ID NO: 322 - protein, 224 aa, synthetic construct
  REGION 1..224: note = Description of Artificial Sequence: Synthetic polypeptide
EVQLVESGGGLVQPGGSLRLSCAASGFSLSSYGVTWVRQAPGKGLEWIGYITSNYGVSYYASWAKSRSTISRDTSKNTVYLQMGSLRAEDMAVYYCARENPDYGYAYDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 323 - protein, 217 aa, synthetic construct; identical to SEQ ID NO: 269
  REGION 1..217: note = Description of Artificial Sequence: Synthetic polypeptide

SEQ ID NO: 324 - protein, 223 aa, synthetic construct
  REGION 1..223: note = Description of Artificial Sequence: Synthetic polypeptide
EVQLVESGGGLIQPGGSLRLSCAASGFSLSSYGVSWVRQAPGKGLEYVGFIGSGGFAYYASWAKSRFTISRDNSKNTLYLQMNSLRAEDTAVYYCARDDVGGGKSLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 325 - protein, 217 aa, synthetic construct; identical to SEQ ID NO: 275
  REGION 1..217: note = Description of Artificial Sequence: Synthetic polypeptide

SEQ ID NO: 326 - protein, 229 aa, synthetic construct
  REGION 1..229: note = Description of Artificial Sequence: Synthetic polypeptide
EVQLLESGGGLVQPGGSLRLSCAVSGFSLNDYGVSWVRQAPGKGLEWVGAIGSSGVAWYANFAKGRFTISRDNSKNTLYLQMNSLRAEDTAVYYCARDRDYGYRADDATSGMDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 327 - protein, 217 aa, synthetic construct; identical to SEQ ID NO: 280
  REGION 1..217: note = Description of Artificial Sequence: Synthetic polypeptide

SEQ ID NO: 328 - protein, 227 aa, synthetic construct
  REGION 1..227: note = Description of Artificial Sequence: Synthetic polypeptide
IINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 329 - protein, 23 aa, synthetic construct
  REGION 1..23: note = Description of Artificial Sequence: Synthetic peptide
  VARIANT 2
AXRMTQSPSSFSASTGDRVTITC

SEQ ID NO: 330 - protein, 15 aa, synthetic construct
  REGION 1..15: note = Description of Artificial Sequence: Synthetic peptide
  VARIANT 9
WYQQKPGKXPKLLIY

SEQ ID NO: 331 - protein, 32 aa, synthetic construct
  REGION 1..32: note = Description of Artificial Sequence: Synthetic polypeptide
  VARIANT 21
  VARIANT 24
GVPSRFSGSGSGTDFTLTISXLQXEDFATYYC

SEQ ID NO: 332 - protein, 10 aa, synthetic construct
  REGION 1..10: note = Description of Artificial Sequence: Synthetic peptide
  VARIANT 6
  VARIANT 8
  VARIANT 9
FGGGTXVXXK

SEQ ID NO: 333 - protein, 30 aa, synthetic construct
  REGION 1..30: note = Description of Artificial Sequence: Synthetic polypeptide
  VARIANT 2
  VARIANT 24
EXQLVESGGGLVQPGGSLRLSCAXSGFSLS

SEQ ID NO: 334 - protein, 14 aa, synthetic construct
  REGION 1..14: note = Description of Artificial Sequence: Synthetic peptide
  MOD_RES 12: note = Trp or Tyr
  MOD_RES 13: note = Ile or Val
  MOD_RES 14: note = Gly or Ser
WVRQAPGKGLEXXX

SEQ ID NO: 335 - protein, 32 aa, synthetic construct
  REGION 1..32: note = Description of Artificial Sequence: Synthetic polypeptide
  VARIANT 2
  VARIANT 7
  VARIANT 8
  VARIANT 9
  VARIANT 10
  VARIANT 13
  VARIANT 29
RXTISRXXXXNTXYLQMGSLRAEDMAVYXCAR

SEQ ID NO: 336 - protein, 11 aa, synthetic construct
  REGION 1..11: note = Description of Artificial Sequence: Synthetic peptide
  VARIANT 3
WGXGTTVTVSS

SEQ ID NO: 337 - protein, 23 aa, synthetic construct
  REGION 1..23: note = Description of Artificial Sequence: Synthetic peptide
DIQMTQSPSSLSASVGDRVTITC

SEQ ID NO: 338 - protein, 15 aa, synthetic construct
  REGION 1..15: note = Description of Artificial Sequence: Synthetic peptide
  VARIANT 9
  VARIANT 15
WYQQKPGKXPKLLIX

SEQ ID NO: 339 - protein, 32 aa, synthetic construct
  REGION 1..32: note = Description of Artificial Sequence: Synthetic polypeptide
GVPSRFSGSGSGTDFTLTISSLQPEDFATYYC

SEQ ID NO: 340 - protein, 10 aa, synthetic construct
  REGION 1..10: note = Description of Artificial Sequence: Synthetic peptide
  VARIANT 6
  VARIANT 8
  VARIANT 9
FGGGTXVXXK

SEQ ID NO: 341 - protein, 30 aa, synthetic construct
  REGION 1..30: note = Description of Artificial Sequence: Synthetic polypeptide
  VARIANT 2
  VARIANT 24
EXQLVESGGGLIQPGGSLRLSCAXSGFSLS

SEQ ID NO: 342 - protein, 14 aa, synthetic construct
  REGION 1..14: note = Description of Artificial Sequence: Synthetic peptide
  VARIANT 12
  VARIANT 13
  VARIANT 14
WVRQAPGKGLEXXX

SEQ ID NO: 343 - protein, 32 aa, synthetic construct
  REGION 1..32: note = Description of Artificial Sequence: Synthetic polypeptide
  VARIANT 2
  VARIANT 7
  VARIANT 8
  VARIANT 9
  VARIANT 10
  VARIANT 13
  VARIANT 29
RXTISRXXXXNTXYLQMNSLRAEDTAVYXCAR

SEQ ID NO: 344 - protein, 11 aa, synthetic construct
  REGION 1..11: note = Description of Artificial Sequence: Synthetic peptide
  VARIANT 3
WGXGTLVTVSS

SEQ ID NO: 345 - protein, 23 aa, synthetic construct
  REGION 1..23: note = Description of Artificial Sequence: Synthetic peptide
  VARIANT 2
AXRMTQSPSSFSASTGDRVTITC

SEQ ID NO: 346 - protein, 15 aa, synthetic construct
  REGION 1..15: note = Description of Artificial Sequence: Synthetic peptide
  VARIANT 9
WYQQKPGKXPKLLIY

SEQ ID NO: 347 - protein, 32 aa, synthetic construct; identical to SEQ ID NO: 331
  REGION 1..32: note = Description of Artificial Sequence: Synthetic polypeptide
  VARIANT 21
  VARIANT 24

SEQ ID NO: 348 - protein, 10 aa, synthetic construct
  REGION 1..10: note = Description of Artificial Sequence: Synthetic peptide
  VARIANT 6
  VARIANT 8
  VARIANT 9
FGGGTXVXXK

SEQ ID NO: 349 - protein, 30 aa, synthetic construct
  REGION 1..30: note = Description of Artificial Sequence: Synthetic polypeptide
  VARIANT 2
  VARIANT 5
  VARIANT 24
EXQLXESGGGLVQPGGSLRLSCAXSGFSLN

SEQ ID NO: 350 - protein, 14 aa, synthetic construct
  REGION 1..14: note = Description of Artificial Sequence: Synthetic peptide
  VARIANT 13
  VARIANT 14
WVRQAPGKGLEWXX

SEQ ID NO: 351 - protein, 32 aa, synthetic construct; identical to SEQ ID NO: 343
  REGION 1..32: note = Description of Artificial Sequence: Synthetic polypeptide
  VARIANT 2
  VARIANT 7
  VARIANT 8
  VARIANT 9
  VARIANT 10
  VARIANT 13
  VARIANT 29

SEQ ID NO: 352 - protein, 11 aa, synthetic construct
  REGION 1..11: note = Description of Artificial Sequence: Synthetic peptide
  VARIANT 3
WGXGTLVTVSS

SEQ ID NO: 353 - protein, 275 aa, synthetic construct
  REGION 1..275: note = Description of Artificial Sequence: Synthetic polypeptide
NNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANSGNSDYKDDDDK

SEQ ID NO: 354 - protein, 4 aa, synthetic construct
  REGION 1..4: note = Description of Artificial Sequence: Synthetic peptide
LLVY

SEQ ID NO: 355 - protein, 7 aa, synthetic construct
  REGION 1..7: note = Description of Artificial Sequence: Synthetic peptide
EEAQGDK

SEQ ID NO: 356 - protein, 10 aa, synthetic construct
  REGION 1..10: note = Description of Artificial Sequence: Synthetic peptide
  MOD_RES 7: note = Norvaline
  MOD_RES 10: note = Lysine (2,4-dinitrophenyl)
RPKPVEXWRK